(19) United States
(12) Patent Application Publication
(10) Pub. No.: US 2025/0228969 A1
(43) Pub. Date: Jul. 17, 2025

(54) GENE EDITING TO IMPROVE JOINT FUNCTION

(71) Applicant: ORTHOBIO THERAPEUTICS, INC., Vail, CO (US)

(72) Inventors: Peter J. Millet, Edwards, CO (US); Matthew J. Allen, Swaffham Bulbeck (GB); George Gentsch, Cambridge (GB)

(21) Appl. No.: 18/832,443

(22) PCT Filed: Jan. 26, 2023
(86) PCT No.: PCT/US2023/061407

§ 371 Date: Jul. 23, 2024

Related U.S. Application Data
(60) Provisional application No. 63/303,290, filed on Jan. 26, 2022.

(51) Int. Cl. A61K 48/00; C12N 9/22; C12N 15/11; C12N 15/86; C12N 15/88

(52) U.S. Cl. CPC A61K 48/005; A61K 48/0075; C12N 9/22; C12N 15/111; C12N 15/86; C12N 15/88; C12N 2310/20; C12N 2750/14143

(57) ABSTRACT

Provided herein are compositions and methods for ablating intracellular signaling through specific reactive oxygen species (ROS)-responsive effector proteins as means of treatment for various conditions of a pro-inflammatory character. In some aspects, the compositions and methods are to prevent the progression of osteoarthritis and other arthritides and to treat osteoarthritis and other arthritides in a mammalian joint.

### CROSS REFERENCE TO RELATED APPLICATIONS

This application claims priority to U.S. Provisional Patent Application No. 63/303,290, filed Jan. 26, 2022, the contents of which are hereby incorporated by reference herein, in their entireties, for all purposes.

### BACKGROUND OF THE INVENTION

Reactive oxygen species are naturally occurring molecules that are generated by various cellular processes. In most instances, these molecules are detected by cells and destroyed. See generally, Bayr, H. (2005). Reactive oxygen species. Critical Care Medicine, 33(12), S498-S501. However, in certain disease states, such as gout or arthritis, this typical cycle is disturbed locally, such that ROS detection in, for example, the diseased joint, leads to heightened inflammatory signaling, thereby contributing to or exacerbating the disease.

Though small molecule drugs, such as inhibitors, could be employed to target particular proteins that transduce signals between ROS and inflammation, the versatile cellular functions these factors play in cellular homeostasis make such systemic treatments unsuitable. Therefore, new, more targeted approaches are needed to address these factors' contribution to disease without disrupting their roles in healthy cells throughout an organism.

### BRIEF SUMMARY OF THE INVENTION

Provided herein are compositions and methods for silencing the intracellular signaling of downstream effectors of reactive oxygen species (ROS) in an animal in need thereof to treat a disease, illness or condition caused by aberrant or excessive activity of said effector. In some embodiments, ROS effector intracellular signaling is silenced by CRISPR editing of the gene encoding the effector protein.

### BRIEF DESCRIPTION OF THE DRAWINGS

The presently disclosed embodiments will be further explained with reference to the attached drawings. The drawings shown are not necessarily to scale, with emphasis instead generally being placed upon illustrating the principles of the presently disclosed embodiments.

FIG. 1 illustrates transcriptome analysis of canine monocytes treated with MSU crystals relative to PBS control, with upregulated genes shaded.

FIGS. 2A, 2B, 2C, 2D, 2E, 2F, 2G, 2H, 2I, 2J, 2K, 2L, 2M, 2N, 20, 2P, 2Q, 2R, 2S, 2T, 2U, 2V, 2W, 2X, 2Y, 2Z, 2A1, 2B1, 2C1, 2D1, 2E1, 2F1, 2G1, 2H1, 2I1, 2J1, 2K1, 2L1, 2M1, 2N1, 201, 2P1, 2Q1, 2R1, 2S1, 2T1, 2U1, 2V1, 2W1, 2X1, 2Y1, 2Z1, 2A2, 2B2, 2C2, 2D2, 2E2, 2F2, 2G2, 2H2, 212, 2J2, 2K2, 2L2, 2M2, 2N2, 202, 2P2, 2Q2, 2R2, 2S2, 2T2, 2U2, 2V2, 2W2, 2X2, 2Y2, 2Z2, 2A3, 2B3, 2C3, 2D3, 2E3, 2F3, 2G3, 2H3, 2I3, 2J3, 2K3, and 2L3 collectively illustrate example CRISPR/Cas9 crRNA sequences designed for editing genes associated with the production, blocking, or removal of reactive oxygen species (ROS), in accordance with some embodiments of the present disclosure. The crRNA sequences were generated using the CRISPick server available online at the URL portals.broadinstitute.org/gppx/crispick/public. The algorithm was run against build GRCh38 (NCBI RefSeq v.109.20210514) of the human genome, and the algorithm optimized for a CRISPR knock-out (CRISPRko) mechanism and SpyoCas9 enzyme. The example sequences listed in FIG. 2 are not intended to be limiting.

FIGS. 3A, 3B and 3C collectively illustrate example CRISPR/Cas9 crRNA sequences designed for editing genes associated with the production, blocking, or removal of reactive oxygen species (ROS), in accordance with some embodiments of the present disclosure, e.g., NOX4. The crRNA sequences were generated using the CRISPick server available online at the URL portals.broadinstitute.org/gppx/crispick/public. The algorithm was run against build GRCh38 (NCBI RefSeq v.109.20210514) of the human genome, and the algorithm optimized for a CRISPR knock-out (CRISPRko) mechanism (FIG. 3A), a CRISPRRa mechanism (FIG. 3B), and a CRISPRi mechanism (FIG. 3C), and SpyoCas9 enzyme. The example sequences listed in FIG. 3 are not intended to be limiting.

FIGS. 4A and 4B, 4C, and 4D collectively illustrate example CRISPR/Cas9 crRNA sequences designed for editing genes associated with the production, blocking, or removal of reactive oxygen species (ROS), in accordance with some embodiments of the present disclosure. e.g., NOX4. The crRNA sequences were generated using the CRISPick server available online at the URL portals.broadinstitute.org/gppx/crispick/public. The algorithm was run against build GRCh38 (NCBI RefSeq v.109.20210514) of the human genome, and the algorithm optimized for a CRISPR knock-out (CRISPRko) mechanism, and SpyoCas9 enzyme (FIG. 4A), SaurCas9 enzyme (FIG. 4B), AsCas12 enzyme (FIG. 4C), and enAsCas12a enzyme (FIG. 4D). The example sequences listed in FIG. 4 are not intended to be limiting.

FIGS. 5A, 5B, 5C, 5D, 5E, 5F, 5G, 5H, 5I, 5J, 5K, 5L, 5M, and 5N illustrate SEQ ID NOs: 1-328 (A-G) the crRNA sequences generated by the bioinformatic methods herein described that target human NFKB1 to generate a genetic knockout and (H-N) additional information regarding the genomic coordinates of the bound DNA, DNA strand targeted, exon targeted, and several quality control parameters.

FIGS. 6A, 6B, 6C, 6D, 6E, 6F, 6G, 6H, 6I, 6J, 6K, 6L, 6M, 6N, 60, 6P, 6Q, and 6R collectively illustrate SEQ ID NOs: 329-680 and 5323-5410 (A-I) the crRNA sequences generated by the bioinformatic methods herein described that target human NFKB2 to generate a genetic knockout and (J-R) additional information regarding the genomic coordinates of the bound DNA, DNA strand targeted, exon targeted, and several quality control parameters.

### DETAILED DESCRIPTION OF THE INVENTION

### I. Introduction

Provided herein are compositions and methods for silencing the signaling functionality of one or more cellular receptors in an animal in need thereof to treat a disease, illness or condition caused by aberrant or excessive signaling through said receptor.

TABLE 1
Exemplary list of ROS effectors to be targeted

6-phosphogluconate dehydrogenase (6PGD)
Alcohol dehydrogenase (ADH)
Aldehyde dehydrogenase (ALDH2)
AP-1
B-cell lymphoma-extra large (Bcl-XL)
BCL2 apoptosis regulator (Bcl-2)
Bcl-2-associated X protein (BAX)
Catalase (CAT)
C-Jun N-terminal kinase (JNK)
Coenzyme Q10 (CoQ10)
CYP2E1
Cytochrome c (Cyt c)
F1Fo-ATP synthase
Ferritin heavy chain (FHC)
Glucose-6-phosphate dehydrogenase (G6PD)
Glutamylcysteine synthetase (GCS)
Glutathione (GSH) synthase
Glutathione Peroxidase 1 (GPX1)
Glutathione Peroxidase 2 (GPX2)
Glutathione Peroxidase 3 (GPX3)
Glutathione Peroxidase 4 (GPX4)
Glutathione Peroxidase 5 (GPX5)
Glutathione Peroxidase 6 (GPX6)
Glutathione Peroxidase 7 (GPX7)
Glutathione Peroxidase 8 (GPX8)
Glutathione reductase (GR)
Glycerol 3-phosphate dehydrogenase
Growth arrest and DNA damage (GADD 45)
Hypoxia-inducible factor 1-alpha (HIF-1α)
Mitogen-activated protein kinase (MAPK)
NADH-ubiquinone oxidoreductase
NADPH oxidase 4 (NOX4)
NADPH oxidase 5 (NOX5)
Nuclear factor kappa-light-chain-enhancer of activated B cells (NF-κB)
Nuclear factor κB (NF-κB) essential modulator (NEMO)
p46Shc (SHC isoform)
p52Shc (SHC isoform)
p53 upregulated modulator of apoptosis (PUMA)
p66Shc (SHC isoform)
Phosphoinositide 3-kinase (PI3-K)
Proline oxidase (PIG6, POX)
Quinone oxidoreductase (PIG3, NQO1)
Respiratory complexes I
Respiratory complexes II
Respiratory complexes III
Respiratory complexes IV
Sestrin 1 (SESN1)
Sestrin 2 (SESN2)
SHC adaptor protein 1 (SHC1)
Superoxide dismutase 1 (SOD1)
Superoxide dismutase 2 (SOD2)
Superoxide dismutase 3 (SOD3)
TNF alpha induced protein 3 (TNFAIP3)
Tumor protein 53 (p53)
Tumor protein p53 inducible nuclear protein 1 (TP53INP1)
Ubiquinol-cytochrome c oxidoreductase
2B4
ABCA1
ACP5
ADAR-1
ADSS
AIG1
AIM2
APOBEC3
ARRB2
B2M
BCAS3
BMP4
C10orf32
C21orf33
CASP1
CCL5
CD160
Cd53
CDKN2A
CHEK1
CNNM2
CNTNAP2
CSMD1
CTLA-4
CTSB
C-type lectin receptors CLRs
CXCL10
CYP17A1
DDX60
DYNC1I1
FOXO3a
GPC6
GRN
HCK
HECW1
HLA
IFI30
IFI44L
IFI6
IFITM
IFITM1/3
IFITM2
IFITM3
IL-18
IL-1α
IL-1β
interferon-γ
interleukin-12 (IL-12)
IRF
IRF-1
IRF3
IRF7
LAG-3
LIPC
MDA5/IFIH1
MPAK
MYH9
MYO16
MYO5A
NAIP
NF-κB
NLRC4
NLRP3
NOD2
NPL
NR
nucleotide oligomerization and binding domain NOD-like receptors
OAS1
OAS2
OASL
parkin gene (PARK2)
PD-1
PLEKHG1
PRKCA
PTBP1
PYCARD
Pyrin-HIN (PYHIN) domain containing receptors (e.g. AIM2)
reactive oxygen species (ROS)
retinoic acid inducible gene-I (RIG-I)-like receptors (RLRs)
RFC3
RGS1
RIG-I/DDX58
SAMHD1
SF3A1/SF3B1
SFXN2
SLAMF7
SLC41A1
SLC8A1
SLCO3A1
STAT1
Tetherin
TLR5
TLR7
TLR9
Toll-like receptors (TLRs)
TREM2
TREX1
TRIM5
TTLL7
TYROBP

In some embodiments, receptor signaling is silenced by CRISPR editing of the gene encoding the receptor. In some embodiments, the CRISPR editing results in ablation of a transmembrane domain (i.e., generation of a soluble decoy receptor). In some embodiments, the CRISPR editing results in ablation of a cytoplasmic domain (i.e., generation of a membrane-bound decoy receptor).

### II. Definitions

Unless defined otherwise, all technical and scientific terms used herein have the same meaning as is commonly understood by one of skill in the art to which this invention belongs. All patents and publications referred to herein are incorporated by reference in their entireties.

The terms “Nuclear Factor Kappa B Subunit 1” or “NFKB1” refer to the genes (NCBI Gene ID: 4790 [human], NCBI Gene ID: 442859 [canine], NCBI Gene ID: 100067894 [equine], NCBI Gene ID: 100142683 [feline]) or an encoded gene product (e.g., UniProt: P19838; NP_001158884.1 [human], XP_038299710.1 [canine], XP_005608646.3 [equine], XP_023108672.2 [feline]), as well as sequence variants, isoforms, proteins harboring conservative amino acid substitutions, and glycoforms thereof. Canonically, the proteins encoded by the genes listed above are a 105 kDa protein, a Rel protein-specific transcription inhibitor, which may undergo cotranslational processing by the 26S proteasome to produce a 50 kDa protein. This 50 kDa protein is a DNA binding subunit of the NF-kappa-B (NFKB) protein complex that is responsible for transcriptional regulation of various genes upon activation by any number of stimuli, including cytokines, oxidant-free radicals, ultraviolet irradiation, or detection of pathogenic molecular patterns. The activated NFKB heterodimer translocates into the nucleus to directly stimulate expression of genes involved in a wide variety of biological functions. Inappropriate activation of NFKB has been associated with a number of inflammatory diseases, while persistent inhibition of NFKB can lead to inappropriate immune cell development or delayed cell growth. In some instances, and merely for the sake of disambiguation, a prefix is added when referring to the protein or gene of a particular species (with h, c, e, and f, referring to the human, canine, equine, and feline forms, respectively).

In certain embodiments, any region of an NFKB1 gene (e.g., 5′ untranslated region [UTR], exon 1, exon 2, exon 3, exon 4, exon 5, exon 6, exon 7, exon 8, exon 9, exon 10, exon 11, exon 12, exon 13, exon 14, exon 15, exon 16, exon 17, exon 18, exon 19, exon 20, exon 21, exon 22, exon 23, exon 24, exon 25, exon 26, exon 27, exon 28, any intervening intronic regions, intron/exon junctions, the 3′ UTR, or polyadenylation signal) is targeted by an RNA-guided nuclease to alter the gene. In some embodiments, the NFKB1 gene targeted by an RNA-guided nuclease is from a mammal. In some embodiments, the NFKB1 gene targeted by an RNA-guided nuclease is from a human (hNFKB1). In some embodiments, the NFKB1 gene targeted by an RNA-guided nuclease is from a dog (cNFKB1). In some embodiments, the NFKB1 gene targeted by an RNA-guided nuclease is from a horse (eNFKB1). In some embodiments, the NFKB1 gene targeted by an RNA-guided nuclease is from a cat (fNFKB1).

The terms “Nuclear Factor Kappa B Subunit 2” or “NFKB2” refer to the genes (NCBI Gene ID: 4791 [human], NCBI Gene ID: 486858 [canine], NCBI Gene ID: 100069984 [equine], NCBI Gene ID: 101080578 [feline]) or an encoded gene product (e.g., UniProt: Q00653; NP_001070962.1 [human], XP_038296333.1 [canine], XP_001916453.3 [equine], XP_006938194.2 [feline]), as well as sequence variants, isoforms, proteins harboring conservative amino acid substitutions, and glycoforms thereof. Canonically, the proteins encoded by the genes listed above are co-translationally processed from a p100 full-length protein into a p52 active form. This p52 protein can act as a transcriptional repressor (as a homodimer) or a transcriptional activator (as part of the heterodimeric RelB-p52 complex). The activator complex is responsible for transcriptional regulation of various genes upon activation by any number of stimuli, including cytokines, oxidant-free radicals, ultraviolet irradiation, or detection of pathogenic molecular patterns, stimulating expression of genes involved in a wide variety of biological functions. Inappropriate activation of NFKB pathways has been associated with a number of inflammatory diseases, while persistent inhibition of NFKB can lead to inappropriate immune cell development or delayed cell growth. In some instances, and merely for the sake of disambiguation, a prefix is added when referring to the protein or gene of a particular species (with h, c, e, and f, referring to the human, canine, equine, and feline forms, respectively).

In certain embodiments, any region of an NFKB2 gene (e.g., 5′ untranslated region [UTR], exon 1, exon 2, exon 3, exon 4, exon 5, exon 6, exon 7, exon 8, exon 9, exon 10, exon 11, exon 12, exon 13, exon 14, exon 15, exon 16, exon 17, exon 18, exon 19, exon 20, exon 21, exon 22, exon 23, exon 24, any intervening intronic regions, intron/exon junctions, the 3′ UTR, or polyadenylation signal) is targeted by an RNA-guided nuclease to alter the gene. In some embodiments, the NFKB2 gene targeted by an RNA-guided nuclease is from a mammal. In some embodiments, the NFKB2 gene targeted by an RNA-guided nuclease is from a human (hNFKB2). In some embodiments, the NFKB2 gene targeted by an RNA-guided nuclease is from a dog (cNFKB2). In some embodiments, the NFKB2 gene targeted by an RNA-guided nuclease is from a horse (eNFKB2). In some embodiments, the NFKB2 gene targeted by an RNA-guided nuclease is from a cat (fNFKB2).

The term “reactive oxygen species” or “ROS” refers to any number of naturally-occurring unstable molecules that contain oxygen and that easily react with other molecules in a cell, often due to the presence of one or more unpaired electron. See generally, Bayr, H. (2005). Reactive oxygen species. Critical Care Medicine, 33(12), 5498-5501. Non-exhaustive examples include superoxide and its reduced oxygen derivatives and peroxynitrite.

The term “treatment” refers to obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. For example, a composition, method, or system of the present disclosure may be administered as a prophylactic treatment to a subject that has a predisposition for a given condition (e.g., arthritis). “Treatment”, as used herein, covers any treatment of a disease in a mammal, particularly in a human, canine, feline, or equine, and includes: (a) preventing the disease from occurring in a subject which may be predisposed to the disease but has not yet been diagnosed as having it; (b) inhibiting the disease, i.e., arresting its development or progression; and (c) relieving the disease, i.e., causing regression of the disease and/or relieving one or more disease symptoms.

“Treatment” is also meant to encompass delivery of an agent in order to provide for a pharmacologic effect, even in the absence of a disease or condition. For example, “treatment” encompasses delivery of a composition that can elicit an immune response or confer immunity in the absence of a disease condition, e.g., in the case of a vaccine. It is understood that compositions and methods of the present disclosure are applicable to treat all mammals, including, but not limited to human, canine, feline, equine, and bovine subjects.

The term “therapeutically effective” refers to the amount of a composition or combination of compositions as described herein that is sufficient to effect the intended application including, but not limited to, disease treatment. A therapeutically effective amount may vary depending upon the intended application (in vitro or in vivo), or the subject and disease condition being treated (e.g., the weight, age and gender of the subject), the severity of the disease condition, or the manner of administration. The term also applies to a dose that will induce a particular response in target cells (e.g., the reduction of platelet adhesion and/or cell migration). The specific dose will vary depending on the particular composition(s) chosen, the dosing regimen to be followed, whether the composition is administered in combination with other compositions or compounds, timing of administration, the tissue to which it is administered, and the physical delivery system in which the composition is carried.

The term “joint disease” is defined as measurable abnormalities in the cells or tissues of the joint that could lead to illness, for example, metabolic and molecular derangements triggering anatomical and/or physiological changes in the joint. Including, but not limited to, radiographic detection of joint space narrowing, subchondral sclerosis, subchondral cysts, and osteophyte formation.

“Joint illness” is defined in human subjects as symptoms that drive the subject to seek medical intervention, for example, subject reported pain, stiffness, swelling, or immobility. For non-human mammals, “joint illness” is defined, for example, as lameness, observable changes in gait, weight bearing, allodynia, or exploratory behavior.

The terms “polynucleotide,” “nucleotide,” and “nucleic acid” are used interchangeably herein to refer to all forms of nucleic acid, oligonucleotides, including deoxyribonucleic acid (DNA) and ribonucleic acid (RNA). Polynucleotides include genomic DNA, cDNA and antisense DNA, and spliced or unspliced mRNA, rRNA, tRNA, lncRNA, RNA antagomirs, and inhibitory DNA or RNA (RNAi, e.g., small or short hairpin (sh)RNA, microRNA (miRNA), aptamers, small or short interfering (si)RNA, trans-splicing RNA, or antisense RNA). Polynucleotides also include non-coding RNA, which include for example, but are not limited to, RNAi, miRNAs, lncRNAs, RNA antagomirs, aptamers, and any other non-coding RNAs known to those of skill in the art. Polynucleotides include naturally occurring, synthetic, and intentionally altered or modified polynucleotides as well as analogues and derivatives. The term “polynucleotide” also refers to a polymeric form of nucleotides of any length, including deoxyribonucleotides or ribonucleotides, or analogs thereof, and is synonymous with nucleic acid sequence. A polynucleotide may comprise modified nucleotides, such as methylated nucleotides and nucleotide analogs, and may be interrupted by non-nucleotide components. If present, modifications to the nucleotide structure may be imparted before or after assembly of the polymer. The term polynucleotide, as used herein, refers interchangeably to double- and single-stranded molecules. Unless otherwise specified or required, any embodiment as described herein encompassing a polynucleotide encompasses both the double-stranded form and each of two complementary single-stranded forms known or predicted to make up the double-stranded form. Polynucleotides can be single, double, or triplex, linear or circular, and can be of any length. In discussing polynucleotides, a sequence or structure of a particular polynucleotide may be described herein according to the convention of providing the sequence in the 5′ to 3′ direction.

The term “gene” or “nucleotide sequence encoding a polypeptide” refers to the segment of DNA involved in producing a polypeptide chain. The DNA segment may include regions preceding and following the coding region (leader and trailer) involved in the transcription/translation of the gene product and the regulation of the transcription/translation, as well as intervening sequences (introns) between individual coding segments (exons). For example, a gene includes a polynucleotide containing at least one open reading frame capable of encoding a particular protein or polypeptide after being transcribed and translated.

The term “receptor” refers to a protein capable of binding another cognate protein (i.e., its ligand) with high affinity. This receptor-ligand interaction may be 1:1, or result in multimerization, wherein numerous proteins aggregate to bind one or more ligands. Receptors are generally present at the cell surface, such that they may most efficiently encounter a ligand and initiate intracellular signaling.

The term “intracellular signaling” refers to cellular changes that result due to events occurring at the cell surface. Typically, a soluble ligand binds its receptor at the cell surface, which can induce changes in the receptor, such that associated intracellular factors are also affected. These factors may then impact others within the cell, and this cascade continues until, in many cases, a particular factor is able to alter gene expression in the nucleus in response to the stimulus at the surface.

The term “RNA-guided nuclease” refers to an enzyme capable of breaking the backbone of, for example, a DNA molecule. The activity of RNA-guided nucleases is directed by a nucleic acid molecule (i.e., guide RNA). Once properly oriented to form a functional ribonucleoprotein complex, the enzyme locates a specific position within a target nucleic acid (e.g., a gene or locus) via sequence complementarity with a portion of the guide RNA. Non-exhaustive examples of RNA-guided nucleases include Cas9, Cas12 and Cas12a (previously known as Cpf1).

The term “Cas9” refers to an RNA-guided, double-stranded DNA-binding nuclease protein or nickase protein, or a variant thereof and may be used to refer to either naturally-occurring or recombinant Cas9 nucleases variants (e.g., ES-Cas9, HF-Cas9, PE-Cas9, and AR-Cas9). The wildtype Cas9 nuclease has two functional domains, e.g., RuvC and HNH, that simultaneously cut both strands of double stranded DNA, resulting in a double-strand break. Cas9 enzymes described herein may comprise a HNH or HNH-like nuclease domain and/or a RuvC or RuvC-like nuclease domain without impacts on the ability to induce double-strand breaks in genomic DNA (e.g., at a target locus) when both functional domains are active. The Cas9 enzyme may comprise one or more catalytic domains of a Cas9 protein derived from bacteria belonging to the group consisting of Corynebacteria, Sutterella, Legionella, Treponema, Filifactor, Eubacterium, Streptococcus, Lactobacillus, Mycoplasma, Bacteroides, Flaviivola, Flavobacterium, Sphaerochaeta, Azospirillum, Gluconacetobacter, Neisseria, Roseburia, Parvibaculum, Staphylococcus, Nitratifractor, and Campylobacter. In some embodiments, the two catalytic domains are derived from different bacteria species.

As used herein, “PAM” refers to a Protospacer Adjacent Motif and is necessary for an RNA-guided nuclease to bind a target nucleic acid. In many instances, the PAM directly abuts the complementary sequence in the target. Naturally-occurring Cas9, for example, molecules recognize specific PAM sequences (see, e.g., Table 2). In some embodiments, a Cas9 molecule has the same PAM specificities as a naturally occurring Cas9 molecule. In other embodiments, a Cas9 molecule has a PAM specificity not associated with a naturally occurring Cas9 molecule. In other embodiments, a Cas9 molecule's PAM specificity is not associated with the naturally occurring Cas9 molecule to which it has the closest sequence homology. For example, a naturally occurring Cas9 molecule can be altered such that the PAM sequence recognition is altered to decrease off target sites, improve specificity, or eliminate a PAM recognition requirement. In an embodiment, a Cas9 molecule may be altered (e.g., to lengthen a PAM recognition sequence, improve Cas9 specificity to high level of identity, to decrease off target sites, and/or increase specificity). In an embodiment, the length of the PAM recognition sequence is at least 4, 5, 6, 7, 8, 9, 10 or 15 amino acids in length. In some embodiments, a Cas9 molecule may be altered to ablate PAM recognition.

The terms “guide RNA,” “gRNA” or “sgRNA” may be used interchangeably and refer to an RNA molecule, preferably a synthetic RNA molecule, composed of a targeting (crRNA) sequence and scaffold. These molecules, once loaded onto a functional RNA-guided nuclease can direct sequence-specific cleavage of a target nucleic acid.

An sgRNA can be administered or formulated, e.g., as a synthetic RNA, or as a nucleic acid comprising a sequence encoding the gRNA, which is then expressed in the target cells. As would be evident to one of ordinary skill in the art, various tools may be used in the design and/or optimization of an sgRNA in order to, for example, increase specificity and/or precision of genomic editing at a particular site.

In general, candidate sgRNAs may be designed and identified by first locating suitable PAMs within a genomic sequence. Then additional calculations may be utilized to predict on-target and off-target efficiencies. Available web-based tools to aid in the initial design and modeling of candidate sgRNAs include, without limitation, CRISPRseek, CRISPR Design Tool, Cas-OFFinder, E-CRISP, ChopChop, CasOT, CRISPR direct, CRISPOR, BREAKING-CAS, CrispRGold, and CCTop. See, e.g., Safari, F. et al. (2017). Current Pharmaceutical Biotechnology, 18(13):1038-54, which is incorporated by reference herein in its entirety for all purposes. Such tools are also described, for example, in PCT Publication No. WO2014093701A1 and Liu, G. et al. (2020). Computational approaches for effective CRISPR guide RNA design and evaluation. Computational and Structural Biotechnology Journal, 18: 35-44, each of which is incorporated by reference herein in its entirety for all purposes. Candidate sgRNAs may be further assessed by experimental screening or other methodologies.

The terms “CRISPR RNA” or “crRNA” refer to the portion of an sgRNA molecule with complementarity to the target nucleic acid.

The phrase “pharmaceutically acceptable” refers to those compounds, materials, compositions, and/or dosage forms that are, within the scope of sound medical judgment, suitable for use in contact with the tissues of human beings and animals without excessive toxicity, irritation, allergic response, or other problems or complications commensurate with a reasonable benefit/risk ratio.

The terms “pharmaceutically acceptable carrier” or “pharmaceutically acceptable excipient” are intended to include any and all solvents, dispersion media, coatings, antibacterial and antifungal agents, isotonic and absorption delaying agents, and inert ingredients. The use of such pharmaceutically acceptable carriers or pharmaceutically acceptable excipients for active pharmaceutical ingredients is well known in the art. Except insofar as any conventional pharmaceutically acceptable carrier or pharmaceutically acceptable excipient is incompatible with the active pharmaceutical ingredient, its use in the therapeutic compositions of the disclosure is contemplated. Additional active pharmaceutical ingredients, such as other drugs, can also be incorporated into the described compositions and methods.

The term “pharmaceutically acceptable excipient” is intended to include vehicles and carriers capable of being co-administered with a compound to facilitate the performance of its intended function. The use of such media for pharmaceutically active substances is well known in the art. Examples of such vehicles and carriers include solutions, solvents, dispersion media, delay agents, emulsions and the like. Any other conventional carrier suitable for use with the multi-binding compounds also falls within the scope of the present disclosure.

As used herein, the term “a”, “an”, or “the” generally is construed to cover both the singular and the plural forms.

The terms “about” and “approximately” mean within a statistically meaningful range of a value. Such a range can be within an order of magnitude, preferably within 50%, more preferably within 20%, more preferably still within 10%, and even more preferably within 5% of a given value or range. The allowable variation encompassed by the terms “about” or “approximately” depends on the particular system under study, and can be readily appreciated by one of ordinary skill in the art. Moreover, as used herein, the terms “about” and “approximately” mean that compositions, amounts, formulations, parameters, shapes and other quantities and characteristics are not and need not be exact, but may be approximate and/or larger or smaller, as desired, reflecting tolerances, conversion factors, rounding off, measurement error and the like, and other factors known to those of skill in the art. In general, a dimension, size, formulation, parameter, shape or other quantity or characteristic is “about” or “approximate,” whether or not expressly stated to be such. It is noted that embodiments of very different sizes, shapes and dimensions may employ the described arrangements.

The term “substantially” as used herein can refer to a majority of, or mostly, as in at least about 50%, 60%, 70%, 80%, 90%, 95%, 96%, 97%, 98%, 99%, 99.5%, 99.9%, 99.99%, or at least about 99.999% or more.

The transitional terms “comprising,” “consisting essentially of,” and “consisting of,” when used in the appended claims, in original and amended form, define the claim scope with respect to what unrecited additional claim elements or steps, if any, are excluded from the scope of the claim(s). The term “comprising” is intended to be inclusive or open-ended and does not exclude any additional, unrecited element, method, step or material. The term “consisting of” excludes any element, step or material other than those specified in the claim and, in the latter instance, impurities ordinary associated with the specified material(s). The term “consisting essentially of” limits the scope of a claim to the specified elements, steps or material(s) and those that do not materially affect the basic and novel characteristic(s) of the claimed methods and compositions. All compositions, methods, and kits described herein that embody the present disclosure can, in alternate embodiments, be more specifically defined by any of the transitional terms “comprising,” “consisting essentially of,” and “consisting of.”

### III. Methods

### A. CRISPR

Unless otherwise defined, all technical and scientific terms used herein have the same meaning as commonly understood by one of ordinary skill in the art to which this disclosure belongs. Although methods and materials similar or equivalent to those described herein may be used in the practice or testing of the present disclosure, suitable methods and materials are described below. All publications, patent applications, patents, and other references mentioned herein are incorporated by reference in their entirety. In addition, the materials, methods, and examples are illustrative only and not intended to be limiting.

In one aspect, the present disclosure encompasses compositions relating to clustered regularly interspaced short palindromic repeats (CRISPR) and CRISPR-associated RNA-guided nucleases and associated methods, components, and compositions (hereafter, CRISPR/Cas systems). Such systems minimally require at least one isolated or non-naturally-occurring RNA-guided nuclease (e.g., a Cas9 protein) and at least one isolated or non-naturally-occurring guide RNA (e.g., an sgRNA) to effectuate augmentation of a nucleic acid sequence (e.g., genomic DNA).

In some embodiments, a CRISPR/Cas system effectuates the alteration of a targeted gene or locus in a eukaryotic cell by effecting an alteration of the sequence at a target position (e.g., by creating an insertion or deletion (collectively, an indel) resulting in loss-of-function of (i.e., knocking out) the affected gene or allele; e.g., a nucleotide substitution resulting in a truncation, nonsense mutation, or other type of loss-of-function of an encoded product of, for example, one or more NFKB1 or NFKB2 gene (i.e., mRNA or protein); a deletion of one or more nucleotides resulting in a truncation, nonsense mutation, or other type of loss-of-function of an encoded product of, for example, one or more NFKB1 or NFKB2 gene; e.g., loss-of-function of the encoded mRNA or protein by a single nucleotide, double nucleotide, or other frame-shifting deletion, or a deletion resulting in a premature stop codon; or an insertion resulting in a truncation, nonsense mutation, or other type of loss-of-function of an encoded gene product, such as an encoded gene product of, for example, one or more NFKB1 or NFKB2 gene (i.e., mRNA or protein); e.g., a single nucleotide, double nucleotide, or other frame-shifting insertions, or an insertion resulting in a premature stop codon. In some embodiments, a CRISPR/Cas system of the present disclosure provides for the alteration of a gene and/or encoded product of a gene, such that the altered product has a resultant loss-of-function and becomes a dominant negative or decoy (e.g., a transmembrane receptor incapable of initiating intracellular signaling or a soluble receptor).

In one aspect, CRISPR/Cas systems effectuate changes to the sequence of a nucleic acid through nuclease activity. For example, in the case of genomic DNA, the RNA-guided-nuclease locates a target position within a targeted gene or locus by sequence complementarity with the target genomic sequence (e.g., CRISPR RNA (crRNA) or a complementary component of a synthetic single guide RNA (sgRNA)) and cleaves the genomic DNA upon recognition of a particular, nuclease-specific motif called the protospacer adjacent motif (PAM). See generally, Collias, D., & Beisel, C. L. (2021). Nature Communications, 12(1), 1-12.

Nuclease activity (i.e., cleavage) induces a double-strand break (DSB) in the case of genomic DNA. Endogenous cellular mechanisms of DSB repair, namely non-homologous end joining (NHEJ), microhomology-mediated end joining (MMEJ), and homologous recombination, result in erroneous repair at a given target position with some calculable frequency as a result of interference from said components of the CRISPR/Cas system, thereby introducing substitutions or indels into the genomic DNA. See generally Scully, R., et al. (2019). Nature Reviews Molecular Cell Biology, 20(11), 698-714. At some frequency, these indels and/or substitutions may result in frameshifts, nonsense mutations (i.e., early stop codons) or truncations that impact the availability of gene products, such as mRNA and/or protein. In certain embodiments, the CRISPR/Cas system may induce a homology-directed repair (HDR) mechanism leading to insertions of non-random sequences at a target position through the use of templates (e.g., an HDR template) provided to the cell as part of the system along with the nuclease and gRNA. See Bloh, K., & Rivera-Torres, N. (2021). International Journal of Molecular Sciences, 22(8), 3834.

In general, the minimum requirements of the CRISPR/Cas system will be dependent upon the nuclease (i.e., Cas protein) provided therewith. To this extent, these bacterially-derived nucleases have been functionally divided into Types I, III, and V, which all fall into Class 1 and Types II, IV, and VI that are grouped into Class 2.

### Class 1 CRISPR Cas Systems:

The exact components, compositions, and methods for effectuating a change in a targeted nucleic acid sequence using a Class 1 CRISPR/Cas system will vary, but should minimally include: a nuclease (selected from at least Types I, and III), at least one guide RNA selected from 1) sgRNA or 2) a combination of crRNA and tracrRNA. These CRISPR/Cas systems have been categorized together as Class 1 CRISPR/Cas systems due to their similarities in requirements and mode of action within a eukaryotic cell. To this end, compositions, components, and methods among Class 1 constituents may be considered functionally interchangeable, and the following details, provided merely for exemplary purposes, do not represent an exhaustive list of class members:

Cas3 (see Table 2) is the prototypical Type I DNA nuclease that functions as the effector protein as part of a larger complex (the Cascade complex comprising Cse1, Cse2), that is capable of genome editing. See generally He, L., et al. (2020). Genes, 11(2), 208. Unlike other CRISPR/Cas systems, Type I systems localize to the DNA target without the Cas3 nuclease via the Cascade complex, which then recruits Cas3 to cleave DNA upon binding and locating the 3′ PAM. The Cascade complex is also responsible for processing crRNAs such that they can be used to guide it to the target position. Because of this functionality, Cascade has the ability to process multiple arrayed crRNAs from a single molecule. See. Luo, M. (2015). Nucleic Acids Research, 43(1), 674-681. As such, Type I system may be used to edit multiple targeted genes or loci from a single molecule.

Because the natural Cas3 substrate is ssDNA, its function in genomic editing is thought to be as a nickase; however, when targeted in tandem, the resulting edit is a result of blunt end cuts to opposing strands to approximate a blunt-cutting endonuclease, such as Cas9. See Pickar-Oliver, A., & Gersbach, C. A. (2019). Nature Reviews Molecular Cell Biology, 20(8), 490-507.

Like Type I nucleases, the Type III system relies upon a complex of proteins to effect nucleic acid cleavage. Particularly, Cas10 possesses the nuclease activity to cleave ssDNA in prokaryotes. See Tamulaitis, G. Trends in Microbiology, 25(1), 49-61 (2017). Interestingly, this CRISPR/Cas system, native to archaea, exhibits dual specificity and targets both ssDNA and ssRNA. Aside from this change, the system functions much like Type I in that the crRNA targets an effector complex (similar to Cascade) in a sequence-dependent manner. Similarly, the effector complex processes crRNAs prior to association. The dual nature of this nuclease makes its applications to genomic editing potentially more powerful, as both genomic DNA and, in some cases, mRNAs with the same sequence may be targeted to silence particular targeted genes.

### Class 2 CRISPR Cas Systems:

The exact components, compositions, and methods for effectuating a change in a targeted nucleic acid sequence using a Class 2 CRISPR/Cas system will vary but should minimally include: a nuclease (selected from at least Types II, and V), at least one guide RNA selected from 1) sgRNA or 2) a combination of crRNA and tracrRNA. These CRISPR/Cas systems have been categorized together as Class 2 CRISPR/Cas systems due to their similarities in requirements and mode of action within a eukaryotic cell. To this end, compositions, components, and methods among Class 2 constituents may be considered functionally interchangeable, and the following details, provided merely for exemplary purposes, do not represent an exhaustive list of class members:

Type II nucleases are the best-characterized CRISPR/Cas systems, particularly the canonical genomic editing nuclease Cas9 (see Table 2). Multiple Cas9 proteins, derived from various bacterial species, have been isolated. The primary distinction between these nucleases is the PAM, a required recognition site within the targeted dsDNA. After association with a gRNA molecule, the crRNA (or targeting domain of a sgRNA) orients the nuclease at the proper position, but the protein's recognition of the PAM is what induces a cleavage event near that site, resulting in a blunt DSB.

In addition to the naturally-derived Cas9 proteins, several engineered variants have similarly been reported. These range from Cas9 with enhanced specific (i.e., less off-target activity), such as espCas9. Others have been catalytically modified via point mutations in the RuvC (e.g., D10A) and HNH (e.g., H840A) domains such that they induce only single-strand breaks (i.e., Cas9 nickases). See Frock, R. et al. (2015). Nature Biotechnology, 33(2), 179-186. These have also been shown to be less error-prone in editing. Such mitigation of off-target effects becomes paramount when selecting for a desired insertion (i.e., a knock in mutation, in which a desired nucleotide sequence is introduced into a target nucleic acid molecule) rather than a deletion. Indeed, less off-target effects may aid in the preferred DNA repair mechanism (HDR, in most instances for knock in mutations). See generally Naeem, M., et al. (2020). Cells, 9(7), 1608.

Additional exemplary further engineered variants of canonical Cas proteins (e.g., mutants, chimeras, and include the following (each of which are hereby incorporated by reference in their entireties for all purposes): WO2015035162A2, WO2019126716A1, WO2019126774A1, WO2014093694A1, WO2014150624A1, US20190225955A1, U.S. patent Ser. No. 11/427,818, U.S. patent Ser. No. 11/242,542, U.S. patent Ser. No. 11/098,297, U.S. patent Ser. No. 10/876,100, U.S. patent Ser. No. 10/767,193, U.S. patent Ser. No. 10/494,621, and U.S. patent Ser. No. 10/100,291.

For the avoidance of doubt, SpCas9 collectively refers to any one of the group consisting of espCas9 (also referred to herein as ES-Cas9 or esCas9), HF-Cas9, PE-Cas9, ARCas9 (also referred to as AR-Cas9).

Like the canonical Cas9 systems, Type V nucleases only require a synthetic sgRNA with a targeting domain complementary to a genomic sequence to carry out genomic editing. These nucleases contain a RuvC domain but lack the HNH domain of Type II nucleases. Further, Cas12, for example, leaves a staggered cut in the dsDNA substrate distal to the PAM, as compared to Cas9's blunt cut next to the PAM. Both Cas12a, also known as Cpf1, and Cas12b, also known as C2c1 (see Table 2), act as part of larger complex of two gRNA-associated nucleases that acts on dsDNA as a quaternary structure, nicking each strand simultaneously. See Zetsche, B. et al. (2015). Cell, 163(3):759-771; see also Liu, L. et al. (2017). Molecular Cell, 65(2):310-322. Additionally, Cas12b (C2c1) is a highly accurate nuclease with little tolerance for mismatches. See Yang, H. et al. (2016). Cell, 167(7):1814-1828.e12.

TABLE 2
Exemplary list of Cas nucleases and their requirements

| | | Type of end | |
|---|---|---|---|
| | | generated | Spacer |
| | PAM | (nucleic acid | length |
| Nuclease (Species) | (5′→3′) | target) | (nt) |
| Cas9 (S. pyogenes) | NGG | Blunt (dsDNA) | 20 |
| Cas9 (S. aureus) | NNGRRT | Blunt (dsDNA) | 20 |
| Cas9 (C. jejuni) | NNNNRYAC | Blunt (dsDNA) | 22 |
| Cas9 (S. thermophilus) | NNAGAAW | Blunt (dsDNA) | 20 |
| Cas9 (N. meningitidis) | NNNNGATT | Blunt (dsDNA) | 24 |
| Cas9 (F. novicida) | NGG | Blunt (dsDNA) | 21 |
| Cas12a (L. bacterium) | TTTV | 5′ staggered | 23-25 |
| | | (dsDNA/ssDNA) | |
| Cas12a | TTTN | 5′ staggered | 24 |
| (Acidaminococcus sp.) | | (dsDNA/ssDNA) | |
| Cas3 (E. coli) | CTT/ | None/blunt | 32 |
| | CCT/ | (ssDNA) | |
| | CAT/ | | |
| | CTC | | |

See generally Wang, J., Zhang, C., & Feng, B. (2020). Journal of Cellular and Molecular Medicine, 24(6), 3256-3270, where N = any nucleotide; R = any purine (A or G); Y = any pyrimidine (C or T); W = A or T ; V = A, C or G.

In one aspect, the CRISPR/Cas system of the present disclosure comprises at least one RNA-guided nuclease (e.g. a Cas protein) derived from one or more of the following selected bacterial genera: Corynebacterium, Sutterella, Legionella, Treponema, Filifactor, Eubacterium, Streptococcus, Lactobacillus, Mycoplasma, Bacteroides, Flavobacterium, Spirochaeta, Azospirillum, Gluconacetobacter, Neisseria, Roseburia, Parvibaculum, Nitratifractor, Campylobacter, Pseudomonas, Streptomyces, Staphylococcus, Francisella, Acidaminococcus, Lachnospiraceae, Leptotrichia, and Prevotella. In some embodiments, the Cas protein is derived from Deltaproteobacteria or Planctomycetes bacterial species.

Some aspects of the present disclosure provide strategies, methods, compositions, and treatment modalities for altering a targeted sequence within a gene locus (e.g., altering the sequence of wild type and/or of a mutant sequence within a cell or within a mammal) by insertion or deletion of one or more nucleotides mediated by an RNA-guided nuclease and one or more guide RNAs (gRNAs), resulting in loss of function of the targeted gene product. In some embodiments, the loss of function results in “knocking out” the gene of interest (i.e., generation of a “knock out”) by ablating gene expression. In some embodiments, the loss function results in a non-functional gene product (i.e., a gene product without all functionality of the wildtype gene product). In some embodiments, the loss of function results in expression of gene product with different characteristics (e.g., different binding affinity or different cellular localization).

In certain embodiments, the targeted gene is selected from NFKB1, NFKB2, and combinations thereof. In some embodiments, any region of the targeted gene (e.g., a promoter region, a 5′ untranslated region, a 3′ untranslated region, an exon, an intron, or an exon/intron border) is targeted by an RNA-guided nuclease to alter the gene. In some embodiments, a non-coding region of the targeted gene (e.g., an enhancer region, a promoter region, an intron, 5′ UTR, 3′ UTR, polyadenylation signal) is targeted to alter the gene.

### CRISPR Guide RNAs:

In one aspect, the CRISPR/Cas system of the present disclosure further provides a gRNA molecule (e.g., an isolated or non-naturally occurring RNA molecule) that interacts with the RNA-guided nuclease. In certain embodiments, the gRNA is an sgRNA comprising a crRNA sequence comprising a nucleotide sequence which is complementary to a sequence in a target nucleic acid. In some embodiments, the sgRNA further comprises an RNA scaffolding portion (i.e. tracrRNA) that interacts with the RNA-guided nuclease, such that the crRNA is positioned to scan a target nucleic acid for complementarity. In some embodiments, the system is further, optionally, comprised of an oligonucleotide—an HDR template with homology to either side of the target position. See Bloh, K., & Rivera-Torres, N. (2021). International Journal of Molecular Sciences, 22(8):3834.

In an embodiment, the RNA-guided nuclease and sgRNA are configured to orient an associated nuclease such that a cleavage event, (e.g., a double strand break or a single strand break) occurs sufficiently close to a complementary sequence in the targeted nucleic acid, thereby facilitating an alteration in the nucleic acid sequence. In some embodiments, the crRNA is 20 nucleotides in length. In some embodiments, the crRNA is 14, 15, 16, 17, 18, 19, 20, 21, 22, 23, 24, 25, 26, 27, 28, 29, or 30 nucleotides in length.

In some embodiments, the crRNA orients the RNA-guided nuclease such that a cleavage event occurs within 1, 2, 3, 4, 5, 10, 15, 20, 25, 30, 35, 40, 45, 50, 55, 60, 65, 70, 75, 80, 85, 90, 95, 100, 125, 150, 175, or 200 nucleotides away from the complementary sequence in the targeted nucleic acid. The double- or single-strand break may be positioned upstream or downstream of the complementary sequence in the targeted nucleic acid. In some embodiments, the cleavage event occurs within a targeted gene. In some embodiments, the cleavage event occurs upstream of a targeted gene.

In certain embodiments, a second gRNA molecule, comprising a second crRNA orients a second RNA-guided nuclease, such that a cleavage event occurs sufficiently close to a complementary sequence in the targeted nucleic acid, thereby facilitating an alteration in the nucleic acid sequence. In some embodiments, the first gRNA and the second gRNA promote a cleavage event within a single targeted gene. In some embodiments, the first gRNA and the second gRNA promote a cleavage event within different targeted genes. In some embodiments, the second crRNA is 20 nucleotides in length. In some embodiments, the second crRNA is 14, 15, 16, 17, 18, 19, 20, 21, 22, 23, 24, 25, 26, 27, 28, 29, or 30 nucleotides in length.

In some embodiments, the second crRNA orients the RNA-guided nuclease such that a cleavage event occurs within 1, 2, 3, 4, 5, 10, 15, 20, 25, 30, 35, 40, 45, 50, 55, 60, 65, 70, 75, 80, 85, 90, 95, 100, 125, 150, 175, or 200 nucleotides away from the complementary sequence in the targeted nucleic acid. The double- or single-strand break may be positioned upstream or downstream of the complementary sequence in the targeted nucleic acid. In some embodiments, the cleavage event occurs within a targeted gene. In some embodiments, the cleavage event occurs upstream of a targeted gene.

In some embodiments, the targeting domains of the first gRNA and the second gRNA are configured such that a cleavage event is positioned, independently for each of the gRNA molecules, within 1, 2, 3, 4, 5, 10, 15, 20, 25, 30, 35, 40, 45, 50, 55, 60, 65, 70, 75, 80, 85, 90, 95, 100, 125, 150, 175, or 200 nucleotides of the others cleavage event. In some embodiments, the first gRNA and the second gRNA molecules alter the targeted nucleic acid sequences simultaneously. In some embodiments, the first gRNA and the second gRNA molecules alter the targeted nucleic acid sequences sequentially.

In some embodiments, a single-strand break is accompanied by a second single-strand break, positioned by the crRNA of a first gRNA and a second gRNA, respectively. For example, the crRNA may orient the associated RNA-guided nucleases such that a cleavage event, (e.g., the two single-strand breaks), are positioned within 1, 2, 3, 4, 5, 10, 15, 20, 25, 30, 35, 40, 45, 50, 55, 60, 65, 70, 75, 80, 85, 90, 95, 100, 125, 150, 175, or 200 nucleotides of one another. In some embodiments, a first crRNA and a second crRNA are configured to orient associated RNA-guided nucleases such that, for example, two single-strand breaks occurs at the same position, or within 1, 2, 3, 4, 5, 10, 15, 20, 25, 30, 35, 40, 45, 50 nucleotides of one another, on opposing strands of genomic DNA, thereby essentially approximating a double strand break.

In some embodiments, the nucleic acid encoding one or more crRNAs is selected from any of SEQ ID NOs: 5323-6090. In some embodiments, the nucleic acid encoding one or more crRNAs target hNFKB1 and is selected from any of SEQ ID NOs: 5323-5650.

In some embodiments, the nucleic acid encoding one or more crRNAs is selected from any of SEQ ID NOs: 5323-6090. In some embodiments, the nucleic acid encoding one or more crRNAs target hNFKB2 and is selected from any of SEQ ID NOs: 5651-6090.

In some embodiments a nucleic acid encodes a second sgRNA molecule. In some embodiments, a nucleic acid encodes a third sgRNA molecule. In some embodiments, a nucleic acid encodes a fourth sgRNA molecule.

In certain embodiments, a nucleic acid may comprise (a) a sequence encoding a first sgRNA, comprising a crRNA that is complementary with a sequence in a targeted gene, (b) a sequence encoding a second sgRNA, comprising a crRNA that is complementary with a sequence in a second targeted gene, and (c) a sequence encoding an RNA-guided nuclease (e.g., Cas9). Optionally, (d) and (e) are sequences encoding a third sgRNA and a fourth sgRNA, respectively. In some embodiments, the second targeted gene is the same as the first targeted gene. In other embodiments, the second targeted gene is different from the first targeted gene. In some embodiments, (a), (b), and (c) are encoded within the same nucleic acid molecule (e.g., the same vector). In some embodiments, (a) and (b) are encoded within the same nucleic acid molecule. In some embodiments, (a), (b) and (d) are encoded within the same nucleic acid molecule. In some embodiments, (a), (b) and (e) are encoded within the same nucleic acid molecule. In some embodiments, (a), (b), (d) and (e) are encoded within the same nucleic acid molecule. In some embodiments, (a), (b), and (c) are encoded within separate nucleic acid molecules. When more than two sgRNAs are used, any combination of (a), (b), (c), (d) and (e) may be encoded within a single or separate nucleic acid molecules.

In one aspect, the nucleic acid molecules (i.e., those encoding (a), (b), (c), (d) or (e)) are delivered to a target cell (i.e., any combination of the encoded RNA-guided nuclease of (c) and at least one encoded gRNA molecule of (a), (b), (d), or (e) contact a target cell). In some embodiments, said nucleic acid molecules are delivered to a target cell in vivo. In other embodiments, said nucleic acid molecules are delivered to a target cell ex vivo. In some embodiments, said nucleic acid molecules are delivered to a target cell in vitro. In certain embodiments, said nucleic acid molecules are delivered to a target cell as DNA. In other embodiments, said nucleic acid molecules are delivered to a target cell as RNA (e.g., mRNA). In some embodiments, the products of said nucleic acid molecules are delivered as an assembled ribonucleoprotein (RNP).

In some embodiments, contacting a target cell comprises delivering said RNA-guided nuclease of (c), as a protein with at least one said nucleic acid molecules selected from (a), (b), (d), and (e). In some embodiments, contacting a target cell comprises delivering said encoded RNA-guided nuclease of (c), as DNA with at least one said nucleic acid molecules selected from (a), (b), (d), and (e). In some embodiments, contacting a target cell comprises delivering said encoded RNA-guided nuclease of (c), as mRNA with at least one said nucleic acid molecules selected from (a), (b), (d), and (e).

In certain embodiments, CRISPR components are delivered to a target cell via nanoparticles. Exemplary nanoparticles that may be used with all CRISPR/Cas systems disclosed herein include, at least, lipid nanoparticles or liposomes, hydrogel nanoparticles, metalorganic nanoparticles, gold nanoparticles, magnetic nanoparticles and virus-like particles. See generally Xu, C. F. et al. (2021). Advanced Drug Delivery Reviews, 168:3-29.

### B. Talen

In one aspect, the present disclosure contemplates use of methods, components, and compositions relating to Transcription Activator-Like Effector Nucleases (TALENs) to effectuate augmentation of a nucleic acid sequence (e.g., a targeted gene.

TALE stands for “Transcription Activator-Like Effector” proteins, which include TALENs (“Transcription Activator-Like Effector Nucleases”). A method of using a TALE system for gene editing may also be referred to herein as a TALE method. TALEs are naturally occurring proteins from the plant pathogenic bacteria genus Xanthomonas, and contain DNA-binding domains composed of a series of 33-35-amino-acid repeat domains that each recognizes a single base pair. TALE specificity is determined by two hypervariable amino acids that are known as the repeat-variable di-residues (RVDs). Modular TALE repeats are linked together to recognize contiguous DNA sequences. A specific RVD in the DNA-binding domain recognizes a base in the target locus, providing a structural feature to assemble predictable DNA-binding domains. The DNA binding domains of a TALE are fused to the catalytic domain of a type IIS FokI endonuclease to make a targetable TALE nuclease. To induce site-specific mutation, two individual TALEN arms, separated by a 14-20 base pair spacer region, bring FokI monomers in close proximity to dimerize and produce a targeted double-strand break.

Several large, systematic studies utilizing various assembly methods have indicated that TALE repeats can be combined to recognize virtually any user-defined sequence. Custom-designed TALE arrays are also commercially available through Cellectis Bioresearch (Paris, France), Transposagen Biopharmaceuticals (Lexington, KY, USA), and Life Technologies (Grand Island, NY, USA). TALE and TALEN methods suitable for use in the present disclosure are described in U.S. Patent Application Publication Nos. US 2011/0201118 A1; US 2013/0117869 A1; US 2013/0315884 A1; US 2015/0203871 A1 and US 2016/0120906 A1, the disclosures of which are incorporated by reference herein.

Non-limiting examples of genes that may be silenced or inhibited by permanently gene-editing via a TALE method include NFKB1, NFKB2, and combinations thereof. Non-limiting examples of genes that may be augmented such that their resultant products function as decoys or dominant negatives by permanently gene-editing via a TALE method include NFKB1, NFKB2, and combinations thereof. Non-limiting examples of genes that may be enhanced by permanently gene-editing via a TALE method include NFKB1, NFKB2, and combinations thereof. In an aspect, the disclosure provides compositions for up-regulation of protein receptors (including wildtype or genetically edited), including those that bind to anti-inflammatory cytokines via a TALE method.

Examples of systems, methods, and compositions for altering the expression of a target gene sequence by a TALE method, and which may be used in accordance with embodiments of the present disclosure, are described in U.S. Pat. No. 8,586,526, which is incorporated by reference herein.

### C. Zinc-Finger Nucleases (ZFN)

In one aspect, the present disclosure contemplates use of methods, components, and compositions relating to zinc-finger nucleases (ZFNs) to effectuate augmentation of a nucleic acid sequence (e.g., a targeted gene).

An individual zinc finger contains approximately 30 amino acids in a conserved ββα configuration. Several amino acids on the surface of the (-helix typically contact 3 bp in the major groove of DNA, with varying levels of selectivity. Zinc fingers have two protein domains. The first domain is the DNA binding domain, which includes eukaryotic transcription factors and contain the zinc finger. The second domain is the nuclease domain, which includes the FokI restriction enzyme and is responsible for the catalytic cleavage of DNA.

The DNA-binding domains of individual ZFNs typically contain between three and six individual zinc finger repeats and can each recognize between 9 and 18 base pairs. If the zinc finger domains are specific for their intended target site then even a pair of 3-finger ZFNs that recognize a total of 18 base pairs can, in theory, target a single locus in a mammalian genome. One method to generate new zinc-finger arrays is to combine smaller zinc-finger “modules” of known specificity. The most common modular assembly process involves combining three separate zinc fingers that can each recognize a 3 base pair DNA sequence to generate a 3-finger array that can recognize a 9 base pair target site. Alternatively, selection-based approaches, such as oligomerized pool engineering (OPEN) can be used to select for new zinc-finger arrays from randomized libraries that take into consideration context-dependent interactions between neighboring fingers. Engineered zinc fingers are available commercially; Sangamo Biosciences (Richmond, CA, USA) has developed a propriety platform (CompoZr®) for zinc-finger construction in partnership with Sigma-Aldrich (St. Louis, MO, USA).

Non-limiting examples of genes that may be silenced or inhibited by permanently gene-editing via a zinc finger method include NFKB1, NFKB2, and combinations thereof. Non-limiting examples of genes that may be augmented such that their resultant products function as decoys or dominant negatives by permanently gene-editing via a zinc finger method include NFKB1, NFKB2, and combinations thereof. Non-limiting examples of genes that may be enhanced by permanently gene-editing via a zinc finger method include NFKB1, NFKB2, and combinations thereof. In an aspect, the disclosure provides compositions for up-regulation of protein receptors (including wildtype or genetically edited), including those that bind to anti-inflammatory cytokines via a zinc finger method.

Examples of systems, methods, and compositions for altering the expression of a target gene sequence by a zinc finger method, which may be used in accordance with embodiments of the present disclosure, are described in U.S. Pat. Nos. 6,534,261, 6,607,882, 6,746,838, 6,794,136, 6,824,978, 6,866,997, 6,933,113, 6,979,539, 7,013,219, 7,030,215, 7,220,719, 7,241,573, 7,241,574, 7,585,849, 7,595,376, 6,903,185, and 6,479,626, which are incorporated by reference herein.

Other examples of systems, methods, and compositions for altering the expression of a target gene sequence by a zinc finger method, which may be used in accordance with embodiments of the present disclosure, are described in Beane, et al., Mol. Therapy, 2015, 23 1380-1390, the disclosure of which is incorporated by reference herein.

### IV. Joint Disease or Illness

### A. Introduction

As described herein, embodiments of the present disclosure provide compositions and methods for improving joint function and treating joint disease. In particular embodiments, compositions and methods are provided to gene-edit synovial fibroblasts, synoviocytes, chondrocytes, tissue (resident) macrophages, or other cells to reduce pro-inflammatory signaling mediated by the binding of inflammatory cytokines-including, but not limited to, IL1α, IL1β, TNFα, IL6, IL8, IL18, IL33, matrix metalloproteinases (MMPs), TGFβ1, TGFβ2, and combinations thereof—to their cognate receptor(s) as a result of ROS and their cellular effectors. Some embodiments are used for treating various forms of arthritis and other inflammatory joint diseases. Some embodiments are further useful for treating canine lameness due to osteoarthritis. Some embodiments are further useful for treating equine lameness due to joint disease. Some embodiments are further useful for treating feline lameness due to joint disease. Some embodiments are also useful for treating post-traumatic arthritis, gout, pseudogout, psoriatic arthritis, and other inflammation-mediated or immune-mediated joint diseases.

Treatment of osteoarthritis, degenerative joint disease, and other joint dysfunctions is complex, and few long-term options exist for either symptomatic relief or restoring joint function. Osteoarthritis (OA) is the leading cause of disability due to pain. See, Neogi, T. (2013). Osteoarthritis Cartilage, 21(9):1145-53. OA and similar diseases impact all mammal species, including working animals, domestic pets, and their owners. The common mechanistic thread among joint diseases is the presence of acute of chronic inflammation, which is driven by increased levels of pro-inflammatory cytokine signaling. Joint diseases tend to take a progressive course that encompasses discomfort, pain, and—especially in the case of OA—disability, depending on the degree of disease progression.

Psoriatic arthritis (PsA) is another chronic inflammatory joint disease, in which the joint symptoms are accompanied by skin lesions, such as those commonly associated with psoriasis. See, Boehncke, W. et al. (2014). British Journal of Dermatology, 170(4):772-786. Like other forms of arthritis, such as OA, PsA is caused by pro-inflammatory signaling of a host of cytokines, including IL-1. Indeed, PsA morbidity has been shown to correlate with single nucleotide polymorphisms (SNPs) that impact the activity of the IL-1 gene locus. See, Rahman, P. et al. (2006). Arthritis and Rheumatism, 54(7):2321-2325. These studies also implicate inflammatory cytokine signaling, in general, and IL-1 more specifically, in disease progression.

Gout is a chronic inflammatory condition that affects joints. The underlying cause is monosodium urate (MSU) crystal deposition and the resultant host response, particularly in joint structures (as well as subcutaneous tissues and other sites). See, Dalbeth, N., & Stamp, L. (2014). Annals of the Rheumatic Diseases, 73(9):1598-1600. The clinical manifestations include recurrent acute flares of severe inflammatory arthritis and tendinobursitis. IL-1 and other pro-inflammatory mediators are a major contributor to this host response. See, Dinarello, C. A. (2014). Molecular Medicine, 20(1):S43-S58. To this end, effective blockade of these signaling pathways may provide relief to gout patients.

The current standard of care for many joint disease patients includes anti-inflammatory medications (e.g., NSAIDs) or anti-rheumatics (e.g., methotrexate [inhibitor of AICAR] or adalimumab [anti-TNF alpha monoclonal antibody]). See, Friedman, B., & Cronstein, B. (2019). Joint Bone Spine, 86(3):301-307. All of these treatments require repeated dosing for continued effectiveness, which may lead to toxicity issues or tolerance over time. As such, new methods and compositions to treat joint disease and illness are acutely needed to treat these chronic conditions.

In one aspect, the compositions and methods herein described are directed to treat joint disease or illness in a mammal in need thereof. In some embodiments, the joint disease or illness is osteoarthritis. In some embodiments, the joint disease or illness is psoriatic arthritis. In some embodiments, the joint disease or illness is gout.

Among the advantages of the present disclosure over treatments currently available for mammals afflicted with one or more joint disease or illness include the period of relief from symptoms. Upon genetic editing of a cell within a joint, pro-inflammatory signaling is silenced through the targeted gene for the life of that cell and any mitotic progeny. By contrast, biologic treatments require periodic dosing, which may magnify the impact of the host of potentially severe side effects. Among various genetic approaches, the present disclosure is also superior due to, among other reasons, a resistance to leakiness by virtue of modifying a protein receptor, rather than ablating expression of a ligand, which may result in compensatory effects (e.g., buildup of other factors due to lack of negative feedback).

In some embodiments, the present disclosure includes a method for the treatment or prevention of a joint disease or condition in a subject in need thereof, the method comprising administering, to a joint of the subject, a pharmaceutical composition comprising a therapeutically effective amount of a Clustered Regularly Interspaced Short Palindromic Repeats (CRISPR) gene-editing system, the system comprising (i) a CRISPR Associated (Cas) protein; and (ii) at least one guide RNA targeting an NFKB1 gene, an NFKB2 gene, or a combination thereof. In some embodiments, the joint disease or condition is osteoarthritis. In some embodiments, the joint disease or condition is psoriatic arthritis. In some embodiments, the joint disease or condition is gout.

In some embodiments, the present disclosure includes a method for the treatment or prevention of an arthritis. Non-limiting examples of arthritis the can be treated using the compositions and methods described herein include post-traumatic arthritis, osteoarthritis (a degenerative condition that affects the joints, most commonly the hips, knees, and hands), rheumatoid arthritis (an autoimmune disorder that causes inflammation in the joints and surrounding tissue), psoriatic arthritis (a type of arthritis that occurs in people with psoriasis, a skin condition characterized by scaly red patches), gout (a type of arthritis caused by the buildup of uric acid crystals in the joints), lupus (a chronic autoimmune disorder that can cause inflammation and damage to the joints, as well as other organs), ankylosing spondylitis (a type of arthritis that primarily affects the spine, causing inflammation and stiffness), reactive arthritis (a type of arthritis that occurs as a reaction to an infection in the body), septic arthritis (a type of arthritis caused by an infection in the joint), juvenile idiopathic arthritis (a form of arthritis that affects children under the age of 16), and fibromyalgia (a chronic pain disorder that can cause widespread pain and stiffness, including in the joints).

In some embodiments, the present disclosure includes a method for the treatment or prevention of pseudogout, Crystal arthropathies (caused by the formation of crystals in the joints, such as gout and pseudogout), or CPPD disease (calcium pyrophosphate deposition disease) also called chondrocalcinosis.

In some embodiments, the present disclosure includes a method for the treatment or prevention of rheumatoid arthritis, psoriasis, asthma, inflammatory bowel disease, multiple sclerosis, Alzheimer's disease, Type 2 diabetes, cardiovascular disease, or cancer. In some embodiments, these disorders are treated by administering a CRISPR composition, as described herein, targeting NFKB1 or NFKB2.

In some embodiments, the disclosure provides a method for treating or preventing free oxygen radicals in a subject in need thereof by administering a therapeutically effective amount of a composition, wherein the composition comprises: (i) an RNA-guided nuclease or a nucleic acid encoding an RNA-guided nuclease; and (ii) at least one guide RNA or a nucleic acid encoding at least one guide RNA targeting a NFKB1 or NFKB2 gene. In some embodiments, the RNA-guided nuclease or a nucleic acid encoding an RNA-guided nuclease and at least one guide RNA or a nucleic acid encoding at least one guide RNA are formulated in a lipid nanoparticle (LNP). In some embodiments, the at least one guide RNA comprises a crRNA sequence selected from SEQ ID NOS: 1-680 and 5323-5410. In some embodiments, the at least one guide RNA comprises a crRNA sequence selected from SEQ ID NOS: 1-148 and 329-497. In some embodiments, the at least one guide RNA comprises a crRNA sequence selected from SEQ ID NOS: 1-45 and 329-384. In some embodiments, the at least one guide RNA comprises a crRNA sequence selected from SEQ ID NOS: 1-13 and 329-335. In some embodiments, the pharmaceutical composition or method is for treating a joint disorder. In some embodiments, the pharmaceutical composition or method is for treating arthritis, osteoarthritis, rheumatoid arthritis, post-traumatic arthritis, gout, pseudogout, canine lameness, equine lameness, or feline lameness.

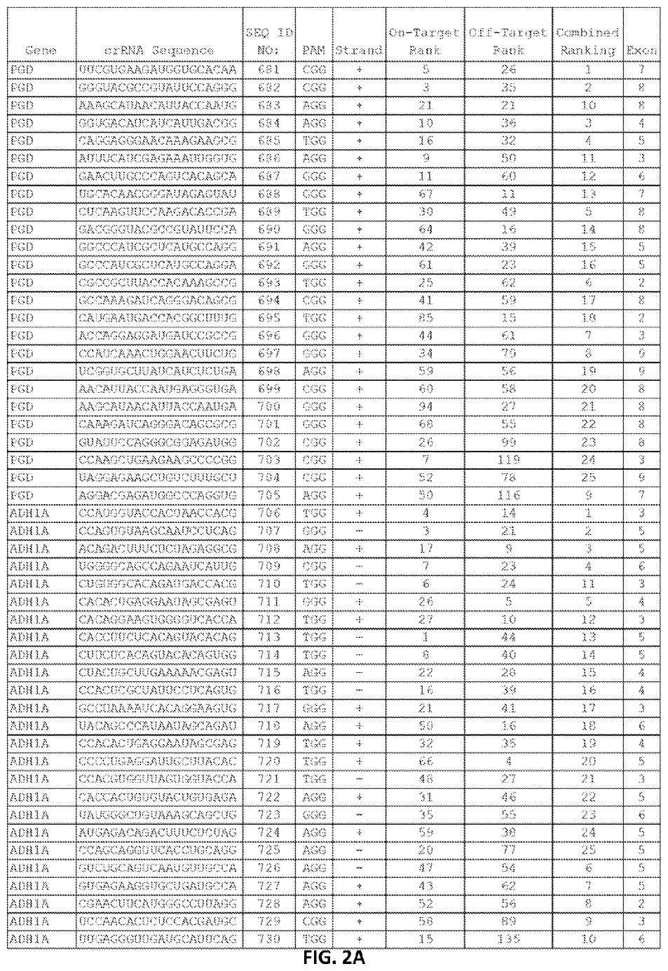

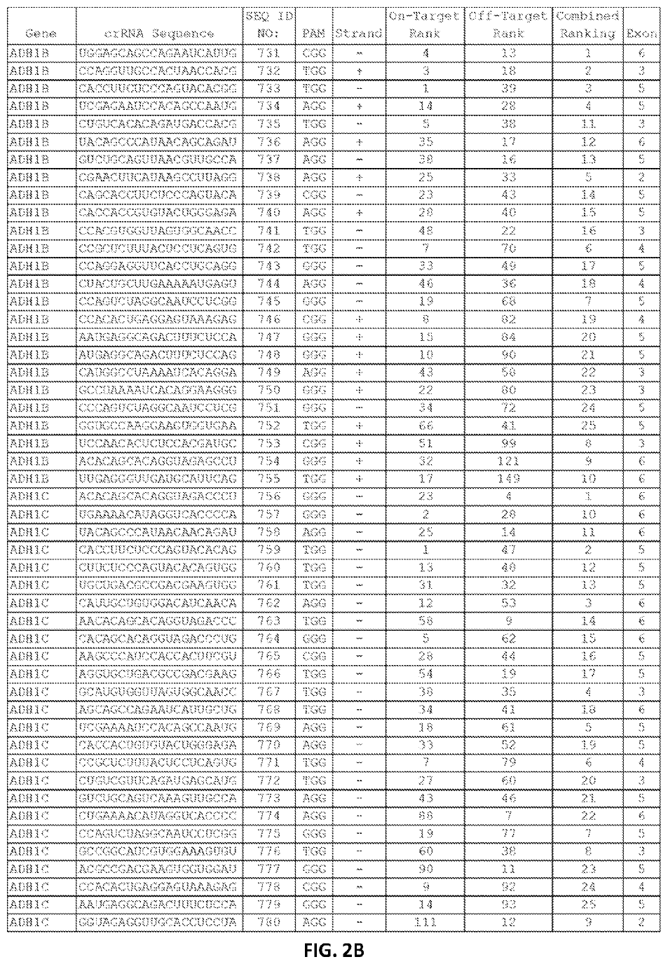

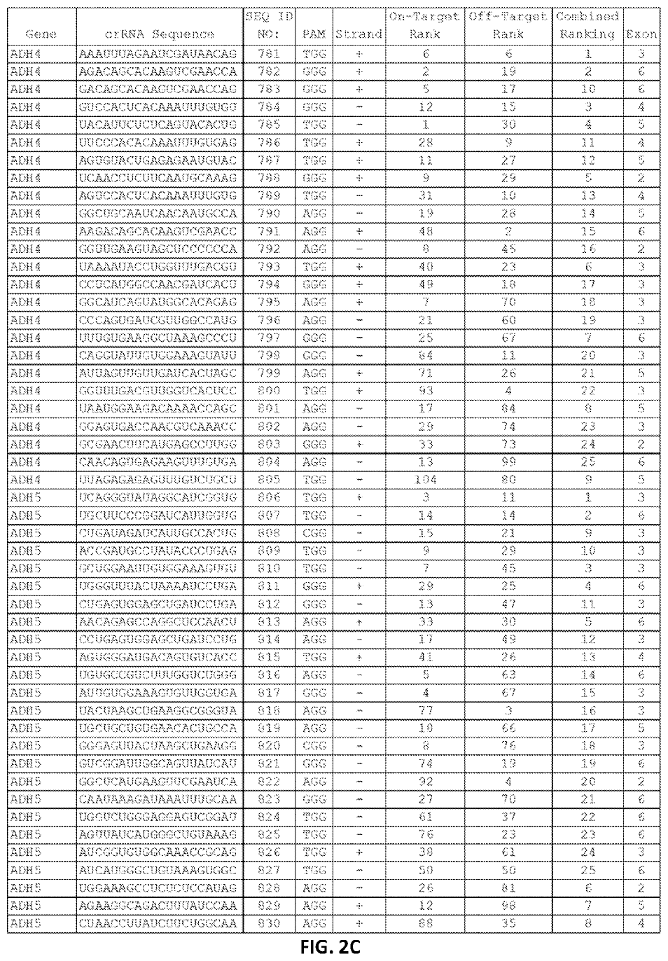

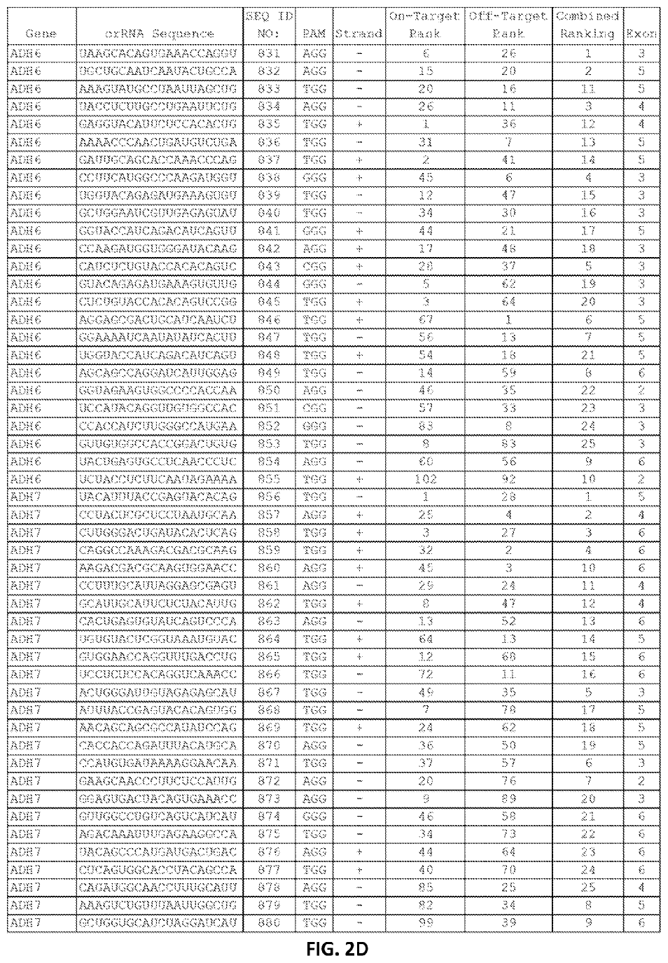

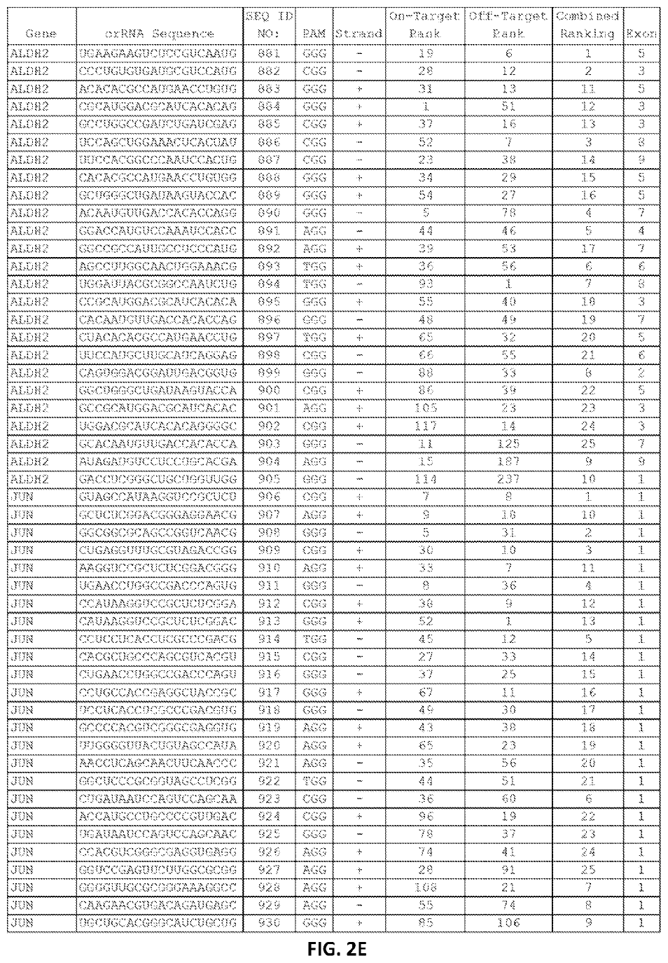

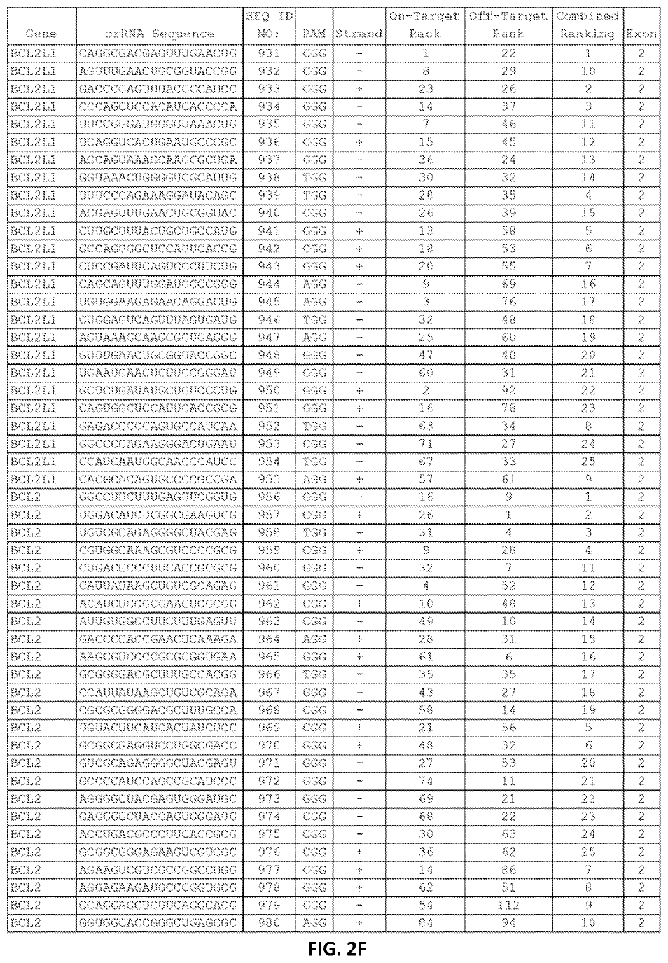

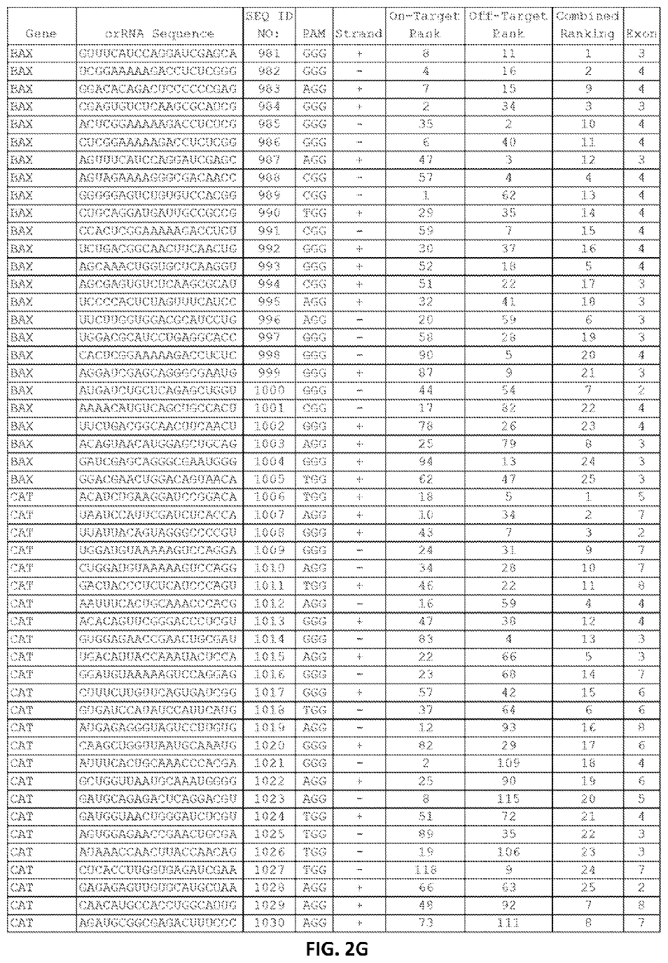

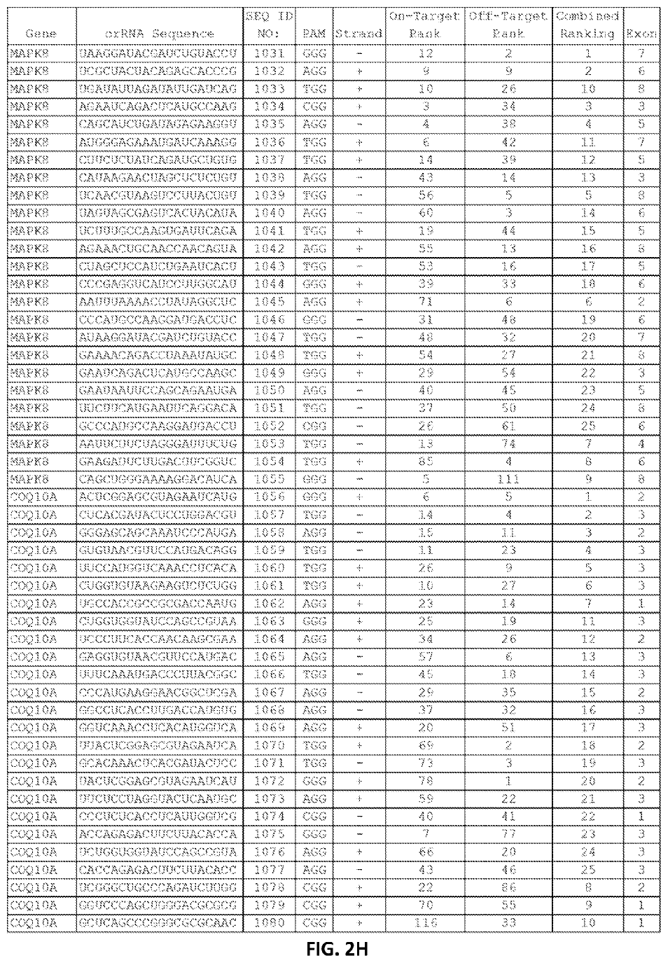

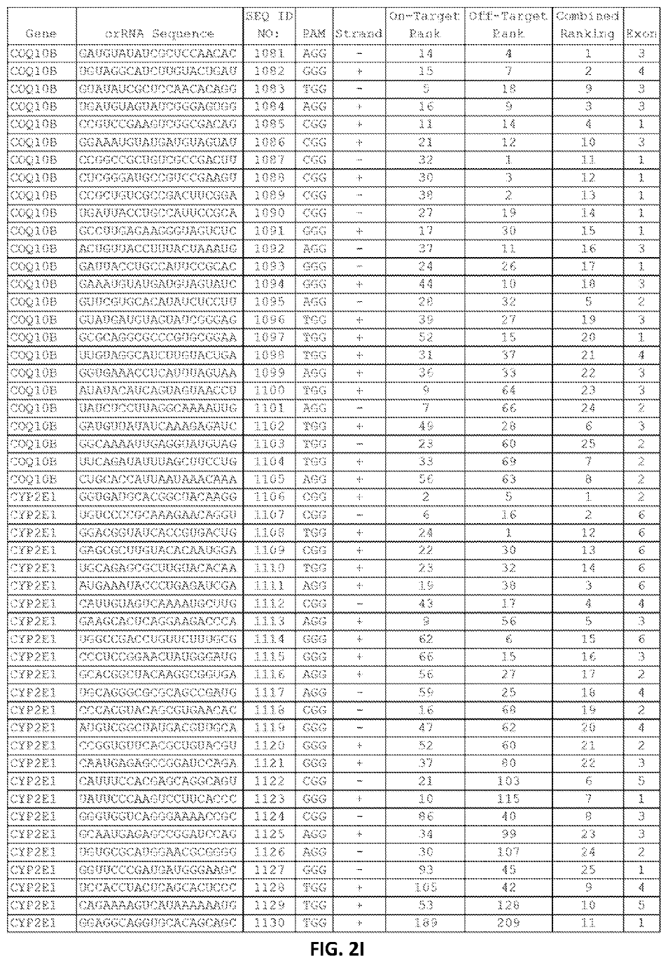

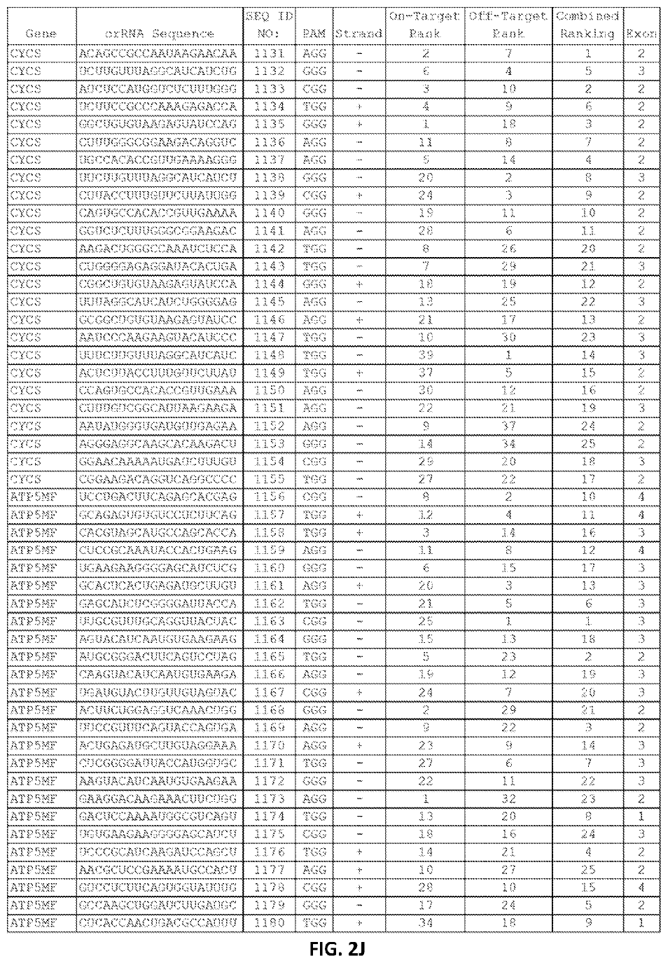

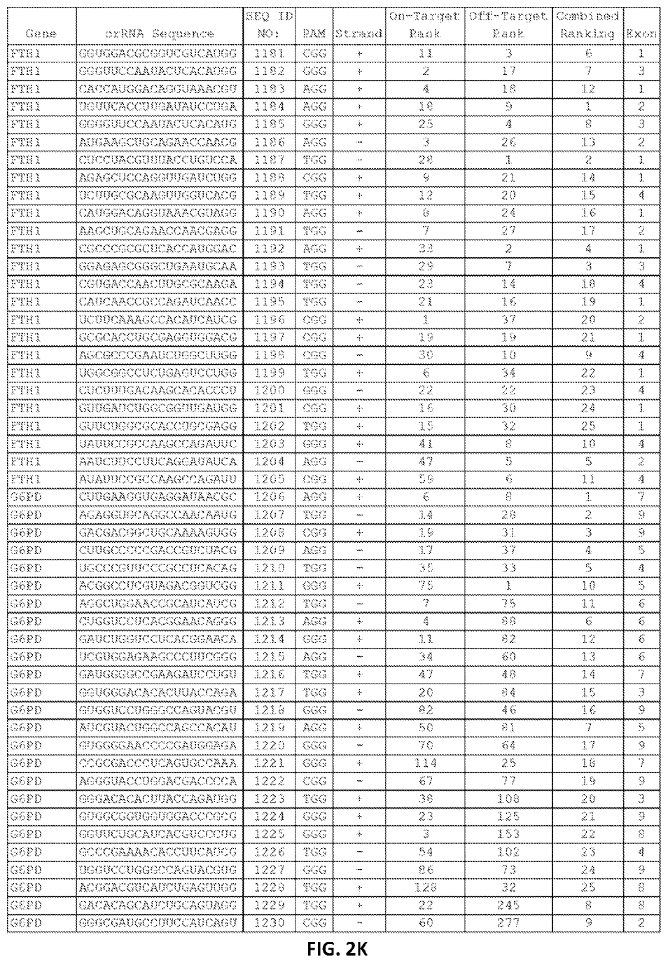

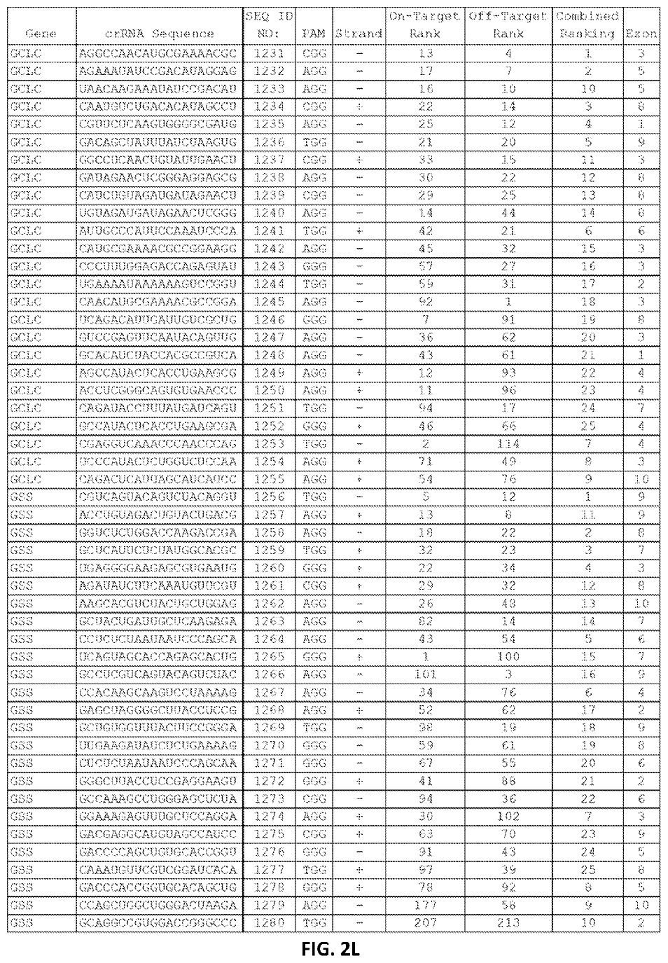

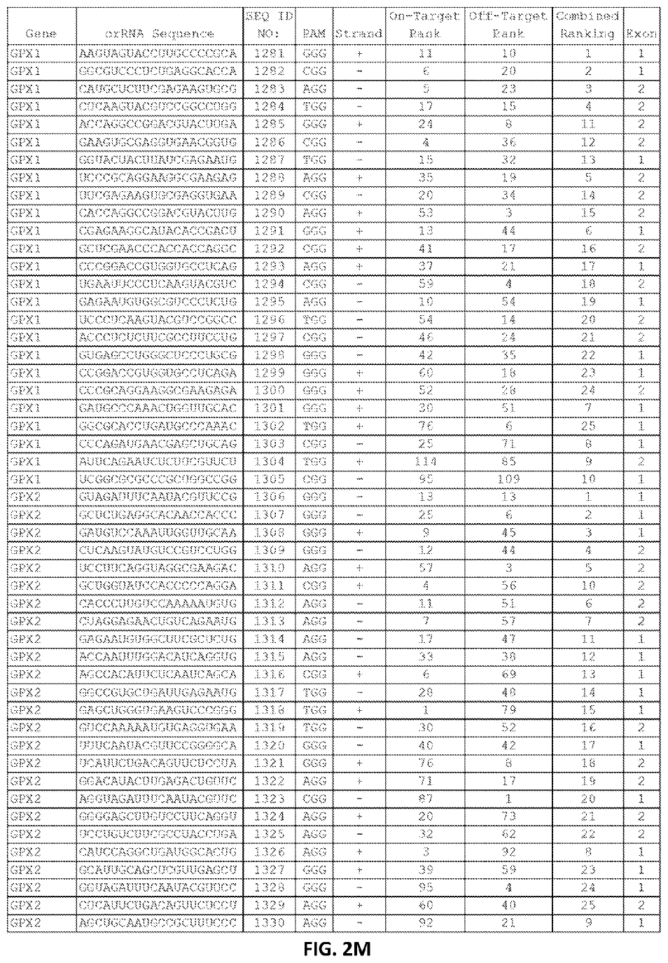

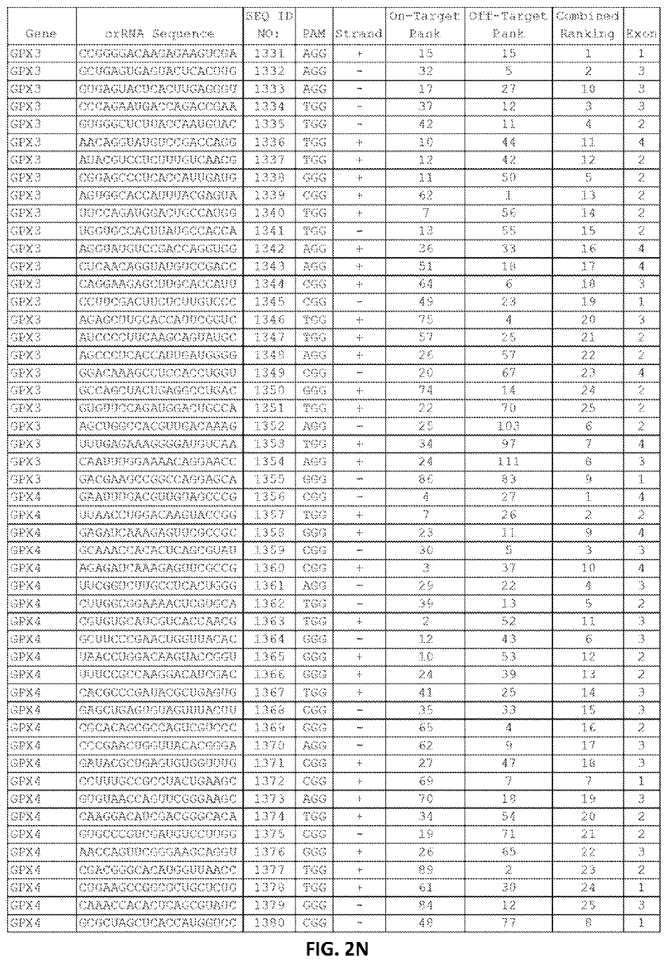

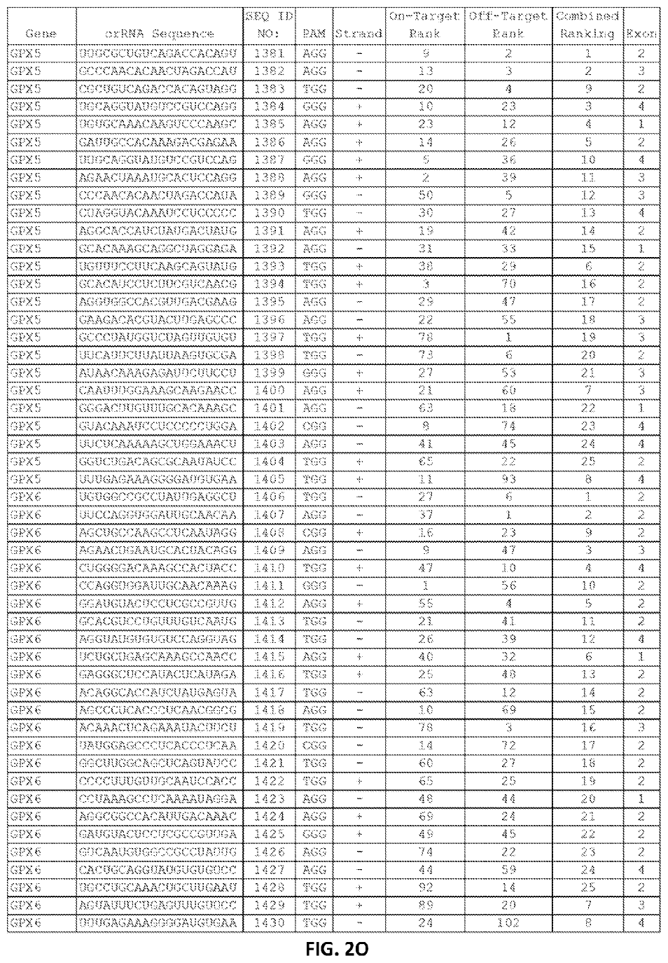

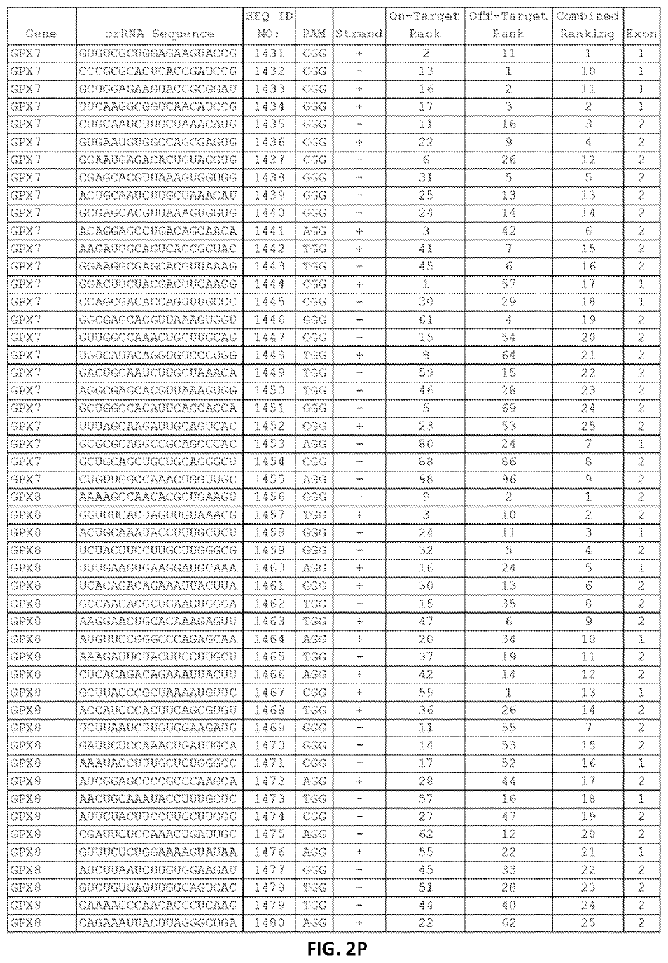

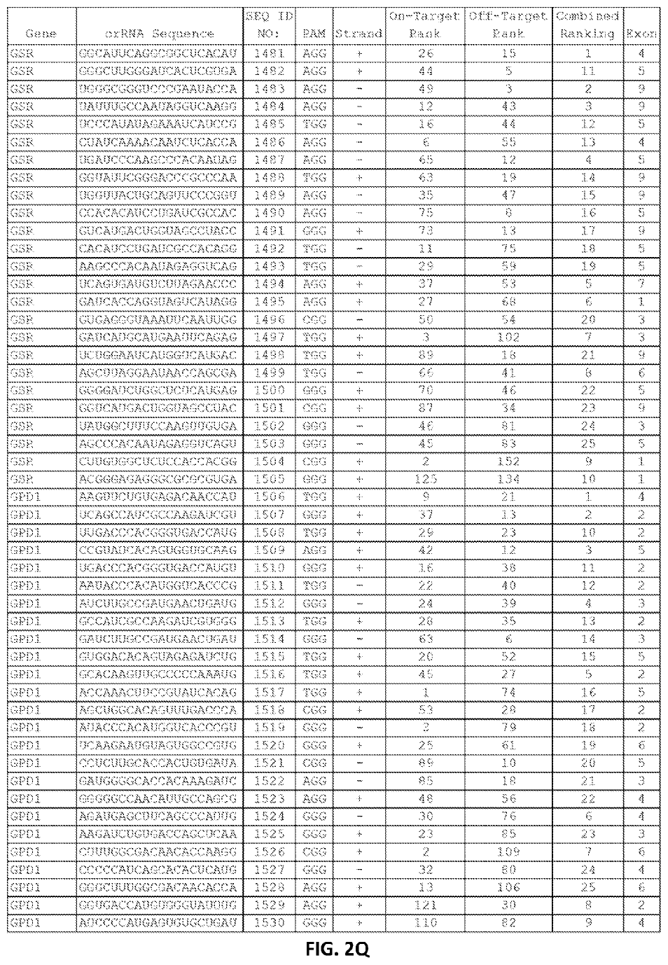

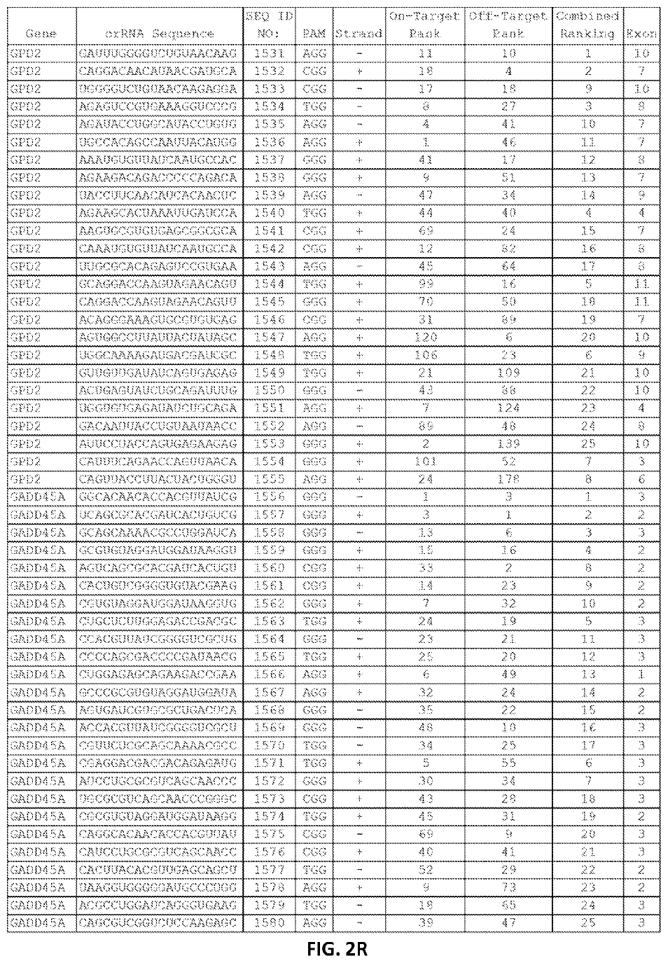

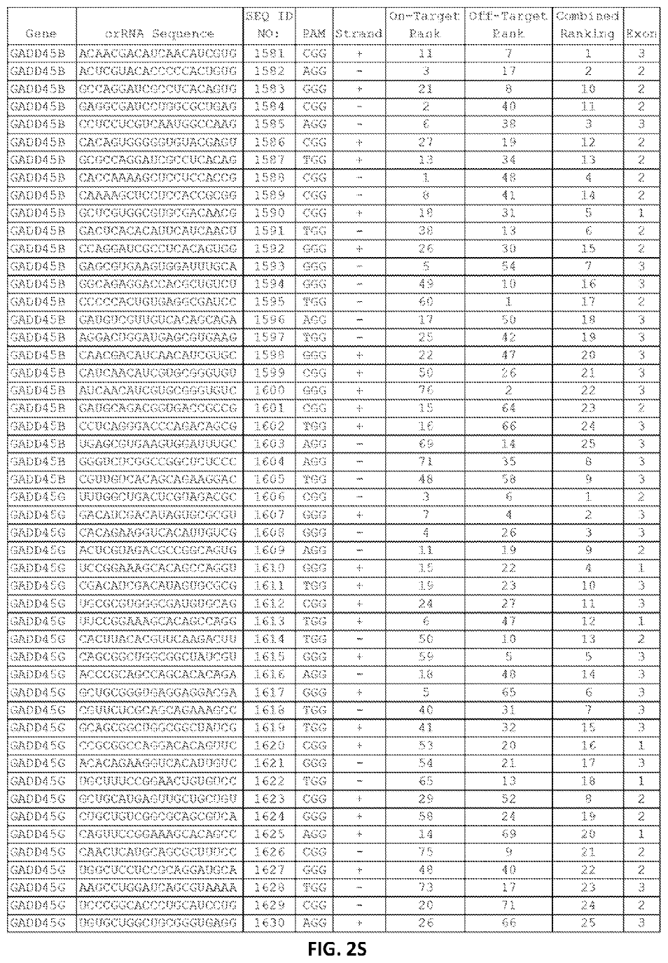

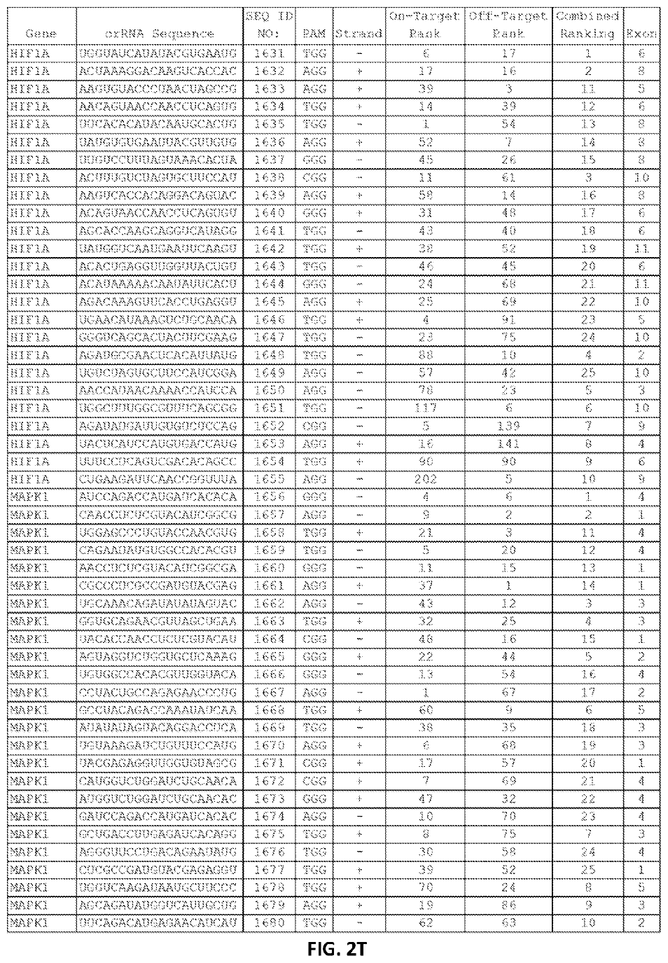

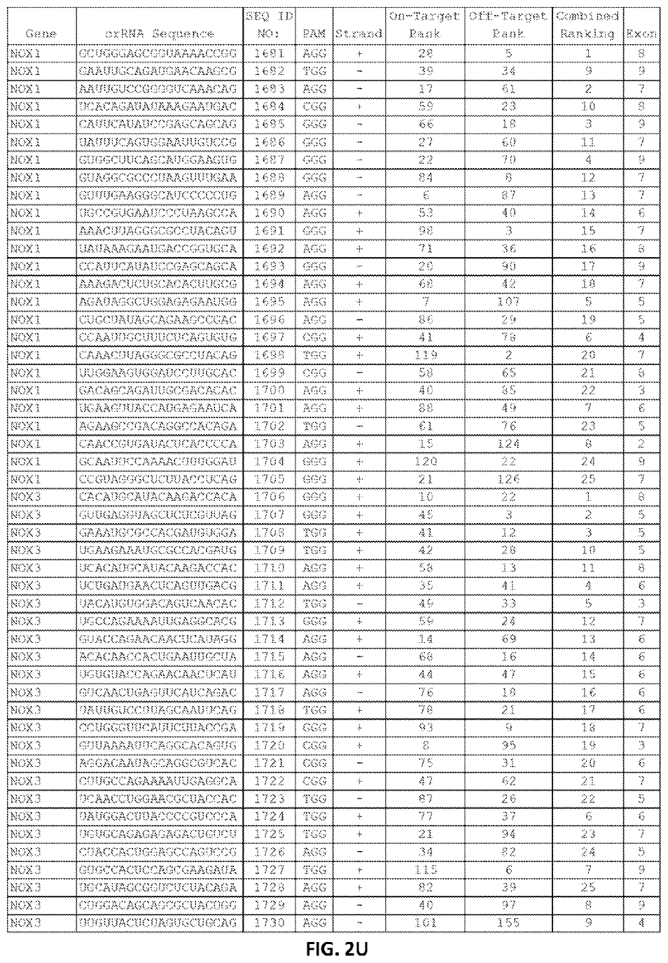

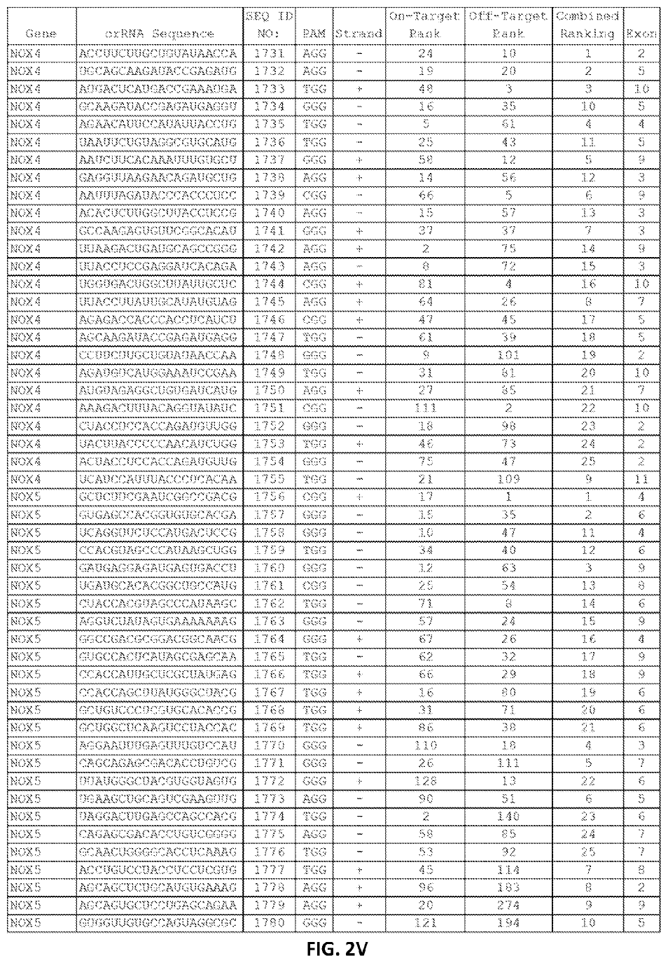

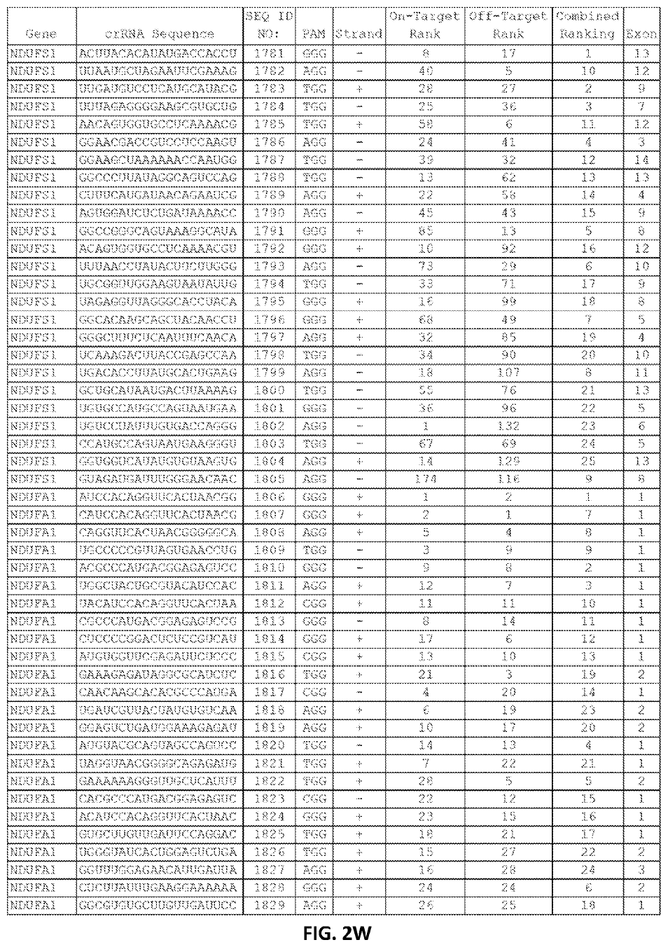

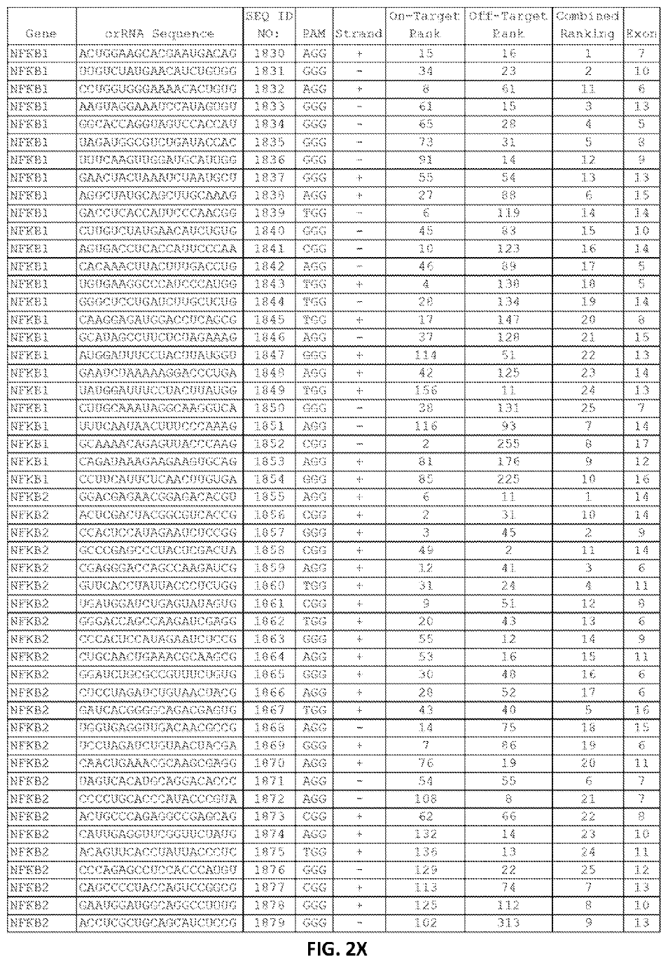

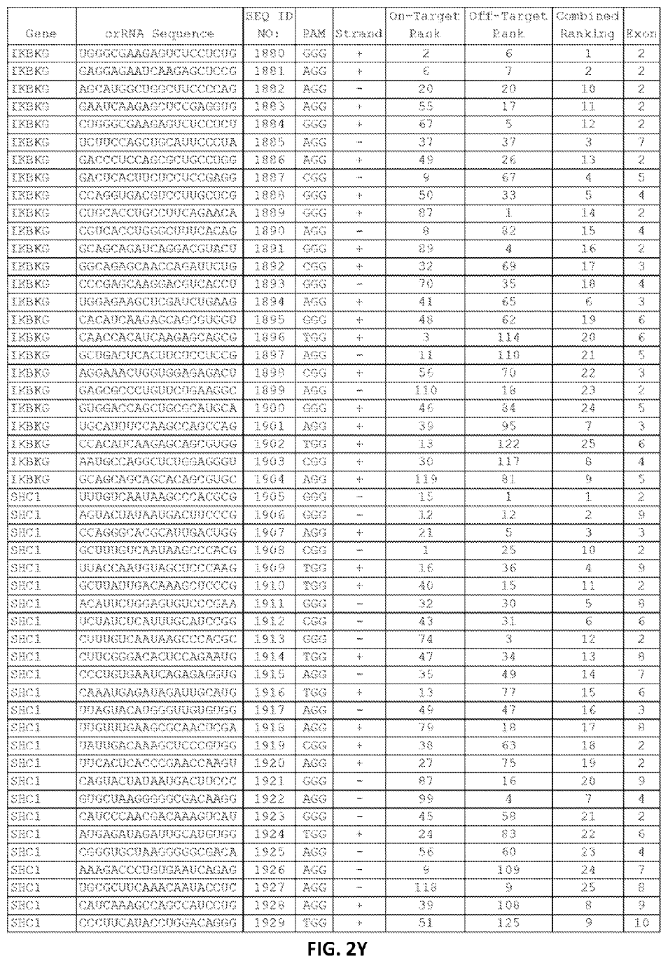

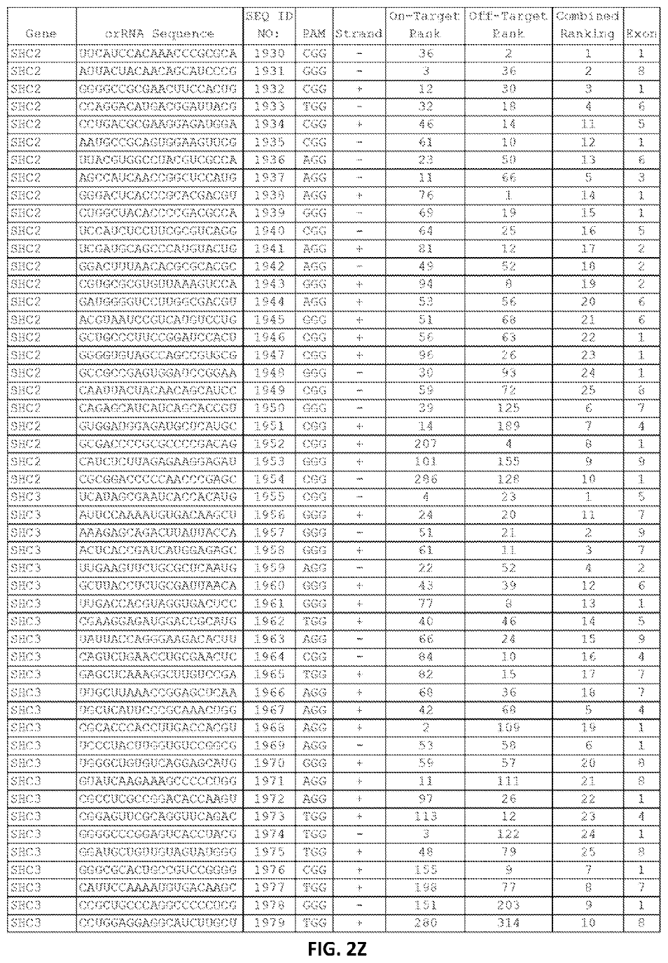

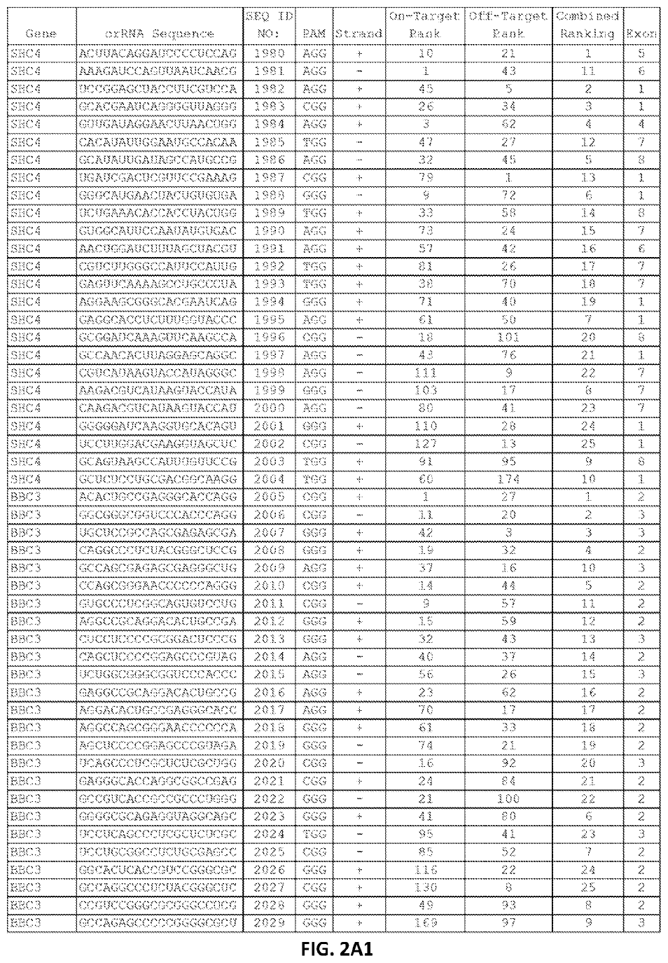

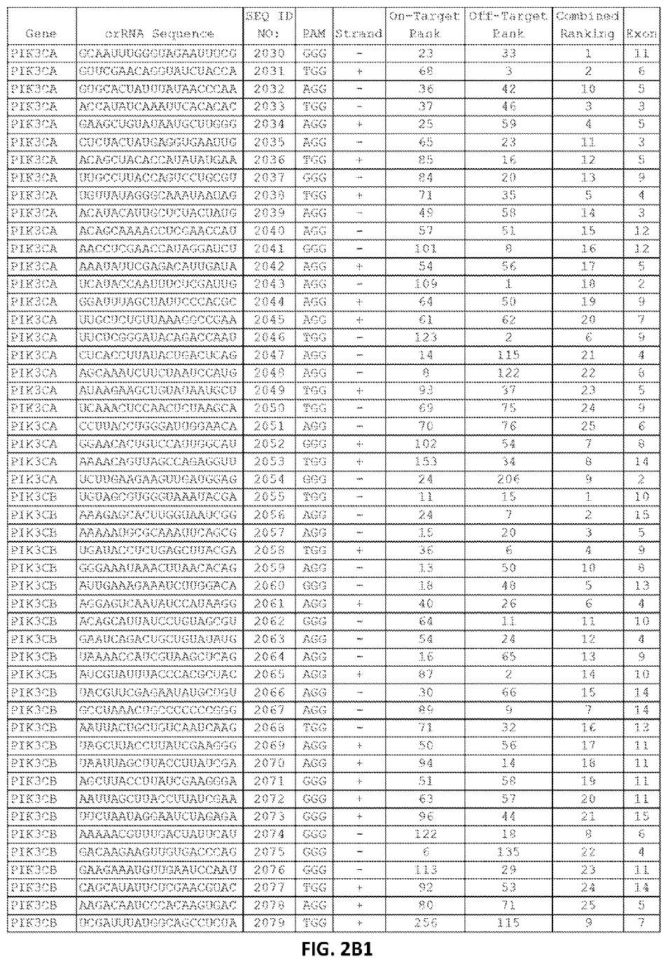

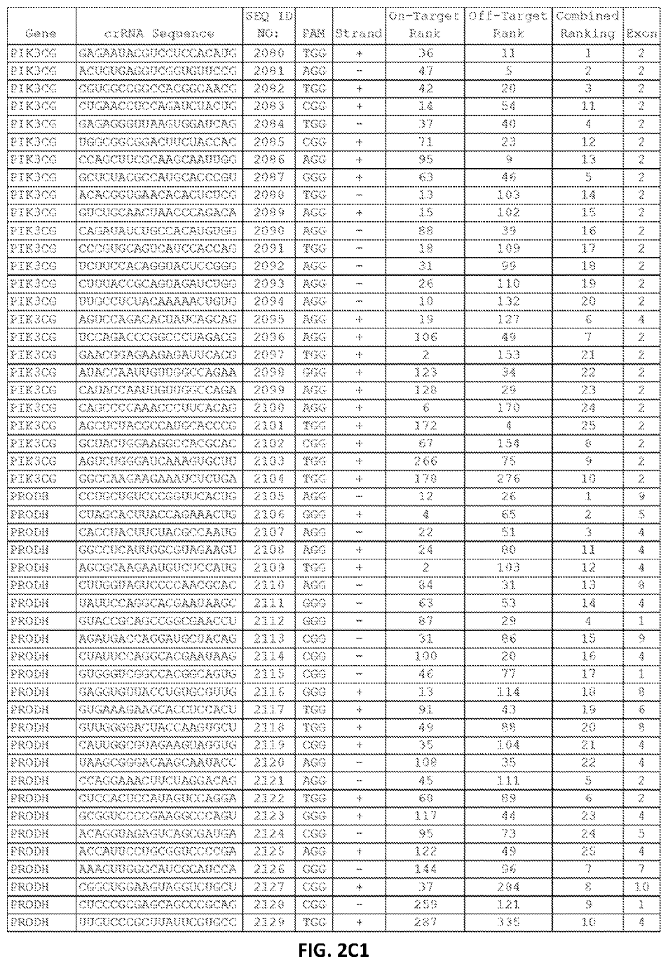

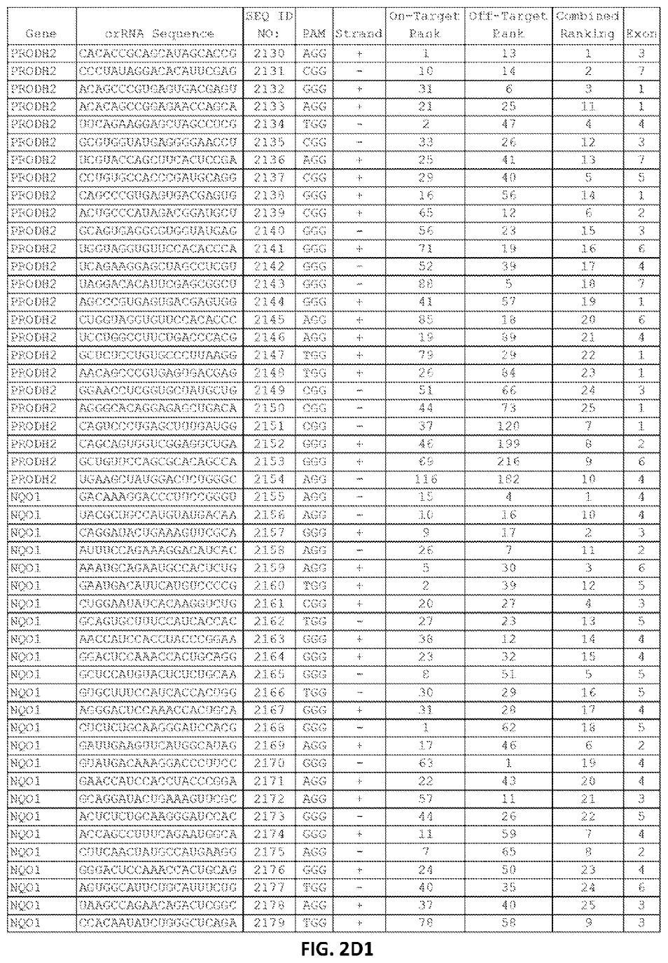

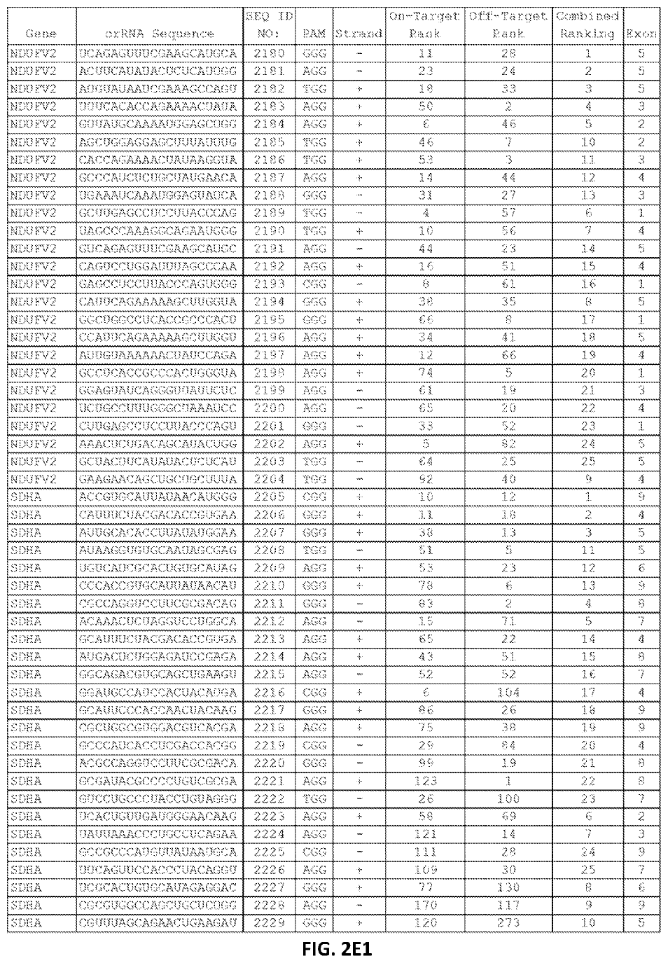

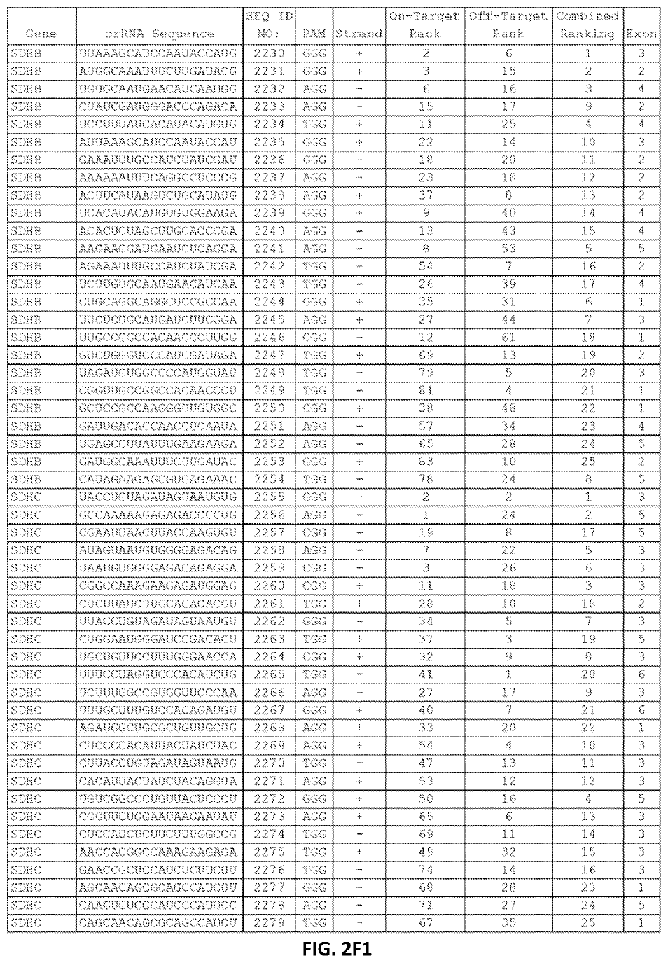

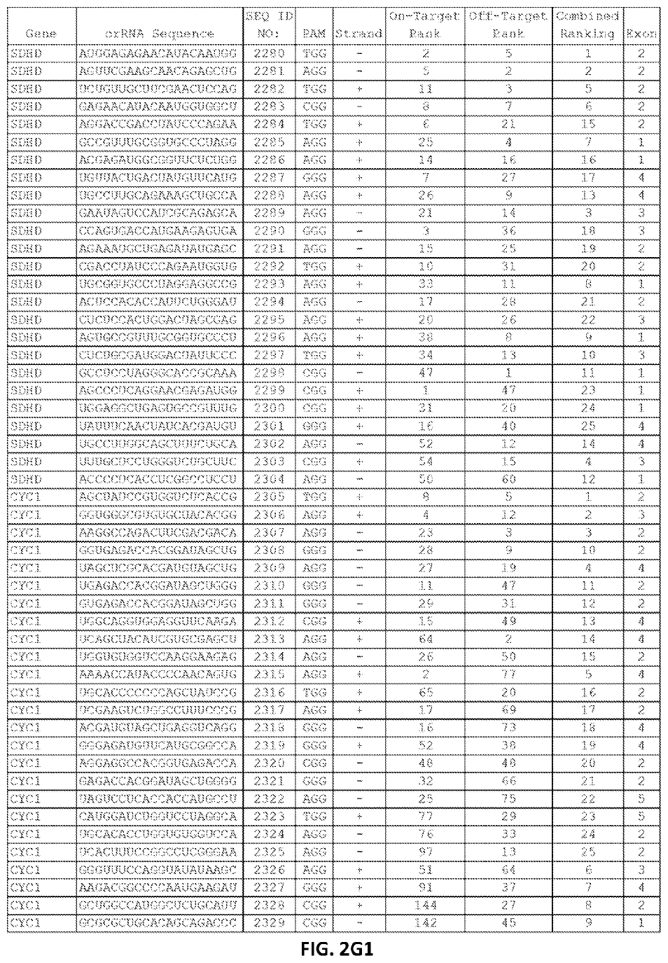

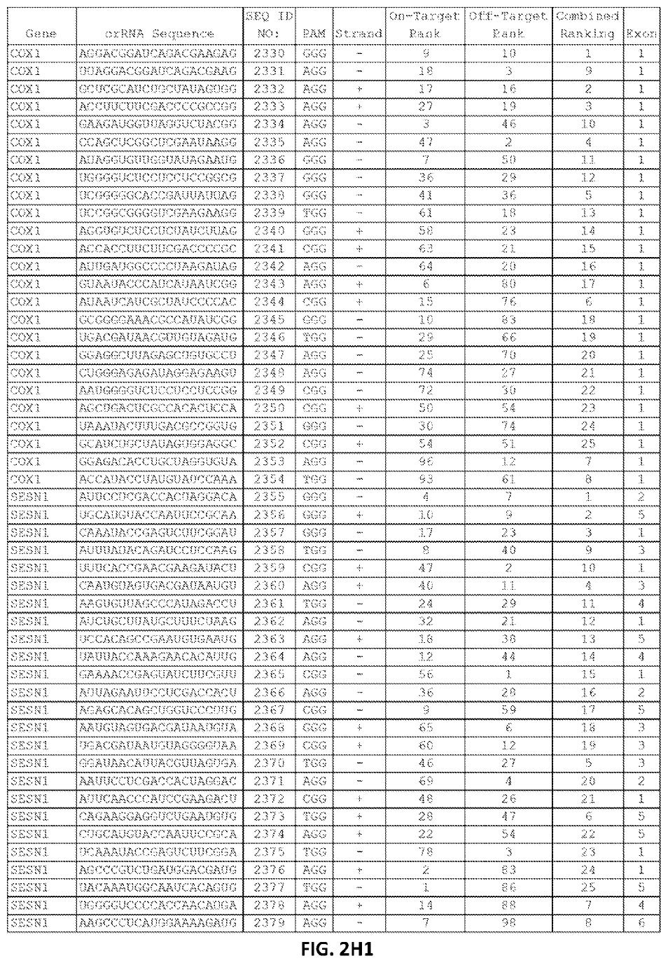

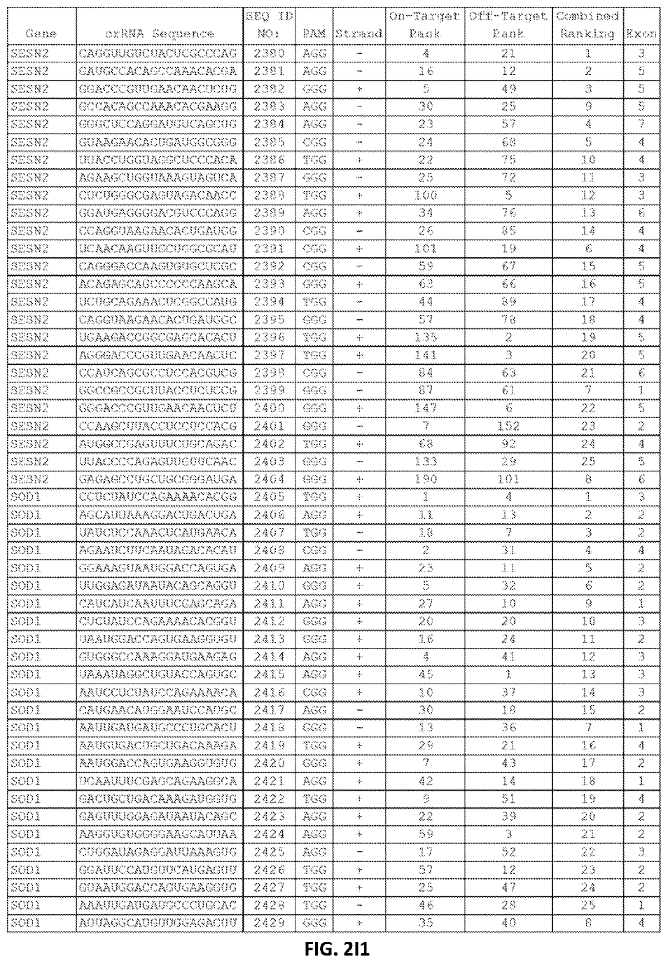

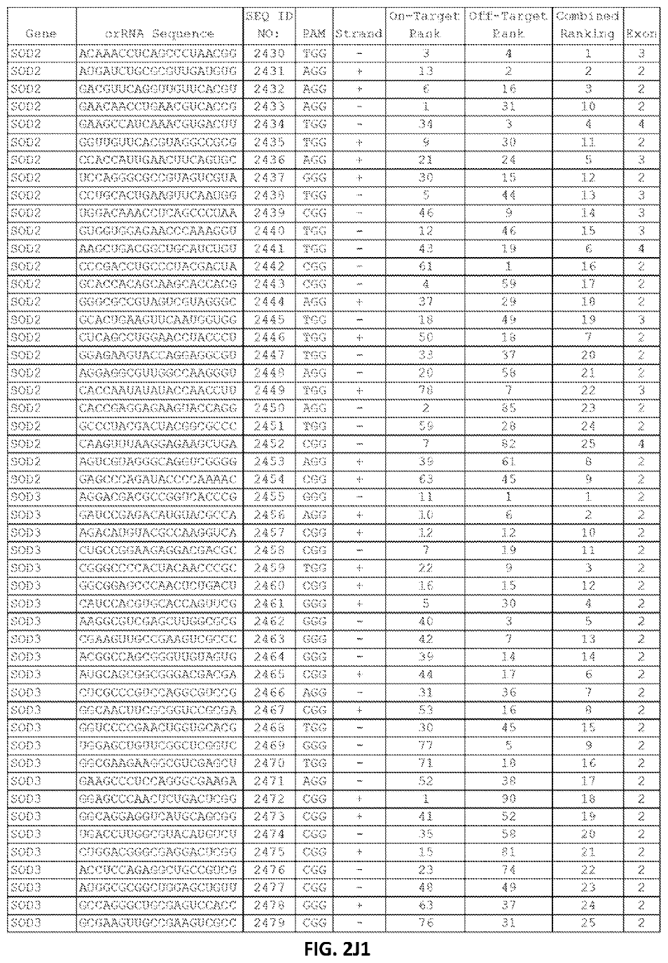

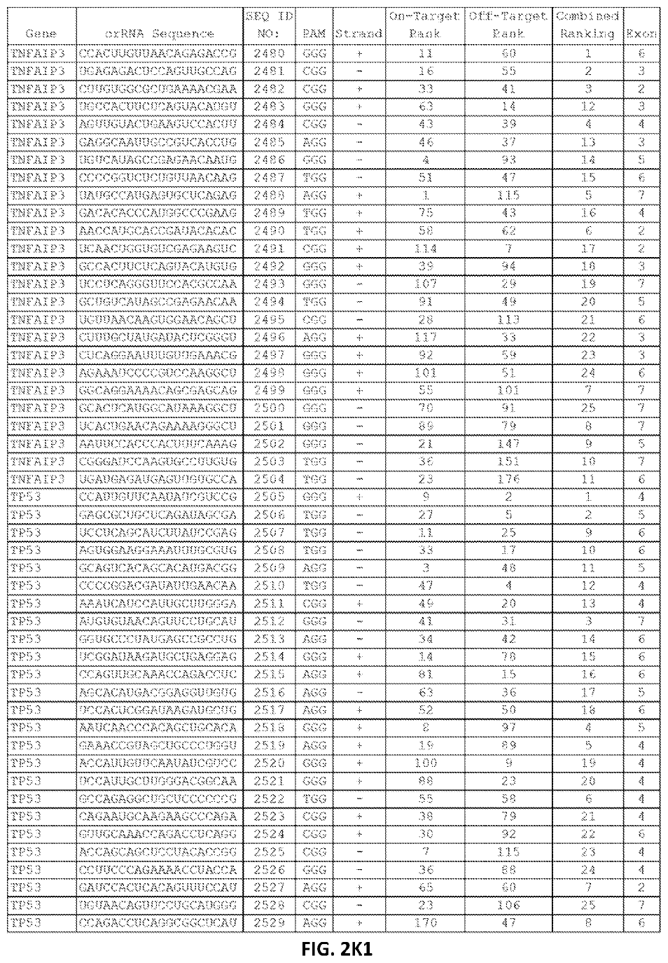

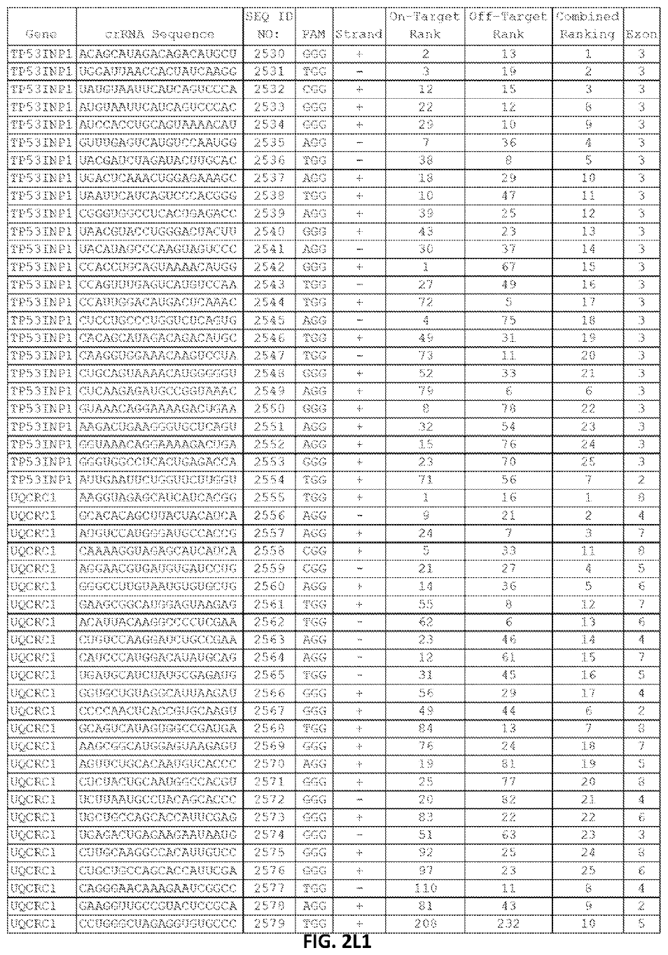

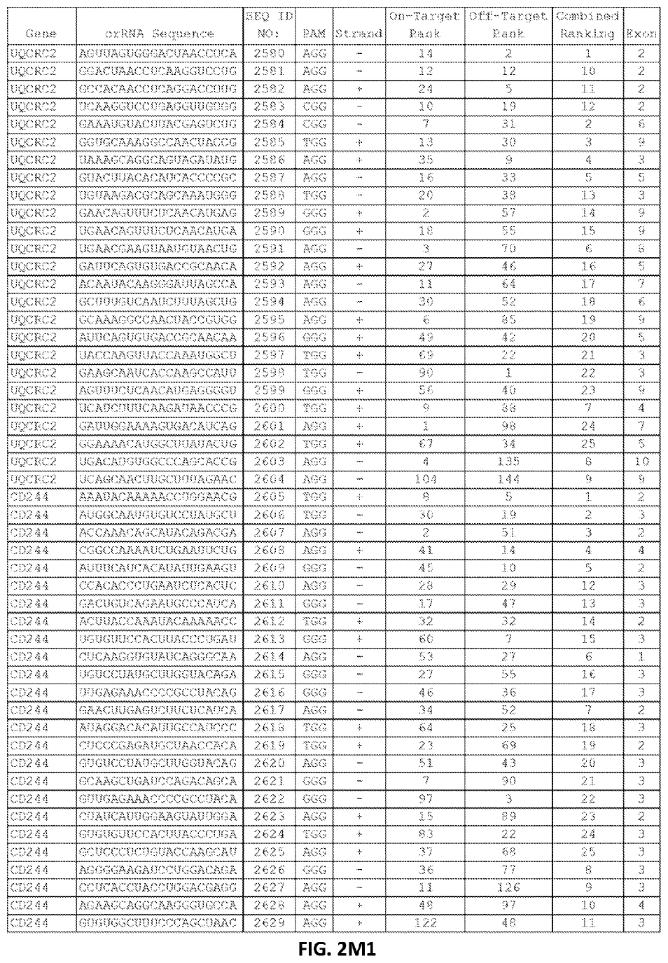

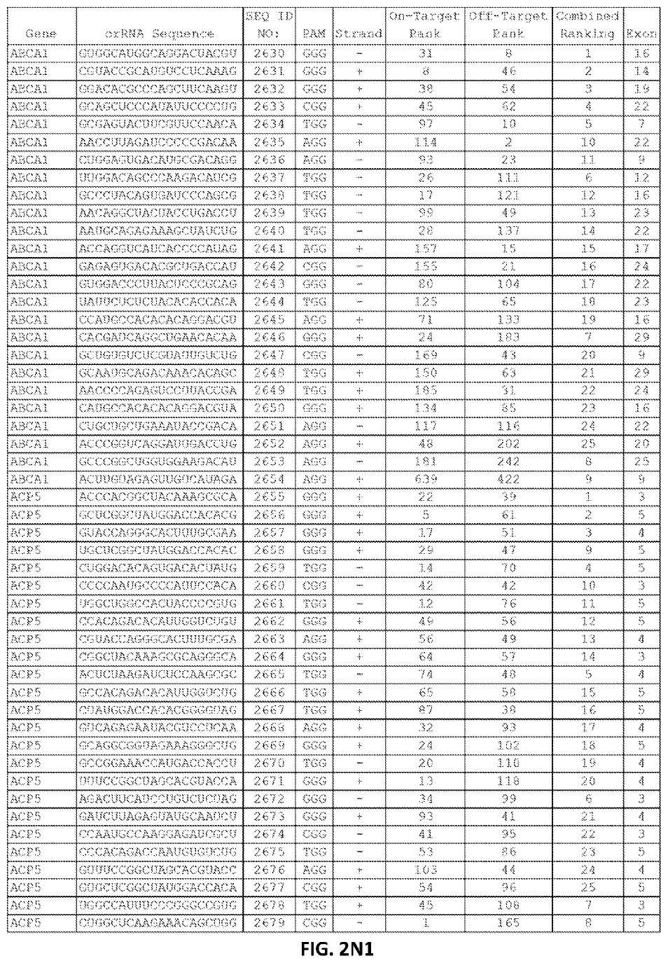

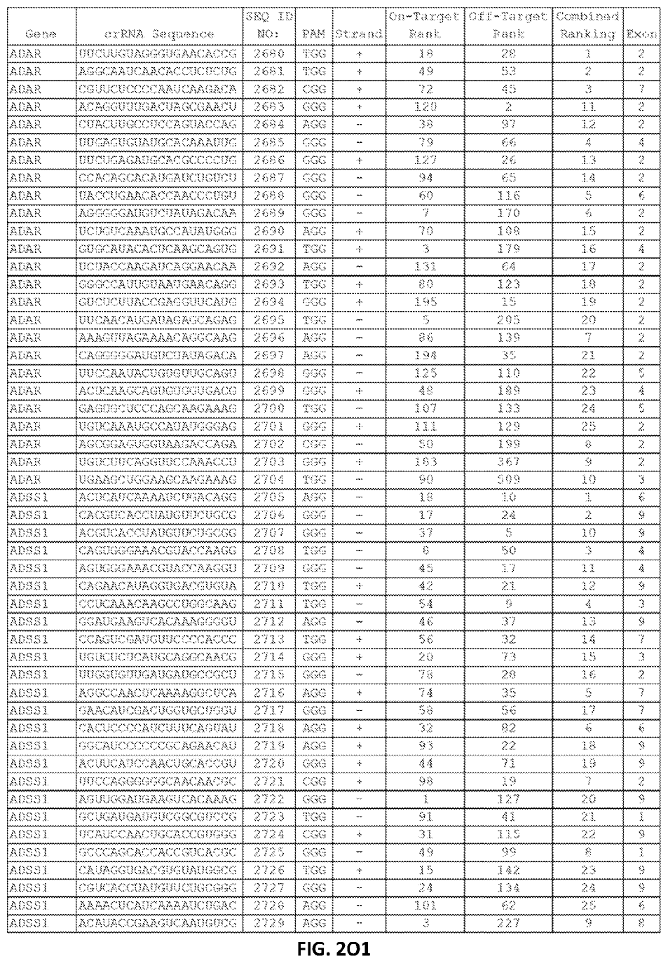

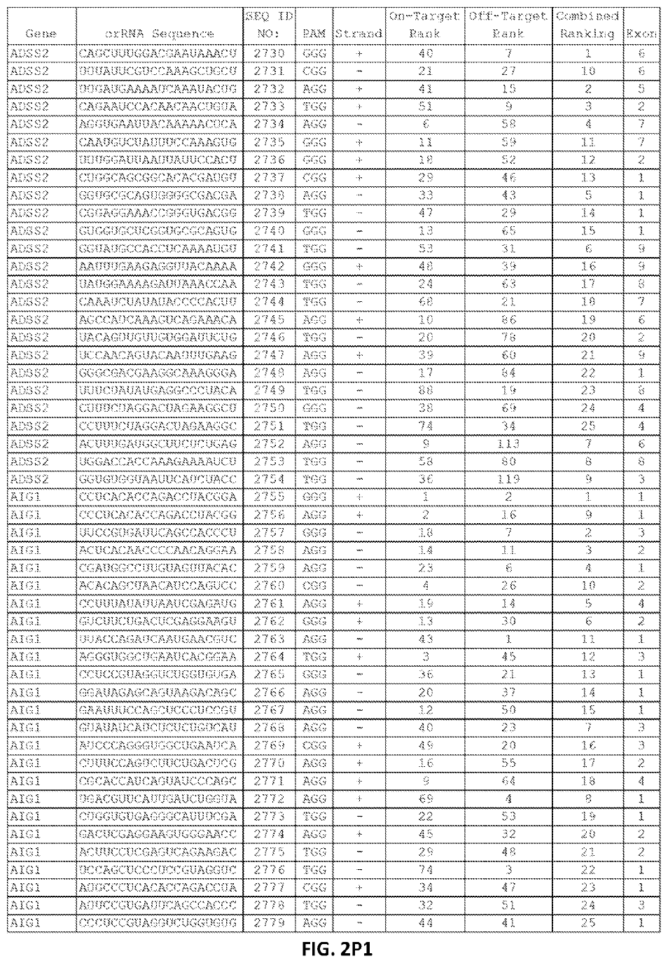

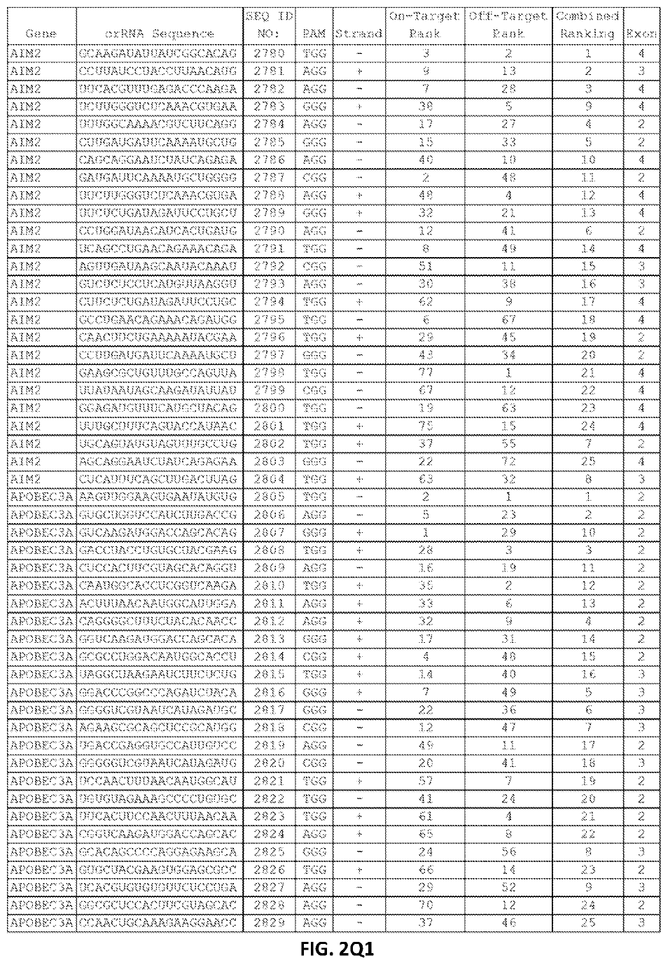

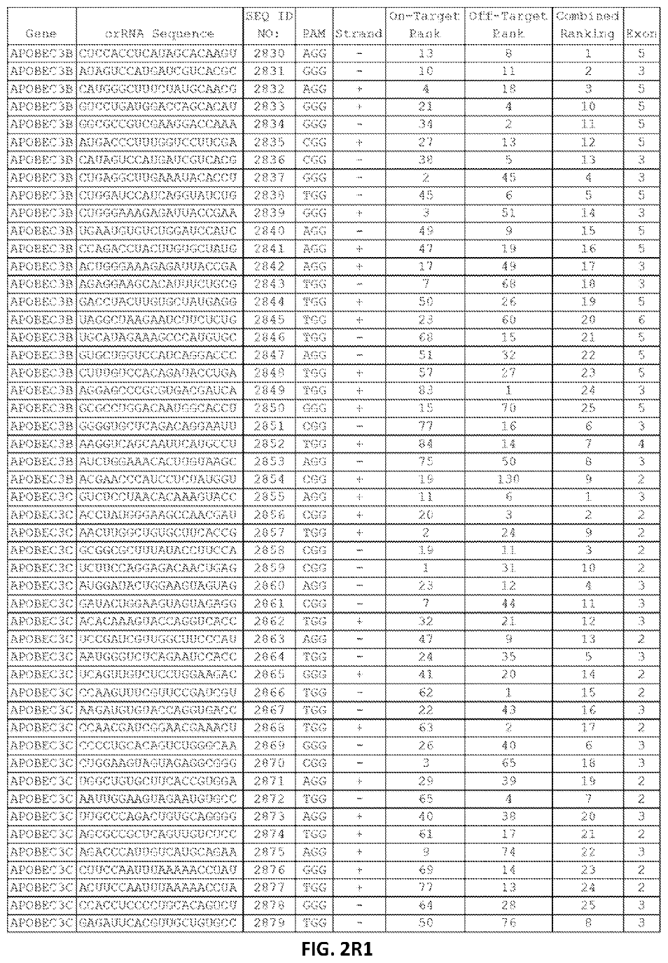

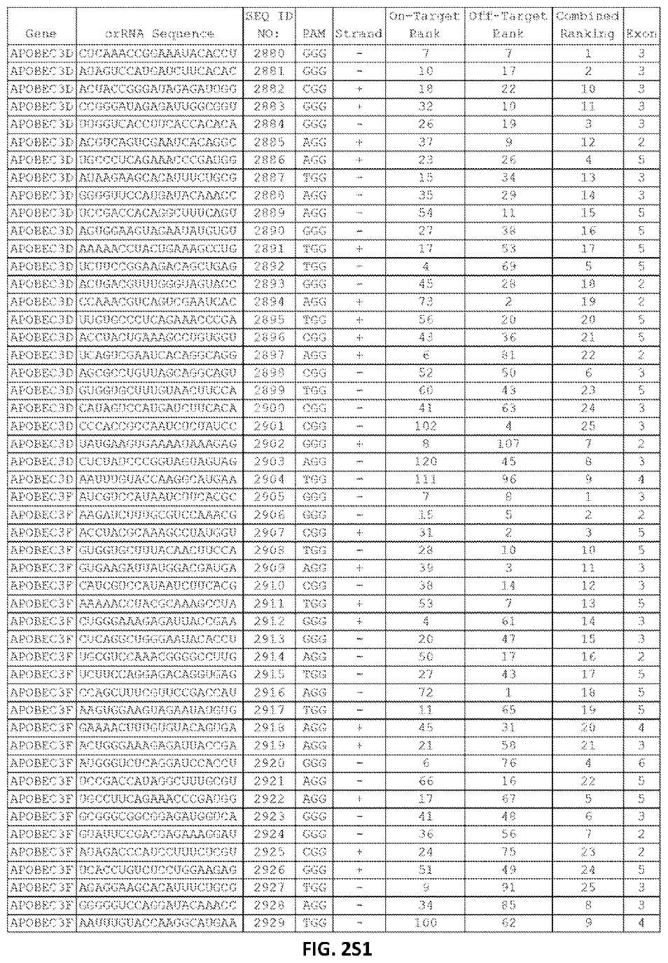

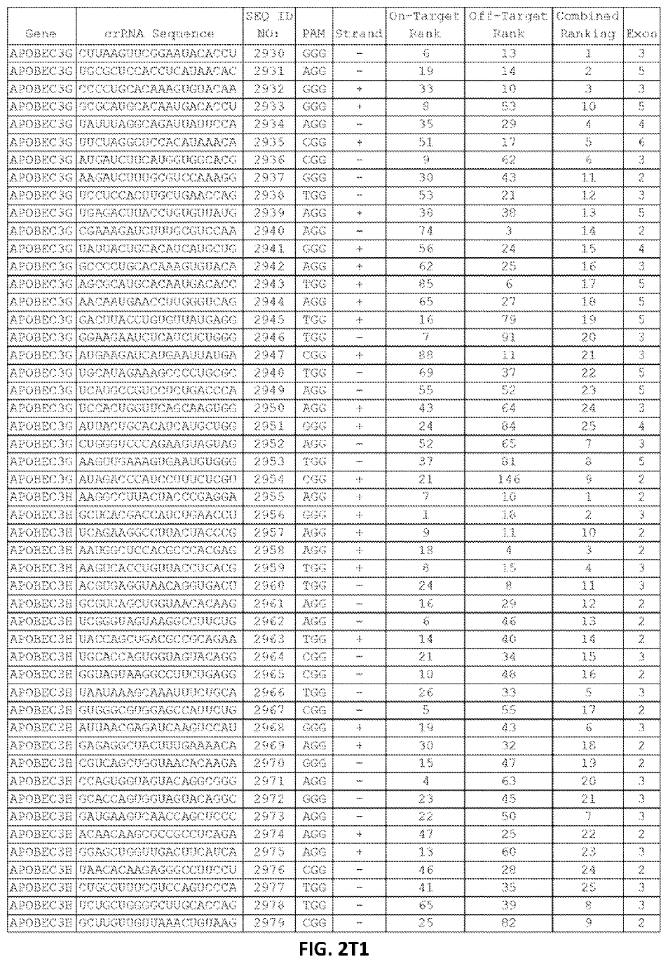

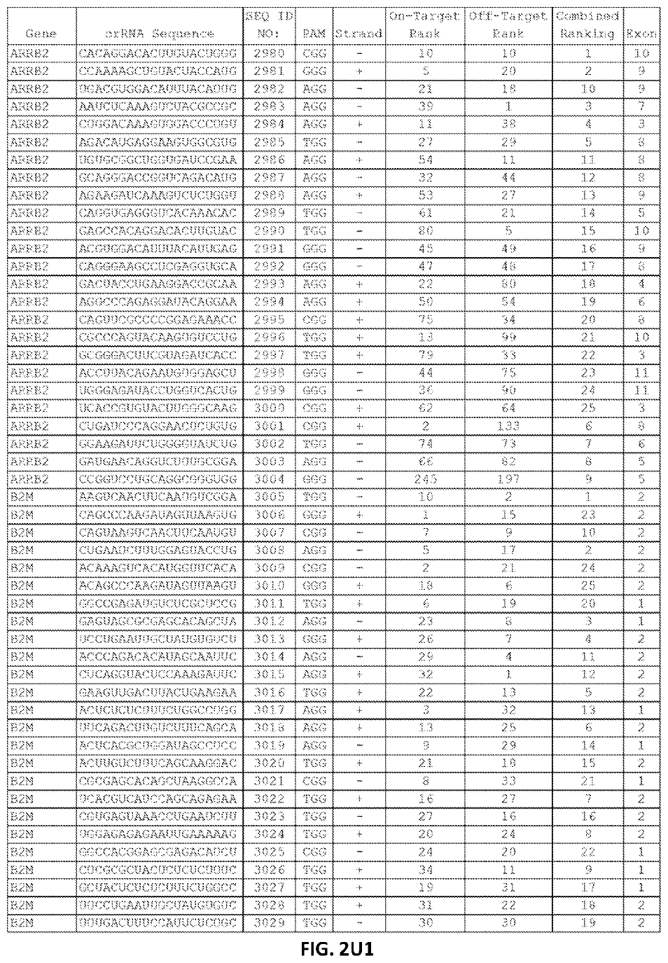

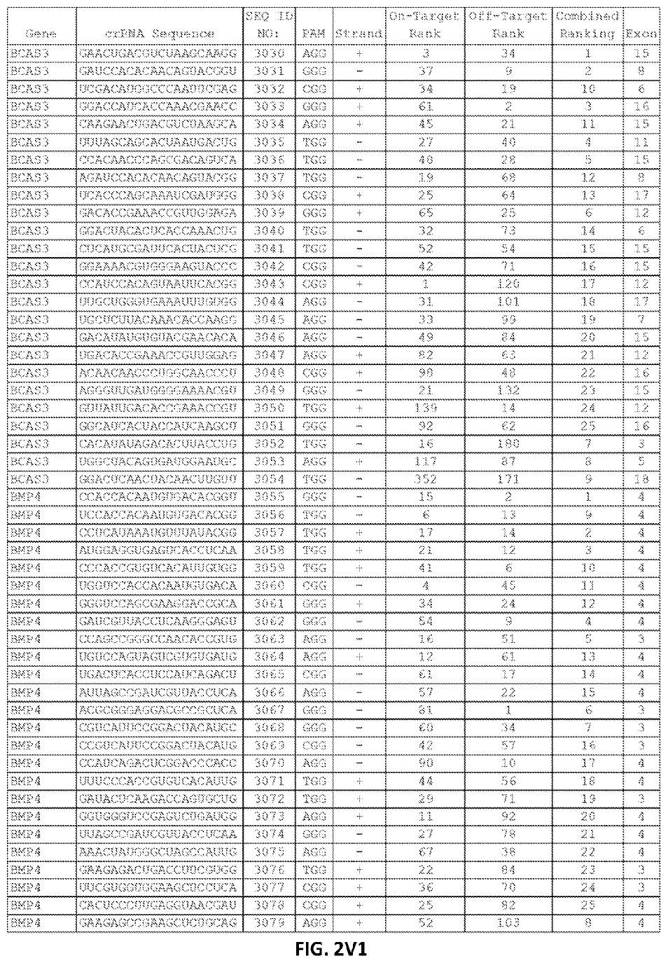

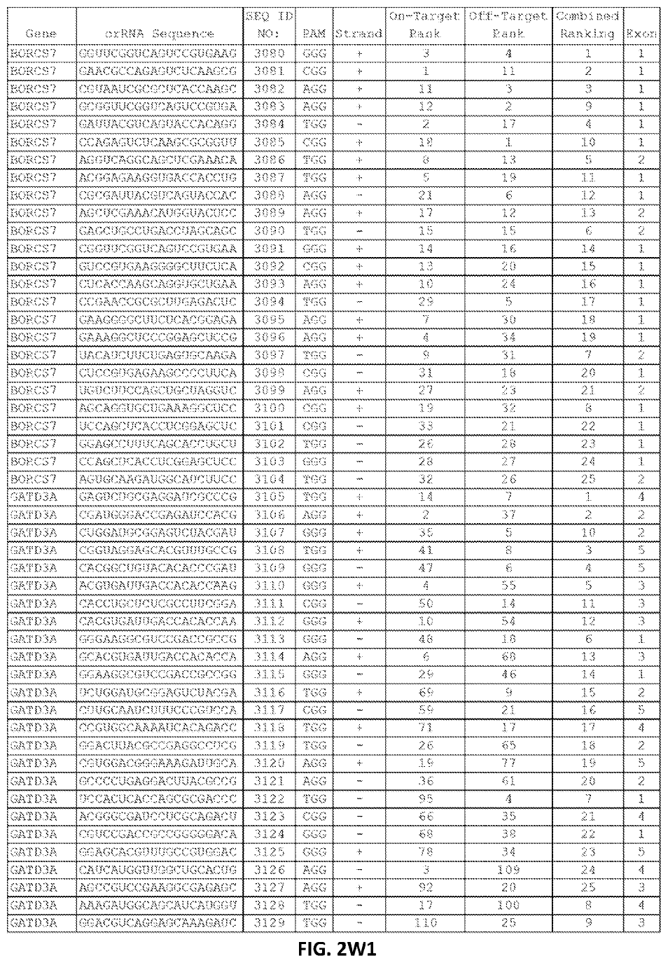

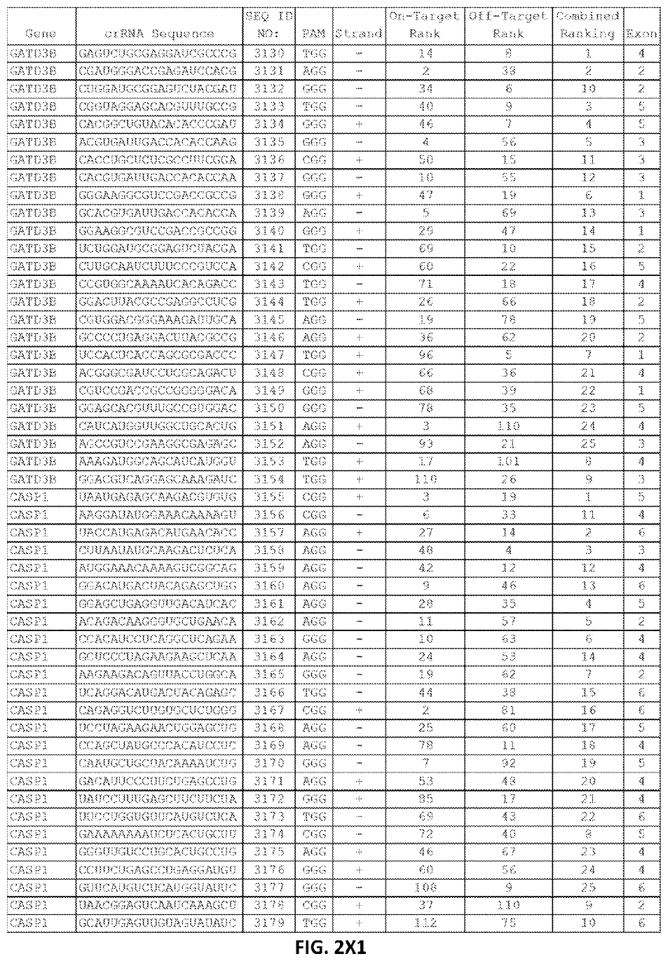

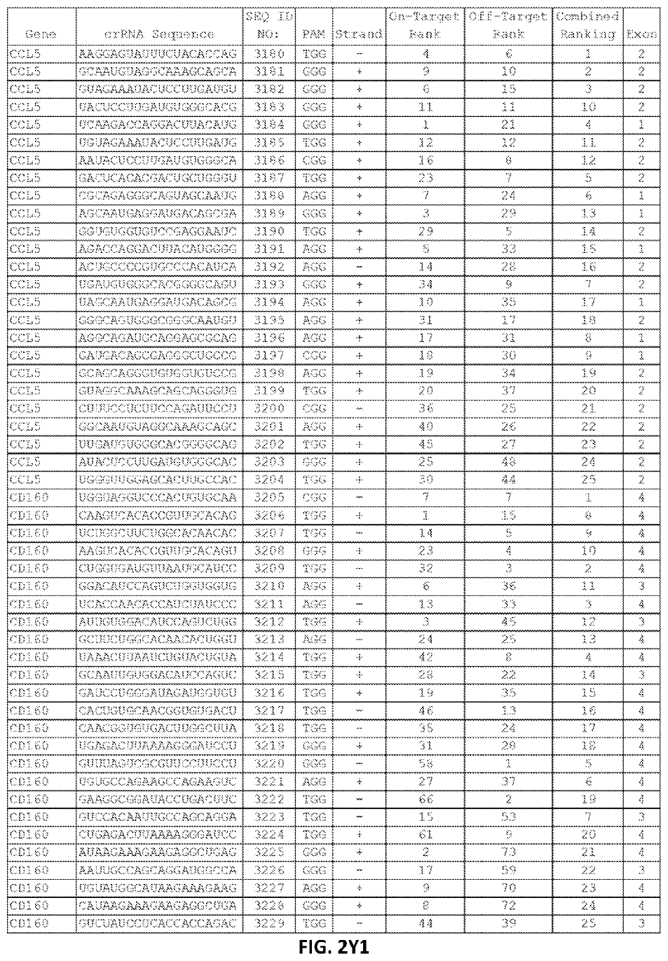

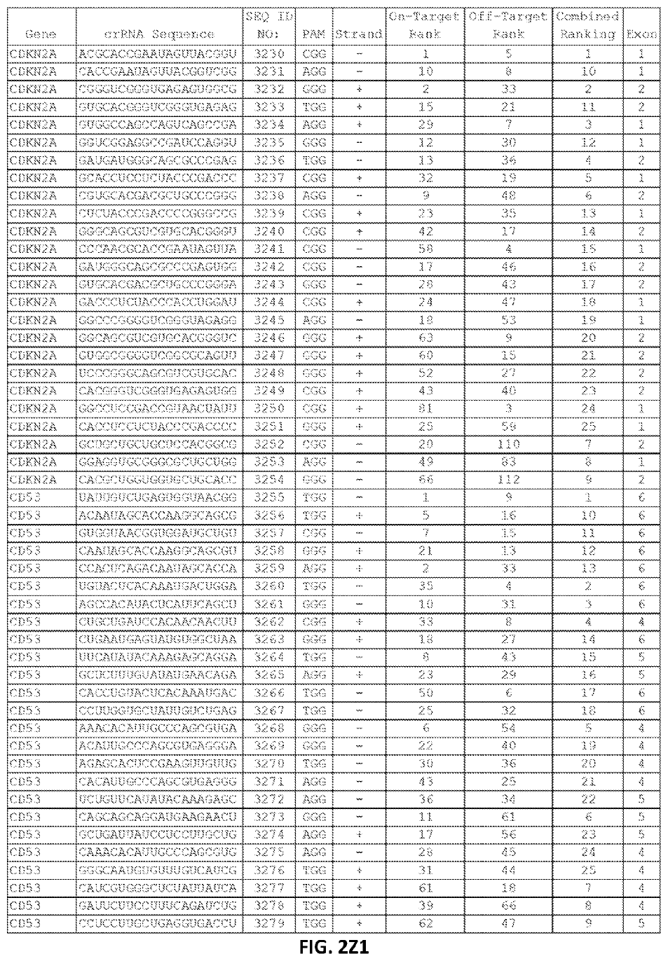

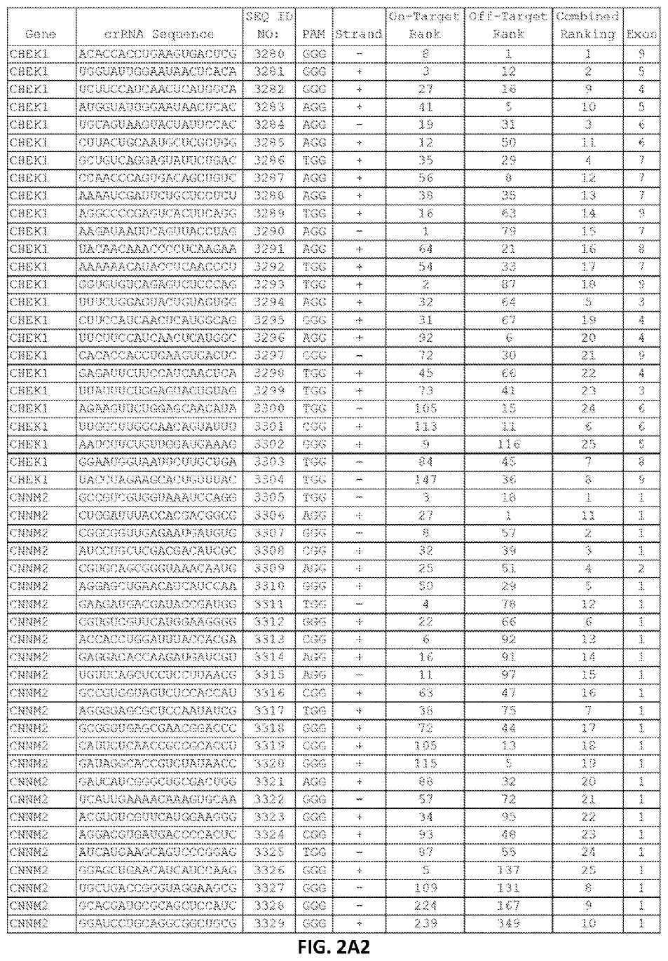

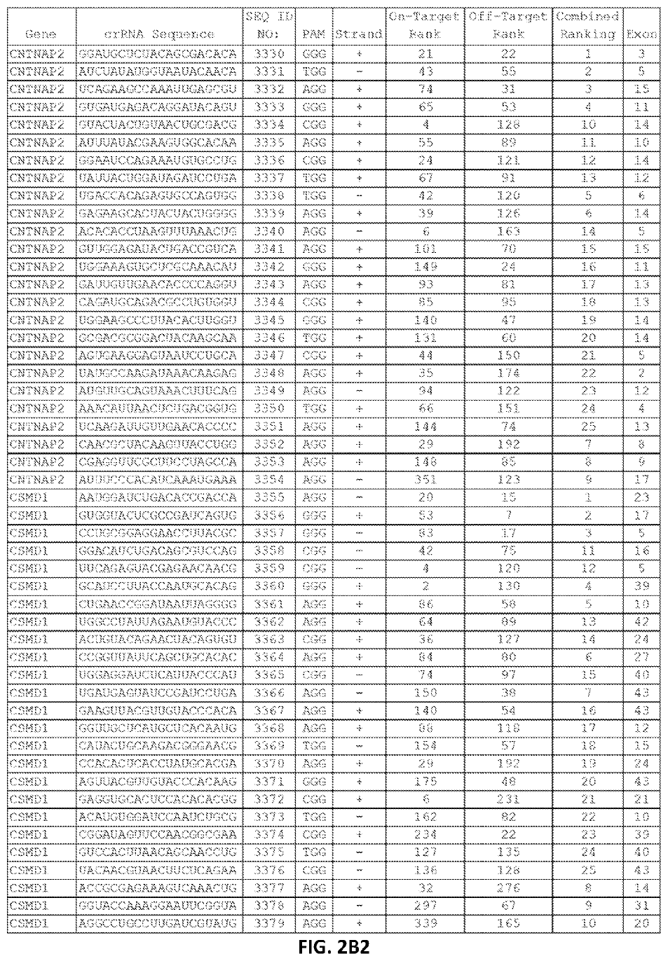

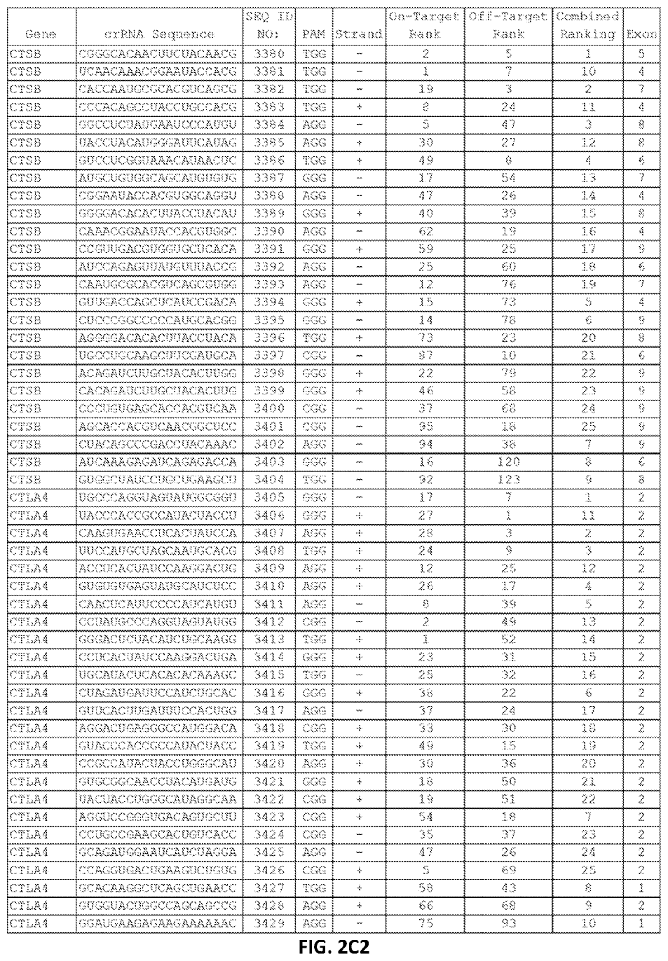

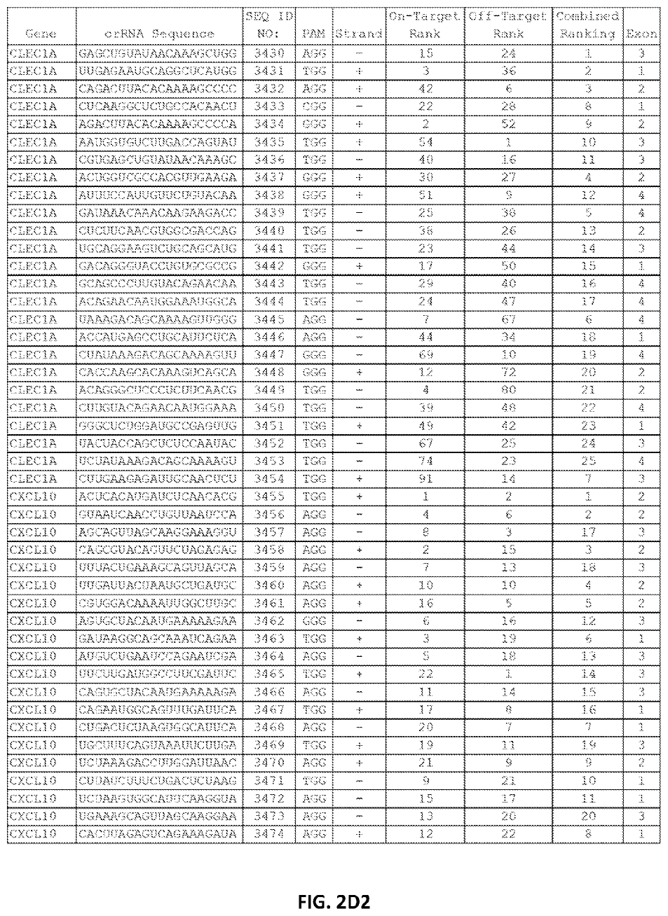

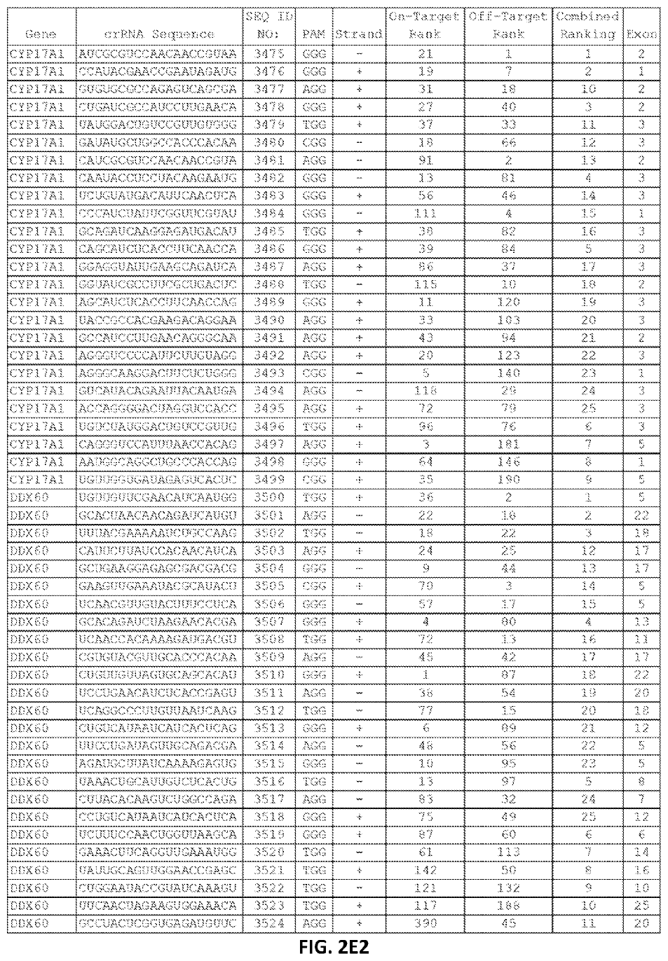

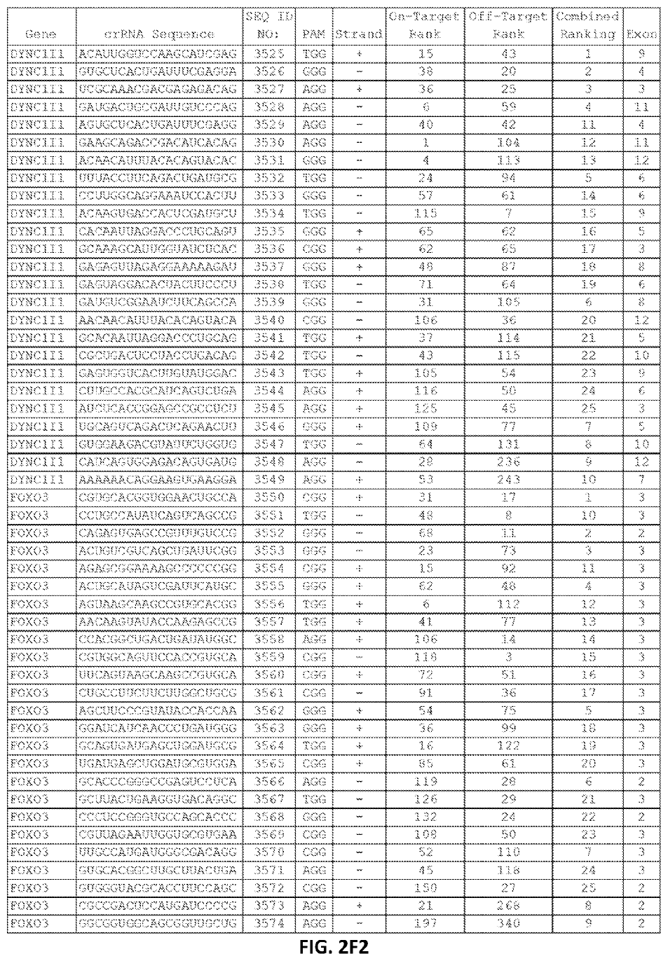

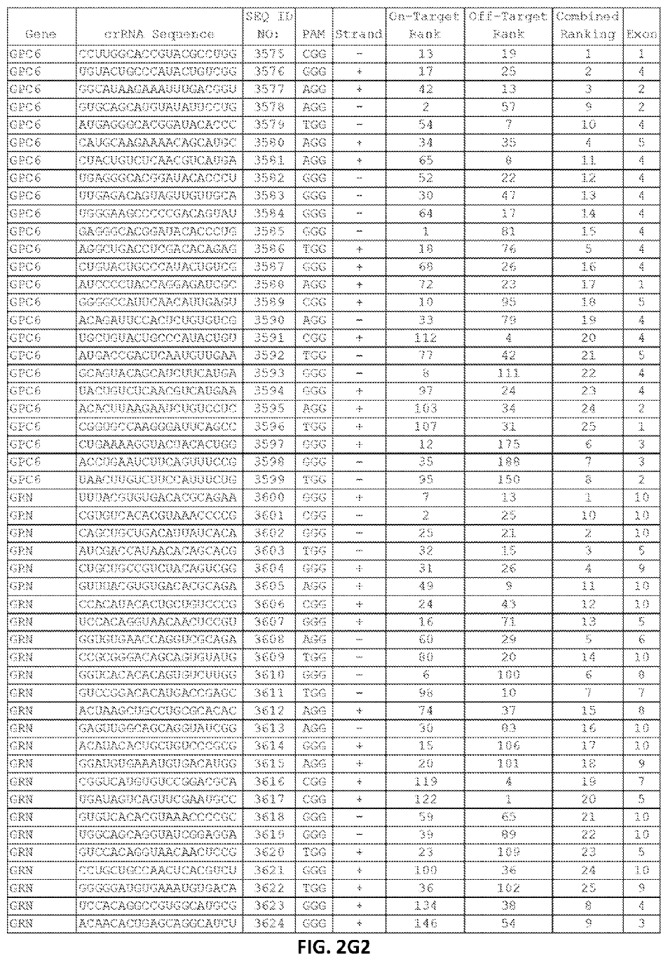

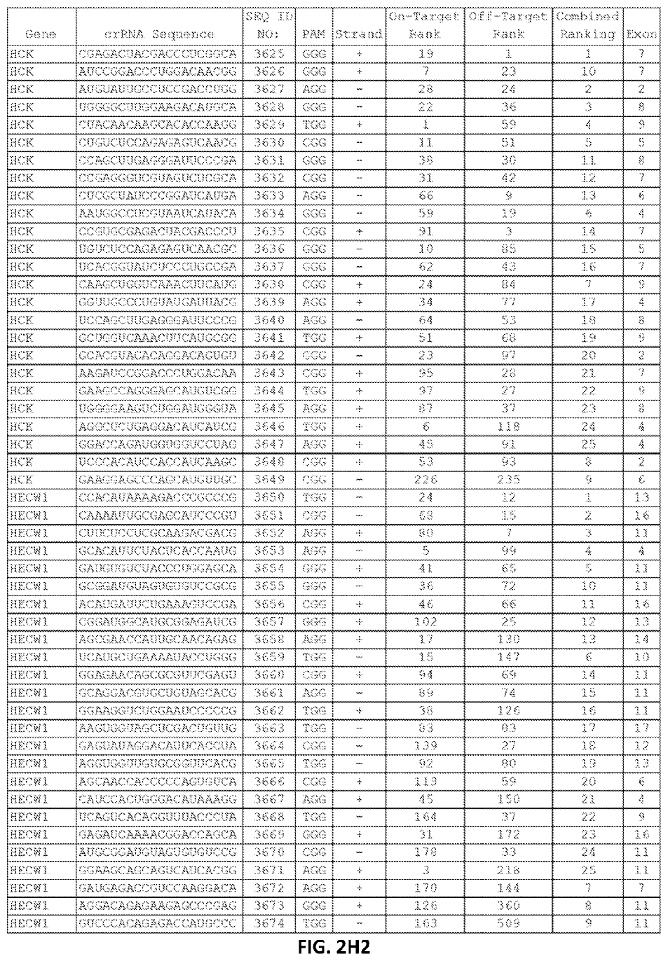

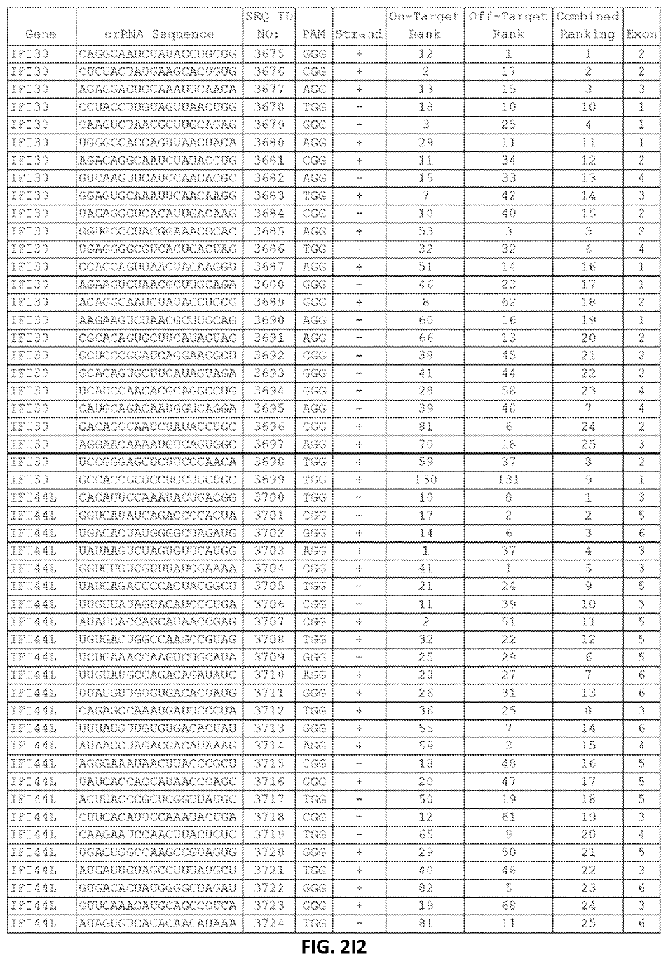

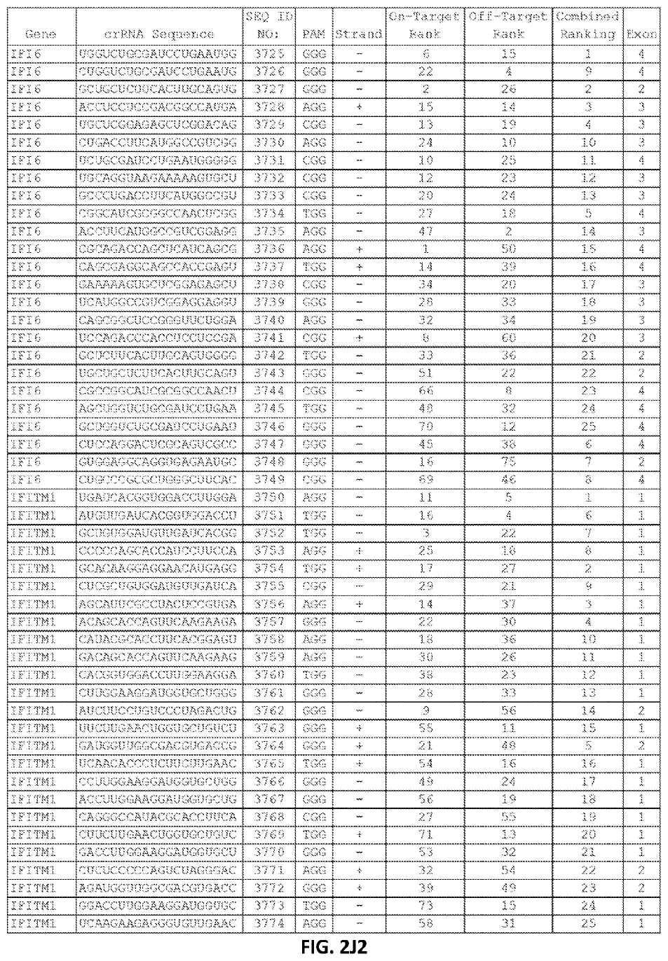

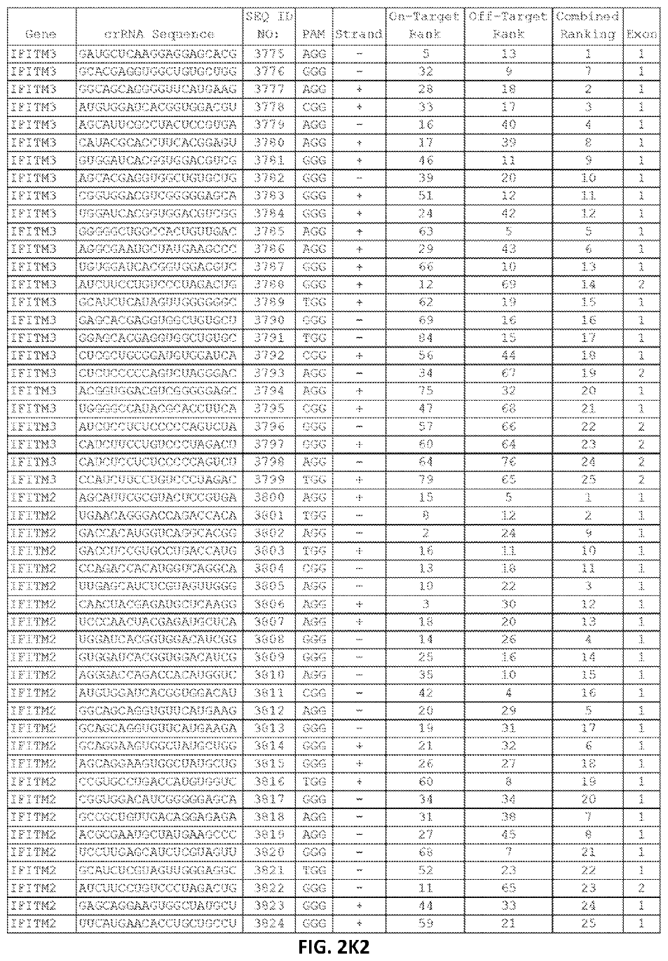

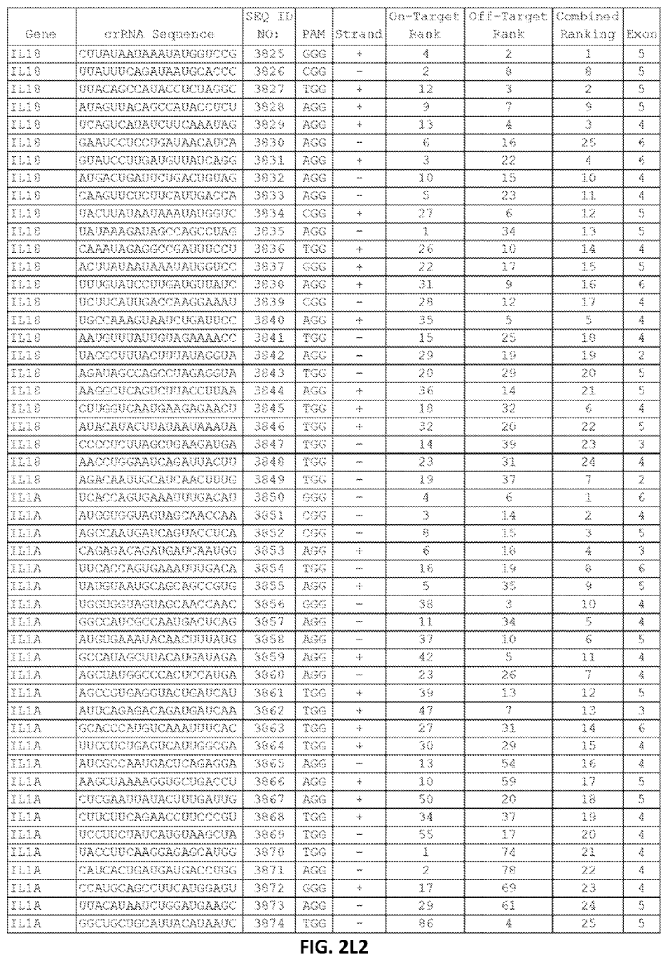

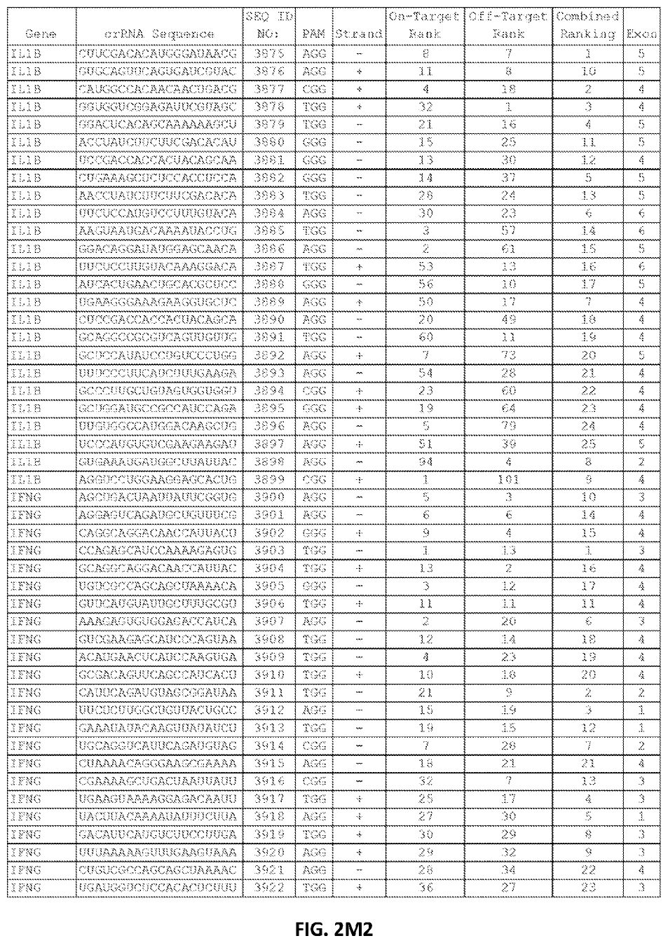

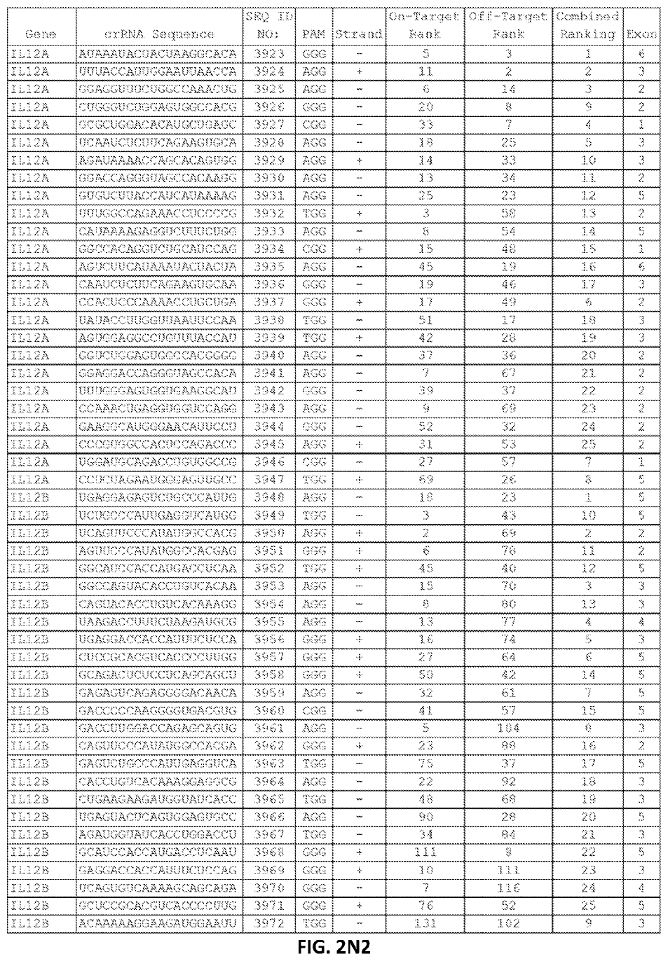

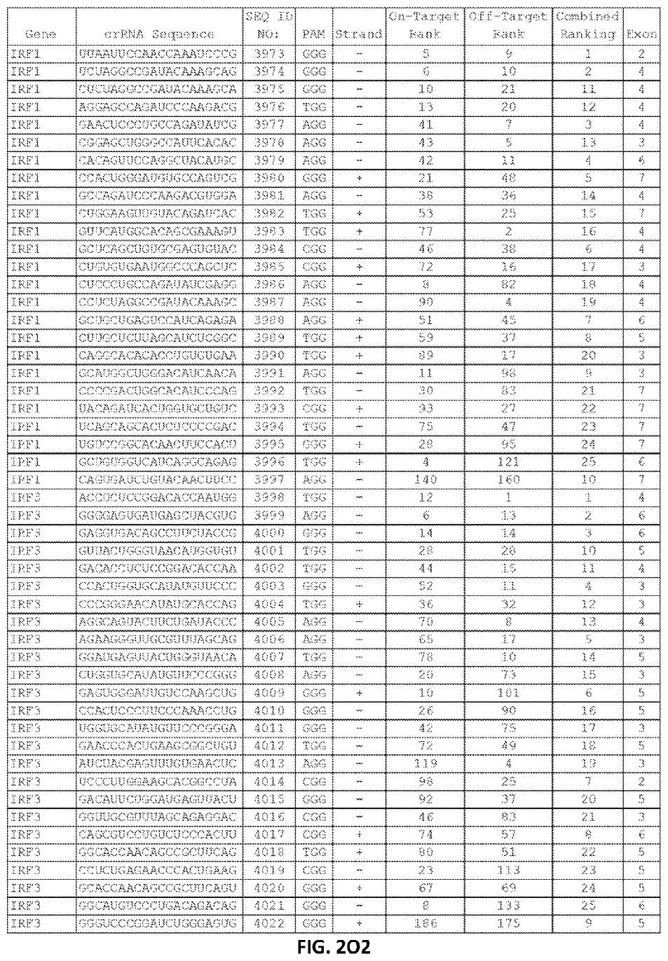

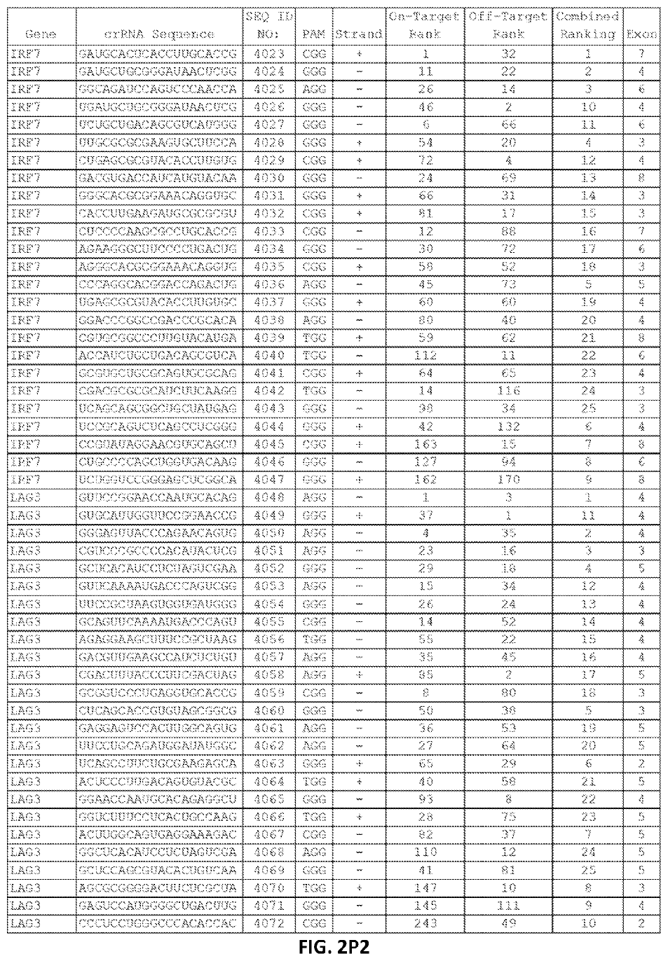

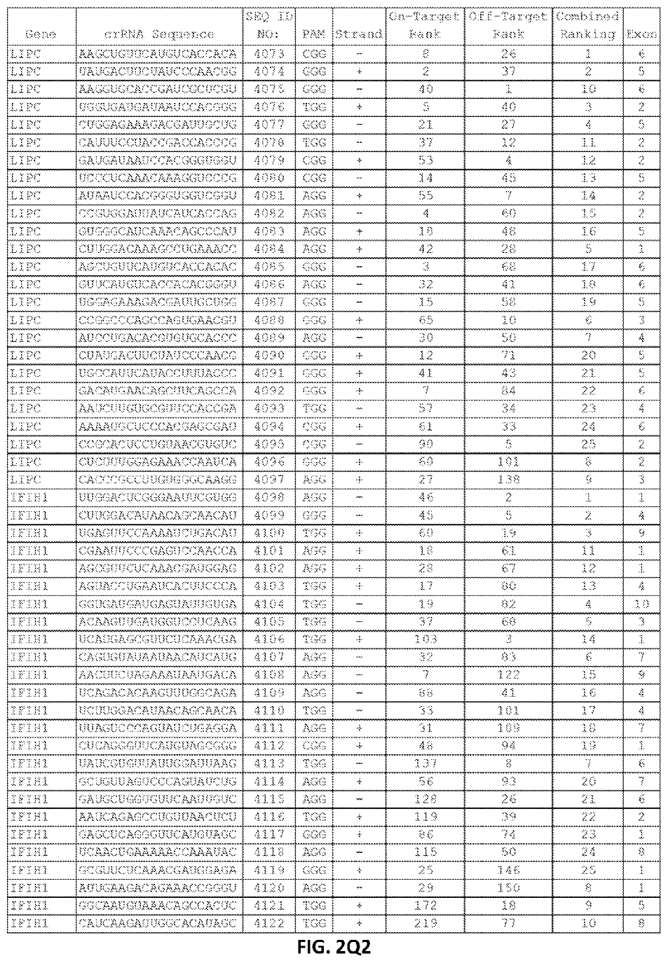

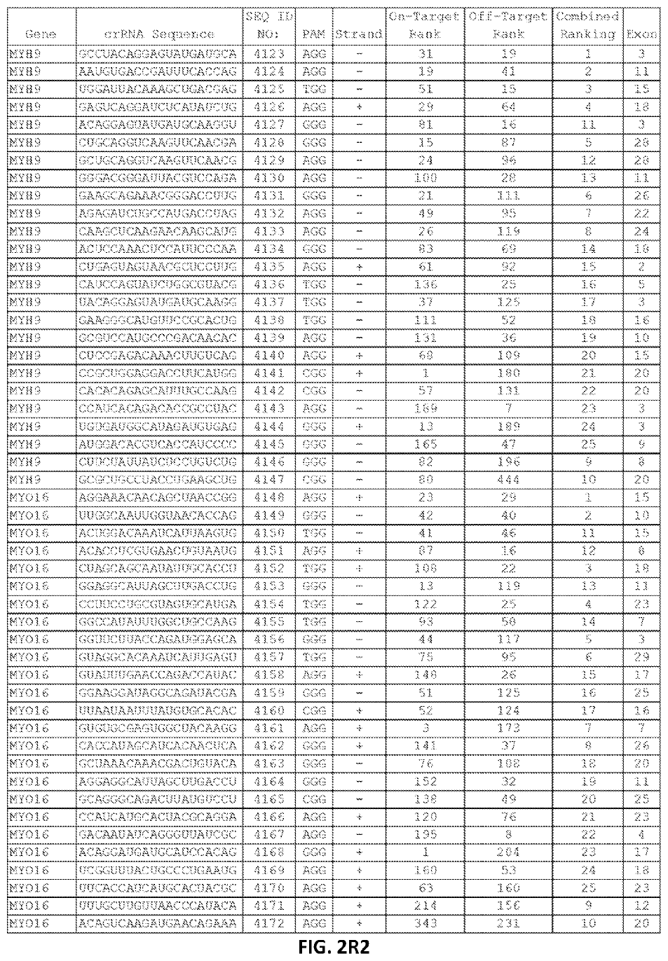

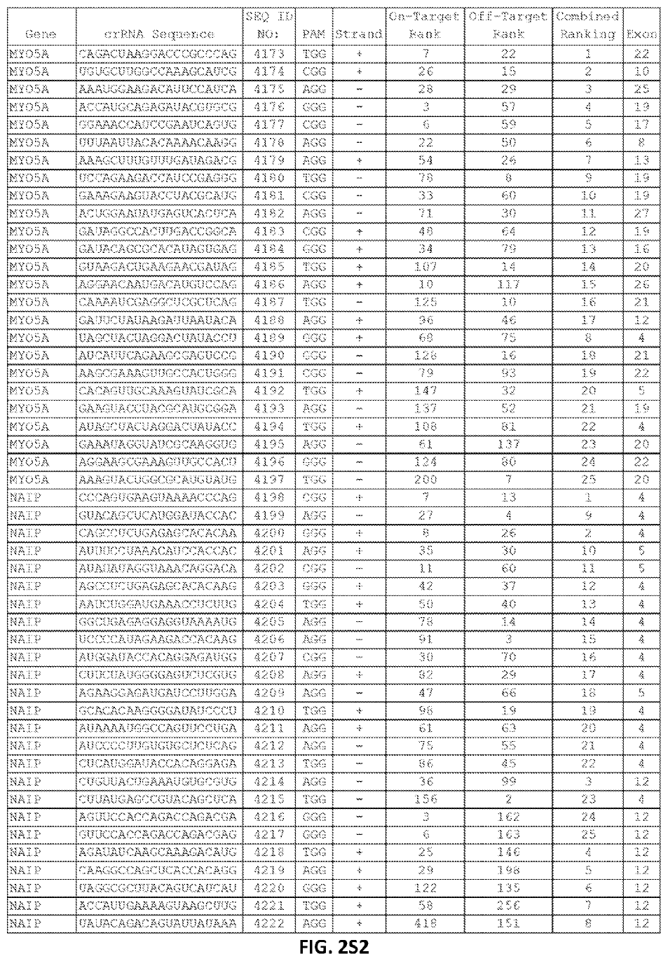

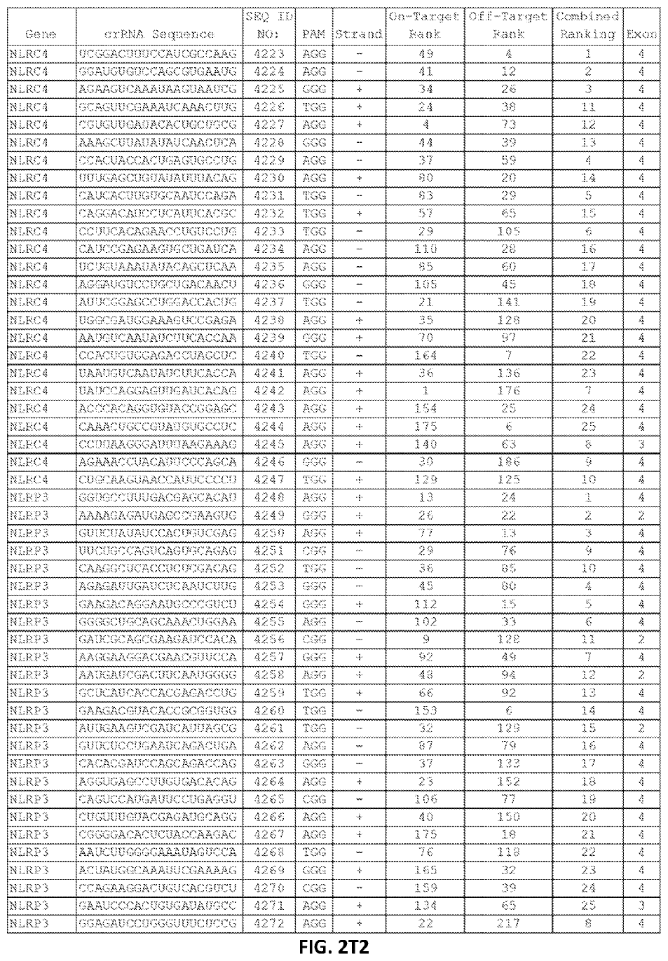

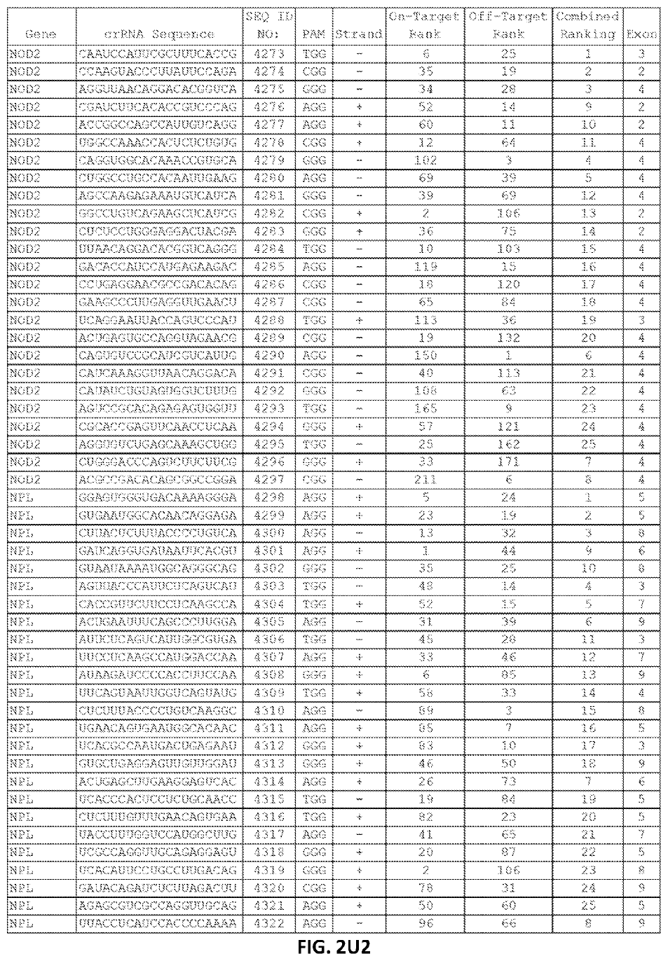

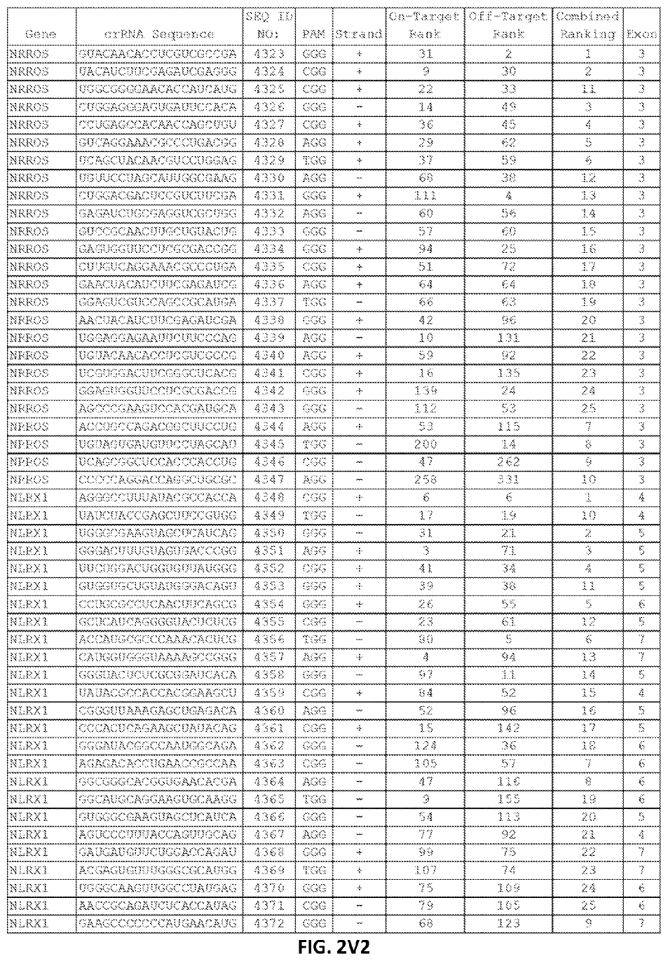

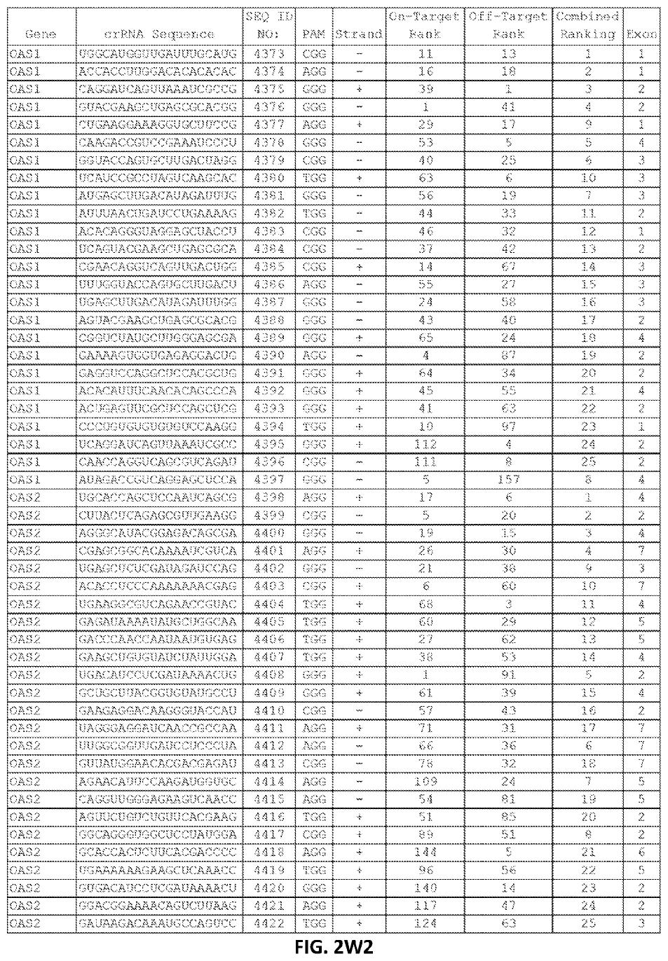

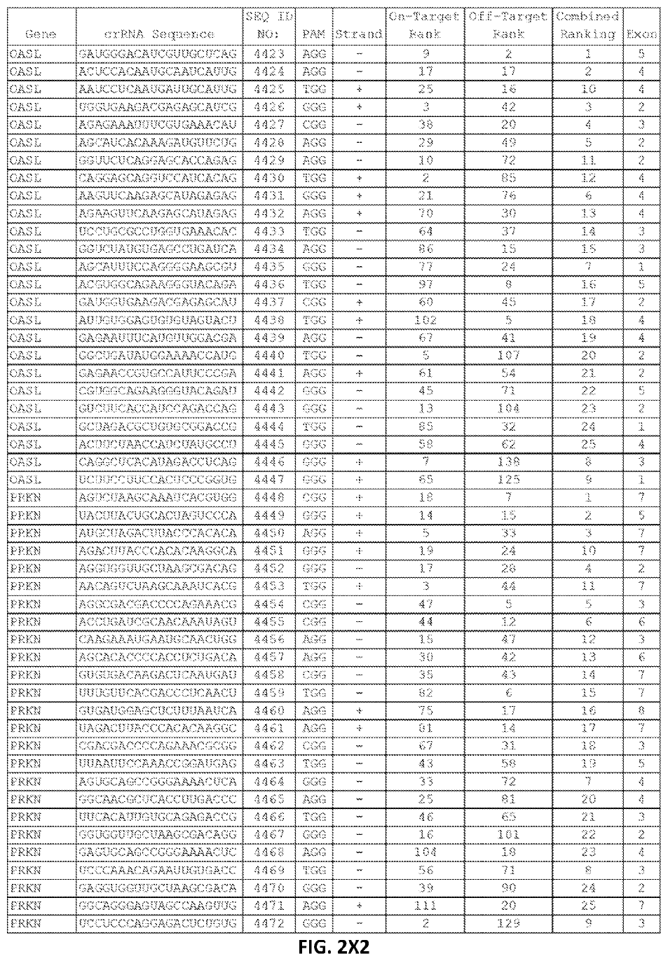

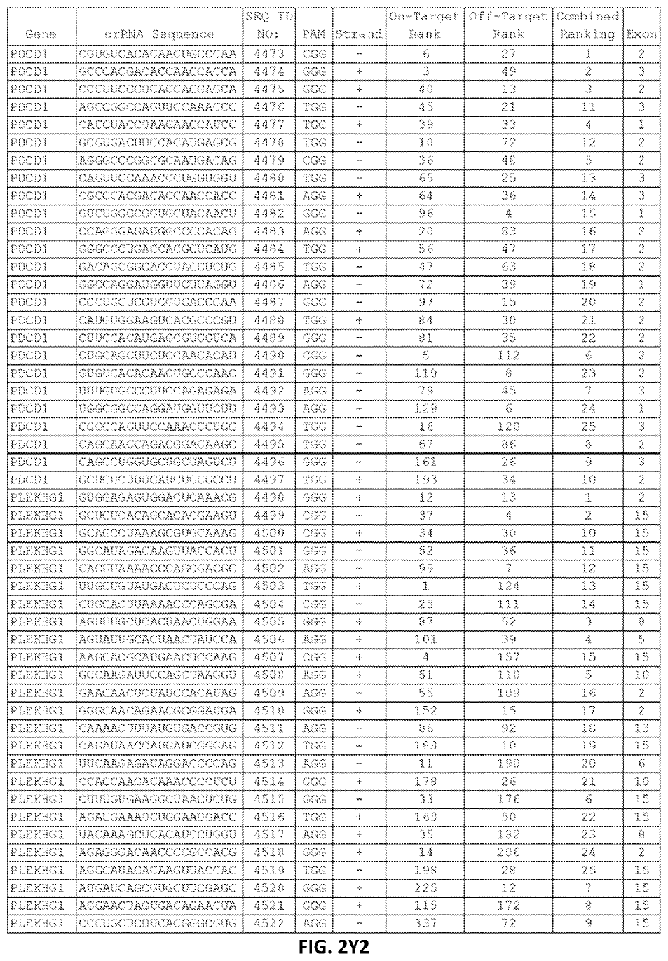

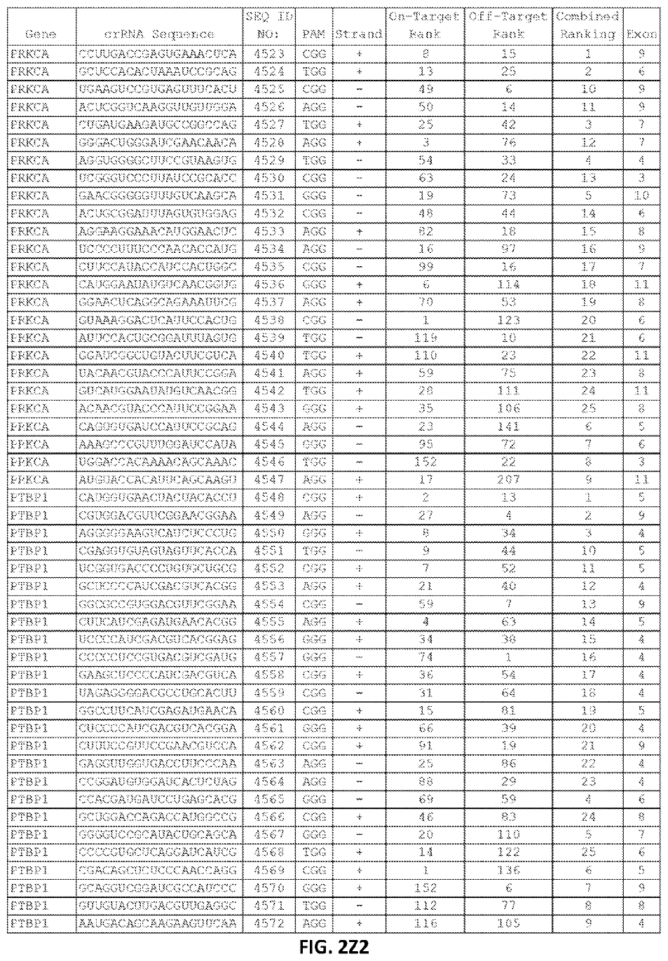

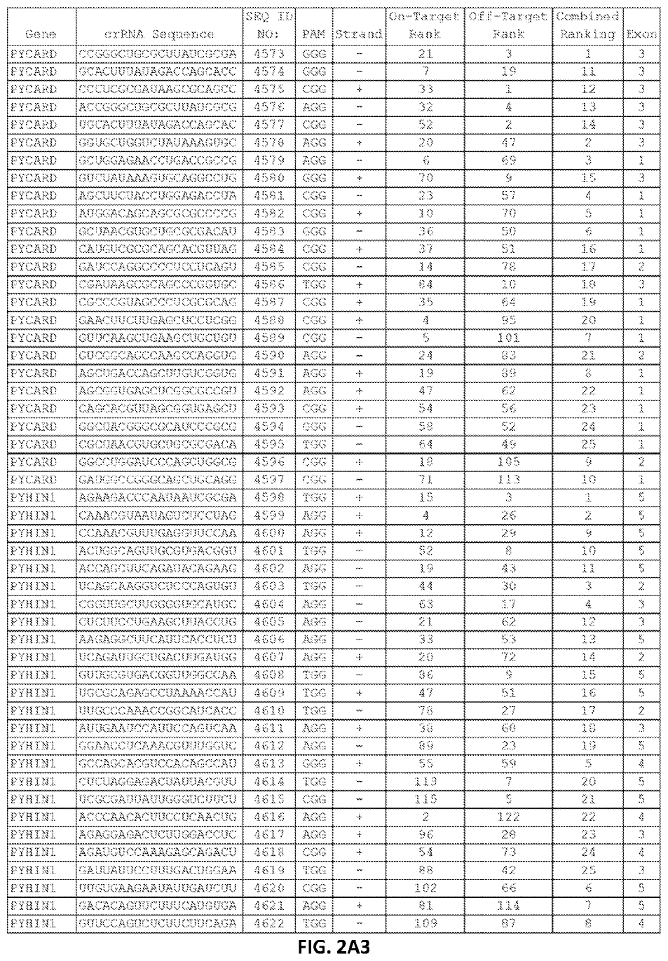

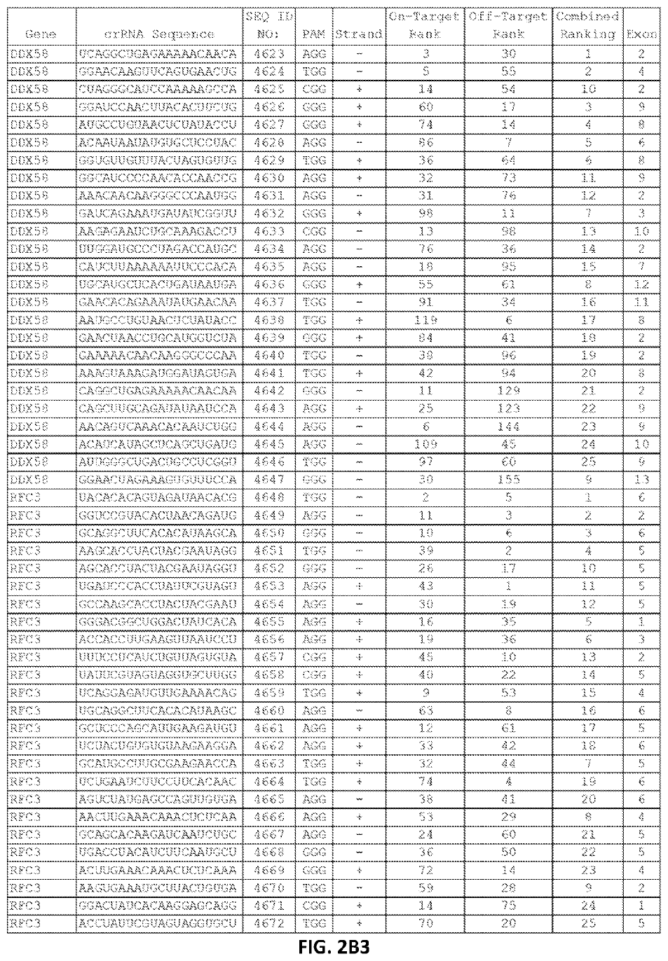

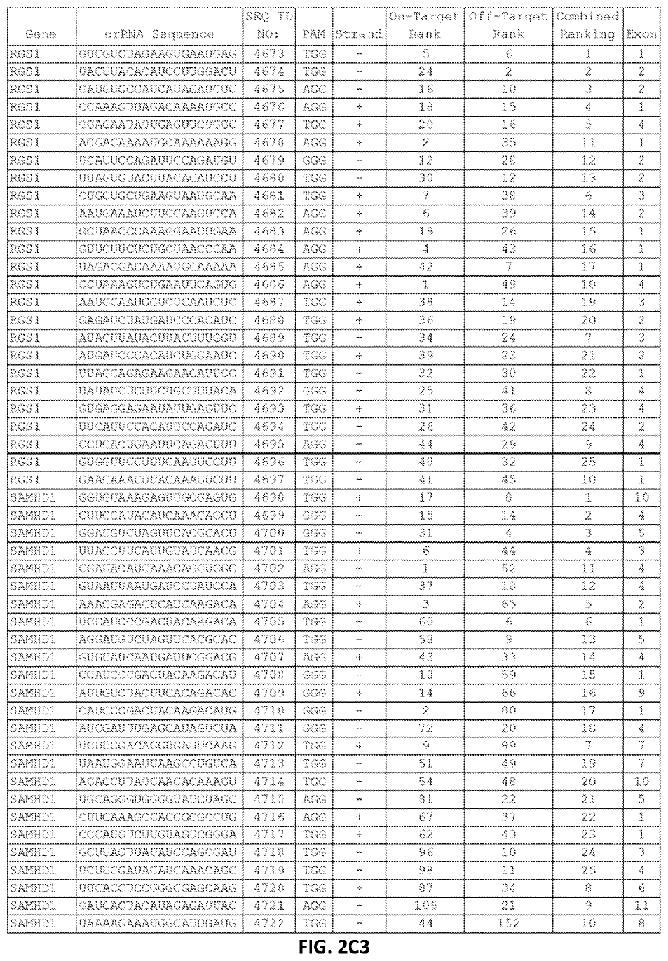

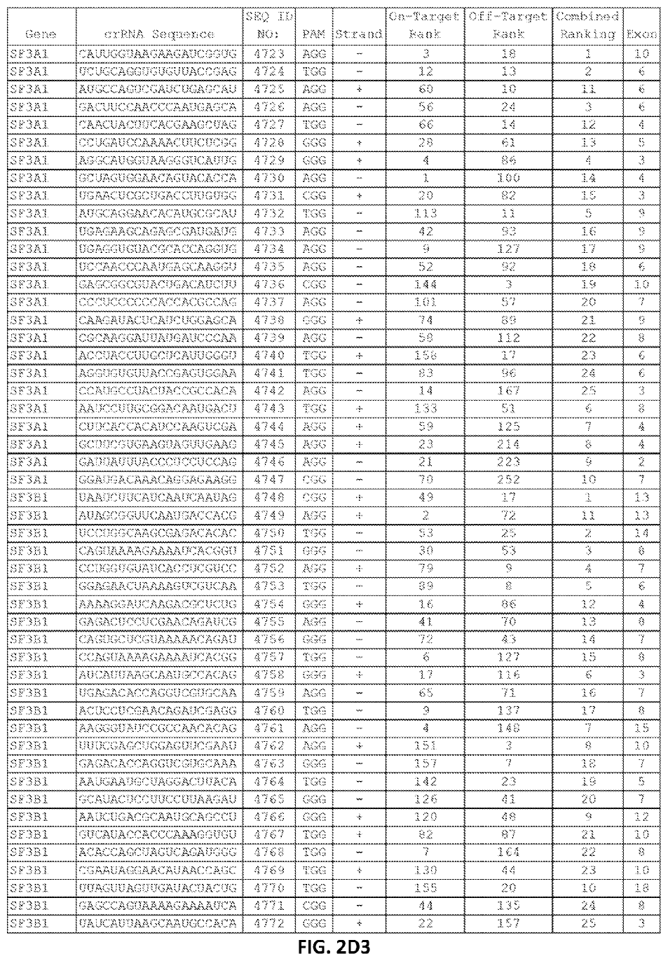

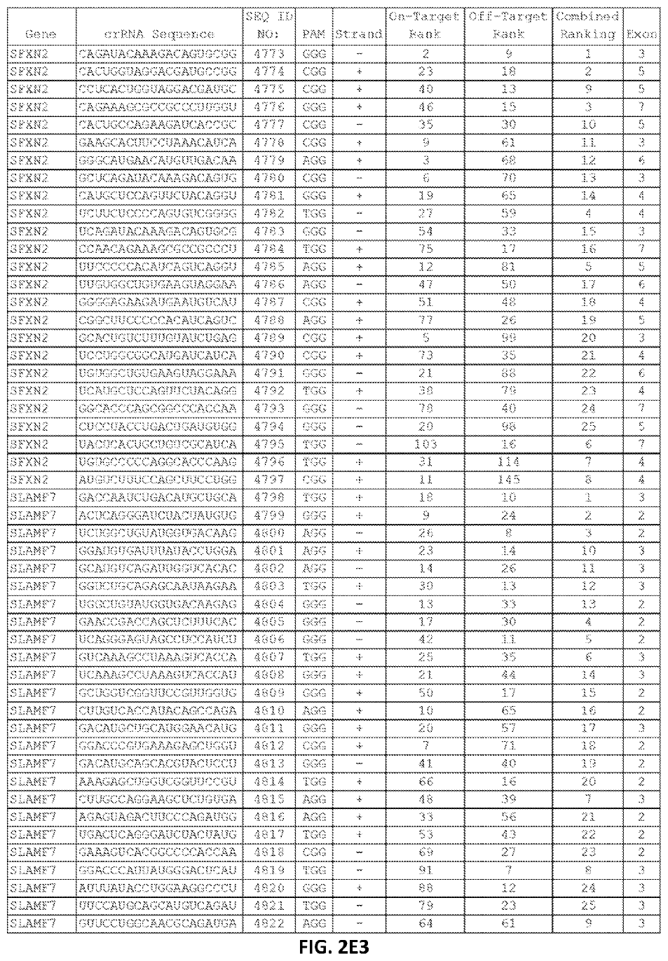

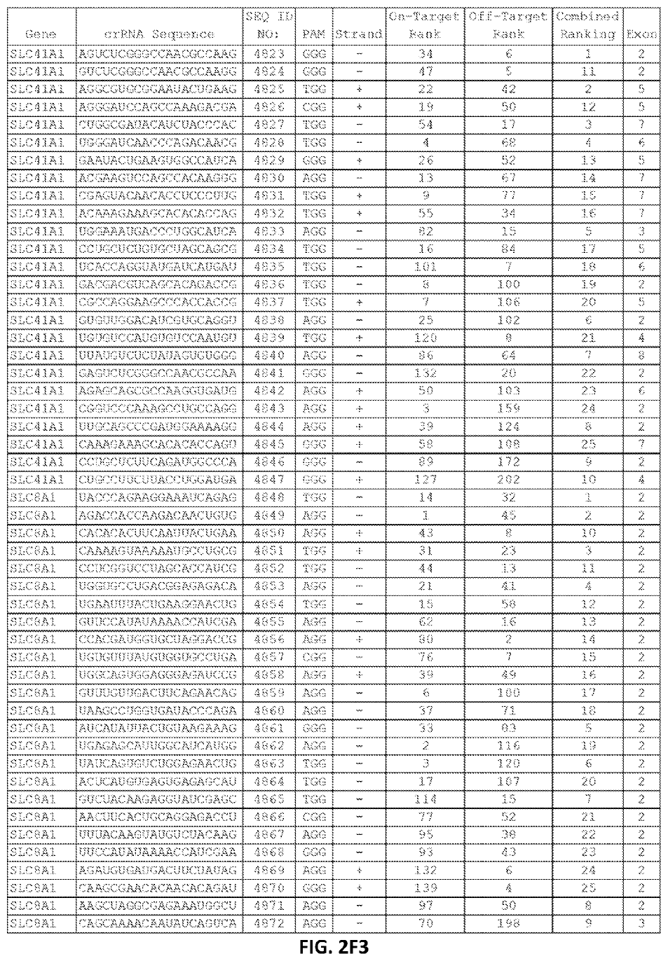

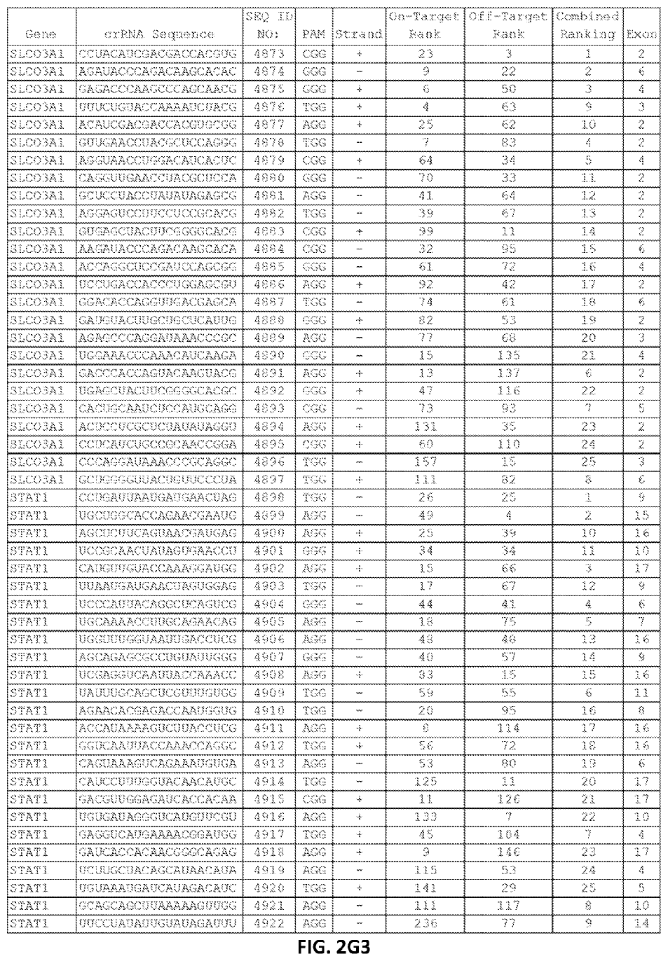

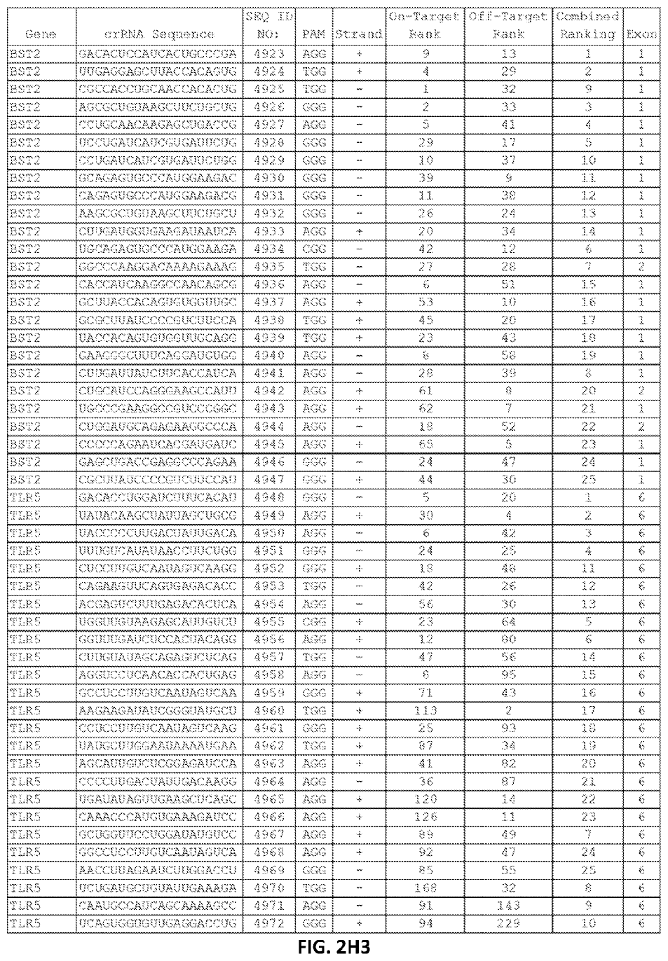

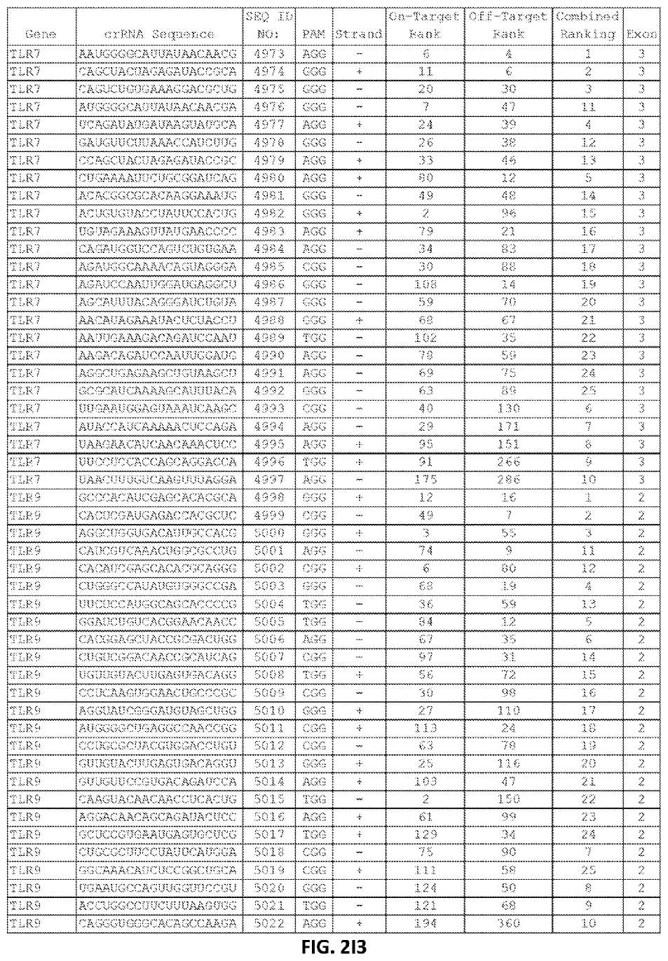

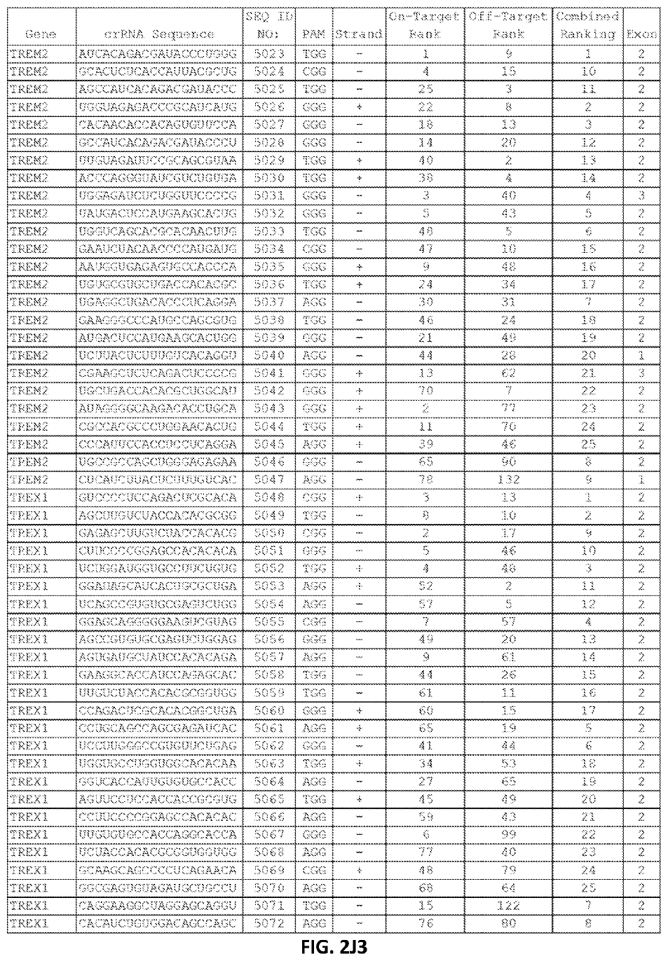

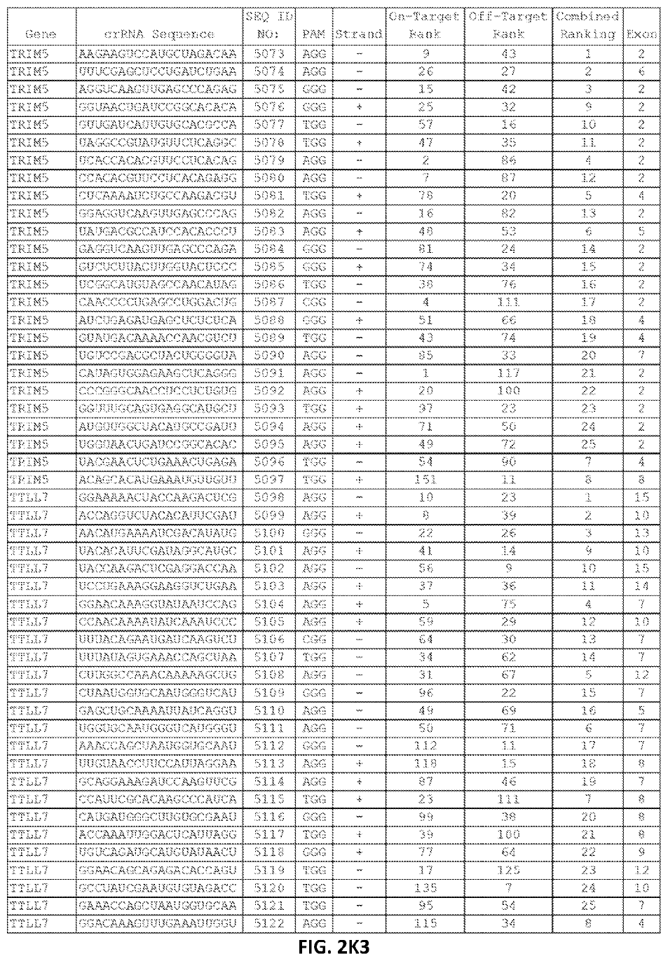

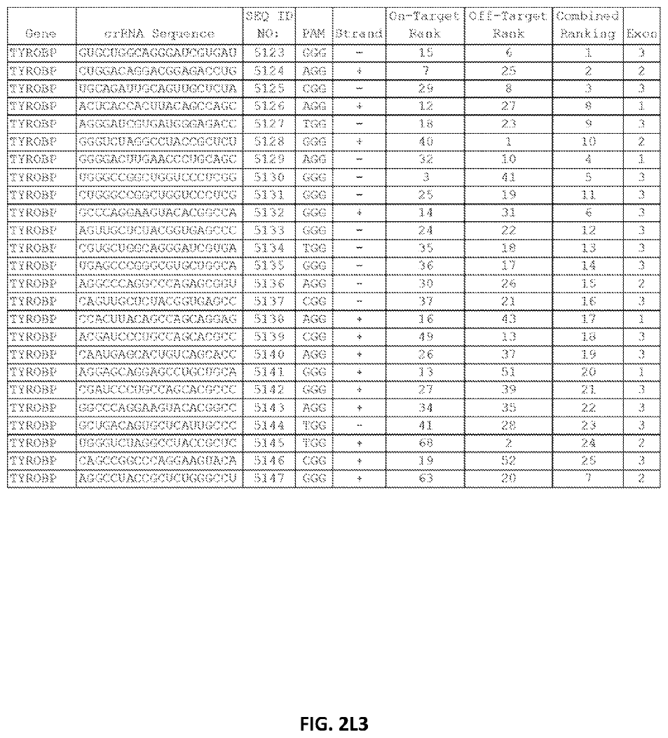

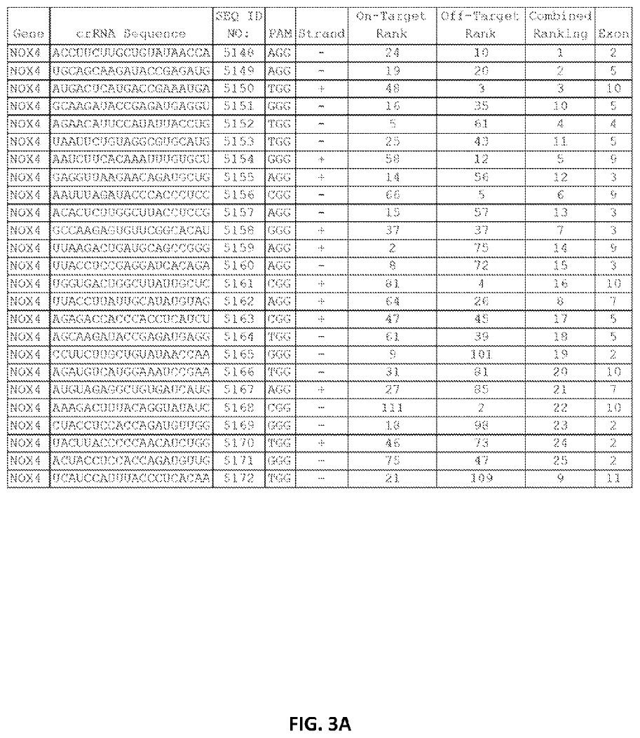

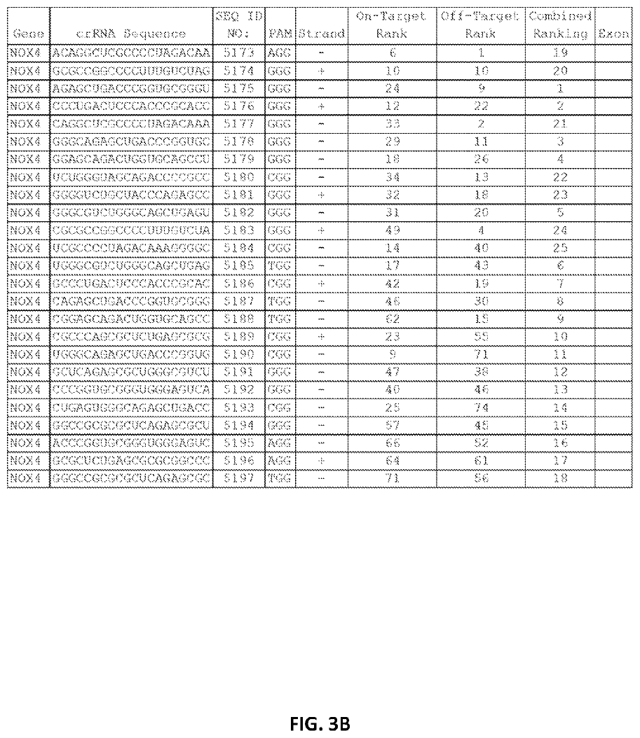

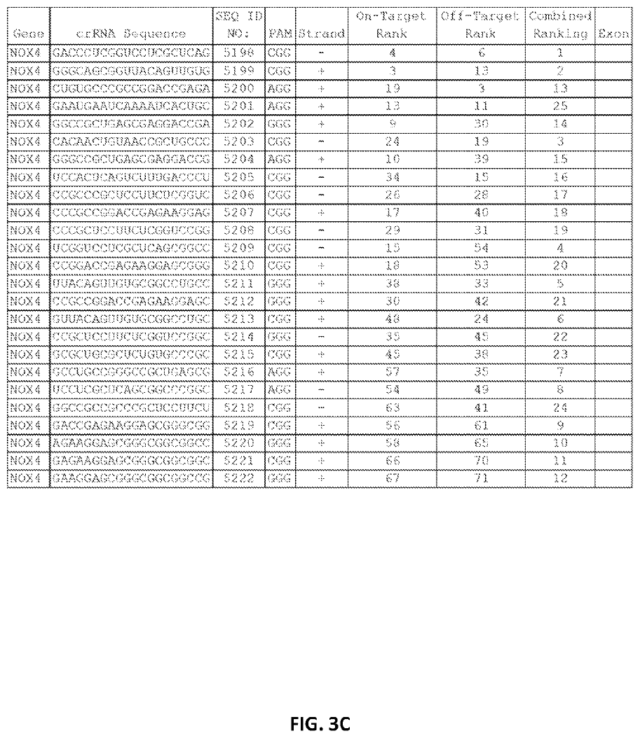

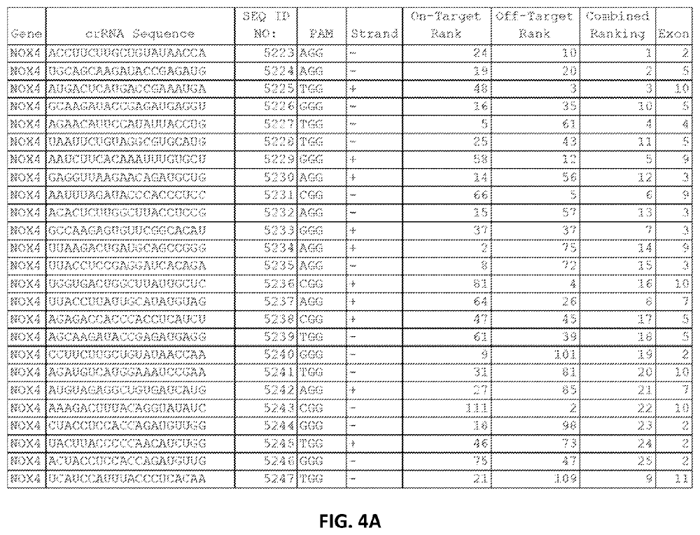

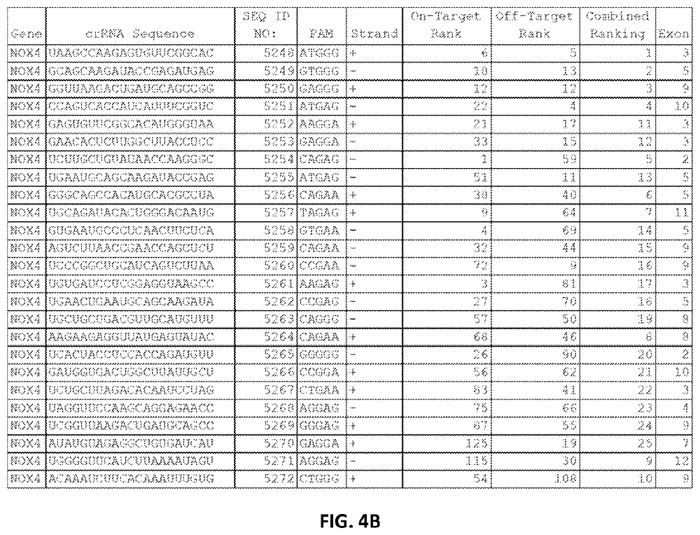

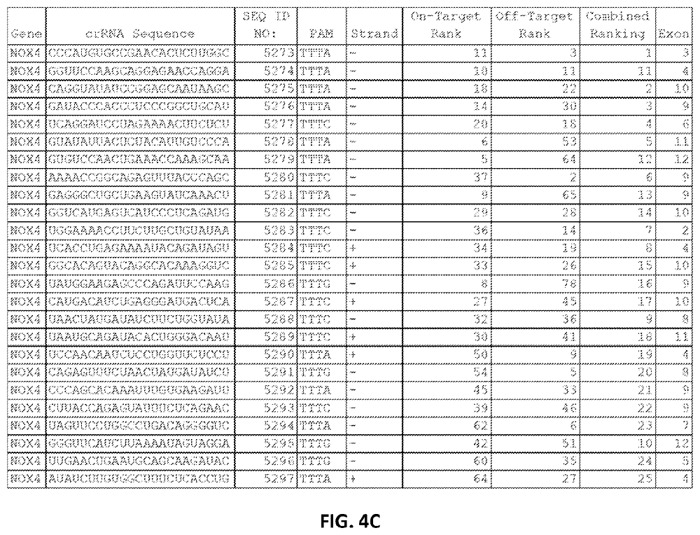

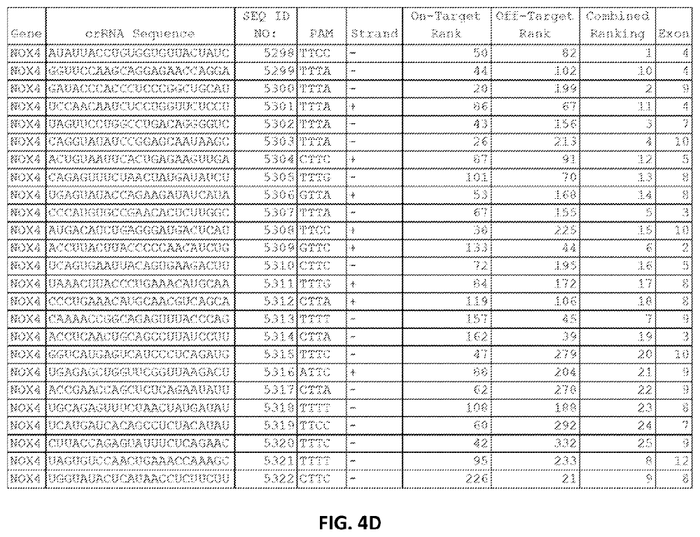

In some embodiments, the disclosure provides a method for treating or preventing free oxygen radicals in a subject in need thereof by administering a therapeutically effective amount of a composition, wherein the composition comprises: (i) an RNA-guided nuclease or a nucleic acid encoding an RNA-guided nuclease; and (ii) at least one guide RNA or a nucleic acid encoding at least one guide RNA targeting a gene selected from the group consisting of a 6-phosphogluconate dehydrogenase (6PGD) gene, an alcohol dehydrogenase (ADH) gene, an aldehyde dehydrogenase (ALDH2) gene, an AP-1 gene, a B-cell lymphoma-extra large (Bcl-XL) gene, a BCL2 apoptosis regulator (Bcl-2) gene, a Bcl-2-associated X protein (BAX) gene, a catalase (CAT) gene, a c-Jun N-terminal kinase (JNK) gene, a coenzyme Q10 gene, a CYP2E1 gene, a cytochrome c (Cyt c) gene, a F1Fo-ATP synthase gene, a ferritin heavy chain (FHC) gene, a glucose-6-phosphate dehydrogenase (G6PD) gene, a glutamylcysteine synthetase (GCS) gene, a glutathione (GSH) synthase gene, a glutathione peroxidase 1 (GPX1) gene, a glutathione peroxidase 2 (GPX2) gene, a glutathione peroxidase 3 (GPX3) gene, a glutathione peroxidase 4 (GPX4) gene, a glutathione peroxidase 5 (GPX5) gene, a glutathione peroxidase 6 (GPX6) gene, a glutathione peroxidase 7 (GPX7) gene, a glutathione peroxidase 8 (GPX8) gene, a glutathione reductase (GR) gene, a glycerol 3-phosphate dehydrogenase gene, a growth arrest and DNA damage (GADD 45) gene, a hypoxia-inducible factor 1-alpha (HIF-1α) gene, a mitogen-activated protein kinase (MAPK) gene, an NADH-ubiquinone oxidoreductase gene, an NADPH oxidase 4 (NOX4) gene, an NADPH oxidase 5 (NOX5) gene, a nuclear factor kappa-light-chain-enhancer of activated B cells (NF-κB) gene, a nuclear factor κB (NF-κB) essential modulator (NEMO) gene, a p46Shc (SHC isoform) gene, a p52Shc (SHC isoform) gene, a p53 upregulated modulator of apoptosis (PUMA) gene, a p66Shc (SHC isoform) gene, a phosphoinositide 3-kinase (PI3-K) gene, a proline oxidase (PIG6, POX) gene, a quinone oxidoreductase (PIG3, NQO1) gene, a respiratory complexes I gene, a respiratory complexes II gene, a respiratory complexes III gene, a respiratory complexes IV gene, a sestrin 1 (SESN1) gene, a sestrin 2 (SESN2) gene, an SHC adaptor protein 1 (SHC1) gene, a superoxide dismutase 1 (SOD1) gene, a superoxide dismutase 2 (SOD2) gene, a superoxide dismutase 3 (SOD3) gene, a TNF alpha induced protein 3 (TNFAIP3) gene, a tumor protein 53 (p53) gene, a tumor protein p53 inducible nuclear protein 1 (TP53INP1) gene, a ubiquinol-cytochrome c oxidoreductase gene, a 2B4 gene, an ABCA1 gene, an ACP5 gene, an ADAR-1 gene, an ADSS gene, an AIG1 gene, an AIM2 gene, an APOBEC3 gene, an ARRB2 gene, a B2M gene, a BCAS3 gene, a BMP4 gene, a C10orf32 gene, a C21orf33 gene, a CASP1 gene, a CCL5 gene, a CD160 gene, a Cd53 gene, a CDKN2A gene, a CHEK1 gene, a CNNM2 gene, a CNTNAP2 gene, a CSMD1 gene, a CTLA-4 gene, a CTSB gene, a C-type lectin receptors CLRs gene, a CXCL10 gene, a CYP17A1 gene, a DDX60 gene, a DYNC1I1 gene, a FOXO3a gene, a GPC6 gene, a GRN gene, an HCK gene, an HECW1 gene, an HLA gene, an IFI30 gene, an IFI44L gene, an IFI6 gene, an IFITM gene, an IFITM1/3 gene, an IFITM2 gene, an IFITM3 gene, an IL-18 gene, an IL-1α gene, an IL-1β gene, an interferon-γ gene, an interleukin-12 (IL-12) gene, an IRF gene, an IRF-1 gene, an gene, an IRF3 gene, an IRF7 gene, a LAG-3 gene, a LIPC gene, a MDA5/IFIH1 gene, a MPAK gene, a MYH9 gene, a MYO16 gene, a MYO5A gene, an NAIP gene, an NF-κB gene, an NLRC4 gene, an gene, an NLRP3 gene, a NOD2 gene, an gene, an NPL gene, an NR gene, a nucleotide oligomerization and binding domain NOD-like receptors gene, an OAS1 gene, an OAS2 gene, an OASL gene, a parkin gene (PARK2) gene, a PD-1 gene, a PLEKHG1 gene, av PRKCA gene, a PTBP1 gene, a PYCARD gene, a Pyrin-HIN (PYHIN) domain containing receptor gene (e.g. AIM2), a reactive oxygen species (ROS) gene, a retinoic acid inducible gene-I (RIG-I)-like receptor (RLRs) gene, an RFC3 gene, a RGS1 gene, a RIG-I/DDX58 gene, a SAMHD1 gene, a SF3A1/SF3B1 gene, a SFXN2 gene, a SLAMF7 gene, a SLC41A1 gene, a SLC8A1 gene, a SLCO3A1 gene, a STAT1 gene, a tetherin gene, a TLR5 gene, a TLR7 gene, a TLR9 gene, a Toll-like receptor (TLR) gene, a TREM2 gene, a TREX1 gene, a TRIM5 gene, a TTLL7 gene, and a TYROBP gene. In some embodiments, the guide RNA has a crRNA sequence selected from those sequences shown in FIG. 2. In some embodiments, the RNA-guided nuclease or a nucleic acid encoding an RNA-guided nuclease and at least one guide RNA or a nucleic acid encoding at least one guide RNA are formulated in a lipid nanoparticle (LNP). In some embodiments, the pharmaceutical composition or method is for treating a joint disorder. In some embodiments, the pharmaceutical composition or method is for treating arthritis, osteoarthritis, rheumatoid arthritis, post-traumatic arthritis, gout, pseudogout, canine lameness, equine lameness, or feline lameness.

### B. Osteoarthritis

In one aspect, the present disclosure encompasses treatments for osteoarthritis (OA). In some embodiments, OA treatment comprises a therapeutically effective amount of a Clustered Regularly Interspaced Short Palindromic Repeats (CRISPR) gene-editing system, the system comprising: (i) a CRISPR Associated (Cas) protein; and (ii) at least one guide RNA targeting NFKB1. In some embodiments, the OA treatment comprises a CRISPR gene-editing system targeting hNFKB1. In some embodiments, the OA treatment comprises a CRISPR gene-editing system targeting cNFKB1. In some embodiments, the OA treatment comprises a CRISP gene-editing system targeting eNFKB1. In some embodiments, the OA treatment comprises a CRISPR gene-editing system targeting fNFKB1.

In some embodiments, the CRISPR gene-editing system for the treatment of OA comprises one or more sgRNAs targeting exon 1 of hNFKB1. In some embodiments, the CRISPR gene-editing system for the treatment of OA comprises one or more sgRNAs targeting exon 2 of hNFKB1. In some embodiments, the CRISPR gene-editing system for the treatment of OA comprises one or more sgRNAs targeting exon 3 of hNFKB1. In some embodiments, the CRISPR gene-editing system for the treatment of OA comprises one or more sgRNAs targeting exon 4 of hNFKB1. In some embodiments, the CRISPR gene-editing system for the treatment of OA comprises one or more sgRNAs targeting exon 5 of hNFKB1. In some embodiments, the CRISPR gene-editing system for the treatment of OA comprises one or more sgRNAs targeting exon 6 of hNFKB1. In some embodiments, the CRISPR gene-editing system for the treatment of OA comprises one or more sgRNAs targeting exon 7 of hNFKB1. In some embodiments, the CRISPR gene-editing system for the treatment of OA comprises one or more sgRNAs targeting exon 8 of hNFKB1. In some embodiments, the CRISPR gene-editing system for the treatment of OA comprises one or more sgRNAs targeting exon 9 of hNFKB1. In some embodiments, the CRISPR gene-editing system for the treatment of OA comprises one or more sgRNAs targeting exon 10 of hNFKB1. In some embodiments, the CRISPR gene-editing system for the treatment of OA comprises one or more sgRNAs targeting exon 11 of hNFKB1. In some embodiments, the CRISPR gene-editing system for the treatment of OA comprises one or more sgRNAs targeting exon 12 of hNFKB1. In some embodiments, the CRISPR gene-editing system for the treatment of OA comprises one or more sgRNAs targeting exon 13 of hNFKB1. In some embodiments, the CRISPR gene-editing system for the treatment of OA comprises one or more sgRNAs targeting exon 14 of hNFKB1. In some embodiments, the CRISPR gene-editing system for the treatment of OA comprises one or more sgRNAs targeting exon 15 of hNFKB1. In some embodiments, the CRISPR gene-editing system for the treatment of OA comprises one or more sgRNAs targeting exon 16 of hNFKB1. In some embodiments, the CRISPR gene-editing system for the treatment of OA comprises one or more sgRNAs targeting exon 17 of hNFKB1. In some embodiments, the CRISPR gene-editing system for the treatment of OA comprises one or more sgRNAs targeting exon 18 of hNFKB1. In some embodiments, the CRISPR gene-editing system for the treatment of OA comprises one or more sgRNAs targeting exon 19 of hNFKB1. In some embodiments, the CRISPR gene-editing system for the treatment of OA comprises one or more sgRNAs targeting exon 20 of hNFKB1. In some embodiments, the CRISPR gene-editing system for the treatment of OA comprises one or more sgRNAs targeting exon 21 of hNFKB1. In some embodiments, the CRISPR gene-editing system for the treatment of OA comprises one or more sgRNAs targeting exon 22 of hNFKB1. In some embodiments, the CRISPR gene-editing system for the treatment of OA comprises one or more sgRNAs targeting exon 23 of hNFKB1. In some embodiments, the CRISPR gene-editing system for the treatment of OA comprises one or more sgRNAs targeting exon 24 of hNFKB1. In some embodiments, the CRISPR gene-editing system for the treatment of OA comprises one or more sgRNAs targeting exon 25 of hNFKB1. In some embodiments, the CRISPR gene-editing system for the treatment of OA comprises one or more sgRNAs targeting exon 26 of hNFKB1. In some embodiments, the CRISPR gene-editing system for the treatment of OA comprises one or more sgRNAs targeting exon 27 of hNFKB1.

In some embodiments, the CRISPR gene-editing system for the treatment of OA comprises one or more sgRNAs targeting exon 1 of cNFKB1. In some embodiments, the CRISPR gene-editing system for the treatment of OA comprises one or more sgRNAs targeting exon 2 of cNFKB1. In some embodiments, the CRISPR gene-editing system for the treatment of OA comprises one or more sgRNAs targeting exon 3 of cNFKB1. In some embodiments, the CRISPR gene-editing system for the treatment of OA comprises one or more sgRNAs targeting exon 4 of cNFKB1. In some embodiments, the CRISPR gene-editing system for the treatment of OA comprises one or more sgRNAs targeting exon 5 of cNFKB1. In some embodiments, the CRISPR gene-editing system for the treatment of OA comprises one or more sgRNAs targeting exon 6 of cNFKB1. In some embodiments, the CRISPR gene-editing system for the treatment of OA comprises one or more sgRNAs targeting exon 7 of cNFKB1. In some embodiments, the CRISPR gene-editing system for the treatment of OA comprises one or more sgRNAs targeting exon 8 of cNFKB1. In some embodiments, the CRISPR gene-editing system for the treatment of OA comprises one or more sgRNAs targeting exon 9 of cNFKB1. In some embodiments, the CRISPR gene-editing system for the treatment of OA comprises one or more sgRNAs targeting exon 10 of cNFKB1. In some embodiments, the CRISPR gene-editing system for the treatment of OA comprises one or more sgRNAs targeting exon 11 of cNFKB1. In some embodiments, the CRISPR gene-editing system for the treatment of OA comprises one or more sgRNAs targeting exon 12 of cNFKB1. In some embodiments, the CRISPR gene-editing system for the treatment of OA comprises one or more sgRNAs targeting exon 13 of cNFKB1. In some embodiments, the CRISPR gene-editing system for the treatment of OA comprises one or more sgRNAs targeting exon 14 of cNFKB1. In some embodiments, the CRISPR gene-editing system for the treatment of OA comprises one or more sgRNAs targeting exon 15 of cNFKB1. In some embodiments, the CRISPR gene-editing system for the treatment of OA comprises one or more sgRNAs targeting exon 16 of cNFKB1. In some embodiments, the CRISPR gene-editing system for the treatment of OA comprises one or more sgRNAs targeting exon 17 of cNFKB1. In some embodiments, the CRISPR gene-editing system for the treatment of OA comprises one or more sgRNAs targeting exon 18 of cNFKB1. In some embodiments, the CRISPR gene-editing system for the treatment of OA comprises one or more sgRNAs targeting exon 19 of cNFKB1. In some embodiments, the CRISPR gene-editing system for the treatment of OA comprises one or more sgRNAs targeting exon 20 of cNFKB1. In some embodiments, the CRISPR gene-editing system for the treatment of OA comprises one or more sgRNAs targeting exon 21 of cNFKB1. In some embodiments, the CRISPR gene-editing system for the treatment of OA comprises one or more sgRNAs targeting exon 22 of cNFKB1. In some embodiments, the CRISPR gene-editing system for the treatment of OA comprises one or more sgRNAs targeting exon 23 of cNFKB1. In some embodiments, the CRISPR gene-editing system for the treatment of OA comprises one or more sgRNAs targeting exon 24 of cNFKB1. In some embodiments, the CRISPR gene-editing system for the treatment of OA comprises one or more sgRNAs targeting exon 25 of cNFKB1.

In some embodiments, the CRISPR gene-editing system for the treatment of OA comprises one or more sgRNAs targeting exon 1 of eNFKB1. In some embodiments, the CRISPR gene-editing system for the treatment of OA comprises one or more sgRNAs targeting exon 2 of eNFKB1. In some embodiments, the CRISPR gene-editing system for the treatment of OA comprises one or more sgRNAs targeting exon 3 of eNFKB1. In some embodiments, the CRISPR gene-editing system for the treatment of OA comprises one or more sgRNAs targeting exon 4 of eNFKB1. In some embodiments, the CRISPR gene-editing system for the treatment of OA comprises one or more sgRNAs targeting exon 5 of eNFKB1. In some embodiments, the CRISPR gene-editing system for the treatment of OA comprises one or more sgRNAs targeting exon 6 of eNFKB1. In some embodiments, the CRISPR gene-editing system for the treatment of OA comprises one or more sgRNAs targeting exon 7 of eNFKB1. In some embodiments, the CRISPR gene-editing system for the treatment of OA comprises one or more sgRNAs targeting exon 8 of eNFKB1. In some embodiments, the CRISPR gene-editing system for the treatment of OA comprises one or more sgRNAs targeting exon 9 of eNFKB1. In some embodiments, the CRISPR gene-editing system for the treatment of OA comprises one or more sgRNAs targeting exon 10 of eNFKB1. In some embodiments, the CRISPR gene-editing system for the treatment of OA comprises one or more sgRNAs targeting exon 11 of eNFKB1. In some embodiments, the CRISPR gene-editing system for the treatment of OA comprises one or more sgRNAs targeting exon 12 of eNFKB1. In some embodiments, the CRISPR gene-editing system for the treatment of OA comprises one or more sgRNAs targeting exon 13 of eNFKB1. In some embodiments, the CRISPR gene-editing system for the treatment of OA comprises one or more sgRNAs targeting exon 14 of eNFKB1. In some embodiments, the CRISPR gene-editing system for the treatment of OA comprises one or more sgRNAs targeting exon 15 of eNFKB1. In some embodiments, the CRISPR gene-editing system for the treatment of OA comprises one or more sgRNAs targeting exon 16 of eNFKB1. In some embodiments, the CRISPR gene-editing system for the treatment of OA comprises one or more sgRNAs targeting exon 17 of eNFKB1. In some embodiments, the CRISPR gene-editing system for the treatment of OA comprises one or more sgRNAs targeting exon 18 of eNFKB1. In some embodiments, the CRISPR gene-editing system for the treatment of OA comprises one or more sgRNAs targeting exon 19 of eNFKB1. In some embodiments, the CRISPR gene-editing system for the treatment of OA comprises one or more sgRNAs targeting exon 20 of eNFKB1. In some embodiments, the CRISPR gene-editing system for the treatment of OA comprises one or more sgRNAs targeting exon 21 of eNFKB1. In some embodiments, the CRISPR gene-editing system for the treatment of OA comprises one or more sgRNAs targeting exon 22 of eNFKB1. In some embodiments, the CRISPR gene-editing system for the treatment of OA comprises one or more sgRNAs targeting exon 23 of eNFKB1. In some embodiments, the CRISPR gene-editing system for the treatment of OA comprises one or more sgRNAs targeting exon 24 of eNFKB1. In some embodiments, the CRISPR gene-editing system for the treatment of OA comprises one or more sgRNAs targeting exon 25 of eNFKB1. In some embodiments, the CRISPR gene-editing system for the treatment of OA comprises one or more sgRNAs targeting exon 26 of eNFKB1. In some embodiments, the CRISPR gene-editing system for the treatment of OA comprises one or more sgRNAs targeting exon 27 of eNFKB1. In some embodiments, the CRISPR gene-editing system for the treatment of OA comprises one or more sgRNAs targeting exon 28 of eNFKB1.

In some embodiments, the CRISPR gene-editing system for the treatment of OA comprises one or more sgRNAs targeting exon 1 of fNFKB1. In some embodiments, the CRISPR gene-editing system for the treatment of OA comprises one or more sgRNAs targeting exon 2 of fNFKB1. In some embodiments, the CRISPR gene-editing system for the treatment of OA comprises one or more sgRNAs targeting exon 3 of fNFKB1. In some embodiments, the CRISPR gene-editing system for the treatment of OA comprises one or more sgRNAs targeting exon 4 of fNFKB1. In some embodiments, the CRISPR gene-editing system for the treatment of OA comprises one or more sgRNAs targeting exon 5 of fNFKB1. In some embodiments, the CRISPR gene-editing system for the treatment of OA comprises one or more sgRNAs targeting exon 6 of fNFKB1. In some embodiments, the CRISPR gene-editing system for the treatment of OA comprises one or more sgRNAs targeting exon 7 of fNFKB1. In some embodiments, the CRISPR gene-editing system for the treatment of OA comprises one or more sgRNAs targeting exon 8 of fNFKB1. In some embodiments, the CRISPR gene-editing system for the treatment of OA comprises one or more sgRNAs targeting exon 9 of fNFKB1. In some embodiments, the CRISPR gene-editing system for the treatment of OA comprises one or more sgRNAs targeting exon 10 of fNFKB1. In some embodiments, the CRISPR gene-editing system for the treatment of OA comprises one or more sgRNAs targeting exon 11 of fNFKB1. In some embodiments, the CRISPR gene-editing system for the treatment of OA comprises one or more sgRNAs targeting exon 12 of fNFKB1. In some embodiments, the CRISPR gene-editing system for the treatment of OA comprises one or more sgRNAs targeting exon 13 of fNFKB1. In some embodiments, the CRISPR gene-editing system for the treatment of OA comprises one or more sgRNAs targeting exon 14 of fNFKB1. In some embodiments, the CRISPR gene-editing system for the treatment of OA comprises one or more sgRNAs targeting exon 15 of fNFKB1. In some embodiments, the CRISPR gene-editing system for the treatment of OA comprises one or more sgRNAs targeting exon 16 of fNFKB1. In some embodiments, the CRISPR gene-editing system for the treatment of OA comprises one or more sgRNAs targeting exon 17 of fNFKB1. In some embodiments, the CRISPR gene-editing system for the treatment of OA comprises one or more sgRNAs targeting exon 18 of fNFKB1. In some embodiments, the CRISPR gene-editing system for the treatment of OA comprises one or more sgRNAs targeting exon 19 of fNFKB1. In some embodiments, the CRISPR gene-editing system for the treatment of OA comprises one or more sgRNAs targeting exon 20 of fNFKB1. In some embodiments, the CRISPR gene-editing system for the treatment of OA comprises one or more sgRNAs targeting exon 21 of fNFKB1. In some embodiments, the CRISPR gene-editing system for the treatment of OA comprises one or more sgRNAs targeting exon 22 of fNFKB1. In some embodiments, the CRISPR gene-editing system for the treatment of OA comprises one or more sgRNAs targeting exon 23 of fNFKB1. In some embodiments, the CRISPR gene-editing system for the treatment of OA comprises one or more sgRNAs targeting exon 24 of fNFKB1. In some embodiments, the CRISPR gene-editing system for the treatment of OA comprises one or more sgRNAs targeting exon 25 of fNFKB1.

In some embodiments, the CRISPR gene-editing system for the treatment of OA comprises one or more sgRNAs targeting NFKB2. In some embodiments, the CRISPR gene-editing system for the treatment of OA comprises one or more sgRNAs targeting hNFKB2. In some embodiments, the CRISPR gene-editing system for the treatment of OA comprises one or more sgRNAs targeting cNFKB2. In some embodiments, the CRISPR gene-editing system for the treatment of OA comprises one or more sgRNAs targeting eNFKB2. In some embodiments, the CRISPR gene-editing system for the treatment of OA comprises one or more sgRNAs targeting fNFKB2.

In some embodiments, the CRISPR gene-editing system for the treatment of OA comprises one or more sgRNAs targeting exon 1 of hNFKB2. In some embodiments, the CRISPR gene-editing system for the treatment of OA comprises one or more sgRNAs targeting exon 2 of hNFKB2. In some embodiments, the CRISPR gene-editing system for the treatment of OA comprises one or more sgRNAs targeting exon 3 of hNFKB2. In some embodiments, the CRISPR gene-editing system for the treatment of OA comprises one or more sgRNAs targeting exon 4 of hNFKB2. In some embodiments, the CRISPR gene-editing system for the treatment of OA comprises one or more sgRNAs targeting exon 5 of hNFKB2. In some embodiments, the CRISPR gene-editing system for the treatment of OA comprises one or more sgRNAs targeting exon 6 of hNFKB2. In some embodiments, the CRISPR gene-editing system for the treatment of OA comprises one or more sgRNAs targeting exon 7 of hNFKB2. In some embodiments, the CRISPR gene-editing system for the treatment of OA comprises one or more sgRNAs targeting exon 8 of hNFKB2. In some embodiments, the CRISPR gene-editing system for the treatment of OA comprises one or more sgRNAs targeting exon 9 of hNFKB2. In some embodiments, the CRISPR gene-editing system for the treatment of OA comprises one or more sgRNAs targeting exon 10 of hNFKB2. In some embodiments, the CRISPR gene-editing system for the treatment of OA comprises one or more sgRNAs targeting exon 11 of hNFKB2. In some embodiments, the CRISPR gene-editing system for the treatment of OA comprises one or more sgRNAs targeting exon 12 of hNFKB2. In some embodiments, the CRISPR gene-editing system for the treatment of OA comprises one or more sgRNAs targeting exon 13 of hNFKB2. In some embodiments, the CRISPR gene-editing system for the treatment of OA comprises one or more sgRNAs targeting exon 14 of hNFKB2. In some embodiments, the CRISPR gene-editing system for the treatment of OA comprises one or more sgRNAs targeting exon 15 of hNFKB2. In some embodiments, the CRISPR gene-editing system for the treatment of OA comprises one or more sgRNAs targeting exon 16 of hNFKB2. In some embodiments, the CRISPR gene-editing system for the treatment of OA comprises one or more sgRNAs targeting exon 17 of hNFKB2. In some embodiments, the CRISPR gene-editing system for the treatment of OA comprises one or more sgRNAs targeting exon 18 of hNFKB2. In some embodiments, the CRISPR gene-editing system for the treatment of OA comprises one or more sgRNAs targeting exon 19 of hNFKB2. In some embodiments, the CRISPR gene-editing system for the treatment of OA comprises one or more sgRNAs targeting exon 20 of hNFKB2. In some embodiments, the CRISPR gene-editing system for the treatment of OA comprises one or more sgRNAs targeting exon 21 of hNFKB2. In some embodiments, the CRISPR gene-editing system for the treatment of OA comprises one or more sgRNAs targeting exon 22 of hNFKB2. In some embodiments, the CRISPR gene-editing system for the treatment of OA comprises one or more sgRNAs targeting exon 23 of hNFKB2. In some embodiments, the CRISPR gene-editing system for the treatment of OA comprises one or more sgRNAs targeting exon 24 of hNFKB2.

In some embodiments, the CRISPR gene-editing system for the treatment of OA comprises one or more sgRNAs targeting exon 1 of cNFKB2. In some embodiments, the CRISPR gene-editing system for the treatment of OA comprises one or more sgRNAs targeting exon 2 of cNFKB2. In some embodiments, the CRISPR gene-editing system for the treatment of OA comprises one or more sgRNAs targeting exon 3 of cNFKB2. In some embodiments, the CRISPR gene-editing system for the treatment of OA comprises one or more sgRNAs targeting exon 4 of cNFKB2. In some embodiments, the CRISPR gene-editing system for the treatment of OA comprises one or more sgRNAs targeting exon 5 of cNFKB2. In some embodiments, the CRISPR gene-editing system for the treatment of OA comprises one or more sgRNAs targeting exon 6 of cNFKB2. In some embodiments, the CRISPR gene-editing system for the treatment of OA comprises one or more sgRNAs targeting exon 7 of cNFKB2. In some embodiments, the CRISPR gene-editing system for the treatment of OA comprises one or more sgRNAs targeting exon 8 of cNFKB2. In some embodiments, the CRISPR gene-editing system for the treatment of OA comprises one or more sgRNAs targeting exon 9 of cNFKB2. In some embodiments, the CRISPR gene-editing system for the treatment of OA comprises one or more sgRNAs targeting exon 10 of cNFKB2. In some embodiments, the CRISPR gene-editing system for the treatment of OA comprises one or more sgRNAs targeting exon 11 of cNFKB2. In some embodiments, the CRISPR gene-editing system for the treatment of OA comprises one or more sgRNAs targeting exon 12 of cNFKB2. In some embodiments, the CRISPR gene-editing system for the treatment of OA comprises one or more sgRNAs targeting exon 13 of cNFKB2. In some embodiments, the CRISPR gene-editing system for the treatment of OA comprises one or more sgRNAs targeting exon 14 of cNFKB2. In some embodiments, the CRISPR gene-editing system for the treatment of OA comprises one or more sgRNAs targeting exon 15 of cNFKB2. In some embodiments, the CRISPR gene-editing system for the treatment of OA comprises one or more sgRNAs targeting exon 16 of cNFKB2. In some embodiments, the CRISPR gene-editing system for the treatment of OA comprises one or more sgRNAs targeting exon 17 of cNFKB2. In some embodiments, the CRISPR gene-editing system for the treatment of OA comprises one or more sgRNAs targeting exon 18 of cNFKB2. In some embodiments, the CRISPR gene-editing system for the treatment of OA comprises one or more sgRNAs targeting exon 19 of cNFKB2. In some embodiments, the CRISPR gene-editing system for the treatment of OA comprises one or more sgRNAs targeting exon 20 of cNFKB2. In some embodiments, the CRISPR gene-editing system for the treatment of OA comprises one or more sgRNAs targeting exon 21 of cNFKB2. In some embodiments, the CRISPR gene-editing system for the treatment of OA comprises one or more sgRNAs targeting exon 22 of cNFKB2. In some embodiments, the CRISPR gene-editing system for the treatment of OA comprises one or more sgRNAs targeting exon 23 of cNFKB2.

In some embodiments, the CRISPR gene-editing system for the treatment of OA comprises one or more sgRNAs targeting exon 1 of eNFKB2. In some embodiments, the CRISPR gene-editing system for the treatment of OA comprises one or more sgRNAs targeting exon 2 of eNFKB2. In some embodiments, the CRISPR gene-editing system for the treatment of OA comprises one or more sgRNAs targeting exon 3 of eNFKB2. In some embodiments, the CRISPR gene-editing system for the treatment of OA comprises one or more sgRNAs targeting exon 4 of eNFKB2. In some embodiments, the CRISPR gene-editing system for the treatment of OA comprises one or more sgRNAs targeting exon 5 of eNFKB2. In some embodiments, the CRISPR gene-editing system for the treatment of OA comprises one or more sgRNAs targeting exon 6 of eNFKB2. In some embodiments, the CRISPR gene-editing system for the treatment of OA comprises one or more sgRNAs targeting exon 7 of eNFKB2. In some embodiments, the CRISPR gene-editing system for the treatment of OA comprises one or more sgRNAs targeting exon 8 of eNFKB2. In some embodiments, the CRISPR gene-editing system for the treatment of OA comprises one or more sgRNAs targeting exon 9 of eNFKB2. In some embodiments, the CRISPR gene-editing system for the treatment of OA comprises one or more sgRNAs targeting exon 10 of eNFKB2. In some embodiments, the CRISPR gene-editing system for the treatment of OA comprises one or more sgRNAs targeting exon 11 of eNFKB2. In some embodiments, the CRISPR gene-editing system for the treatment of OA comprises one or more sgRNAs targeting exon 12 of eNFKB2. In some embodiments, the CRISPR gene-editing system for the treatment of OA comprises one or more sgRNAs targeting exon 13 of eNFKB2. In some embodiments, the CRISPR gene-editing system for the treatment of OA comprises one or more sgRNAs targeting exon 14 of eNFKB2. In some embodiments, the CRISPR gene-editing system for the treatment of OA comprises one or more sgRNAs targeting exon 15 of eNFKB2. In some embodiments, the CRISPR gene-editing system for the treatment of OA comprises one or more sgRNAs targeting exon 16 of eNFKB2. In some embodiments, the CRISPR gene-editing system for the treatment of OA comprises one or more sgRNAs targeting exon 17 of eNFKB2. In some embodiments, the CRISPR gene-editing system for the treatment of OA comprises one or more sgRNAs targeting exon 18 of eNFKB2. In some embodiments, the CRISPR gene-editing system for the treatment of OA comprises one or more sgRNAs targeting exon 19 of eNFKB2. In some embodiments, the CRISPR gene-editing system for the treatment of OA comprises one or more sgRNAs targeting exon 20 of eNFKB2. In some embodiments, the CRISPR gene-editing system for the treatment of OA comprises one or more sgRNAs targeting exon 21 of eNFKB2. In some embodiments, the CRISPR gene-editing system for the treatment of OA comprises one or more sgRNAs targeting exon 22 of eNFKB2. In some embodiments, the CRISPR gene-editing system for the treatment of OA comprises one or more sgRNAs targeting exon 23 of eNFKB2.

In some embodiments, the CRISPR gene-editing system for the treatment of OA comprises one or more sgRNAs targeting exon 1 of fNFKB2. In some embodiments, the CRISPR gene-editing system for the treatment of OA comprises one or more sgRNAs targeting exon 2 of fNFKB2. In some embodiments, the CRISPR gene-editing system for the treatment of OA comprises one or more sgRNAs targeting exon 3 of fNFKB2. In some embodiments, the CRISPR gene-editing system for the treatment of OA comprises one or more sgRNAs targeting exon 4 of fNFKB2. In some embodiments, the CRISPR gene-editing system for the treatment of OA comprises one or more sgRNAs targeting exon 5 of fNFKB2. In some embodiments, the CRISPR gene-editing system for the treatment of OA comprises one or more sgRNAs targeting exon 6 of fNFKB2. In some embodiments, the CRISPR gene-editing system for the treatment of OA comprises one or more sgRNAs targeting exon 7 of fNFKB2. In some embodiments, the CRISPR gene-editing system for the treatment of OA comprises one or more sgRNAs targeting exon 8 of fNFKB2. In some embodiments, the CRISPR gene-editing system for the treatment of OA comprises one or more sgRNAs targeting exon 9 of fNFKB2. In some embodiments, the CRISPR gene-editing system for the treatment of OA comprises one or more sgRNAs targeting exon 10 of fNFKB2. In some embodiments, the CRISPR gene-editing system for the treatment of OA comprises one or more sgRNAs targeting exon 11 of fNFKB2. In some embodiments, the CRISPR gene-editing system for the treatment of OA comprises one or more sgRNAs targeting exon 12 of fNFKB2. In some embodiments, the CRISPR gene-editing system for the treatment of OA comprises one or more sgRNAs targeting exon 13 of fNFKB2. In some embodiments, the CRISPR gene-editing system for the treatment of OA comprises one or more sgRNAs targeting exon 14 of fNFKB2. In some embodiments, the CRISPR gene-editing system for the treatment of OA comprises one or more sgRNAs targeting exon 15 of fNFKB2. In some embodiments, the CRISPR gene-editing system for the treatment of OA comprises one or more sgRNAs targeting exon 16 of fNFKB2. In some embodiments, the CRISPR gene-editing system for the treatment of OA comprises one or more sgRNAs targeting exon 17 of fNFKB2. In some embodiments, the CRISPR gene-editing system for the treatment of OA comprises one or more sgRNAs targeting exon 18 of fNFKB2. In some embodiments, the CRISPR gene-editing system for the treatment of OA comprises one or more sgRNAs targeting exon 19 of fNFKB2. In some embodiments, the CRISPR gene-editing system for the treatment of OA comprises one or more sgRNAs targeting exon 20 of fNFKB2. In some embodiments, the CRISPR gene-editing system for the treatment of OA comprises one or more sgRNAs targeting exon 21 of fNFKB2. In some embodiments, the CRISPR gene-editing system for the treatment of OA comprises one or more sgRNAs targeting exon 22 of fNFKB2. In some embodiments, the CRISPR gene-editing system for the treatment of OA comprises one or more sgRNAs targeting exon 23 of fNFKB2.

### C. Psoriatic Arthritis

In one aspect, the present disclosure encompasses treatments for psoriatic arthritis (PsA). In some embodiments, the psoriatic arthritis treatment comprises a therapeutically effective amount of a Clustered Regularly Interspaced Short Palindromic Repeats (CRISPR) gene-editing system, the system comprising: (i) a CRISPR Associated (Cas) protein; and (ii) at least one guide RNA targeting NFKB1. In some embodiments, the psoriatic arthritis treatment comprises a CRISPR gene-editing system targeting hNFKB1. In some embodiments, the psoriatic arthritis treatment comprises a CRISPR gene-editing system targeting cNFKB1. In some embodiments, the psoriatic arthritis treatment comprises a CRISPR gene-editing system targeting eNFKB1. In some embodiments, the psoriatic arthritis treatment comprises a CRISPR gene-editing system targeting fNFKB1.

In some embodiments, the CRISPR gene-editing system for the treatment of psoriatic arthritis comprises one or more sgRNAs targeting exon 1 of hNFKB1. In some embodiments, the CRISPR gene-editing system for the treatment of psoriatic arthritis comprises one or more sgRNAs targeting exon 2 of hNFKB1. In some embodiments, the CRISPR gene-editing system for the treatment of psoriatic arthritis comprises one or more sgRNAs targeting exon 3 of hNFKB1. In some embodiments, the CRISPR gene-editing system for the treatment of psoriatic arthritis comprises one or more sgRNAs targeting exon 4 of hNFKB1. In some embodiments, the CRISPR gene-editing system for the treatment of psoriatic arthritis comprises one or more sgRNAs targeting exon 5 of hNFKB1. In some embodiments, the CRISPR gene-editing system for the treatment of psoriatic arthritis comprises one or more sgRNAs targeting exon 6 of hNFKB1. In some embodiments, the CRISPR gene-editing system for the treatment of psoriatic arthritis comprises one or more sgRNAs targeting exon 7 of hNFKB1. In some embodiments, the CRISPR gene-editing system for the treatment of psoriatic arthritis comprises one or more sgRNAs targeting exon 8 of hNFKB1. In some embodiments, the CRISPR gene-editing system for the treatment of psoriatic arthritis comprises one or more sgRNAs targeting exon 9 of hNFKB1. In some embodiments, the CRISPR gene-editing system for the treatment of psoriatic arthritis comprises one or more sgRNAs targeting exon 10 of hNFKB1. In some embodiments, the CRISPR gene-editing system for the treatment of psoriatic arthritis comprises one or more sgRNAs targeting exon 11 of hNFKB1. In some embodiments, the CRISPR gene-editing system for the treatment of psoriatic arthritis comprises one or more sgRNAs targeting exon 12 of hNFKB1. In some embodiments, the CRISPR gene-editing system for the treatment of psoriatic arthritis comprises one or more sgRNAs targeting exon 13 of hNFKB1. In some embodiments, the CRISPR gene-editing system for the treatment of psoriatic arthritis comprises one or more sgRNAs targeting exon 14 of hNFKB1. In some embodiments, the CRISPR gene-editing system for the treatment of psoriatic arthritis comprises one or more sgRNAs targeting exon 15 of hNFKB1. In some embodiments, the CRISPR gene-editing system for the treatment of psoriatic arthritis comprises one or more sgRNAs targeting exon 16 of hNFKB1. In some embodiments, the CRISPR gene-editing system for the treatment of psoriatic arthritis comprises one or more sgRNAs targeting exon 17 of hNFKB1. In some embodiments, the CRISPR gene-editing system for the treatment of psoriatic arthritis comprises one or more sgRNAs targeting exon 18 of hNFKB1. In some embodiments, the CRISPR gene-editing system for the treatment of psoriatic arthritis comprises one or more sgRNAs targeting exon 19 of hNFKB1. In some embodiments, the CRISPR gene-editing system for the treatment of psoriatic arthritis comprises one or more sgRNAs targeting exon 20 of hNFKB1. In some embodiments, the CRISPR gene-editing system for the treatment of psoriatic arthritis comprises one or more sgRNAs targeting exon 21 of hNFKB1. In some embodiments, the CRISPR gene-editing system for the treatment of psoriatic arthritis comprises one or more sgRNAs targeting exon 22 of hNFKB1. In some embodiments, the CRISPR gene-editing system for the treatment of psoriatic arthritis comprises one or more sgRNAs targeting exon 23 of hNFKB1. In some embodiments, the CRISPR gene-editing system for the treatment of psoriatic arthritis comprises one or more sgRNAs targeting exon 24 of hNFKB1. In some embodiments, the CRISPR gene-editing system for the treatment of psoriatic arthritis comprises one or more sgRNAs targeting exon 25 of hNFKB1. In some embodiments, the CRISPR gene-editing system for the treatment of psoriatic arthritis comprises one or more sgRNAs targeting exon 26 of hNFKB1. In some embodiments, the CRISPR gene-editing system for the treatment of psoriatic arthritis comprises one or more sgRNAs targeting exon 27 of hNFKB1.

In some embodiments, the CRISPR gene-editing system for the treatment of psoriatic arthritis comprises one or more sgRNAs targeting exon 1 of cNFKB1. In some embodiments, the CRISPR gene-editing system for the treatment of psoriatic arthritis comprises one or more sgRNAs targeting exon 2 of cNFKB1. In some embodiments, the CRISPR gene-editing system for the treatment of psoriatic arthritis comprises one or more sgRNAs targeting exon 3 of cNFKB1. In some embodiments, the CRISPR gene-editing system for the treatment of psoriatic arthritis comprises one or more sgRNAs targeting exon 4 of cNFKB1. In some embodiments, the CRISPR gene-editing system for the treatment of psoriatic arthritis comprises one or more sgRNAs targeting exon 5 of cNFKB1. In some embodiments, the CRISPR gene-editing system for the treatment of psoriatic arthritis comprises one or more sgRNAs targeting exon 6 of cNFKB1. In some embodiments, the CRISPR gene-editing system for the treatment of psoriatic arthritis comprises one or more sgRNAs targeting exon 7 of cNFKB1. In some embodiments, the CRISPR gene-editing system for the treatment of psoriatic arthritis comprises one or more sgRNAs targeting exon 8 of cNFKB1. In some embodiments, the CRISPR gene-editing system for the treatment of psoriatic arthritis comprises one or more sgRNAs targeting exon 9 of cNFKB1. In some embodiments, the CRISPR gene-editing system for the treatment of psoriatic arthritis comprises one or more sgRNAs targeting exon 10 of cNFKB1. In some embodiments, the CRISPR gene-editing system for the treatment of psoriatic arthritis comprises one or more sgRNAs targeting exon 11 of cNFKB1. In some embodiments, the CRISPR gene-editing system for the treatment of psoriatic arthritis comprises one or more sgRNAs targeting exon 12 of cNFKB1. In some embodiments, the CRISPR gene-editing system for the treatment of psoriatic arthritis comprises one or more sgRNAs targeting exon 13 of cNFKB1. In some embodiments, the CRISPR gene-editing system for the treatment of psoriatic arthritis comprises one or more sgRNAs targeting exon 14 of cNFKB1. In some embodiments, the CRISPR gene-editing system for the treatment of psoriatic arthritis comprises one or more sgRNAs targeting exon 15 of cNFKB1. In some embodiments, the CRISPR gene-editing system for the treatment of psoriatic arthritis comprises one or more sgRNAs targeting exon 16 of cNFKB1. In some embodiments, the CRISPR gene-editing system for the treatment of psoriatic arthritis comprises one or more sgRNAs targeting exon 17 of cNFKB1. In some embodiments, the CRISPR gene-editing system for the treatment of psoriatic arthritis comprises one or more sgRNAs targeting exon 18 of cNFKB1. In some embodiments, the CRISPR gene-editing system for the treatment of psoriatic arthritis comprises one or more sgRNAs targeting exon 19 of cNFKB1. In some embodiments, the CRISPR gene-editing system for the treatment of psoriatic arthritis comprises one or more sgRNAs targeting exon 20 of cNFKB1. In some embodiments, the CRISPR gene-editing system for the treatment of psoriatic arthritis comprises one or more sgRNAs targeting exon 21 of cNFKB1. In some embodiments, the CRISPR gene-editing system for the treatment of psoriatic arthritis comprises one or more sgRNAs targeting exon 22 of cNFKB1. In some embodiments, the CRISPR gene-editing system for the treatment of psoriatic arthritis comprises one or more sgRNAs targeting exon 23 of cNFKB1. In some embodiments, the CRISPR gene-editing system for the treatment of psoriatic arthritis comprises one or more sgRNAs targeting exon 24 of cNFKB1. In some embodiments, the CRISPR gene-editing system for the treatment of psoriatic arthritis comprises one or more sgRNAs targeting exon 25 of cNFKB1.

In some embodiments, the CRISPR gene-editing system for the treatment of psoriatic arthritis comprises one or more sgRNAs targeting exon 1 of eNFKB1. In some embodiments, the CRISPR gene-editing system for the treatment of psoriatic arthritis comprises one or more sgRNAs targeting exon 2 of eNFKB1. In some embodiments, the CRISPR gene-editing system for the treatment of psoriatic arthritis comprises one or more sgRNAs targeting exon 3 of eNFKB1. In some embodiments, the CRISPR gene-editing system for the treatment of psoriatic arthritis comprises one or more sgRNAs targeting exon 4 of eNFKB1. In some embodiments, the CRISPR gene-editing system for the treatment of psoriatic arthritis comprises one or more sgRNAs targeting exon 5 of eNFKB1. In some embodiments, the CRISPR gene-editing system for the treatment of psoriatic arthritis comprises one or more sgRNAs targeting exon 6 of eNFKB1. In some embodiments, the CRISPR gene-editing system for the treatment of psoriatic arthritis comprises one or more sgRNAs targeting exon 7 of eNFKB1. In some embodiments, the CRISPR gene-editing system for the treatment of psoriatic arthritis comprises one or more sgRNAs targeting exon 8 of eNFKB1. In some embodiments, the CRISPR gene-editing system for the treatment of psoriatic arthritis comprises one or more sgRNAs targeting exon 9 of eNFKB1. In some embodiments, the CRISPR gene-editing system for the treatment of psoriatic arthritis comprises one or more sgRNAs targeting exon 10 of eNFKB1. In some embodiments, the CRISPR gene-editing system for the treatment of psoriatic arthritis comprises one or more sgRNAs targeting exon 11 of eNFKB1. In some embodiments, the CRISPR gene-editing system for the treatment of psoriatic arthritis comprises one or more sgRNAs targeting exon 12 of eNFKB1. In some embodiments, the CRISPR gene-editing system for the treatment of psoriatic arthritis comprises one or more sgRNAs targeting exon 13 of eNFKB1. In some embodiments, the CRISPR gene-editing system for the treatment of psoriatic arthritis comprises one or more sgRNAs targeting exon 14 of eNFKB1. In some embodiments, the CRISPR gene-editing system for the treatment of psoriatic arthritis comprises one or more sgRNAs targeting exon 15 of eNFKB1. In some embodiments, the CRISPR gene-editing system for the treatment of psoriatic arthritis comprises one or more sgRNAs targeting exon 16 of eNFKB1. In some embodiments, the CRISPR gene-editing system for the treatment of psoriatic arthritis comprises one or more sgRNAs targeting exon 17 of eNFKB1. In some embodiments, the CRISPR gene-editing system for the treatment of psoriatic arthritis comprises one or more sgRNAs targeting exon 18 of eNFKB1. In some embodiments, the CRISPR gene-editing system for the treatment of psoriatic arthritis comprises one or more sgRNAs targeting exon 19 of eNFKB1. In some embodiments, the CRISPR gene-editing system for the treatment of psoriatic arthritis comprises one or more sgRNAs targeting exon 20 of eNFKB1. In some embodiments, the CRISPR gene-editing system for the treatment of psoriatic arthritis comprises one or more sgRNAs targeting exon 21 of eNFKB1. In some embodiments, the CRISPR gene-editing system for the treatment of psoriatic arthritis comprises one or more sgRNAs targeting exon 22 of eNFKB1. In some embodiments, the CRISPR gene-editing system for the treatment of psoriatic arthritis comprises one or more sgRNAs targeting exon 23 of eNFKB1. In some embodiments, the CRISPR gene-editing system for the treatment of psoriatic arthritis comprises one or more sgRNAs targeting exon 24 of eNFKB1. In some embodiments, the CRISPR gene-editing system for the treatment of psoriatic arthritis comprises one or more sgRNAs targeting exon 25 of eNFKB1. In some embodiments, the CRISPR gene-editing system for the treatment of psoriatic arthritis comprises one or more sgRNAs targeting exon 26 of eNFKB1. In some embodiments, the CRISPR gene-editing system for the treatment of psoriatic arthritis comprises one or more sgRNAs targeting exon 27 of eNFKB1. In some embodiments, the CRISPR gene-editing system for the treatment of psoriatic arthritis comprises one or more sgRNAs targeting exon 28 of eNFKB1.

In some embodiments, the CRISPR gene-editing system for the treatment of psoriatic arthritis comprises one or more sgRNAs targeting exon 1 of fNFKB1. In some embodiments, the CRISPR gene-editing system for the treatment of psoriatic arthritis comprises one or more sgRNAs targeting exon 2 of fNFKB1. In some embodiments, the CRISPR gene-editing system for the treatment of psoriatic arthritis comprises one or more sgRNAs targeting exon 3 of fNFKB1. In some embodiments, the CRISPR gene-editing system for the treatment of psoriatic arthritis comprises one or more sgRNAs targeting exon 4 of fNFKB1. In some embodiments, the CRISPR gene-editing system for the treatment of psoriatic arthritis comprises one or more sgRNAs targeting exon 5 of fNFKB1. In some embodiments, the CRISPR gene-editing system for the treatment of psoriatic arthritis comprises one or more sgRNAs targeting exon 6 of fNFKB1. In some embodiments, the CRISPR gene-editing system for the treatment of psoriatic arthritis comprises one or more sgRNAs targeting exon 7 of fNFKB1. In some embodiments, the CRISPR gene-editing system for the treatment of psoriatic arthritis comprises one or more sgRNAs targeting exon 8 of fNFKB1. In some embodiments, the CRISPR gene-editing system for the treatment of psoriatic arthritis comprises one or more sgRNAs targeting exon 9 of fNFKB1. In some embodiments, the CRISPR gene-editing system for the treatment of psoriatic arthritis comprises one or more sgRNAs targeting exon 10 of fNFKB1. In some embodiments, the CRISPR gene-editing system for the treatment of psoriatic arthritis comprises one or more sgRNAs targeting exon 11 of fNFKB1. In some embodiments, the CRISPR gene-editing system for the treatment of psoriatic arthritis comprises one or more sgRNAs targeting exon 12 of fNFKB1. In some embodiments, the CRISPR gene-editing system for the treatment of psoriatic arthritis comprises one or more sgRNAs targeting exon 13 of fNFKB1. In some embodiments, the CRISPR gene-editing system for the treatment of psoriatic arthritis comprises one or more sgRNAs targeting exon 14 of fNFKB1. In some embodiments, the CRISPR gene-editing system for the treatment of psoriatic arthritis comprises one or more sgRNAs targeting exon 15 of fNFKB1. In some embodiments, the CRISPR gene-editing system for the treatment of psoriatic arthritis comprises one or more sgRNAs targeting exon 16 of fNFKB1. In some embodiments, the CRISPR gene-editing system for the treatment of psoriatic arthritis comprises one or more sgRNAs targeting exon 17 of fNFKB1. In some embodiments, the CRISPR gene-editing system for the treatment of psoriatic arthritis comprises one or more sgRNAs targeting exon 18 of fNFKB1. In some embodiments, the CRISPR gene-editing system for the treatment of psoriatic arthritis comprises one or more sgRNAs targeting exon 19 of fNFKB1. In some embodiments, the CRISPR gene-editing system for the treatment of psoriatic arthritis comprises one or more sgRNAs targeting exon 20 of fNFKB1. In some embodiments, the CRISPR gene-editing system for the treatment of psoriatic arthritis comprises one or more sgRNAs targeting exon 21 of fNFKB1. In some embodiments, the CRISPR gene-editing system for the treatment of psoriatic arthritis comprises one or more sgRNAs targeting exon 22 of fNFKB1. In some embodiments, the CRISPR gene-editing system for the treatment of psoriatic arthritis comprises one or more sgRNAs targeting exon 23 of fNFKB1. In some embodiments, the CRISPR gene-editing system for the treatment of psoriatic arthritis comprises one or more sgRNAs targeting exon 24 of fNFKB1. In some embodiments, the CRISPR gene-editing system for the treatment of psoriatic arthritis comprises one or more sgRNAs targeting exon 25 of fNFKB1.

In some embodiments, the CRISPR gene-editing system for the treatment of psoriatic arthritis comprises one or more sgRNAs targeting NFKB2. In some embodiments, the CRISPR gene-editing system for the treatment of psoriatic arthritis comprises one or more sgRNAs targeting hNFKB2. In some embodiments, the CRISPR gene-editing system for the treatment of psoriatic arthritis comprises one or more sgRNAs targeting cNFKB2. In some embodiments, the CRISPR gene-editing system for the treatment of psoriatic arthritis comprises one or more sgRNAs targeting eNFKB2. In some embodiments, the CRISPR gene-editing system for the treatment of psoriatic arthritis comprises one or more sgRNAs targeting fNFKB2.

In some embodiments, the CRISPR gene-editing system for the treatment of psoriatic arthritis comprises one or more sgRNAs targeting exon 1 of hNFKB2. In some embodiments, the CRISPR gene-editing system for the treatment of psoriatic arthritis comprises one or more sgRNAs targeting exon 2 of hNFKB2. In some embodiments, the CRISPR gene-editing system for the treatment of psoriatic arthritis comprises one or more sgRNAs targeting exon 3 of hNFKB2. In some embodiments, the CRISPR gene-editing system for the treatment of psoriatic arthritis comprises one or more sgRNAs targeting exon 4 of hNFKB2. In some embodiments, the CRISPR gene-editing system for the treatment of psoriatic arthritis comprises one or more sgRNAs targeting exon 5 of hNFKB2. In some embodiments, the CRISPR gene-editing system for the treatment of psoriatic arthritis comprises one or more sgRNAs targeting exon 6 of hNFKB2. In some embodiments, the CRISPR gene-editing system for the treatment of psoriatic arthritis comprises one or more sgRNAs targeting exon 7 of hNFKB2. In some embodiments, the CRISPR gene-editing system for the treatment of psoriatic arthritis comprises one or more sgRNAs targeting exon 8 of hNFKB2. In some embodiments, the CRISPR gene-editing system for the treatment of psoriatic arthritis comprises one or more sgRNAs targeting exon 9 of hNFKB2. In some embodiments, the CRISPR gene-editing system for the treatment of psoriatic arthritis comprises one or more sgRNAs targeting exon 10 of hNFKB2. In some embodiments, the CRISPR gene-editing system for the treatment of psoriatic arthritis comprises one or more sgRNAs targeting exon 11 of hNFKB2. In some embodiments, the CRISPR gene-editing system for the treatment of psoriatic arthritis comprises one or more sgRNAs targeting exon 12 of hNFKB2. In some embodiments, the CRISPR gene-editing system for the treatment of psoriatic arthritis comprises one or more sgRNAs targeting exon 13 of hNFKB2. In some embodiments, the CRISPR gene-editing system for the treatment of psoriatic arthritis comprises one or more sgRNAs targeting exon 14 of hNFKB2. In some embodiments, the CRISPR gene-editing system for the treatment of psoriatic arthritis comprises one or more sgRNAs targeting exon 15 of hNFKB2. In some embodiments, the CRISPR gene-editing system for the treatment of psoriatic arthritis comprises one or more sgRNAs targeting exon 16 of hNFKB2. In some embodiments, the CRISPR gene-editing system for the treatment of psoriatic arthritis comprises one or more sgRNAs targeting exon 17 of hNFKB2. In some embodiments, the CRISPR gene-editing system for the treatment of psoriatic arthritis comprises one or more sgRNAs targeting exon 18 of hNFKB2. In some embodiments, the CRISPR gene-editing system for the treatment of psoriatic arthritis comprises one or more sgRNAs targeting exon 19 of hNFKB2. In some embodiments, the CRISPR gene-editing system for the treatment of psoriatic arthritis comprises one or more sgRNAs targeting exon 20 of hNFKB2. In some embodiments, the CRISPR gene-editing system for the treatment of psoriatic arthritis comprises one or more sgRNAs targeting exon 21 of hNFKB2. In some embodiments, the CRISPR gene-editing system for the treatment of psoriatic arthritis comprises one or more sgRNAs targeting exon 22 of hNFKB2. In some embodiments, the CRISPR gene-editing system for the treatment of psoriatic arthritis comprises one or more sgRNAs targeting exon 23 of hNFKB2. In some embodiments, the CRISPR gene-editing system for the treatment of psoriatic arthritis comprises one or more sgRNAs targeting exon 24 of hNFKB2.

In some embodiments, the CRISPR gene-editing system for the treatment of psoriatic arthritis comprises one or more sgRNAs targeting exon 1 of cNFKB2. In some embodiments, the CRISPR gene-editing system for the treatment of psoriatic arthritis comprises one or more sgRNAs targeting exon 2 of cNFKB2. In some embodiments, the CRISPR gene-editing system for the treatment of psoriatic arthritis comprises one or more sgRNAs targeting exon 3 of cNFKB2. In some embodiments, the CRISPR gene-editing system for the treatment of psoriatic arthritis comprises one or more sgRNAs targeting exon 4 of cNFKB2. In some embodiments, the CRISPR gene-editing system for the treatment of psoriatic arthritis comprises one or more sgRNAs targeting exon 5 of cNFKB2. In some embodiments, the CRISPR gene-editing system for the treatment of psoriatic arthritis comprises one or more sgRNAs targeting exon 6 of cNFKB2. In some embodiments, the CRISPR gene-editing system for the treatment of psoriatic arthritis comprises one or more sgRNAs targeting exon 7 of cNFKB2. In some embodiments, the CRISPR gene-editing system for the treatment of psoriatic arthritis comprises one or more sgRNAs targeting exon 8 of cNFKB2. In some embodiments, the CRISPR gene-editing system for the treatment of psoriatic arthritis comprises one or more sgRNAs targeting exon 9 of cNFKB2. In some embodiments, the CRISPR gene-editing system for the treatment of psoriatic arthritis comprises one or more sgRNAs targeting exon 10 of cNFKB2. In some embodiments, the CRISPR gene-editing system for the treatment of psoriatic arthritis comprises one or more sgRNAs targeting exon 11 of cNFKB2. In some embodiments, the CRISPR gene-editing system for the treatment of psoriatic arthritis comprises one or more sgRNAs targeting exon 12 of cNFKB2. In some embodiments, the CRISPR gene-editing system for the treatment of psoriatic arthritis comprises one or more sgRNAs targeting exon 13 of cNFKB2. In some embodiments, the CRISPR gene-editing system for the treatment of psoriatic arthritis comprises one or more sgRNAs targeting exon 14 of cNFKB2. In some embodiments, the CRISPR gene-editing system for the treatment of psoriatic arthritis comprises one or more sgRNAs targeting exon 15 of cNFKB2. In some embodiments, the CRISPR gene-editing system for the treatment of psoriatic arthritis comprises one or more sgRNAs targeting exon 16 of cNFKB2. In some embodiments, the CRISPR gene-editing system for the treatment of psoriatic arthritis comprises one or more sgRNAs targeting exon 17 of cNFKB2. In some embodiments, the CRISPR gene-editing system for the treatment of psoriatic arthritis comprises one or more sgRNAs targeting exon 18 of cNFKB2. In some embodiments, the CRISPR gene-editing system for the treatment of psoriatic arthritis comprises one or more sgRNAs targeting exon 19 of cNFKB2. In some embodiments, the CRISPR gene-editing system for the treatment of psoriatic arthritis comprises one or more sgRNAs targeting exon 20 of cNFKB2. In some embodiments, the CRISPR gene-editing system for the treatment of psoriatic arthritis comprises one or more sgRNAs targeting exon 21 of cNFKB2. In some embodiments, the CRISPR gene-editing system for the treatment of psoriatic arthritis comprises one or more sgRNAs targeting exon 22 of cNFKB2. In some embodiments, the CRISPR gene-editing system for the treatment of psoriatic arthritis comprises one or more sgRNAs targeting exon 23 of cNFKB2.

In some embodiments, the CRISPR gene-editing system for the treatment of psoriatic arthritis comprises one or more sgRNAs targeting exon 1 of eNFKB2. In some embodiments, the CRISPR gene-editing system for the treatment of psoriatic arthritis comprises one or more sgRNAs targeting exon 2 of eNFKB2. In some embodiments, the CRISPR gene-editing system for the treatment of psoriatic arthritis comprises one or more sgRNAs targeting exon 3 of eNFKB2. In some embodiments, the CRISPR gene-editing system for the treatment of psoriatic arthritis comprises one or more sgRNAs targeting exon 4 of eNFKB2. In some embodiments, the CRISPR gene-editing system for the treatment of psoriatic arthritis comprises one or more sgRNAs targeting exon 5 of eNFKB2. In some embodiments, the CRISPR gene-editing system for the treatment of psoriatic arthritis comprises one or more sgRNAs targeting exon 6 of eNFKB2. In some embodiments, the CRISPR gene-editing system for the treatment of psoriatic arthritis comprises one or more sgRNAs targeting exon 7 of eNFKB2. In some embodiments, the CRISPR gene-editing system for the treatment of psoriatic arthritis comprises one or more sgRNAs targeting exon 8 of eNFKB2. In some embodiments, the CRISPR gene-editing system for the treatment of psoriatic arthritis comprises one or more sgRNAs targeting exon 9 of eNFKB2. In some embodiments, the CRISPR gene-editing system for the treatment of psoriatic arthritis comprises one or more sgRNAs targeting exon 10 of eNFKB2. In some embodiments, the CRISPR gene-editing system for the treatment of psoriatic arthritis comprises one or more sgRNAs targeting exon 11 of eNFKB2. In some embodiments, the CRISPR gene-editing system for the treatment of psoriatic arthritis comprises one or more sgRNAs targeting exon 12 of eNFKB2. In some embodiments, the CRISPR gene-editing system for the treatment of psoriatic arthritis comprises one or more sgRNAs targeting exon 13 of eNFKB2. In some embodiments, the CRISPR gene-editing system for the treatment of psoriatic arthritis comprises one or more sgRNAs targeting exon 14 of eNFKB2. In some embodiments, the CRISPR gene-editing system for the treatment of psoriatic arthritis comprises one or more sgRNAs targeting exon 15 of eNFKB2. In some embodiments, the CRISPR gene-editing system for the treatment of psoriatic arthritis comprises one or more sgRNAs targeting exon 16 of eNFKB2. In some embodiments, the CRISPR gene-editing system for the treatment of psoriatic arthritis comprises one or more sgRNAs targeting exon 17 of eNFKB2. In some embodiments, the CRISPR gene-editing system for the treatment of psoriatic arthritis comprises one or more sgRNAs targeting exon 18 of eNFKB2. In some embodiments, the CRISPR gene-editing system for the treatment of psoriatic arthritis comprises one or more sgRNAs targeting exon 19 of eNFKB2. In some embodiments, the CRISPR gene-editing system for the treatment of psoriatic arthritis comprises one or more sgRNAs targeting exon 20 of eNFKB2. In some embodiments, the CRISPR gene-editing system for the treatment of psoriatic arthritis comprises one or more sgRNAs targeting exon 21 of eNFKB2. In some embodiments, the CRISPR gene-editing system for the treatment of psoriatic arthritis comprises one or more sgRNAs targeting exon 22 of eNFKB2. In some embodiments, the CRISPR gene-editing system for the treatment of psoriatic arthritis comprises one or more sgRNAs targeting exon 23 of eNFKB2.

In some embodiments, the CRISPR gene-editing system for the treatment of psoriatic arthritis comprises one or more sgRNAs targeting exon 1 of fNFKB2. In some embodiments, the CRISPR gene-editing system for the treatment of psoriatic arthritis comprises one or more sgRNAs targeting exon 2 of fNFKB2. In some embodiments, the CRISPR gene-editing system for the treatment of psoriatic arthritis comprises one or more sgRNAs targeting exon 3 of fNFKB2. In some embodiments, the CRISPR gene-editing system for the treatment of psoriatic arthritis comprises one or more sgRNAs targeting exon 4 of fNFKB2. In some embodiments, the CRISPR gene-editing system for the treatment of psoriatic arthritis comprises one or more sgRNAs targeting exon 5 of fNFKB2. In some embodiments, the CRISPR gene-editing system for the treatment of psoriatic arthritis comprises one or more sgRNAs targeting exon 6 of fNFKB2. In some embodiments, the CRISPR gene-editing system for the treatment of psoriatic arthritis comprises one or more sgRNAs targeting exon 7 of fNFKB2. In some embodiments, the CRISPR gene-editing system for the treatment of psoriatic arthritis comprises one or more sgRNAs targeting exon 8 of fNFKB2. In some embodiments, the CRISPR gene-editing system for the treatment of psoriatic arthritis comprises one or more sgRNAs targeting exon 9 of fNFKB2. In some embodiments, the CRISPR gene-editing system for the treatment of psoriatic arthritis comprises one or more sgRNAs targeting exon 10 of fNFKB2. In some embodiments, the CRISPR gene-editing system for the treatment of psoriatic arthritis comprises one or more sgRNAs targeting exon 11 of fNFKB2. In some embodiments, the CRISPR gene-editing system for the treatment of psoriatic arthritis comprises one or more sgRNAs targeting exon 12 of fNFKB2. In some embodiments, the CRISPR gene-editing system for the treatment of psoriatic arthritis comprises one or more sgRNAs targeting exon 13 of fNFKB2. In some embodiments, the CRISPR gene-editing system for the treatment of psoriatic arthritis comprises one or more sgRNAs targeting exon 14 of fNFKB2. In some embodiments, the CRISPR gene-editing system for the treatment of psoriatic arthritis comprises one or more sgRNAs targeting exon 15 of fNFKB2. In some embodiments, the CRISPR gene-editing system for the treatment of psoriatic arthritis comprises one or more sgRNAs targeting exon 16 of fNFKB2. In some embodiments, the CRISPR gene-editing system for the treatment of psoriatic arthritis comprises one or more sgRNAs targeting exon 17 of fNFKB2. In some embodiments, the CRISPR gene-editing system for the treatment of psoriatic arthritis comprises one or more sgRNAs targeting exon 18 of fNFKB2. In some embodiments, the CRISPR gene-editing system for the treatment of psoriatic arthritis comprises one or more sgRNAs targeting exon 19 of fNFKB2. In some embodiments, the CRISPR gene-editing system for the treatment of psoriatic arthritis comprises one or more sgRNAs targeting exon 20 of fNFKB2. In some embodiments, the CRISPR gene-editing system for the treatment of psoriatic arthritis comprises one or more sgRNAs targeting exon 21 of fNFKB2. In some embodiments, the CRISPR gene-editing system for the treatment of psoriatic arthritis comprises one or more sgRNAs targeting exon 22 of fNFKB2. In some embodiments, the CRISPR gene-editing system for the treatment of psoriatic arthritis comprises one or more sgRNAs targeting exon 23 of fNFKB2.

### D. Gout

In one aspect, the present disclosure encompasses treatments for gout and other crystallopathies affecting the joint, e.g., octacalcium phosphate and calcium pyrophosphate dihydrate in horses. In some embodiments, the gout treatment comprises a therapeutically effective amount of a Clustered Regularly Interspaced Short Palindromic Repeats (CRISPR) gene-editing system, the system comprising: (i) a CRISPR Associated (Cas) protein; and (ii) at least one guide RNA targeting NFKB1. In some embodiments, the gout treatment comprises a CRISPR gene-editing system targeting hNFKB1. In some embodiments, the gout treatment comprises a CRISPR gene-editing system targeting cNFKB1. In some embodiments, the gout treatment comprises a CRISPR gene-editing system targeting eNFKB1. In some embodiments, the gout treatment comprises a CRISPR gene-editing system targeting fNFKB1.

In some embodiments, the CRISPR gene-editing system for the treatment of gout comprises one or more sgRNAs targeting exon 1 of hNFKB1. In some embodiments, the CRISPR gene-editing system for the treatment of gout comprises one or more sgRNAs targeting exon 2 of hNFKB1. In some embodiments, the CRISPR gene-editing system for the treatment of gout comprises one or more sgRNAs targeting exon 3 of hNFKB1. In some embodiments, the CRISPR gene-editing system for the treatment of gout comprises one or more sgRNAs targeting exon 4 of hNFKB1. In some embodiments, the CRISPR gene-editing system for the treatment of gout comprises one or more sgRNAs targeting exon 5 of hNFKB1. In some embodiments, the CRISPR gene-editing system for the treatment of gout comprises one or more sgRNAs targeting exon 6 of hNFKB1. In some embodiments, the CRISPR gene-editing system for the treatment of gout comprises one or more sgRNAs targeting exon 7 of hNFKB1. In some embodiments, the CRISPR gene-editing system for the treatment of gout comprises one or more sgRNAs targeting exon 8 of hNFKB1. In some embodiments, the CRISPR gene-editing system for the treatment of gout comprises one or more sgRNAs targeting exon 9 of hNFKB1. In some embodiments, the CRISPR gene-editing system for the treatment of gout comprises one or more sgRNAs targeting exon 10 of hNFKB1. In some embodiments, the CRISPR gene-editing system for the treatment of gout comprises one or more sgRNAs targeting exon 11 of hNFKB1. In some embodiments, the CRISPR gene-editing system for the treatment of gout comprises one or more sgRNAs targeting exon 12 of hNFKB1. In some embodiments, the CRISPR gene-editing system for the treatment of gout comprises one or more sgRNAs targeting exon 13 of hNFKB1. In some embodiments, the CRISPR gene-editing system for the treatment of gout comprises one or more sgRNAs targeting exon 14 of hNFKB1. In some embodiments, the CRISPR gene-editing system for the treatment of gout comprises one or more sgRNAs targeting exon 15 of hNFKB1. In some embodiments, the CRISPR gene-editing system for the treatment of gout comprises one or more sgRNAs targeting exon 16 of hNFKB1. In some embodiments, the CRISPR gene-editing system for the treatment of gout comprises one or more sgRNAs targeting exon 17 of hNFKB1. In some embodiments, the CRISPR gene-editing system for the treatment of gout comprises one or more sgRNAs targeting exon 18 of hNFKB1. In some embodiments, the CRISPR gene-editing system for the treatment of gout comprises one or more sgRNAs targeting exon 19 of hNFKB1. In some embodiments, the CRISPR gene-editing system for the treatment of gout comprises one or more sgRNAs targeting exon 20 of hNFKB1. In some embodiments, the CRISPR gene-editing system for the treatment of gout comprises one or more sgRNAs targeting exon 21 of hNFKB1. In some embodiments, the CRISPR gene-editing system for the treatment of gout comprises one or more sgRNAs targeting exon 22 of hNFKB1. In some embodiments, the CRISPR gene-editing system for the treatment of gout comprises one or more sgRNAs targeting exon 23 of hNFKB1. In some embodiments, the CRISPR gene-editing system for the treatment of gout comprises one or more sgRNAs targeting exon 24 of hNFKB1. In some embodiments, the CRISPR gene-editing system for the treatment of gout comprises one or more sgRNAs targeting exon 25 of hNFKB1. In some embodiments, the CRISPR gene-editing system for the treatment of gout comprises one or more sgRNAs targeting exon 26 of hNFKB1. In some embodiments, the CRISPR gene-editing system for the treatment of gout comprises one or more sgRNAs targeting exon 27 of hNFKB1.

In some embodiments, the CRISPR gene-editing system for the treatment of gout comprises one or more sgRNAs targeting exon 1 of cNFKB1. In some embodiments, the CRISPR gene-editing system for the treatment of gout comprises one or more sgRNAs targeting exon 2 of cNFKB1. In some embodiments, the CRISPR gene-editing system for the treatment of gout comprises one or more sgRNAs targeting exon 3 of cNFKB1. In some embodiments, the CRISPR gene-editing system for the treatment of gout comprises one or more sgRNAs targeting exon 4 of cNFKB1. In some embodiments, the CRISPR gene-editing system for the treatment of gout comprises one or more sgRNAs targeting exon 5 of cNFKB1. In some embodiments, the CRISPR gene-editing system for the treatment of gout comprises one or more sgRNAs targeting exon 6 of cNFKB1. In some embodiments, the CRISPR gene-editing system for the treatment of gout comprises one or more sgRNAs targeting exon 7 of cNFKB1. In some embodiments, the CRISPR gene-editing system for the treatment of gout comprises one or more sgRNAs targeting exon 8 of cNFKB1. In some embodiments, the CRISPR gene-editing system for the treatment of gout comprises one or more sgRNAs targeting exon 9 of cNFKB1. In some embodiments, the CRISPR gene-editing system for the treatment of gout comprises one or more sgRNAs targeting exon 10 of cNFKB1. In some embodiments, the CRISPR gene-editing system for the treatment of gout comprises one or more sgRNAs targeting exon 11 of cNFKB1. In some embodiments, the CRISPR gene-editing system for the treatment of gout comprises one or more sgRNAs targeting exon 12 of cNFKB1. In some embodiments, the CRISPR gene-editing system for the treatment of gout comprises one or more sgRNAs targeting exon 13 of cNFKB1. In some embodiments, the CRISPR gene-editing system for the treatment of gout comprises one or more sgRNAs targeting exon 14 of cNFKB1. In some embodiments, the CRISPR gene-editing system for the treatment of gout comprises one or more sgRNAs targeting exon 15 of cNFKB1. In some embodiments, the CRISPR gene-editing system for the treatment of gout comprises one or more sgRNAs targeting exon 16 of cNFKB1. In some embodiments, the CRISPR gene-editing system for the treatment of gout comprises one or more sgRNAs targeting exon 17 of cNFKB1. In some embodiments, the CRISPR gene-editing system for the treatment of gout comprises one or more sgRNAs targeting exon 18 of cNFKB1. In some embodiments, the CRISPR gene-editing system for the treatment of gout comprises one or more sgRNAs targeting exon 19 of cNFKB1. In some embodiments, the CRISPR gene-editing system for the treatment of gout comprises one or more sgRNAs targeting exon 20 of cNFKB1. In some embodiments, the CRISPR gene-editing system for the treatment of gout comprises one or more sgRNAs targeting exon 21 of cNFKB1. In some embodiments, the CRISPR gene-editing system for the treatment of gout comprises one or more sgRNAs targeting exon 22 of cNFKB1. In some embodiments, the CRISPR gene-editing system for the treatment of gout comprises one or more sgRNAs targeting exon 23 of cNFKB1. In some embodiments, the CRISPR gene-editing system for the treatment of gout comprises one or more sgRNAs targeting exon 24 of cNFKB1. In some embodiments, the CRISPR gene-editing system for the treatment of gout comprises one or more sgRNAs targeting exon 25 of cNFKB1.

In some embodiments, the CRISPR gene-editing system for the treatment of gout comprises one or more sgRNAs targeting exon 1 of eNFKB1. In some embodiments, the CRISPR gene-editing system for the treatment of gout comprises one or more sgRNAs targeting exon 2 of eNFKB1. In some embodiments, the CRISPR gene-editing system for the treatment of gout comprises one or more sgRNAs targeting exon 3 of eNFKB1. In some embodiments, the CRISPR gene-editing system for the treatment of gout comprises one or more sgRNAs targeting exon 4 of eNFKB1. In some embodiments, the CRISPR gene-editing system for the treatment of gout comprises one or more sgRNAs targeting exon 5 of eNFKB1. In some embodiments, the CRISPR gene-editing system for the treatment of gout comprises one or more sgRNAs targeting exon 6 of eNFKB1. In some embodiments, the CRISPR gene-editing system for the treatment of gout comprises one or more sgRNAs targeting exon 7 of eNFKB1. In some embodiments, the CRISPR gene-editing system for the treatment of gout comprises one or more sgRNAs targeting exon 8 of eNFKB1. In some embodiments, the CRISPR gene-editing system for the treatment of gout comprises one or more sgRNAs targeting exon 9 of eNFKB1. In some embodiments, the CRISPR gene-editing system for the treatment of gout comprises one or more sgRNAs targeting exon 10 of eNFKB1. In some embodiments, the CRISPR gene-editing system for the treatment of gout comprises one or more sgRNAs targeting exon 11 of eNFKB1. In some embodiments, the CRISPR gene-editing system for the treatment of gout comprises one or more sgRNAs targeting exon 12 of eNFKB1. In some embodiments, the CRISPR gene-editing system for the treatment of gout comprises one or more sgRNAs targeting exon 13 of eNFKB1. In some embodiments, the CRISPR gene-editing system for the treatment of gout comprises one or more sgRNAs targeting exon 14 of eNFKB1. In some embodiments, the CRISPR gene-editing system for the treatment of gout comprises one or more sgRNAs targeting exon 15 of eNFKB1. In some embodiments, the CRISPR gene-editing system for the treatment of gout comprises one or more sgRNAs targeting exon 16 of eNFKB1. In some embodiments, the CRISPR gene-editing system for the treatment of gout comprises one or more sgRNAs targeting exon 17 of eNFKB1. In some embodiments, the CRISPR gene-editing system for the treatment of gout comprises one or more sgRNAs targeting exon 18 of eNFKB1. In some embodiments, the CRISPR gene-editing system for the treatment of gout comprises one or more sgRNAs targeting exon 19 of eNFKB1. In some embodiments, the CRISPR gene-editing system for the treatment of gout comprises one or more sgRNAs targeting exon 20 of eNFKB1. In some embodiments, the CRISPR gene-editing system for the treatment of gout comprises one or more sgRNAs targeting exon 21 of eNFKB1. In some embodiments, the CRISPR gene-editing system for the treatment of gout comprises one or more sgRNAs targeting exon 22 of eNFKB1. In some embodiments, the CRISPR gene-editing system for the treatment of gout comprises one or more sgRNAs targeting exon 23 of eNFKB1. In some embodiments, the CRISPR gene-editing system for the treatment of gout comprises one or more sgRNAs targeting exon 24 of eNFKB1. In some embodiments, the CRISPR gene-editing system for the treatment of gout comprises one or more sgRNAs targeting exon 25 of eNFKB1. In some embodiments, the CRISPR gene-editing system for the treatment of gout comprises one or more sgRNAs targeting exon 26 of eNFKB1. In some embodiments, the CRISPR gene-editing system for the treatment of gout comprises one or more sgRNAs targeting exon 27 of eNFKB1. In some embodiments, the CRISPR gene-editing system for the treatment of gout comprises one or more sgRNAs targeting exon 28 of eNFKB1.

In some embodiments, the CRISPR gene-editing system for the treatment of gout comprises one or more sgRNAs targeting exon 1 of fNFKB1. In some embodiments, the CRISPR gene-editing system for the treatment of gout comprises one or more sgRNAs targeting exon 2 of fNFKB1. In some embodiments, the CRISPR gene-editing system for the treatment of gout comprises one or more sgRNAs targeting exon 3 of fNFKB1. In some embodiments, the CRISPR gene-editing system for the treatment of gout comprises one or more sgRNAs targeting exon 4 of fNFKB1. In some embodiments, the CRISPR gene-editing system for the treatment of gout comprises one or more sgRNAs targeting exon 5 of fNFKB1. In some embodiments, the CRISPR gene-editing system for the treatment of gout comprises one or more sgRNAs targeting exon 6 of fNFKB1. In some embodiments, the CRISPR gene-editing system for the treatment of gout comprises one or more sgRNAs targeting exon 7 of fNFKB1. In some embodiments, the CRISPR gene-editing system for the treatment of gout comprises one or more sgRNAs targeting exon 8 of fNFKB1. In some embodiments, the CRISPR gene-editing system for the treatment of gout comprises one or more sgRNAs targeting exon 9 of fNFKB1. In some embodiments, the CRISPR gene-editing system for the treatment of gout comprises one or more sgRNAs targeting exon 10 of fNFKB1. In some embodiments, the CRISPR gene-editing system for the treatment of gout comprises one or more sgRNAs targeting exon 11 of fNFKB1. In some embodiments, the CRISPR gene-editing system for the treatment of gout comprises one or more sgRNAs targeting exon 12 of fNFKB1. In some embodiments, the CRISPR gene-editing system for the treatment of gout comprises one or more sgRNAs targeting exon 13 of fNFKB1. In some embodiments, the CRISPR gene-editing system for the treatment of gout comprises one or more sgRNAs targeting exon 14 of fNFKB1. In some embodiments, the CRISPR gene-editing system for the treatment of gout comprises one or more sgRNAs targeting exon 15 of fNFKB1. In some embodiments, the CRISPR gene-editing system for the treatment of gout comprises one or more sgRNAs targeting exon 16 of fNFKB1. In some embodiments, the CRISPR gene-editing system for the treatment of gout comprises one or more sgRNAs targeting exon 17 of fNFKB1. In some embodiments, the CRISPR gene-editing system for the treatment of gout comprises one or more sgRNAs targeting exon 18 of fNFKB1. In some embodiments, the CRISPR gene-editing system for the treatment of gout comprises one or more sgRNAs targeting exon 19 of fNFKB1. In some embodiments, the CRISPR gene-editing system for the treatment of gout comprises one or more sgRNAs targeting exon 20 of fNFKB1. In some embodiments, the CRISPR gene-editing system for the treatment of gout comprises one or more sgRNAs targeting exon 21 of fNFKB1. In some embodiments, the CRISPR gene-editing system for the treatment of gout comprises one or more sgRNAs targeting exon 22 of fNFKB1. In some embodiments, the CRISPR gene-editing system for the treatment of gout comprises one or more sgRNAs targeting exon 23 of fNFKB1. In some embodiments, the CRISPR gene-editing system for the treatment of gout comprises one or more sgRNAs targeting exon 24 of fNFKB1. In some embodiments, the CRISPR gene-editing system for the treatment of gout comprises one or more sgRNAs targeting exon 25 of fNFKB1.

In some embodiments, the CRISPR gene-editing system for the treatment of gout comprises one or more sgRNAs targeting NFKB2. In some embodiments, the CRISPR gene-editing system for the treatment of gout comprises one or more sgRNAs targeting hNFKB2. In some embodiments, the CRISPR gene-editing system for the treatment of gout comprises one or more sgRNAs targeting cNFKB2. In some embodiments, the CRISPR gene-editing system for the treatment of gout comprises one or more sgRNAs targeting eNFKB2. In some embodiments, the CRISPR gene-editing system for the treatment of gout comprises one or more sgRNAs targeting fNFKB2.

In some embodiments, the CRISPR gene-editing system for the treatment of gout comprises one or more sgRNAs targeting exon 1 of hNFKB2. In some embodiments, the CRISPR gene-editing system for the treatment of gout comprises one or more sgRNAs targeting exon 2 of hNFKB2. In some embodiments, the CRISPR gene-editing system for the treatment of gout comprises one or more sgRNAs targeting exon 3 of hNFKB2. In some embodiments, the CRISPR gene-editing system for the treatment of gout comprises one or more sgRNAs targeting exon 4 of hNFKB2. In some embodiments, the CRISPR gene-editing system for the treatment of gout comprises one or more sgRNAs targeting exon 5 of hNFKB2. In some embodiments, the CRISPR gene-editing system for the treatment of gout comprises one or more sgRNAs targeting exon 6 of hNFKB2. In some embodiments, the CRISPR gene-editing system for the treatment of gout comprises one or more sgRNAs targeting exon 7 of hNFKB2. In some embodiments, the CRISPR gene-editing system for the treatment of gout comprises one or more sgRNAs targeting exon 8 of hNFKB2. In some embodiments, the CRISPR gene-editing system for the treatment of gout comprises one or more sgRNAs targeting exon 9 of hNFKB2. In some embodiments, the CRISPR gene-editing system for the treatment of gout comprises one or more sgRNAs targeting exon 10 of hNFKB2. In some embodiments, the CRISPR gene-editing system for the treatment of gout comprises one or more sgRNAs targeting exon 11 of hNFKB2. In some embodiments, the CRISPR gene-editing system for the treatment of gout comprises one or more sgRNAs targeting exon 12 of hNFKB2. In some embodiments, the CRISPR gene-editing system for the treatment of gout comprises one or more sgRNAs targeting exon 13 of hNFKB2. In some embodiments, the CRISPR gene-editing system for the treatment of gout comprises one or more sgRNAs targeting exon 14 of hNFKB2. In some embodiments, the CRISPR gene-editing system for the treatment of gout comprises one or more sgRNAs targeting exon 15 of hNFKB2. In some embodiments, the CRISPR gene-editing system for the treatment of gout comprises one or more sgRNAs targeting exon 16 of hNFKB2. In some embodiments, the CRISPR gene-editing system for the treatment of gout comprises one or more sgRNAs targeting exon 17 of hNFKB2. In some embodiments, the CRISPR gene-editing system for the treatment of gout comprises one or more sgRNAs targeting exon 18 of hNFKB2. In some embodiments, the CRISPR gene-editing system for the treatment of gout comprises one or more sgRNAs targeting exon 19 of hNFKB2. In some embodiments, the CRISPR gene-editing system for the treatment of gout comprises one or more sgRNAs targeting exon 20 of hNFKB2. In some embodiments, the CRISPR gene-editing system for the treatment of gout comprises one or more sgRNAs targeting exon 21 of hNFKB2. In some embodiments, the CRISPR gene-editing system for the treatment of gout comprises one or more sgRNAs targeting exon 22 of hNFKB2. In some embodiments, the CRISPR gene-editing system for the treatment of gout comprises one or more sgRNAs targeting exon 23 of hNFKB2. In some embodiments, the CRISPR gene-editing system for the treatment of gout comprises one or more sgRNAs targeting exon 24 of hNFKB2.

In some embodiments, the CRISPR gene-editing system for the treatment of gout comprises one or more sgRNAs targeting exon 1 of cNFKB2. In some embodiments, the CRISPR gene-editing system for the treatment of gout comprises one or more sgRNAs targeting exon 2 of cNFKB2. In some embodiments, the CRISPR gene-editing system for the treatment of gout comprises one or more sgRNAs targeting exon 3 of cNFKB2. In some embodiments, the CRISPR gene-editing system for the treatment of gout comprises one or more sgRNAs targeting exon 4 of cNFKB2. In some embodiments, the CRISPR gene-editing system for the treatment of gout comprises one or more sgRNAs targeting exon 5 of cNFKB2. In some embodiments, the CRISPR gene-editing system for the treatment of gout comprises one or more sgRNAs targeting exon 6 of cNFKB2. In some embodiments, the CRISPR gene-editing system for the treatment of gout comprises one or more sgRNAs targeting exon 7 of cNFKB2. In some embodiments, the CRISPR gene-editing system for the treatment of gout comprises one or more sgRNAs targeting exon 8 of cNFKB2. In some embodiments, the CRISPR gene-editing system for the treatment of gout comprises one or more sgRNAs targeting exon 9 of cNFKB2. In some embodiments, the CRISPR gene-editing system for the treatment of gout comprises one or more sgRNAs targeting exon 10 of cNFKB2. In some embodiments, the CRISPR gene-editing system for the treatment of gout comprises one or more sgRNAs targeting exon 11 of cNFKB2. In some embodiments, the CRISPR gene-editing system for the treatment of gout comprises one or more sgRNAs targeting exon 12 of cNFKB2. In some embodiments, the CRISPR gene-editing system for the treatment of gout comprises one or more sgRNAs targeting exon 13 of cNFKB2. In some embodiments, the CRISPR gene-editing system for the treatment of gout comprises one or more sgRNAs targeting exon 14 of cNFKB2. In some embodiments, the CRISPR gene-editing system for the treatment of gout comprises one or more sgRNAs targeting exon 15 of cNFKB2. In some embodiments, the CRISPR gene-editing system for the treatment of gout comprises one or more sgRNAs targeting exon 16 of cNFKB2. In some embodiments, the CRISPR gene-editing system for the treatment of gout comprises one or more sgRNAs targeting exon 17 of cNFKB2. In some embodiments, the CRISPR gene-editing system for the treatment of gout comprises one or more sgRNAs targeting exon 18 of cNFKB2. In some embodiments, the CRISPR gene-editing system for the treatment of gout comprises one or more sgRNAs targeting exon 19 of cNFKB2. In some embodiments, the CRISPR gene-editing system for the treatment of gout comprises one or more sgRNAs targeting exon 20 of cNFKB2. In some embodiments, the CRISPR gene-editing system for the treatment of gout comprises one or more sgRNAs targeting exon 21 of cNFKB2. In some embodiments, the CRISPR gene-editing system for the treatment of gout comprises one or more sgRNAs targeting exon 22 of cNFKB2. In some embodiments, the CRISPR gene-editing system for the treatment of gout comprises one or more sgRNAs targeting exon 23 of cNFKB2.

In some embodiments, the CRISPR gene-editing system for the treatment of gout comprises one or more sgRNAs targeting exon 1 of eNFKB2. In some embodiments, the CRISPR gene-editing system for the treatment of gout comprises one or more sgRNAs targeting exon 2 of eNFKB2. In some embodiments, the CRISPR gene-editing system for the treatment of gout comprises one or more sgRNAs targeting exon 3 of eNFKB2. In some embodiments, the CRISPR gene-editing system for the treatment of gout comprises one or more sgRNAs targeting exon 4 of eNFKB2. In some embodiments, the CRISPR gene-editing system for the treatment of gout comprises one or more sgRNAs targeting exon 5 of eNFKB2. In some embodiments, the CRISPR gene-editing system for the treatment of gout comprises one or more sgRNAs targeting exon 6 of eNFKB2. In some embodiments, the CRISPR gene-editing system for the treatment of gout comprises one or more sgRNAs targeting exon 7 of eNFKB2. In some embodiments, the CRISPR gene-editing system for the treatment of gout comprises one or more sgRNAs targeting exon 8 of eNFKB2. In some embodiments, the CRISPR gene-editing system for the treatment of gout comprises one or more sgRNAs targeting exon 9 of eNFKB2. In some embodiments, the CRISPR gene-editing system for the treatment of gout comprises one or more sgRNAs targeting exon 10 of eNFKB2. In some embodiments, the CRISPR gene-editing system for the treatment of gout comprises one or more sgRNAs targeting exon 11 of eNFKB2. In some embodiments, the CRISPR gene-editing system for the treatment of gout comprises one or more sgRNAs targeting exon 12 of eNFKB2. In some embodiments, the CRISPR gene-editing system for the treatment of gout comprises one or more sgRNAs targeting exon 13 of eNFKB2. In some embodiments, the CRISPR gene-editing system for the treatment of gout comprises one or more sgRNAs targeting exon 14 of eNFKB2. In some embodiments, the CRISPR gene-editing system for the treatment of gout comprises one or more sgRNAs targeting exon 15 of eNFKB2. In some embodiments, the CRISPR gene-editing system for the treatment of gout comprises one or more sgRNAs targeting exon 16 of eNFKB2. In some embodiments, the CRISPR gene-editing system for the treatment of gout comprises one or more sgRNAs targeting exon 17 of eNFKB2. In some embodiments, the CRISPR gene-editing system for the treatment of gout comprises one or more sgRNAs targeting exon 18 of eNFKB2. In some embodiments, the CRISPR gene-editing system for the treatment of gout comprises one or more sgRNAs targeting exon 19 of eNFKB2. In some embodiments, the CRISPR gene-editing system for the treatment of gout comprises one or more sgRNAs targeting exon 20 of eNFKB2. In some embodiments, the CRISPR gene-editing system for the treatment of gout comprises one or more sgRNAs targeting exon 21 of eNFKB2. In some embodiments, the CRISPR gene-editing system for the treatment of gout comprises one or more sgRNAs targeting exon 22 of eNFKB2. In some embodiments, the CRISPR gene-editing system for the treatment of gout comprises one or more sgRNAs targeting exon 23 of eNFKB2.

In some embodiments, the CRISPR gene-editing system for the treatment of gout comprises one or more sgRNAs targeting exon 1 of fNFKB2. In some embodiments, the CRISPR gene-editing system for the treatment of gout comprises one or more sgRNAs targeting exon 2 of fNFKB2. In some embodiments, the CRISPR gene-editing system for the treatment of gout comprises one or more sgRNAs targeting exon 3 of fNFKB2. In some embodiments, the CRISPR gene-editing system for the treatment of gout comprises one or more sgRNAs targeting exon 4 of fNFKB2. In some embodiments, the CRISPR gene-editing system for the treatment of gout comprises one or more sgRNAs targeting exon 5 of fNFKB2. In some embodiments, the CRISPR gene-editing system for the treatment of gout comprises one or more sgRNAs targeting exon 6 of fNFKB2. In some embodiments, the CRISPR gene-editing system for the treatment of gout comprises one or more sgRNAs targeting exon 7 of fNFKB2. In some embodiments, the CRISPR gene-editing system for the treatment of gout comprises one or more sgRNAs targeting exon 8 of fNFKB2. In some embodiments, the CRISPR gene-editing system for the treatment of gout comprises one or more sgRNAs targeting exon 9 of fNFKB2. In some embodiments, the CRISPR gene-editing system for the treatment of gout comprises one or more sgRNAs targeting exon 10 of fNFKB2. In some embodiments, the CRISPR gene-editing system for the treatment of gout comprises one or more sgRNAs targeting exon 11 of fNFKB2. In some embodiments, the CRISPR gene-editing system for the treatment of gout comprises one or more sgRNAs targeting exon 12 of fNFKB2. In some embodiments, the CRISPR gene-editing system for the treatment of gout comprises one or more sgRNAs targeting exon 13 of fNFKB2. In some embodiments, the CRISPR gene-editing system for the treatment of gout comprises one or more sgRNAs targeting exon 14 of fNFKB2. In some embodiments, the CRISPR gene-editing system for the treatment of gout comprises one or more sgRNAs targeting exon 15 of fNFKB2. In some embodiments, the CRISPR gene-editing system for the treatment of gout comprises one or more sgRNAs targeting exon 16 of fNFKB2. In some embodiments, the CRISPR gene-editing system for the treatment of gout comprises one or more sgRNAs targeting exon 17 of fNFKB2. In some embodiments, the CRISPR gene-editing system for the treatment of gout comprises one or more sgRNAs targeting exon 18 of fNFKB2. In some embodiments, the CRISPR gene-editing system for the treatment of gout comprises one or more sgRNAs targeting exon 19 of fNFKB2. In some embodiments, the CRISPR gene-editing system for the treatment of gout comprises one or more sgRNAs targeting exon 20 of fNFKB2. In some embodiments, the CRISPR gene-editing system for the treatment of gout comprises one or more sgRNAs targeting exon 21 of fNFKB2. In some embodiments, the CRISPR gene-editing system for the treatment of gout comprises one or more sgRNAs targeting exon 22 of fNFKB2. In some embodiments, the CRISPR gene-editing system for the treatment of gout comprises one or more sgRNAs targeting exon 23 of fNFKB2.

### V. Delivery

### A. Viral Vectors

In one aspect, the present disclosure encompasses methods of delivery of a CRISPR gene-editing system targeting a gene selected from NFKB1, NFKB2, and combinations thereof using one or more recombinant viral particle. In some embodiments, the one of more viral vectors comprise a recombinant virus selected from a retrovirus, an adenovirus, an adeno-associated virus, a lentivirus, and a herpes simplex virus-1. In some embodiments, the one of more viral vectors comprise a recombinant adeno-associated virus (AAV). In some embodiments, the recombinant AAV is of serotype 5 (AAV5). In some embodiments, the recombinant AAV is of serotype 6 (AAV6). In some embodiments, the one or more viral vectors comprise: a first viral vector comprising a first nucleic acid, in the one or more nucleic acids, encoding the Cas protein; and a second viral vector comprising a second nucleic acid, in the one or more nucleic acids, encoding the at least one guide RNA. In some embodiments, the one or more viral vectors comprise a viral vector comprising a single nucleic acid, wherein the single nucleic acid encodes the Cas9 protein and the at least one guide RNA.

### 1. Adeno-Associated Virus (AAV)

A viral vector system useful for delivery of nucleic acids is the adeno-associated virus (AAV). Adeno-associated virus is a naturally occurring defective virus that requires another virus, such as an adenovirus or a herpes virus, as a helper virus for efficient replication and a productive life cycle. For a review see Muzyczka et al., Curr. Topics in Micro. and Immunol. 158:97-129 (1992). It is also one of the few viruses that may integrate its DNA into non-dividing cells, and exhibits a high frequency of stable integration (see for example Flotte et al., Am. J. Respir. Cell. Mol. Biol. 7:349-356 (1992); Samulski et al., J. Virol. 63:3822-3828 (1989); and McLaughlin et al., J. Virol. 62:1963-1973 (1989). Vectors containing as little as 300 base pairs of AAV can be packaged and can integrate. Space for exogenous DNA is limited to about 4.5 kb. An AAV vector such as that described in Tratschin et al., Mol. Cell. Biol. 5:3251-3260 (1985) can be used to introduce DNA into cells. A variety of nucleic acids have been introduced into different cell types using AAV vectors (see for example Hermonat et al., Proc. Natl. Acad. Sci. USA 81:6466-6470 (1984); Tratschin et al., Mol. Cell. Biol. 4:2072-2081 (1985); Wondisford et al., Mol. Endocrinol. 2:32-39 (1988); Tratschin et al., J. Virol. 51:611-619 (1984); and Flotte et al., J. Biol. Chem. 268:3781-3790 (1993). The identification of Staphylococcus aureus (SaCas9) and other smaller Cas9 enzymes that can be packaged into adeno-associated viral (AAV) vectors that are highly stable and effective in vivo, easily produced, approved by FDA, and tested in multiple clinical trials, paves new avenues for therapeutic gene editing.

According to particular embodiments, a CRISPR gene-editing system targeting a gene selected from NFKB1, NFKB2, and combinations thereof further comprise a recombinant AAV vector. In some embodiments, the CRISPR gene-editing system is encoded by a nucleic acid, wherein the nucleic acid is a recombinant AAV genome. In some embodiments, the AAV vector is selected from an AAV1 vector, an AAV2 vector, an AAV3 vector, an AAV4 vector, an AAV5 vector, an AAV6 vector, an AAV7 vector, an AAV8 vector, an AAV9 vector, and an AAV10 vector.

In some aspects, the AAV vector comprises a serotype selected from the group consisting of: AAV1, AAV1(Y705+731F+T492V), AAV2(Y444+500+730F+T491V), AAV3(Y705+731F), AAV4, AAV5, AAV5(Y436+693+719F), AAV6, AAV6 (VP3 variant Y705F/Y731F/T492V), AAV-7m8, AAV8, AAV8(Y733F), AAV9, AAV9 (VP3 variant Y731F), AAV10(Y733F), AAV-ShH10, and AAV-DJ/8. In some aspects, the AAV vector comprises a serotype selected from the group consisting of: AAV1, AAV5, AAV6, AAV6 (Y705F/Y731F/T492V), AAV8, AAV9, and AAV9 (Y731F).

In some embodiments, use of the CRISPR gene-editing system further comprising one or more LNPs to target a gene selected from NFKB1, NFKB2, and combinations thereof is therapeutic.

### 2. Lentivirus

In some aspects, the viral vector is a lentivirus. In an aspect, the lentivirus is selected from the group consisting of: human immunodeficiency-1 (HIV-1), human immunodeficiency-2 (HIV-2), simian immunodeficiency virus (SIV), feline immunodeficiency virus (FIV), bovine immunodeficiency virus (BIV), Jembrana Disease Virus (JDV), equine infectious anemia virus (EIAV), and caprine arthritis encephalitis virus (CAEV).

Lentiviral transduction systems are known in the art and are described, e.g., in Levine, et al., Proc. Nat'l Acad. Sci. 2006, 103, 17372-77; Zufferey, et al., Nat. Biotechnol. 1997, 15, 871-75; Dull, et al., J. Virology 1998, 72, 8463-71, and U.S. Pat. No. 6,627,442, the disclosures of each of which are incorporated by reference herein.

### B. Lipid Nanoparticles (LNP)

In some embodiments, a CRISPR gene-editing system is delivered by a nanoparticle. Without wishing to be bound by any particular theory, in certain embodiments, nucleic acids, when present in the nanoparticle, are resistant in aqueous solution to degradation with a nuclease. In other embodiments, proteins are protected from protease degradation. In some embodiments, proteins and nucleic acids encapsulated by nanoparticles are capable of penetrating the cellular plasma membrane.

Lipid nanoparticles comprising nucleic acids and their method of preparation is disclosed in at least WO2017/019935, WO2017/049074, WO2017/201346, WO2017/218704, WO2018/006052, WO2018/013525, WO2018/089540, WO2018/119115, WO2018/126084, WO2018/157009, WO2018/170336, WO2018/222890, WO2019/046809, WO2019/089828, WO2020/061284, WO2020/061317, WO2020/081938, WO2020/097511, WO2020/097520, WO2020/097540, WO2020/097548, WO2020/214946, WO2020/219941, WO2020/232276, WO2020/227615, WO2020/061295, WO2021/007278, WO2021/016430, WO2021/021988, EP Patent No. EP 2 972 360, US20200155691, US20200237671, U.S. Pat. Nos. 8,058,069, 8,492,359, 8,822,668, 9,364,435, 9,404,127, 9,504,651, 9,593,077, 9,738,593, 9,868,691, 9,868,692, 9,950,068, 10,138,213, 10,166,298, 10,221,127, 10,238,754, 10,266,485, 10,383,952, 10,730,924, 10,766,852, 11,079,379, 11,141,378 and 11,246,933, which are incorporated herein by reference in their entirety for all purposes.

### Lipid Nanoparticle Compositions

In some embodiments, the largest dimension of a nanoparticle composition is 1 micrometer or shorter (e.g., 1 micrometer, 900 nm, 800 nm, 700 nm, 600 nm, 500 nm, 400 nm, 300 nm, 200 nm, 175 nm, 150 nm, 125 nm, 100 nm, 75 nm, 50 nm, or shorter), e.g., when measured by dynamic light scattering (DLS), transmission electron microscopy, scanning electron microscopy, or another method. Nanoparticle compositions include, for example, lipid nanoparticles (LNPs), liposomes, lipid vesicles, and lipoplexes. In some embodiments, nanoparticle compositions are vesicles including one or more lipid bilayers. In certain embodiments, a nanoparticle composition includes two or more concentric bilayers separated by aqueous compartments. Lipid bilayers may be functionalized and/or crosslinked to one another. Lipid bilayers may include one or more ligands, proteins, or channels. In various embodiments, lipid nanoparticles described herein have a mean diameter of from about 30 nm to about 150 nm, from about 40 nm to about 150 nm, from about 50 nm to about 150 nm, from about 60 nm to about 130 nm, from about 70 nm to about 110 nm, from about 70 nm to about 100 nm, from about 80 nm to about 100 nm, from about 90 nm to about 100 nm, from about 70 nm to about 90 nm, from about 80 nm to about 90 nm, from about 70 nm to about 80 nm, or about 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm, and are substantially non-toxic.

In certain embodiments, the lipid nanoparticles described herein comprise one or more components, including a lipid component, and (optionally) a structural component. The lipid component comprises lipids selected from ionizable and/or cationic lipids (i.e., lipids that may have a positive or partial positive charge at physiological pH), neutral lipids (e.g., phospholipids, or sphingolipids), and polymer-conjugated lipids (e.g., PEGylated lipids). In some embodiments, the lipid component comprises a single ionizable lipid. In other embodiments, the lipid component comprises 2, 3, 4, 5, 6, 7, 8, 9, 10, 15, or 20 ionizable lipids. In some embodiments, the lipid component comprises a single neutral lipid. In other embodiments, the lipid component comprises 2, 3, 4, 5, 6, 7, 8, 9, 10, 15, or 20 neutral lipids. In some embodiments, the lipid com-ponent comprises a single polymer-conjugated lipid. In other embodiments, the lipid component comprises 2, 3, 4, 5, 6, 7, 8, 9, 10, 15, or 20 polymer-conjugated lipids. In some embodiments, the structural component comprises a single structural lipid. In other embodiments, the structural component comprises 2, 3, 4, 5, 6, 7, 8, 9, 10, 15, or 20 structural lipids. In some embodiments, the lipid component comprises at least one cationic lipid, at least one neutral lipid, and at least one polymer-conjugated lipid. The present disclosure contemplates that the lipid component may comprise any combination of the foregoing constituents.

### Ionizable/Cationic Lipids

In some embodiments, the lipid component comprises an ionizable lipid. In some embodiments, the ionizable lipid is anionic. In other embodiments, the ionizable lipid is a cationic lipid. In some embodiments, the lipid component comprises cationic lipids including, but not limited to, a cationic lipid selected from the group consisting of 3-(didodecylamino)-N1,N1,4-tridodecyl-1-piperazineethanamine (KL10), N1-[2-(didodecylamino)ethyl]-N1,N4,N4-tridodecyl-1,4-piperazinediethanamine (KL22), 14,25-ditridecyl-15,18,21,24-tetraaza-octatriacontane (KL25), 1,2-dilinoleyloxy-N,N-dimethylaminopropane (DLin-DMA), 2,2-dilinoleyl-4-dimethylaminomethyl-[1,3]-dioxolane (DLin-K-DMA), heptatriaconta-6,9,28,31-tetraen-19-yl 4-(dimethylamino)butanoate (DLin-MC3-DMA), 2,2-dilinoleyl-4-(2-dimethylaminoethyl)-[1,3]-dioxolane (DLin-KC2-DMA), 1,2-dioleyloxy-N,N-dimethylaminopropane (DODMA), 2-({8-[(3.beta.)-cholest-5-en-3-yloxy]octyl}oxy)-N,N-dimethyl-3-[(9Z,12Z)—octadeca-9,12-dien-1-yloxy]propan-1-amine (Octyl-CLinDMA), (2R)-2-({8-[(3.beta.)-cholest-5-en-3-yloxy]octyl}oxy)-N,N-dimethyl-3-[(9Z-,12Z)-octadeca-9,12-dien-1-yloxy]propan-1-amine (Octyl-CLinDMA (2R)), (2S)-2-({8-[(3.beta.)-cholest-5-en-3-yloxy]octyl}oxy)-N,N-dimethyl-3-[(9Z-,12Z)-octadeca-9,12-dien-1-yloxy]propan-1-amine (Octyl-CLinDMA (2S)), a lipid including a cyclic amine group, and mixtures thereof.

### Non-Exhaustive and Non-Limiting Examples of Cationic Lipids Include:

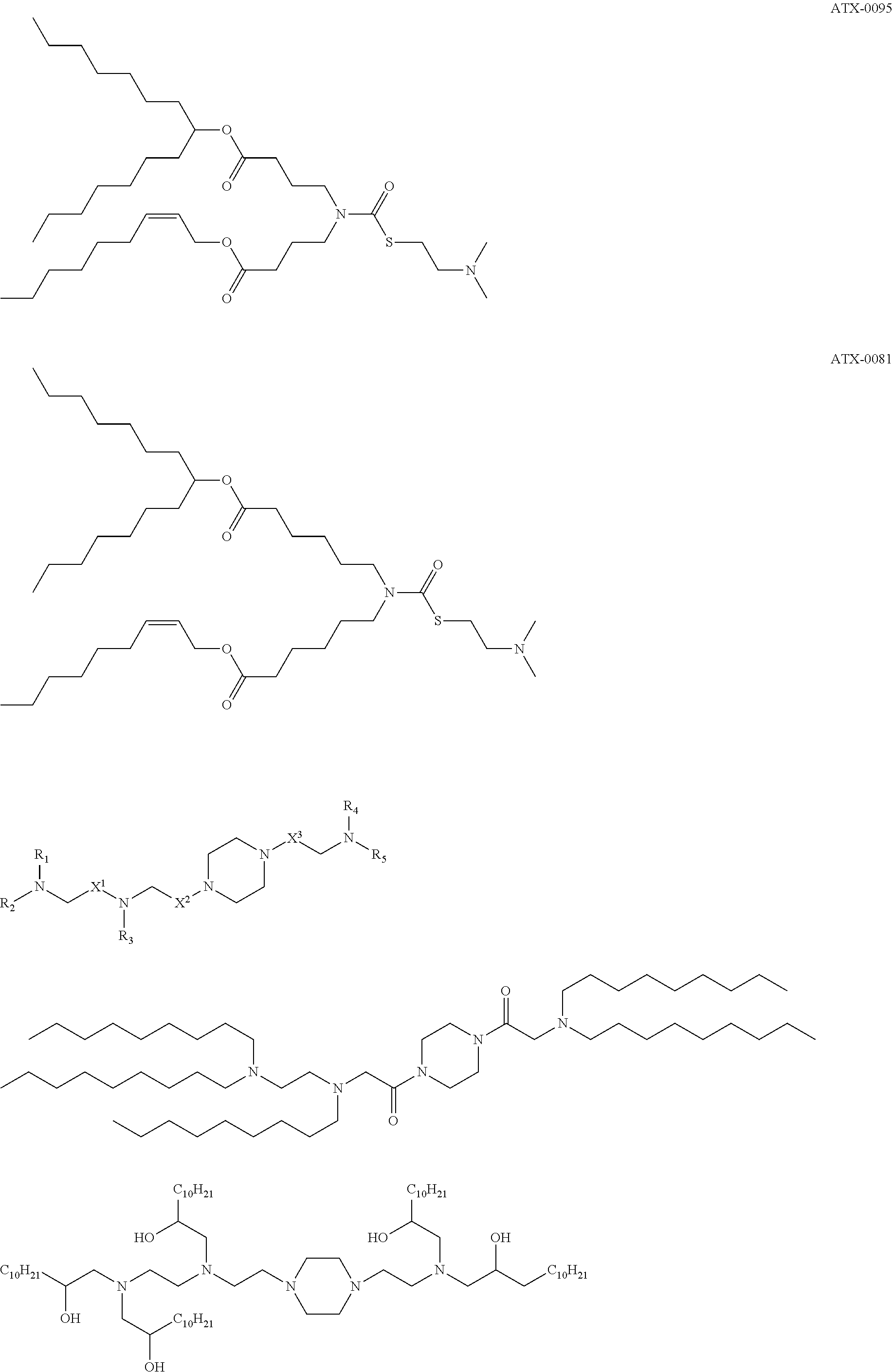

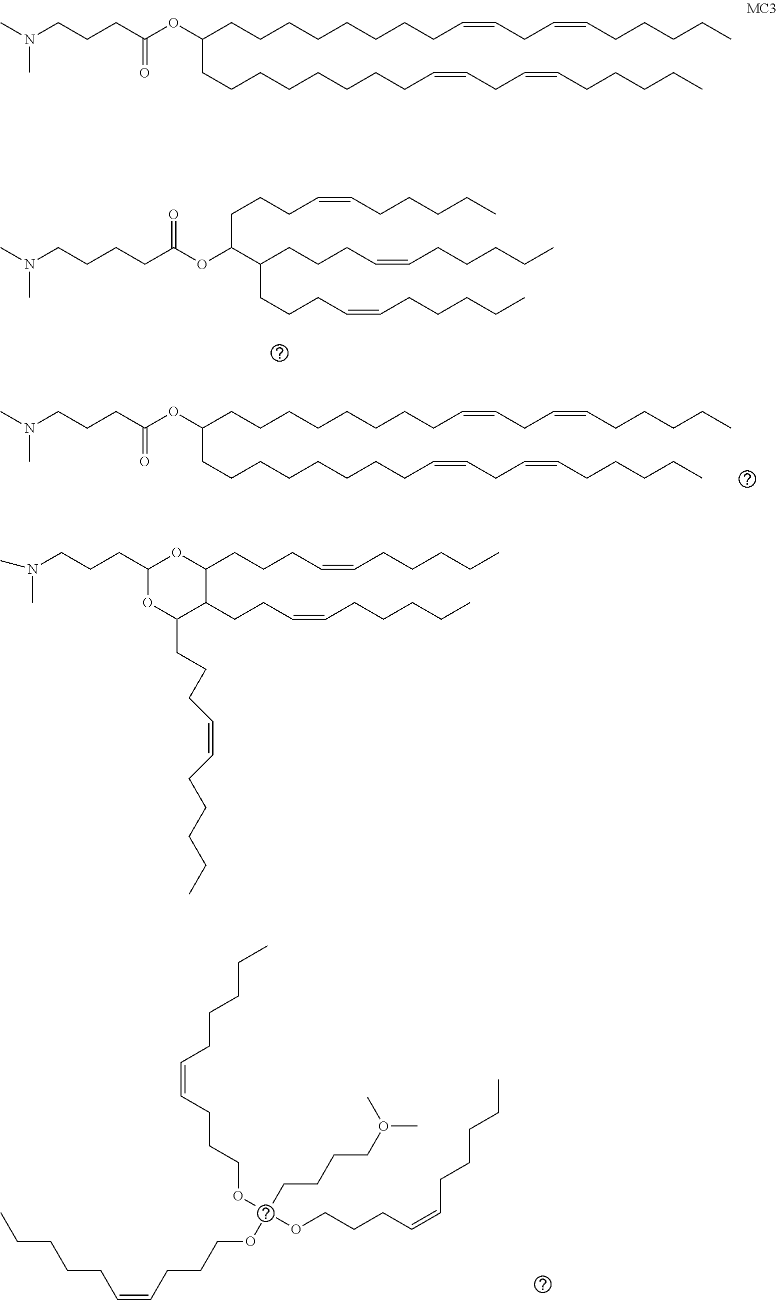

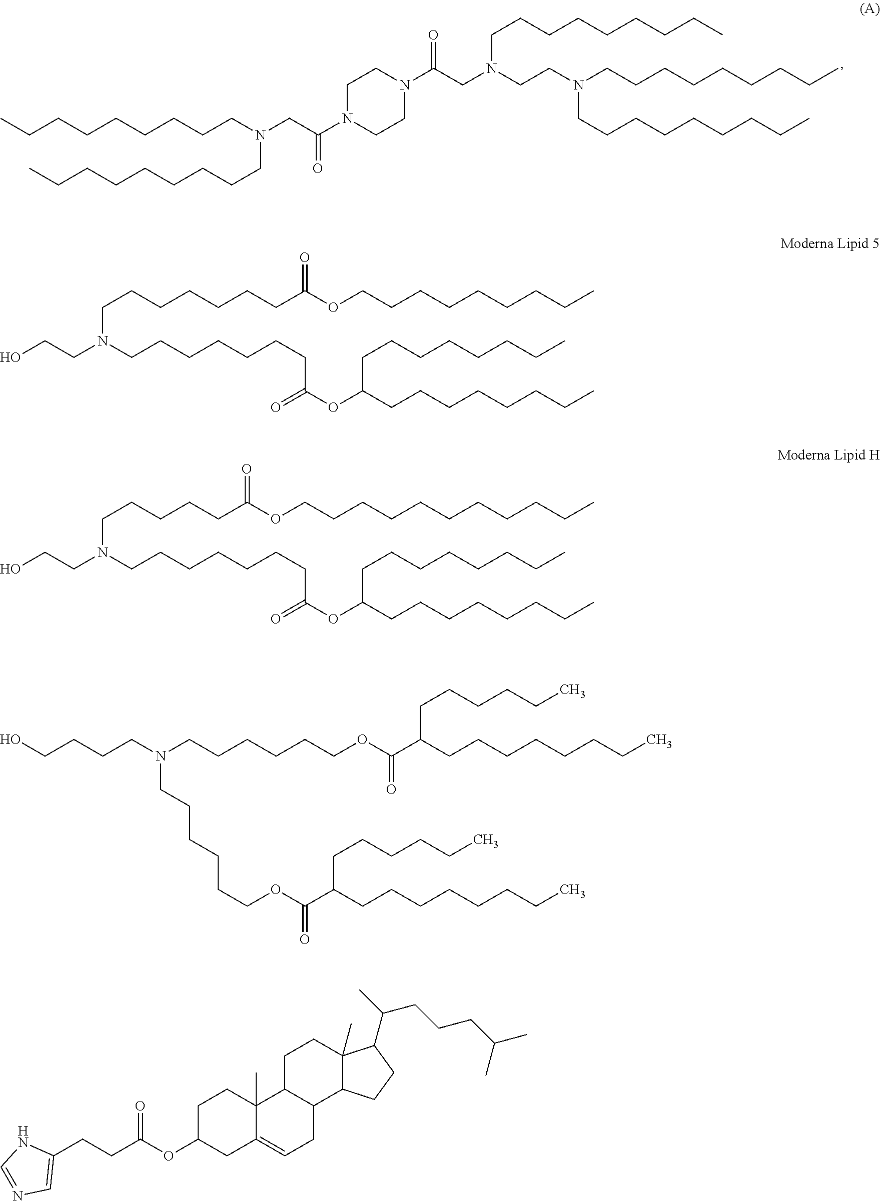 [SMILES: CCCCCCCCCN(CCCCCCCCC)CCN(CCCCCCCCC)CC(=O)N1CCN(C(=O)CN(CCCCCCCCC)CCCCCCCCC)CC1 ; CCCCCCCCCOC(=O)CCCCCCCN(CCO)CCCCCCCC(=O)OC(CCCCCCCC)CCCCCCCC ; CCCCCCCCCCCOC(=O)CCCCCN(CCO)CCCCCCCC(=O)OC(CCCCCCCC)CCCCCCCC ; CCCCCCCCC(CCCCCC)C(=O)OCCCCCCN(CCCCO)CCCCCCOC(=O)C(CCCCCC)CCCCCCCC ; CC(C)CCCC(C)C1CCC2C3CC=C4CC(OC(=O)CCc5cnc[nH]5)CCC4(C)C3CCC12C]

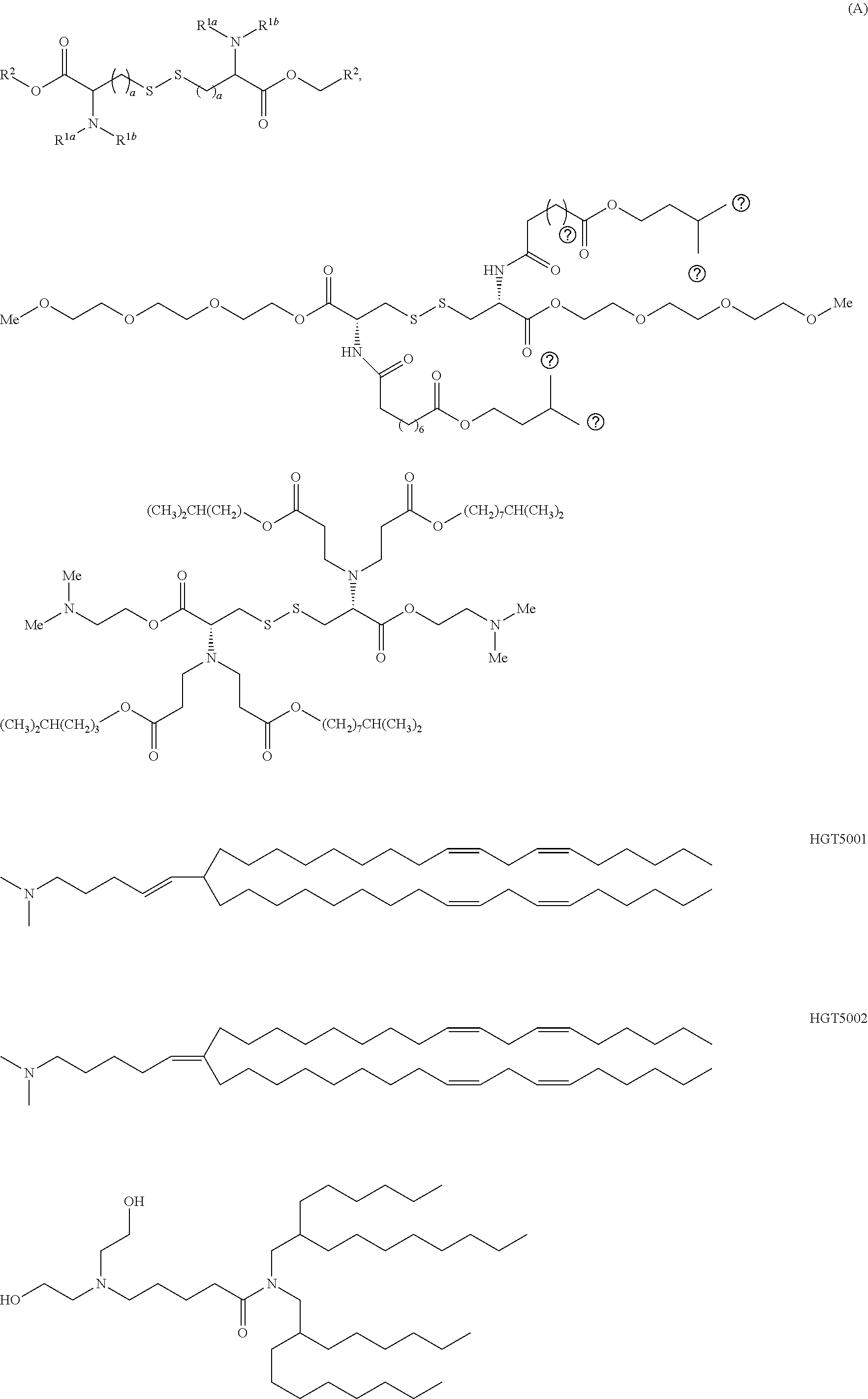

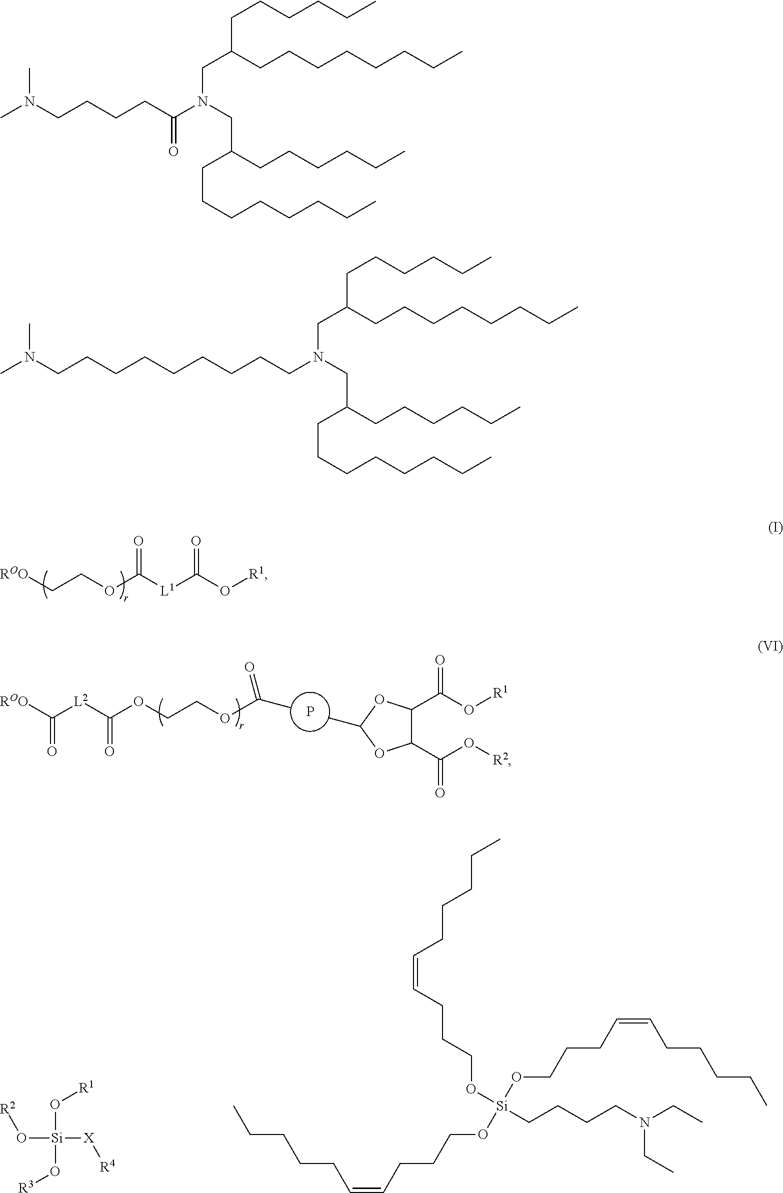

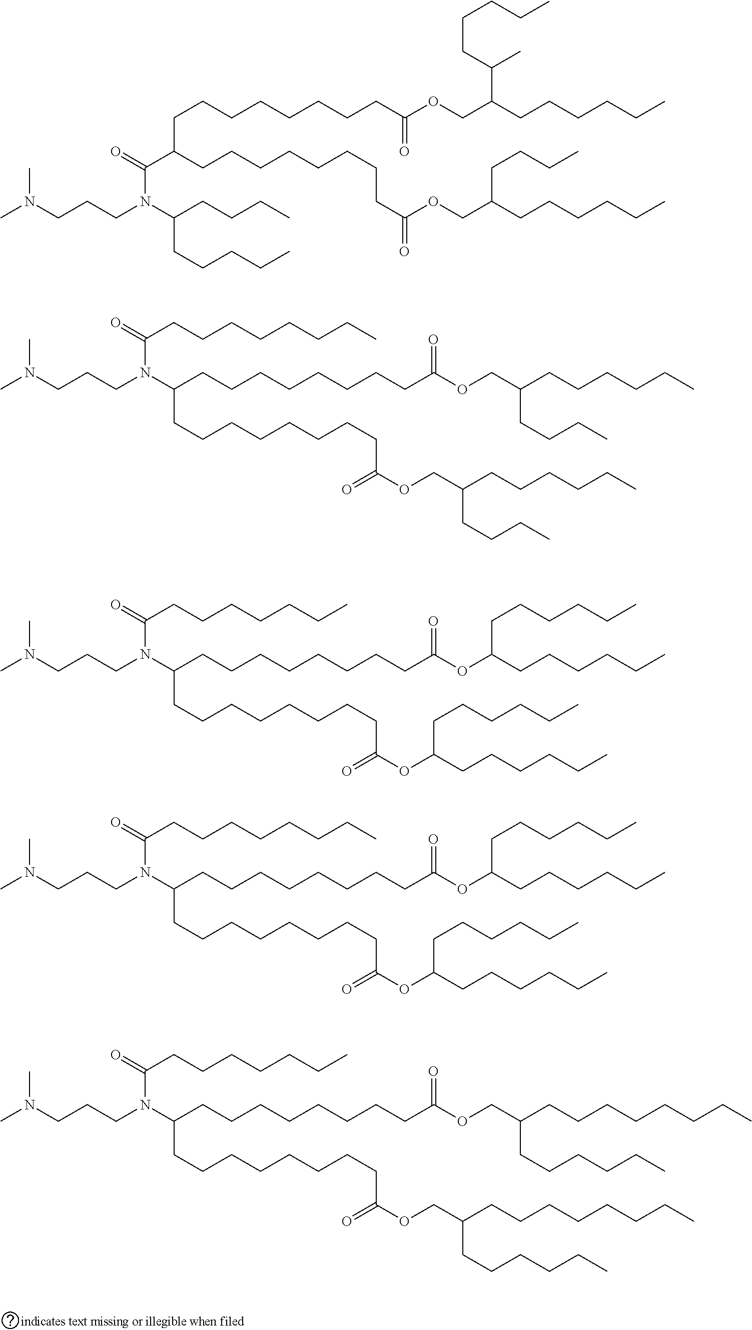

### Neutral Lipids/Phospholipids

In some embodiments, the lipid component further comprises neutral lipids including, but not limited to, a phospholipid selected from the group consisting of 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-dioleoyl-sn-glycero-3-phosphoethanolamine (DOPE), 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), sphingomyelin (SM), and mixtures thereof.

### Polymer-Conjugated Lipids

In some embodiments, the lipid component further comprises polymer-conjugated lipids, including, but not limited to, a PEGylated lipid selected from the group consisting of PEG-modified phosphatidylethanolamines, PEG-modified phosphatidic acids, PEG-modified ceramides, PEG-modified dialkylamines, PEG-modified diacylglycerols, PEG-modified dialkylglycerols, and mixtures thereof. For example, a PEG lipid may be PEG-c-DOMG, PEG-DMG, PEG₂₀₀₀-c-DMG, PEG-DLPE, PEG-DMPE, PEG-DPPC, PEG-DMA or a PEG-DSPE lipid.

### Non-Exhaustive and Non-Limiting Examples of PEG Lipids Include:

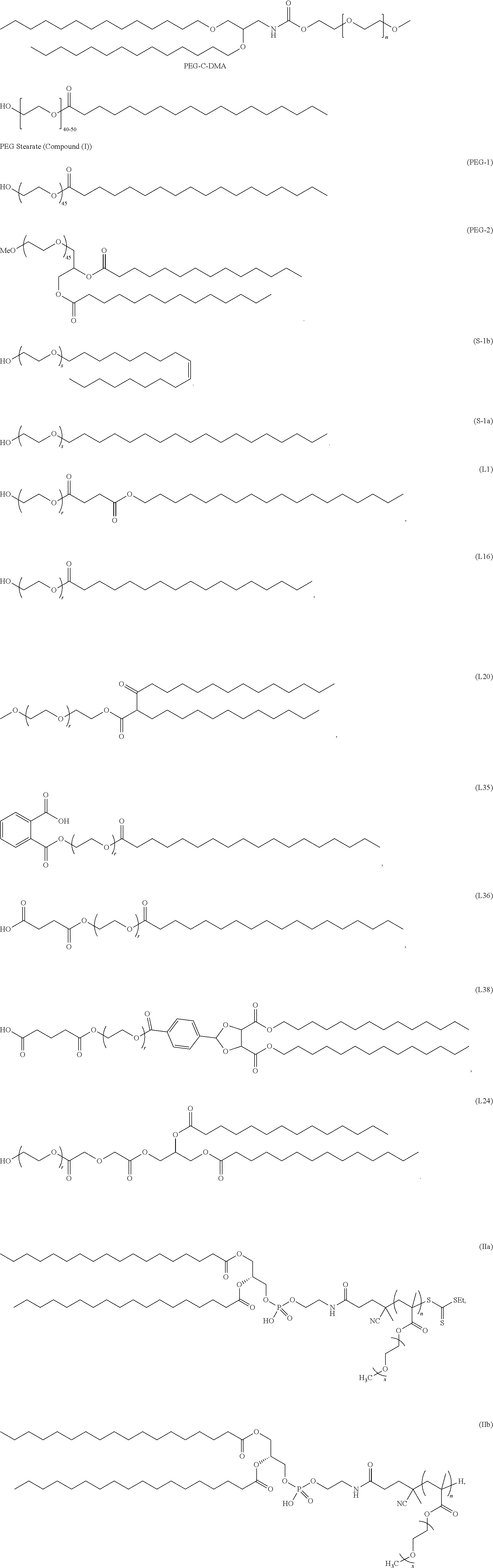

### Structural Lipids/Sterols

In some embodiments, the LNP further comprises a structural component. See generally Patel, S., et al. (2020). Nature Communications, 11(1), 1-13. In some embodiments, the structural component comprises a sterol including, but not limited to, a sterol selected from the group consisting of cholesterol, fecosterol, stigmasterol, stigmastanol, sitosterol, (3-sitosterol, lupeol, betulin, ursolic acid, oleanolic acid, campesterol, fucosterol, brassicasterol, ergosterol, 9,11-dehydroergosterol, tomatidine, tomatine, α-tocopherol, and mixtures thereof. In other embodiments, the structural lipid includes cholesterol and a corticosteroid (e.g., prednisolone, dexamethasone, prednisone, and hydrocortisone), or a combination thereof.

### Non-Exhaustive and Non-Limiting Examples of Structural Lipids Include:

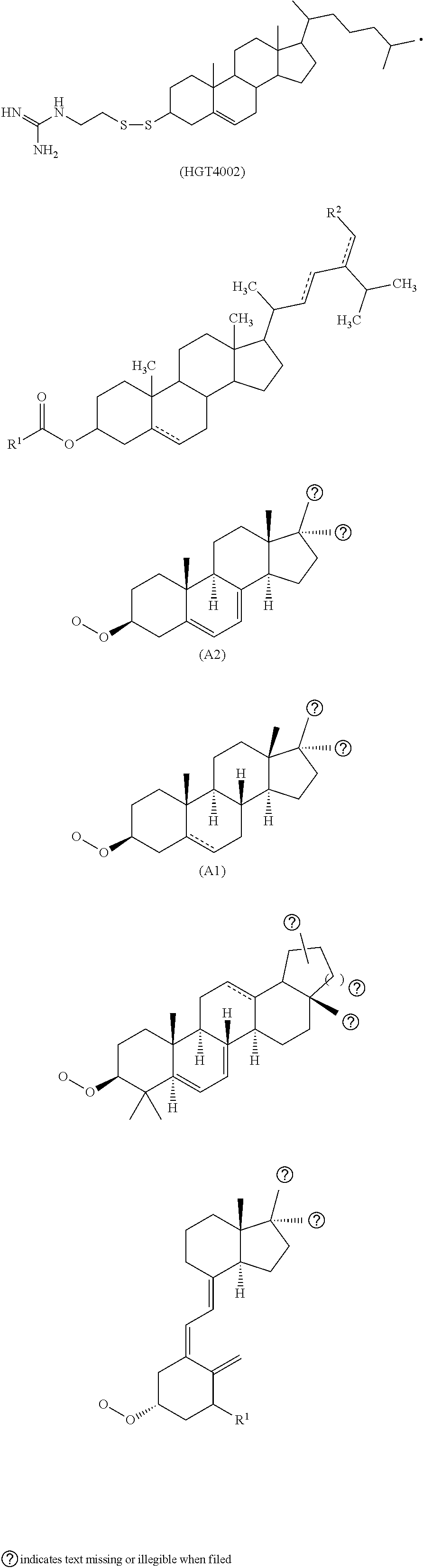

### Formulations

Nanoparticle compositions may include a lipid component and one or more additional components, such as a therapeutic and/or prophylactic. A nanoparticle composition may be designed for one or more specific applications or targets. The elements of a nanoparticle composition may be selected based on a particular application or target, and/or based on the efficacy, toxicity, expense, ease of use, availability, or other feature of one or more elements. Similarly, the particular formulation of a nanoparticle composition may be selected for a particular application or target according to, for example, the efficacy and toxicity of particular combinations of elements.

The lipid component of a nanoparticle composition may include, for example, a cationic lipid, a phospholipid (such as an unsaturated lipid, e.g., DOPE or DSPC), a PEG lipid, and a structural lipid. The elements of the lipid component may be provided in specific fractions.

In some embodiments, the lipid component of a nanoparticle composition includes an ionizable lipid, a phospholipid, a PEG lipid, and a structural lipid. In certain embodiments, the lipid com-ponent of the nanoparticle composition includes about 30 mol % to about 60 mol % ionizable lipid, about 0 mol % to about 30 mol % phospholipid, about 0 mol % to about 10 mol % of PEG lipid, and about 17.5 mol % to about 50 mol % structural lipid, provided that the total mol % does not exceed 100%. In some embodiments, the lipid component of the nanoparticle composition includes about 35 mol % to about 55 mol % compound of ionizable lipid, about 5 mol % to about 25 mol % phospholipid, about 0 mol % to about 10 mol % of PEG lipid, and about 30 mol % to about 40 mol % structural lipid. In a particular embodiment, the lipid component includes about 50 mol % said compound, about 10 mol % phospholipid, about 38.5 mol % structural lipid, and about 1.5 mol % of PEG lipid. In another embodiment, the lipid component includes about 40 mol % said compound, about 20 mol % phospholipid, about 38.5 mol % structural lipid, and about 1.5 mol % of PEG lipid. In some embodiments, the phospholipid may be DOPE or DSPC. In other embodiments, the PEG lipid may be PEG-DMG and/or the structural lipid may be cholesterol.

In some embodiments, the ionizable lipids comprise between about 20 and about 60 mol % of the lipid component. In other embodiments, the ionizable lipids comprise between about 35 and about 55 mol % of the lipid component. In various embodiments, the ionizable lipids comprise about 20, 21, 22, 23, 24, 25, 26, 27, 28, 29, 30, 35, 40, 45, 50, 55, or 60 mol % of the lipid component.

In some embodiments, the neutral lipids comprise between about 0 and about 30 mol % of the lipid component. In other embodiments, the neutral lipids comprise between about 5 and about 25 mol % of the lipid component. In various embodiments, the neutral lipids comprise about 1, 2, 3, 4, 5, 6, 7, 8, 9, 10, 11, 12, 13, 14, 15, 16, 17, 18, 19, 20, 25, or 30 mol % of the lipid component.

In some embodiments, the polymer-conjugated lipids comprise between about 0 and about 15 mol % of the lipid component. In other embodiments, the polymer-conjugated lipids comprise between about 0.5 and about 10 mol % of the lipid component. In various embodiments, the polymer-conjugated lipids comprise about 0.5, 1, 1.5, 2, 2.5, 3, 3.5, 4, 4.5, 5, 5.5, 6, 6.5, 7, 7.5, 8, 8.5 9, 9.5, 10, or 15 mol % of the lipid component.

In some embodiments, the structural component comprises about 17.5 mol % to about 50 mol % of the lipid component. In other embodiments, the structural component comprises about 30 to about 40 mol % of the lipid component. In various embodiments, the structural component comprises about 17.5, 20, 22.5, 25, 27.5, 30, 31, 32, 33, 34, 35, 36, 37, 38, 39, 40, 41, 42, 43, 44, 45, 46, 47, 48, 49, or 50 mol % of the lipid component.

The structural component may alternatively be expressed as a ratio relative to the lipid component. In some embodiments, the structural component is in a ratio of about 1:1 with the lipid component (sterol:lipids). In other embodiments, the structural component is in a ratio of about 1:5 with the lipid component (sterol:lipids). In various embodiments, the structural component is in a ratio of about 1:1, 1:2, 1:3, 1:4, 1:5, 1:6, 1:7, 1:8, 1:9, 1:10, 1:15, 1:20, or 1:25 with the lipid component (sterol:lipids).

Nanoparticle compositions may be designed for one or more specific applications or targets. For example, a nanoparticle composition may be designed to deliver a therapeutic and/or prophylactic such as an RNA to a particular cell, tissue, organ, or system or group thereof in a mammal's body. Physiochemical properties of nanoparticle compositions may be altered in order to increase selectivity for particular bodily targets. For instance, particle sizes may be adjusted based on the fenestration sizes of different organs. The therapeutic and/or prophylactic included in a nanoparticle composition may also be selected based on the desired delivery target or targets. For example, a therapeutic and/or prophylactic may be selected for a particular indication, condition, disease, or disorder and/or for delivery to a particular cell, tissue, organ, or system or group thereof (e.g., localized or specific delivery). In certain embodiments, a nanoparticle composition may include an mRNA encoding a polypeptide of interest capable of being translated within a cell to produce the polypeptide of interest. Such a composition may be designed to be specifically delivered to a particular organ. In some embodiments, a composition may be de-signed to be specifically delivered to a mammalian joint.

The amount of a therapeutic and/or prophylactic in a nanoparticle composition may depend on the size, composition, desired target and/or application, or other properties of the nanoparticle composition as well as on the properties of the therapeutic and/or prophylactic. For example, the amount of an RNA useful in a nanoparticle composition may depend on the size, sequence, and other characteristics of the RNA. The relative amounts of a therapeutic and/or prophylactic and other elements (e.g., lipids) in a nanoparticle composition may also vary. In some embodiments, the wt/wt ratio of the lipid component to a therapeutic and/or prophylactic in a nanoparticle composition may be from about 5:1 to about 60:1, such as 5:1, 6:1, 7:1, 8:1, 9:1, 10:1, 11:1, 12:1, 13:1, 14:1, 15:1, 16:1, 17:1, 18:1, 19:1, 20:1, 25:1, 30:1, 35:1, 40:1, 45:1, 50:1, and 60:1. For example, the wt/wt ratio of the lipid component to a therapeutic and/or prophylactic may be from about 10:1 to about 40:1. In certain embodiments, the wt/wt ratio is about 20:1. The amount of a therapeutic and/or prophylactic in a nanoparticle composition may, for example, be measured using absorption spectroscopy (e.g., ultraviolet-visible spectroscopy).

In some embodiments, the therapeutic and/or prophylactic comprises a nucleic acid component. In some embodiments, the nucleic acid component comprises RNA including, but not limited to, RNA selected from the group consisting of messenger RNA (mRNA), CRISPR RNA (crRNA), tracrRNA, single-guide RNA (sgRNA), short interfering RNA (siRNA), antisense oligonucleotides (ASO), and mixtures thereof. In other embodiments, the nucleic acid component comprises DNA including, but not limited to, DNA selected from the group consisting of linear DNA, plasmid DNA, antisense oligonucleotide, and mixtures thereof.

In some embodiments, a nanoparticle composition includes one or more RNAs, and the one or more RNAs, lipids, and amounts thereof may be selected to provide a specific N:P ratio. The N:P ratio of the composition refers to the molar ratio of nitrogen atoms in one or more lipids to the number of phosphate groups in an RNA. In general, a lower N:P ratio is preferred. The one or more RNA, lipids, and amounts thereof may be selected to provide an N:P ratio from about 2:1 to about 30:1, such as 2:1, 3:1, 4:1, 5:1, 6:1, 7:1, 8:1, 9:1, 10:1, 12:1, 14:1, 16:1, 18:1, 20:1, 22:1, 24:1, 26:1, 28:1, or 30:1. In certain embodiments, the N:P ratio may be from about 2:1 to about 8:1. In other embodiments, the N:P ratio is from about 5:1 to about 8:1. For example, the N:P ratio may be about 5.0:1, about 5.5:1, about 5.67:1, about 6.0:1, about 6.5:1, or about 7.0:1. For example, the N:P ratio may be about 5.67:1.

In some embodiments, the nucleic acid component is comprised of a modified nucleic acid. For example, an RNA may be a modified RNA. That is, an RNA may include one or more nucleobases, nucleosides, nucleotides, or linkers that are non-naturally occurring. A “modified” species may also be referred to herein as an “altered” species. Species may be modified or altered chemically, structurally, or functionally. For example, a modified nucleobase species may include one or more substitutions that are not naturally occurring.

### Physical Properties

The characteristics of a nanoparticle composition may depend on the components thereof. For example, a nanoparticle composition including cholesterol as a structural lipid may have different characteristics than a nanoparticle composition that includes a different structural lipid. Similarly, the characteristics of a nanoparticle composition may depend on the absolute or relative amounts of its components. For instance, a nanoparticle composition including a higher molar fraction of a phospholipid may have different characteristics than a nanoparticle composition including a lower molar fraction of a phospholipid. Characteristics may also vary depending on the method and conditions of preparation of the nanoparticle composition.

Nanoparticle compositions may be characterized by a variety of methods. For example, microscopy (e.g., transmission electron microscopy or scanning electron microscopy) may be used to examine the morphology and size distribution of a nanoparticle composition. Dynamic light scattering or potentiometry (e.g., potentiometric titrations) may be used to measure zeta potentials. Dynamic light scattering may also be utilized to determine particle sizes. Instruments such as the Zetasizer Nano ZS (Malvem Instruments Ltd, Malvern, Worcestershire, UK) may also be used to measure multiple characteristics of a nanoparticle composition, such as particle size, polydispersity index, and zeta potential.

The mean size of a nanoparticle composition may be between 10 nm and 1 micrometer, e.g., measured by dynamic light scattering (DLS). For example, the mean size may be from about 40 nm to about 150 nm, such as about 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm. In some embodiments, the mean size of a nanoparticle composition may be from about 50 nm to about 100 nm, from about 50 nm to about 90 nm, from about 50 nm to about 80 nm, from about 50 nm to about 70 nm, from about 50 nm to about 60 nm, from about 60 nm to about 100 nm, from about 60 nm to about 90 nm, from about 60 nm to about 80 nm, from about 60 nm to about 70 nm, from about 70 nm to about 100 nm, from about 70 nm to about 90 nm, from about 70 nm to about 80 nm, from about 80 nm to about 100 nm, from about 80 nm to about 90 nm, or from about 90 nm to about 100 nm. In certain embodiments, the mean size of a nanoparticle composition may be from about 70 nm to about 100 nm. In a particular embodiment, the mean size may be about 80 nm. In other embodiments, the mean size may be about 100 nm.

A nanoparticle composition may be relatively homogenous. A polydispersity index may be used to indicate the homogeneity of a nanoparticle composition, e.g., the particle size distribution of the nanoparticle compositions. A small (e.g., less than 0.3) polydispersity index generally indicates a narrow particle size distribution. A nanoparticle composition may have a polydispersity index from about 0 to about 0.25, such as 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, or 0.25. In some embodiments, the polydispersity index of a nanoparticle composition may be from about 0.10 to about 0.20.

The zeta potential of a nanoparticle composition may be used to indicate the electrokinetic potential of the composition. For example, the zeta potential may describe the surface charge of a nanoparticle composition. Nanoparticle compositions with relatively low charges, positive or negative, are generally desirable, as more highly charged species may interact undesirably with cells, tissues, and other elements in the body. In some embodiments, the zeta potential of a nanoparticle composition may be from about −10 mV to about +20 mV, from about −10 mV to about +15 mV, from about −10 mV to about +10 mV, from about −10 mV to about +5 mV, from about −10 mV to about 0 mV, from about −10 mV to about −5 mV, from about −5 mV to about +20 mV, from about −5 mV to about +15 mV, from about −5 mV to about +10 mV, from about −5 mV to about +5 mV, from about −5 mV to about 0 mV, from about 0 mV to about +20 mV, from about 0 mV to about +15 mV, from about 0 mV to about +10 mV, from about 0 mV to about +5 mV, from about +5 mV to about +20 mV, from about +5 mV to about +15 mV, or from about +5 mV to about +10 mV.

The efficiency of encapsulation of a therapeutic and/or prophylactic describes the amount of therapeutic and/or prophylactic that is encapsulated or otherwise associated with a nanoparticle composition after preparation, relative to the initial amount provided. The encapsulation efficiency is desirably high (e.g., close to 100%). The encapsulation efficiency may be measured, for example, by comparing the amount of therapeutic and/or prophylactic in a solution containing the nanoparticle composition before and after breaking up the nanoparticle composition with one or more organic solvents or detergents. Fluorescence may be used to measure the amount of free therapeutic and/or prophylactic (e.g., RNA) in a solution. For the nanoparticle compositions described herein, the encapsulation efficiency of a therapeutic and/or prophylactic may be at least 50%, for example 50%, 55%, 60%, 65%, 70%, 75%, 80%, 85%, 90%, 91%, 92%, 93%, 94%, 95%, 96%, 97%, 98%, 99%, or 100%. In some embodiments, the encapsulation efficiency may be at least 80%. In certain embodiments, the encapsulation efficiency may be at least 90%.

A nanoparticle composition may optionally comprise one or more coatings. For example, a nanoparticle composition may be formulated in a capsule, film, or tablet having a coating. A capsule, film, or tablet including a composition described herein may have any useful size, tensile strength, hardness, or density.

Unless otherwise defined, all technical and scientific terms used herein have the same meaning as commonly understood by one of ordinary skill in the art to which this disclosure belongs. Although methods and materials similar or equivalent to those described herein may be used in the practice or testing of the present disclosure, suitable methods and materials are described below. All publications, patent applications, patents, and other references mentioned herein are incorporated by reference in their entirety. In addition, the materials, methods, and examples are illustrative only and not intended to be limiting.

In some embodiments, the CRISPR gene-editing system comprises one or more RNA-containing compositions. In some embodiments, the CRISPR gene-editing system further comprises one or more nanoparticles. In some embodiments, said one or more RNA-containing compositions comprises a guide RNA. In some embodiments, said one or more RNA-containing compositions comprises an mRNA. In some embodiments, said one or more RNA-containing compositions comprises an RNP (e.g., Cas9 and a guide RNA). In some embodiments, said one or more nanoparticles are lipid nanoparticles (LNP).

In some embodiments, the CRISPR gene-editing system comprises one or more LNPs collectively encapsulating (i) the RNA-guided nuclease or the nucleic acid encoding the RNA-guided nuclease and (ii) the at least one guide RNA or the nucleic acid encoding the at least one guide RNA. In some embodiments, the one or more LNPs comprises a first plurality of LNP encapsulating the RNA-guided nuclease or a nucleic acid encoding an RNA-guided nuclease and a second plurality of LNP encapsulating the at least one guide RNA or a nucleic acid encoding at least one guide RNA.

In some embodiments, the one or more LNP comprises a component selected from the group consisting of 3-(didodecylamino)-N1,N1,4-tridodecyl-1-piperazineethanamine (KL10), N1-[2-(didodecylamino)ethyl]-N1,N4,N4-tridodecyl-1,4-piperazinediethanamine (KL22), 14,25-ditridecyl-15,18,21,24-tetraaza-octatriacontane (KL25), 1,2-dilinoleyloxy-N,N-dimethylaminopropane (DLin-DMA), 2,2-dilinoleyl-4-dimethylaminomethyl-[1,3]-dioxolane (DLin-K-DMA), heptatriaconta-6,9,28,31-tetraen-19-yl 4-(dimethylamino)butanoate (DLin-MC3-DMA), 2,2-dilinoleyl-4-(2-dimethylaminoethyl)-[1,3]-dioxolane (DLin-KC2-DMA), 1,2-dioleyloxy-N,N-dimethylaminopropane (DODMA), 2-({8-[(3.beta.)-cholest-5-en-3-yloxy]octyl}oxy)-N,N-dimethyl-3-[(9Z,12Z)—octadeca-9,12-dien-1-yloxy]propan-1-amine (Octyl-CLinDMA), (2R)-2-({8-[(3.beta.)-cholest-5-en-3-yloxy]octyl}oxy)-N,N-dimethyl-3-[(9Z-,12Z)-octadeca-9,12-dien-1-yloxy]propan-1-amine (Octyl-CLinDMA (2R)), (2S)-2-({8-[(3.beta.)-cholest-5-en-3-yloxy]octyl}oxy)-N,N-dimethyl-3-[(9Z-,12Z)-octadeca-9,12-dien-1-yloxy]propan-1-amine (Octyl-CLinDMA (2S)), a lipid including a cyclic amine group, and a mixture thereof.

In some embodiments, the one or more LNP comprises a component selected from the group consisting of 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-dioleoyl-sn-glycero-3-phosphoethanolamine (DOPE), 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), sphingomyelin (SM), and a mixture thereof.

In some embodiments, the one or more LNP comprises a component selected from the group consisting of PEG-modified phosphatidylethanolamines, PEG-modified phosphatidic acids, PEG-modified ceramides, PEG-modified dialkylamines, PEG-modified diacylglycerols, PEG-modified dialkylglycerols, and mixtures thereof. For example, a PEG lipid may be PEG-c-DOMG, PEG-DMG, PEG-DLPE, PEG-DMPE, PEG-DPPC, PEG-DMA, a PEG-DSPE lipid, and a mixture thereof.

In some embodiments, the one or more LNP comprises a component selected from the group consisting of a cholesterol, fecosterol, stigmasterol, stigmastanol, sitosterol, (3-sitosterol, lupeol, betulin, ursolic acid, oleanolic acid, campesterol, fucosterol, brassicasterol, ergosterol, 9,11-dehydroergosterol, tomatidine, tomatine, α-tocopherol, and a mixture thereof.

In some embodiments, use of the CRISPR gene-editing system further comprising one or more LNPs to target a gene selected from NFKB1, NFKB2, and combinations thereof is therapeutic.

### C. Virus-Like Particles

In one aspect, the present disclosure encompasses means for delivering a CRISPR gene-editing system to a mammalian cell via a virus-like particle (VLP). In some embodiments, a CRISPR gene-editing system is delivered by a VLP. Without wishing to be bound by any particular theory, in certain embodiments, nucleic acids, when present in the particle, are resistant in aqueous solution to degradation with a nuclease. In other embodiments, proteins are protected from protease degradation while present in the particle. In some embodiments, proteins and nucleic acids encapsulated by VLPs are capable of penetrating the cellular plasma membrane.

In some embodiments, the CRISPR gene-editing system comprises one or more RNA-containing compositions. In some embodiments, the CRISPR gene-editing system further comprises one or more VLPs. In some embodiments, said one or more RNA-containing compositions comprises a guide RNA. In some embodiments, said one or more RNA-containing compositions comprises an mRNA. In some embodiments, said one or more RNA-containing compositions comprises an RNP (e.g., Cas9 and a guide RNA).

In some embodiments, the CRISPR gene-editing system comprises one or more virus-like particles collectively encapsulating (i) the RNA-guided nuclease or the nucleic acid encoding the RNA-guided nuclease and (ii) the at least one guide RNA or the nucleic acid encoding the at least one guide RNA. In some embodiments, the one or more virus-like particles comprises a first plurality of virus-like particles encapsulating the RNA-guided nuclease or a nucleic acid encoding an RNA-guided nuclease and a second plurality of virus-like particles encapsulating the at least one guide RNA or a nucleic acid encoding at least one guide RNA.

In some embodiments, use of the CRISPR gene-editing system further comprising one or more LNPs to target a gene selected from NFKB1, NFKB2, and combinations thereof is therapeutic.

### D. Miscellaneous Modes of Delivery

### 1. Liposomes

In some embodiments, nucleic acids encoding a CRISPR gene-editing system targeting a gene selected from NFKB1, NFKB2, and combinations thereof (e.g., Cas9 or gRNA) are entrapped in liposomes bearing positive charges on their surface (e.g., lipofectins), which can be tagged with antibodies against cell surface antigens of the target cells. These delivery vehicles can also be used to deliver Cas9 protein/gRNA complexes.

In some embodiments, the CRISPR gene-editing system comprises one or more RNA-containing compositions. In some embodiments, the CRISPR gene-editing system further comprises one or more liposomes. In some embodiments, said one or more RNA-containing compositions comprises a guide RNA. In some embodiments, said one or more RNA-containing compositions comprises an mRNA. In some embodiments, said one or more RNA-containing compositions comprises an RNP (e.g., Cas9 and a guide RNA).

In some embodiments, wherein the composition comprises one or more liposomes collectively encapsulating (i) the RNA-guided nuclease or the nucleic acid encoding the RNA-guided nuclease and (ii) the at least one guide RNA or the nucleic acid encoding the at least one guide RNA. In some embodiments, the one or more liposomes comprises a first plurality of liposomes encapsulating the RNA-guided nuclease or a nucleic acid encoding an RNA-guided nuclease and a second plurality of liposomes encapsulating the at least one guide RNA or a nucleic acid encoding at least one guide RNA.

In some embodiments, use of the CRISPR gene-editing system further comprising one or more LNPs to target a gene selected from NFKB1, NFKB2, and combinations thereof is therapeutic.

### 2. Lipid Nanocrystals (LNC)

In one aspect, the In one aspect, the present disclosure encompasses means for delivering a CRISPR gene-editing system to a mammalian cell via a lipid nanocrystal (LNC). In some embodiments, a CRISPR gene-editing system is delivered by a LNC. Without wishing to be bound by any particular theory, in certain embodiments, nucleic acids, when present in the nanocrystal, are resistant in aqueous solution to degradation with a nuclease. In other embodiments, proteins are protected from protease degradation while present in the nanocrystal. In some embodiments, proteins and nucleic acids encapsulated by nanocrystal are capable of penetrating the cellular plasma membrane.

In some embodiments, the CRISPR gene-editing system comprises one or more RNA-containing compositions. In some embodiments, the CRISPR gene-editing system further comprises one or more nanocrystals. In some embodiments, said one or more RNA-containing compositions comprises a guide RNA. In some embodiments, said one or more RNA-containing compositions comprises an mRNA. In some embodiments, said one or more RNA-containing compositions comprises an RNP (e.g., Cas9 and a guide RNA). In some embodiments, said one or more nanocrystals are lipid nanocrystals (LNC).

In some embodiments, the CRISPR gene-editing system comprises one or more LNCs collectively encapsulating (i) the RNA-guided nuclease or the nucleic acid encoding the RNA-guided nuclease and (ii) the at least one guide RNA or the nucleic acid encoding the at least one guide RNA. In some embodiments, the one or more LNCs comprises a first plurality of LNC encapsulating the RNA-guided nuclease or a nucleic acid encoding an RNA-guided nuclease and a second plurality of LNC encapsulating the at least one guide RNA or a nucleic acid encoding at least one guide RNA.

In some embodiments, use of the CRISPR gene-editing system further comprising one or more LNPs to target a gene selected from NFKB1, NFKB2, and combinations thereof is therapeutic.

### VI. Pharmaceutical Compositions

In one aspect, the present disclosure encompasses pharmaceutical compositions comprising a CRISPR gene-editing system for treatment of a mammal in need thereof. In some embodiments, the CRISPR gene-editing system targets a gene selected from NFKB1, NFKB2, and combinations thereof. In some embodiments, the mammal is selected from a human, a dog, a horse, and a cat.

In some embodiments, the pharmaceutical composition comprising a CRISPR gene-editing system targets NFKB1. In some embodiments, the CRISPR gene-editing system targeting NFKB1 is delivered to a mammalian cell via viral vector. In some embodiments, the CRISPR gene-editing system targeting NFKB1 is delivered to a mammalian cell via an AAV vector. In some embodiments, the CRISPR gene-editing system targeting NFKB1 is delivered to a mammalian cell via a lentiviral vector. In some embodiments, the CRISPR gene-editing system targeting NFKB1 is delivered to a mammalian cell via a lipid nanoparticle. In some embodiments, the CRISPR gene-editing system targeting NFKB1 is delivered to a mammalian cell via a virus-like particle. In some embodiments, the CRISPR gene-editing system targeting NFKB1 is delivered to a mammalian cell via a liposome. In some embodiments, the CRISPR gene-editing system targeting NFKB1 is delivered to a mammalian cell via a lipid nanocrystal.

In some embodiments, the disclosure provides a pharmaceutical composition for treating or preventing a joint disorder, comprising: (i) an RNA-guided nuclease or a nucleic acid encoding an RNA-guided nuclease; and (ii) at least one guide RNA or a nucleic acid encoding at least one guide RNA targeting a gene associated with the production, blocking, or removal of reactive oxygen species (ROS).

In some embodiments, the joint disorder is arthritis.

In some embodiments, the joint disorder is osteoarthritis.

In some embodiments, the joint disorder is rheumatoid arthritis.

In some embodiments, the joint disorder is post-traumatic arthritis.

In some embodiments, the joint disorder is gout

In some embodiments, the joint disorder is pseudogout.

In some embodiments, the joint disorder is canine, equine, or feline lameness.

In some embodiments, the joint disorder is tendinopathy.

In some embodiments, the gene associated with the production, blocking, or removal of ROS is a nuclear factor kappa B subunit 1 (NF-κB1) gene.

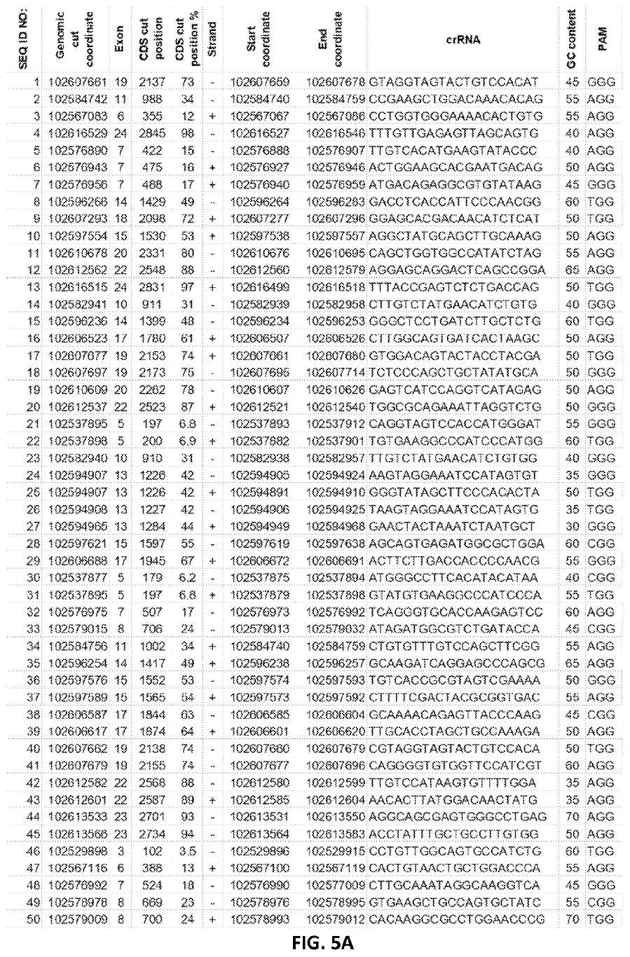

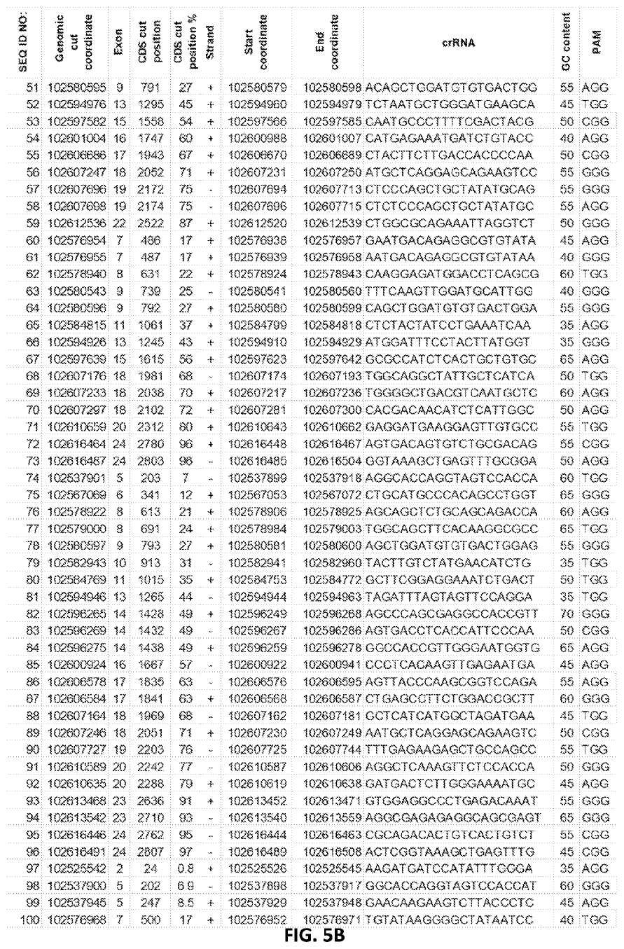

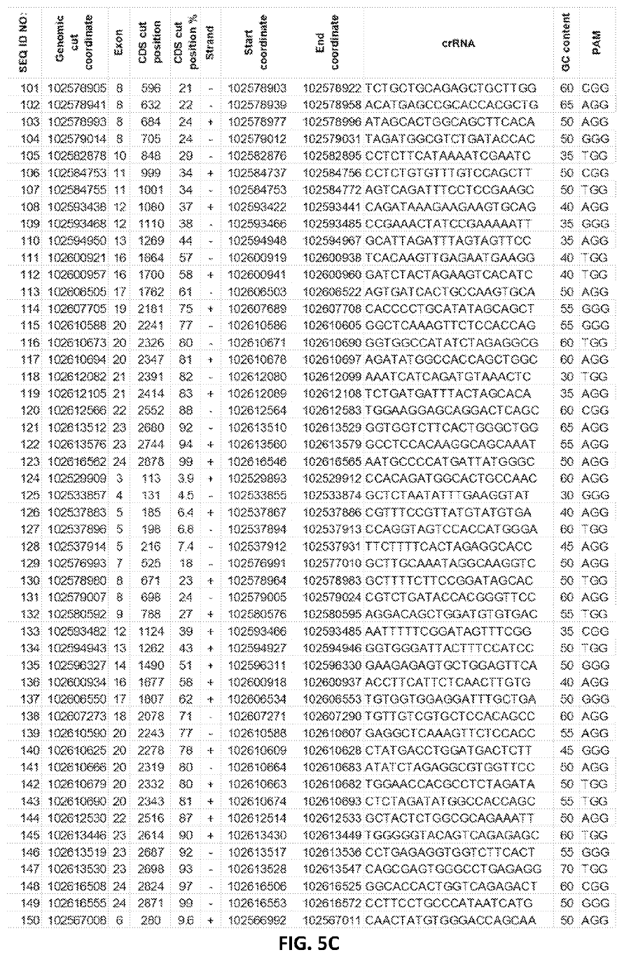

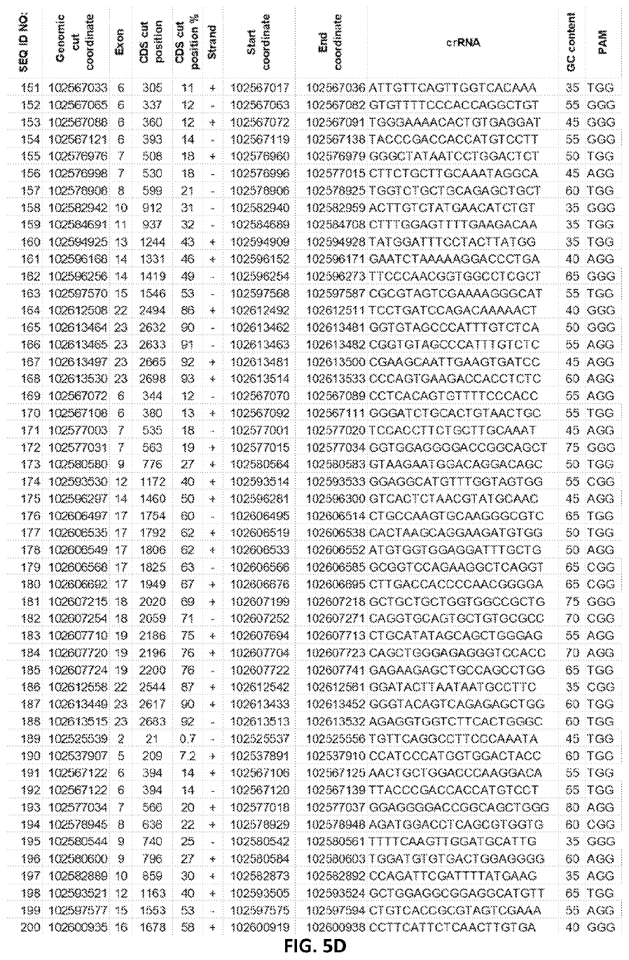

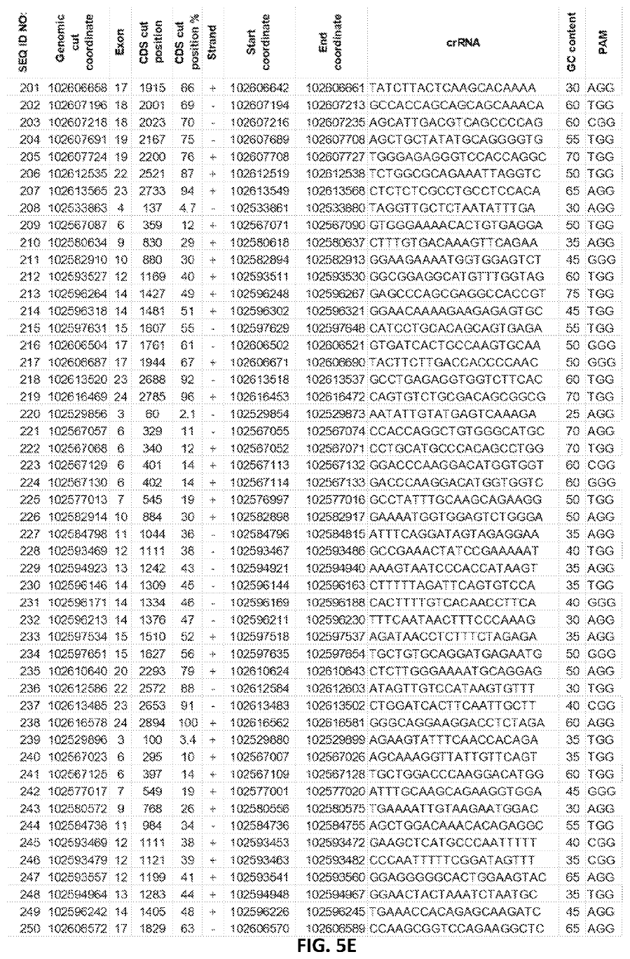

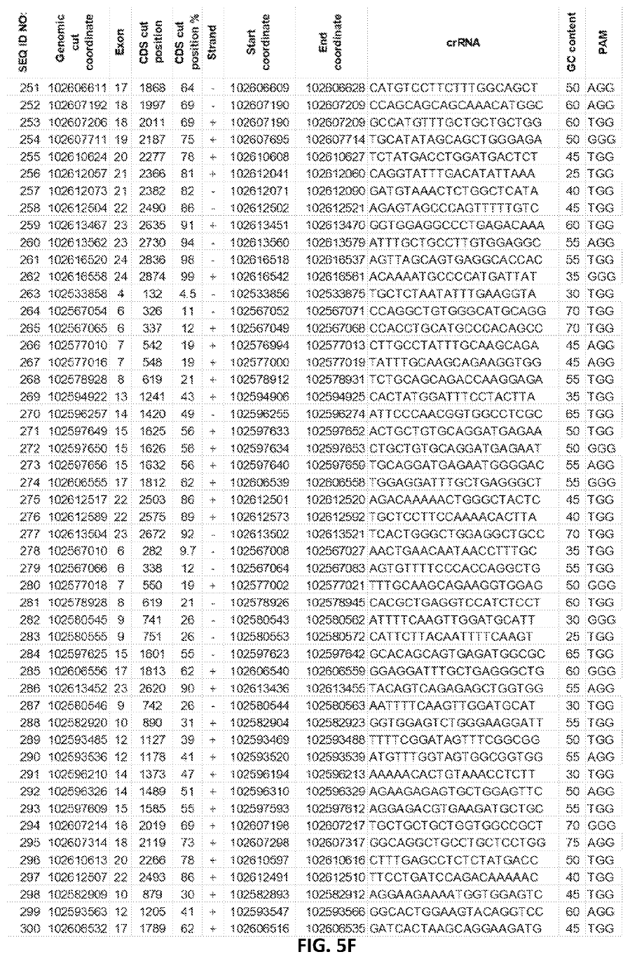

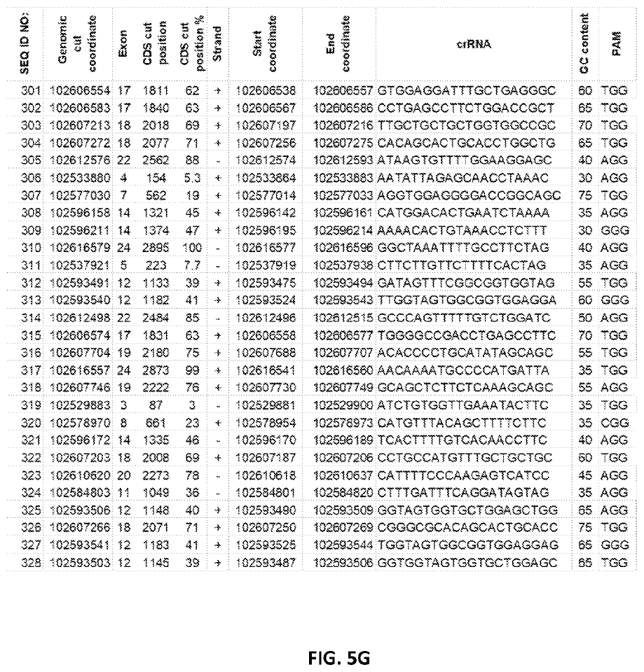

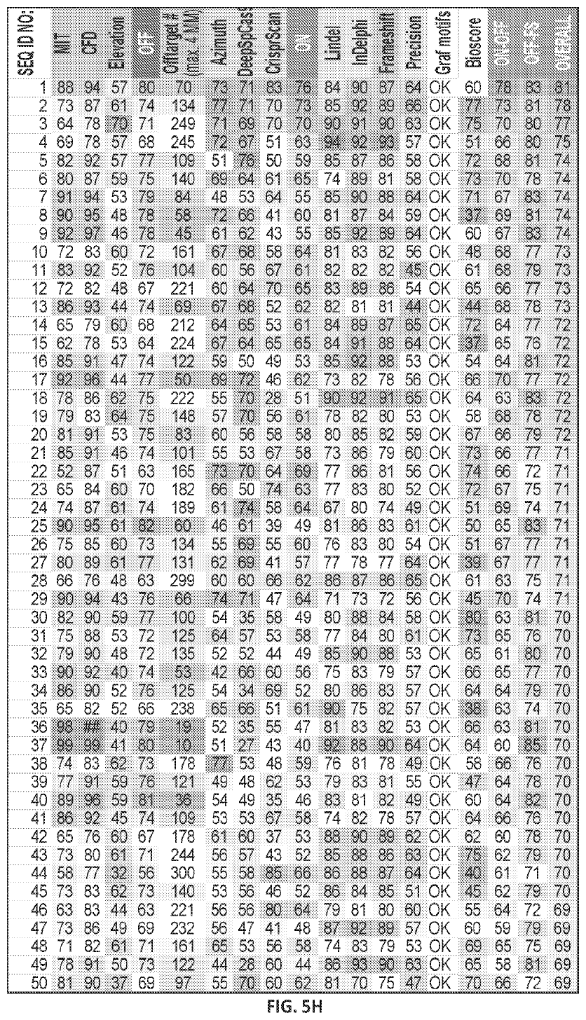

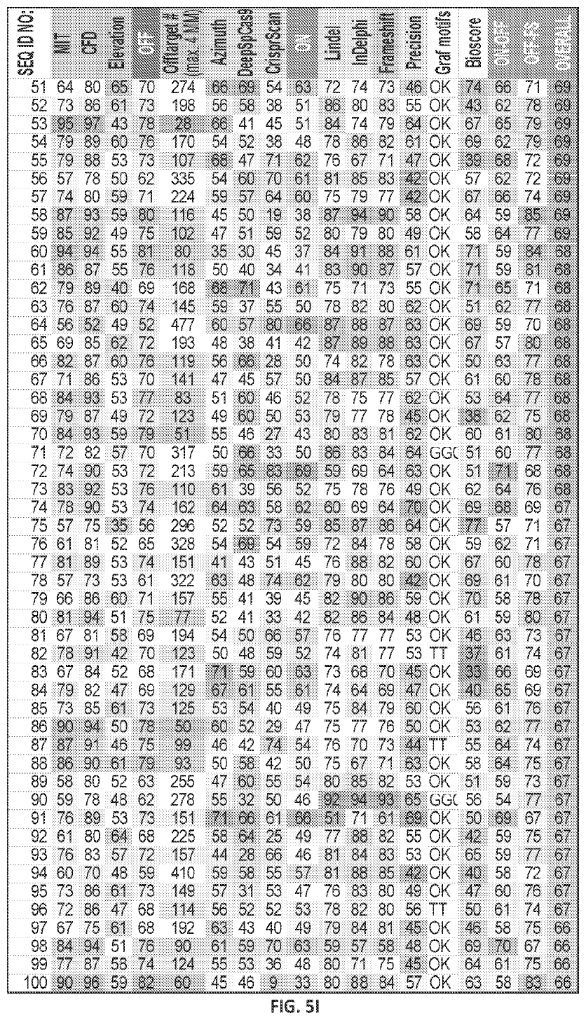

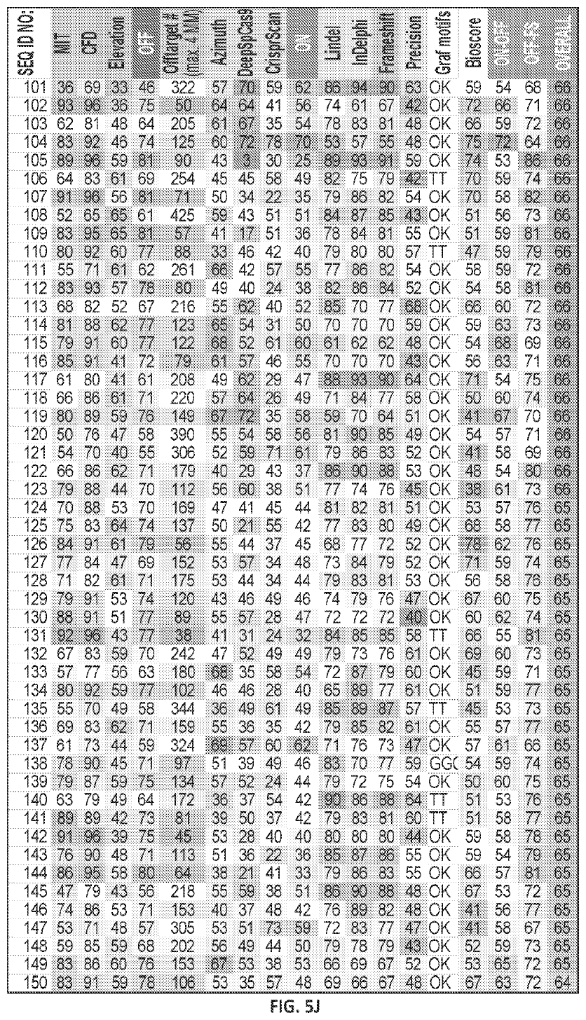

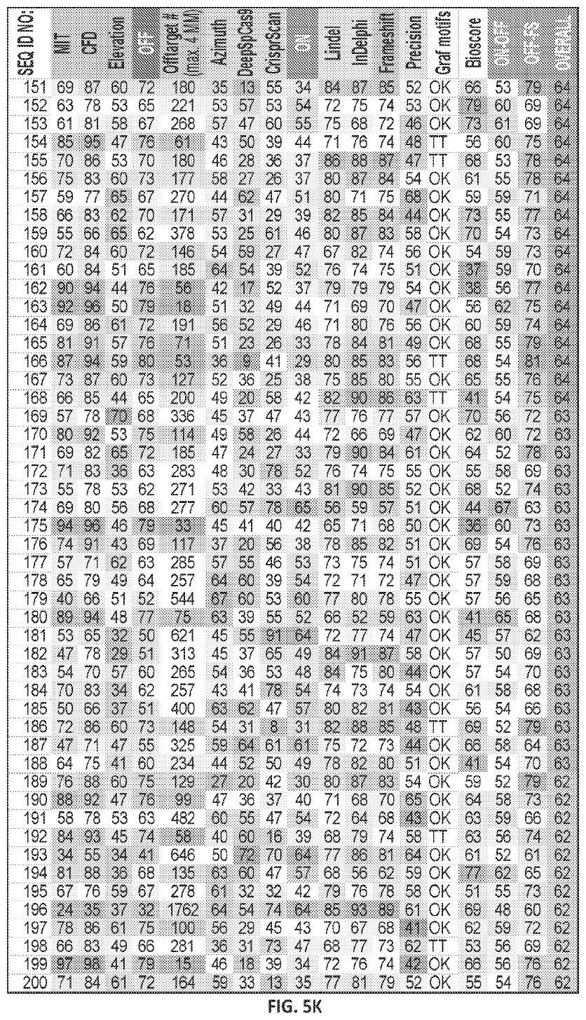

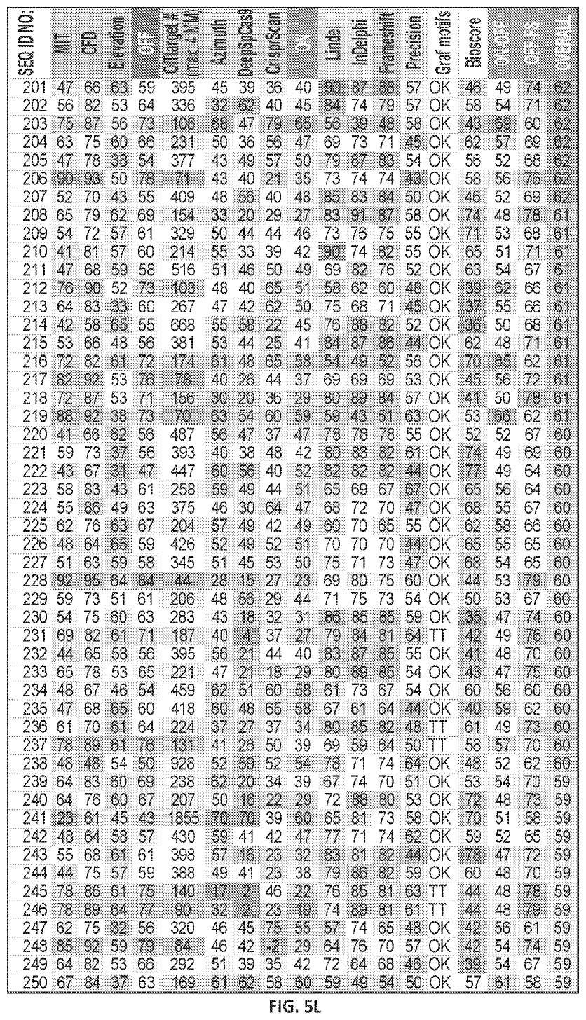

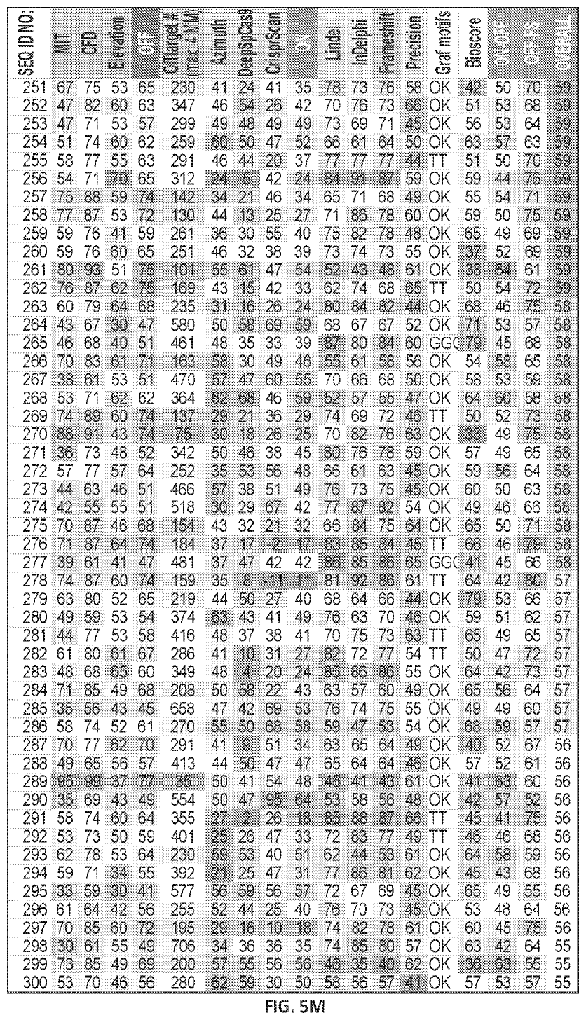

In some embodiments, the at least one guide RNA comprises a crRNA sequence selected from the group consisting of those sequences shown in FIG. 5 (SEQ ID NOS: 1-328).

In some embodiments, the at least one guide RNA comprises a crRNA sequence selected from the group consisting of SEQ ID NOS: 1-149.

In some embodiments, the at least one guide RNA comprises a crRNA sequence selected from the group consisting of SEQ ID NOS: 1-45.

In some embodiments, the at least one guide RNA comprises a crRNA sequence selected from the group consisting of SEQ ID NOS: 1-13.

In some embodiments, the at least one guide RNA comprises a crRNA sequence that targets exon 1 of the NF-κB1 gene.

In some embodiments, the at least one guide RNA comprises a crRNA sequence that targets exon 2 of the NF-κB1 gene.

In some embodiments, the at least one guide RNA comprises a crRNA sequence that targets exon 3 of the NF-κB1 gene.

In some embodiments, the at least one guide RNA comprises a crRNA sequence that targets exon 4 of the NF-κB1 gene.

In some embodiments, the at least one guide RNA comprises a crRNA sequence that targets exon 5 of the NF-κB1 gene.

In some embodiments, the at least one guide RNA comprises a crRNA sequence that targets exon 6 of the NF-κB1 gene.

In some embodiments, the at least one guide RNA comprises a crRNA sequence that targets exon 7 of the NF-κB1 gene.

In some embodiments, the at least one guide RNA comprises a crRNA sequence that targets exon 8 of the NF-κB1 gene.

In some embodiments, the at least one guide RNA comprises a crRNA sequence that targets exon 9 of the NF-κB1 gene.

In some embodiments, the at least one guide RNA comprises a crRNA sequence that targets exon 10 of the NF-κB1 gene.

In some embodiments, the at least one guide RNA comprises a crRNA sequence that targets exon 11 of the NF-κB1 gene.

In some embodiments, the at least one guide RNA comprises a crRNA sequence that targets exon 12 of the NF-κB1 gene.

In some embodiments, the at least one guide RNA comprises a crRNA sequence that targets exon 13 of the NF-κB1 gene.

In some embodiments, the at least one guide RNA comprises a crRNA sequence that targets exon 14 of the NF-κB1 gene.

In some embodiments, the at least one guide RNA comprises a crRNA sequence that targets exon 15 of the NF-κB1 gene.

In some embodiments, the at least one guide RNA comprises a crRNA sequence that targets exon 16 of the NF-κB1 gene.

In some embodiments, the at least one guide RNA comprises a crRNA sequence that targets exon 17 of the NF-κB1 gene.

In some embodiments, the at least one guide RNA comprises a crRNA sequence that targets exon 18 of the NF-κB1 gene.

In some embodiments, the at least one guide RNA comprises a crRNA sequence that targets exon 19 of the NF-κB1 gene.

In some embodiments, the at least one guide RNA comprises a crRNA sequence that targets exon 20 of the NF-κB1 gene.

In some embodiments, the at least one guide RNA comprises a crRNA sequence that targets exon 21 of the NF-κB1 gene.

In some embodiments, the at least one guide RNA comprises a crRNA sequence that targets exon 22 of the NF-κB1 gene.

In some embodiments, the at least one guide RNA comprises a crRNA sequence that targets exon 23 of the NF-κB1 gene.

In some embodiments, the at least one guide RNA comprises a crRNA sequence that targets exon 24 of the NF-κB1 gene.

In some embodiments, the gene associated with the production, blocking, or removal of ROS is a nuclear factor kappa B subunit 2 (NF-κB2) gene.

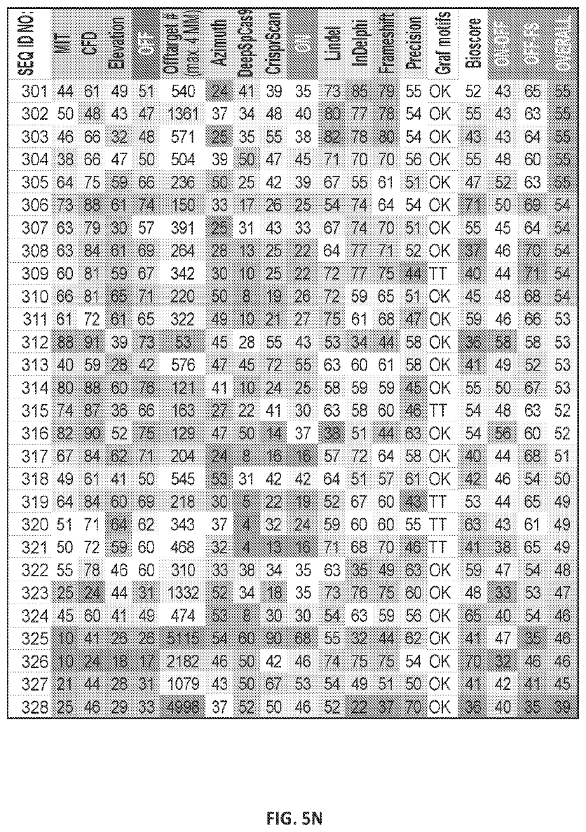

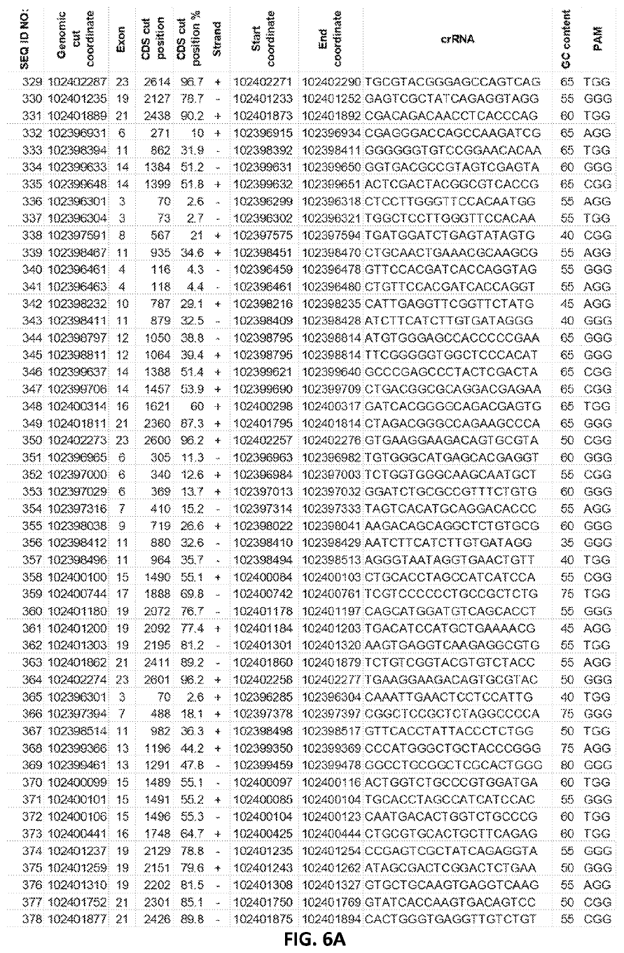

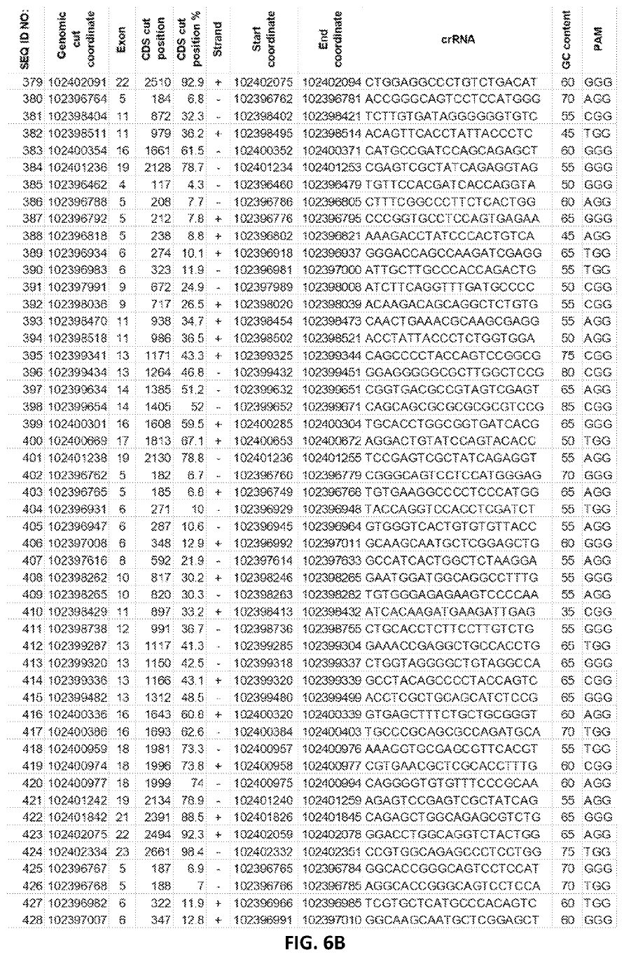

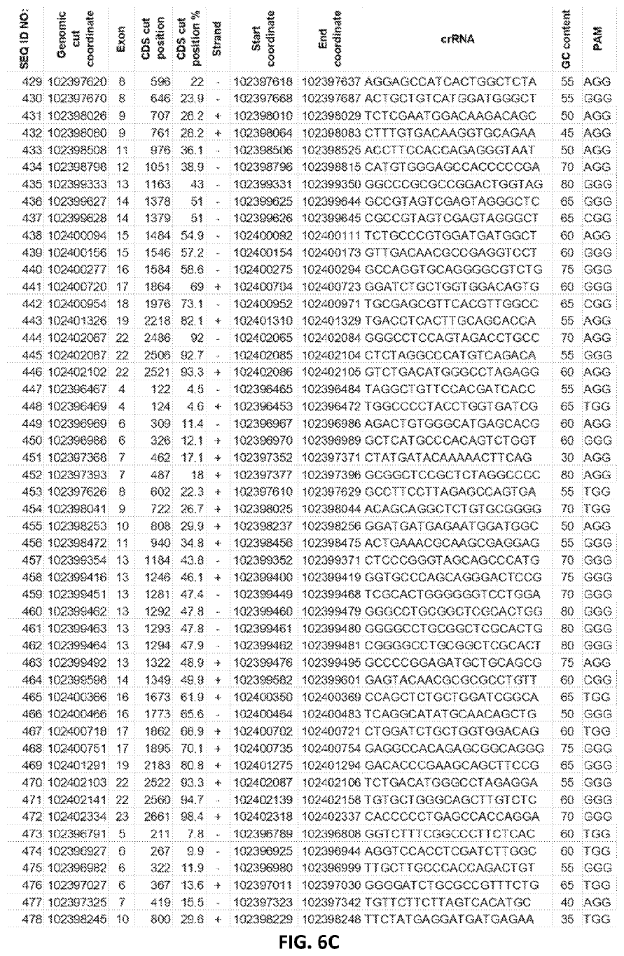

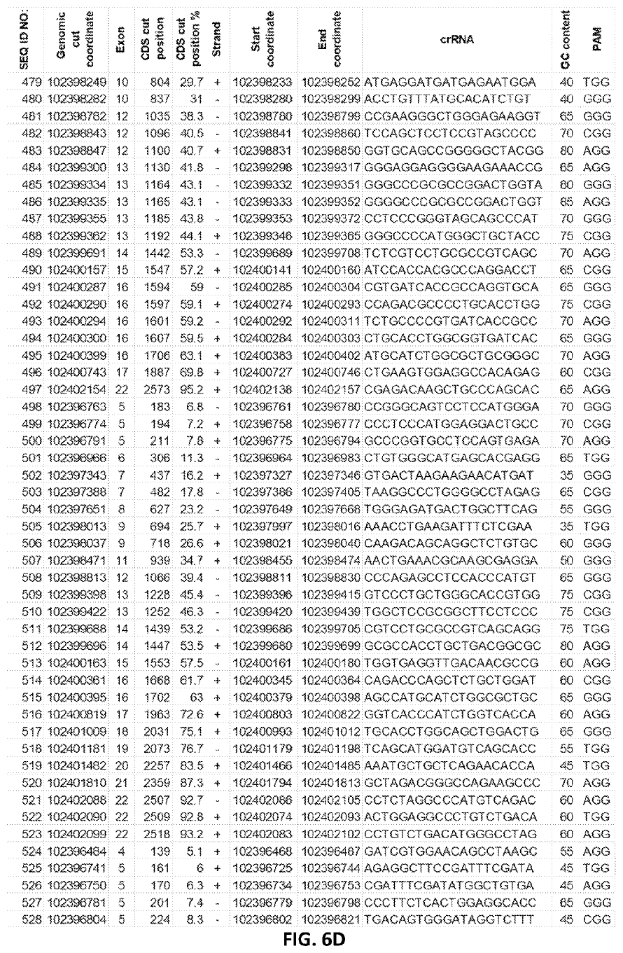

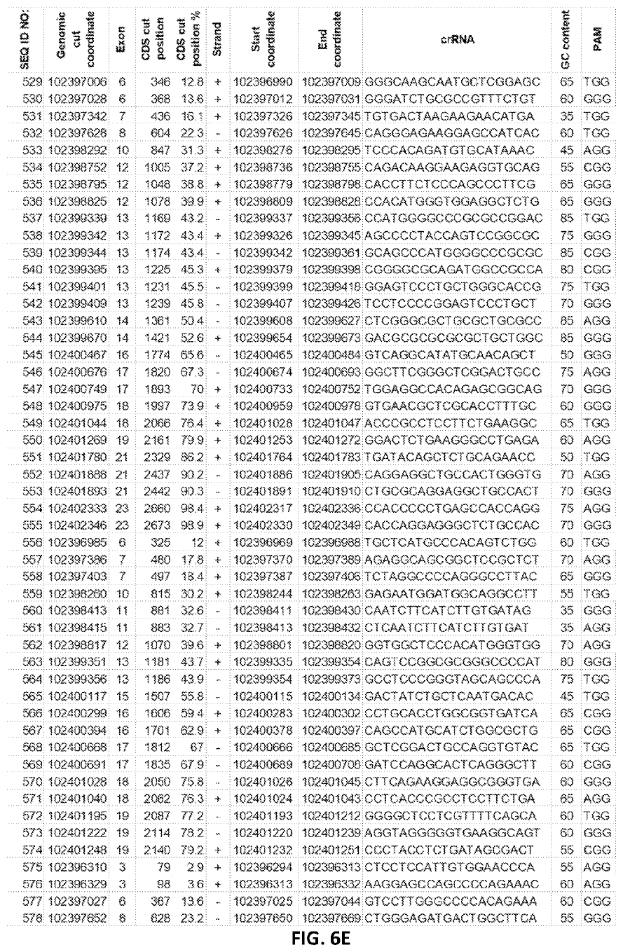

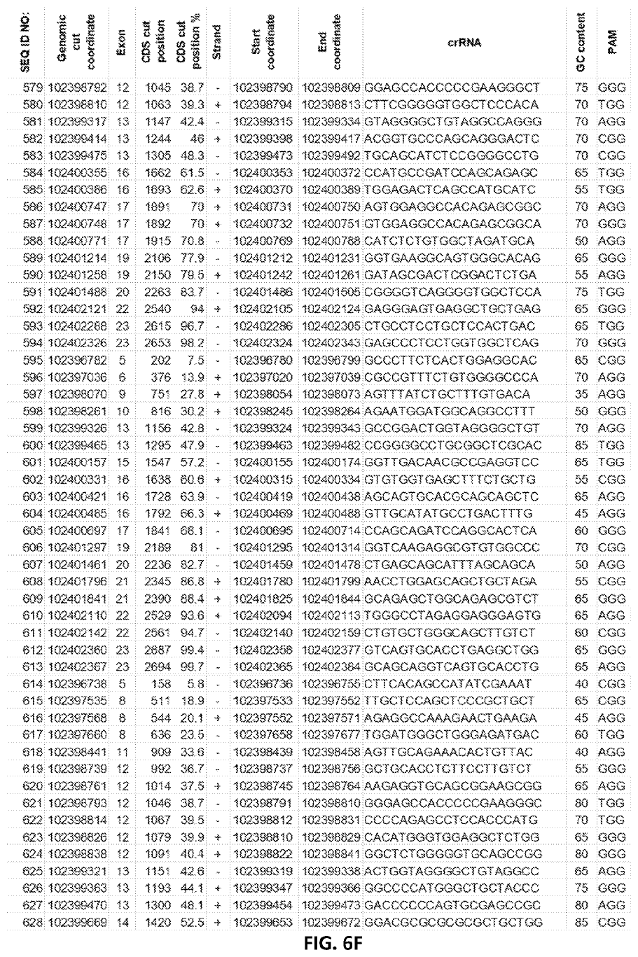

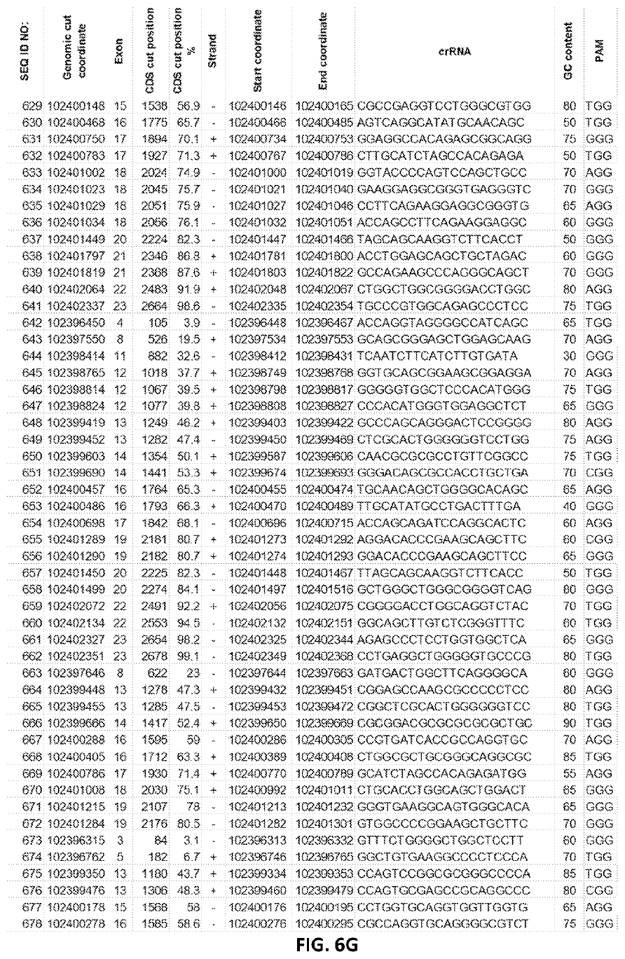

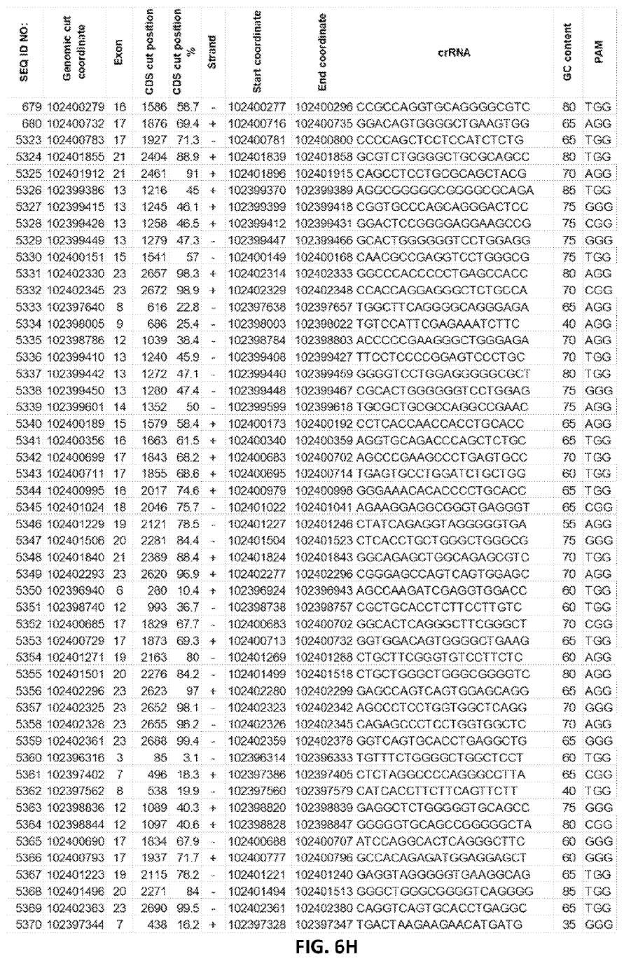

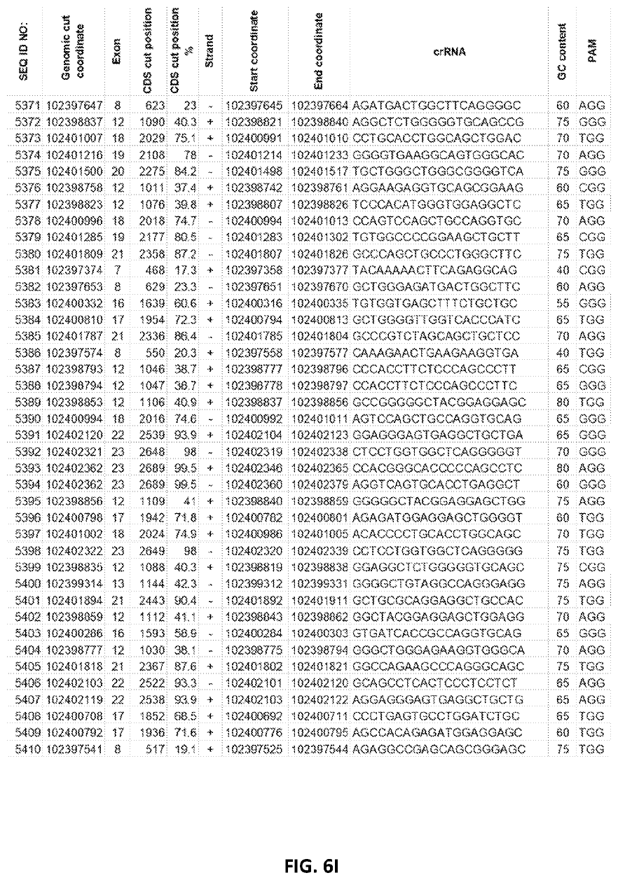

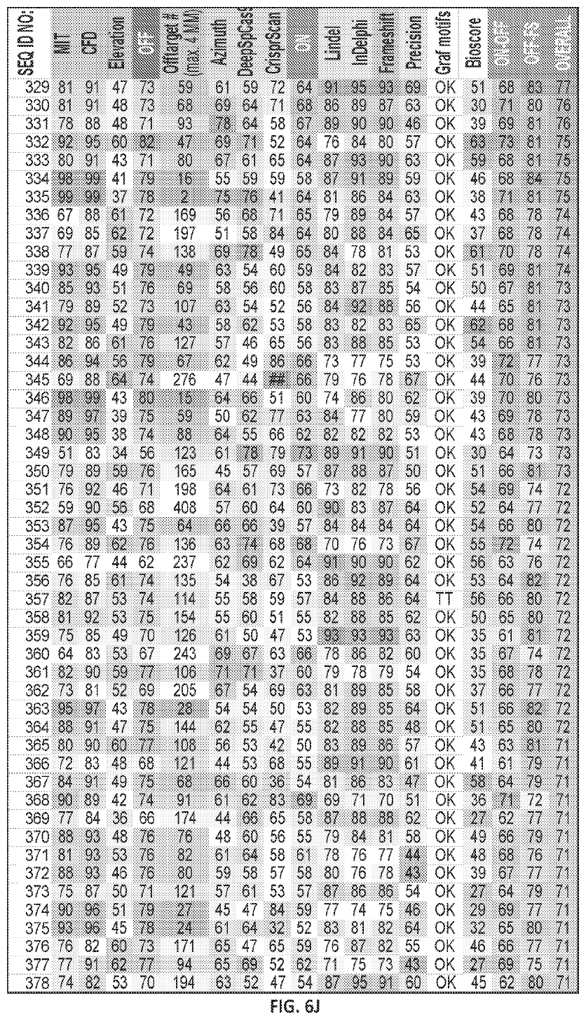

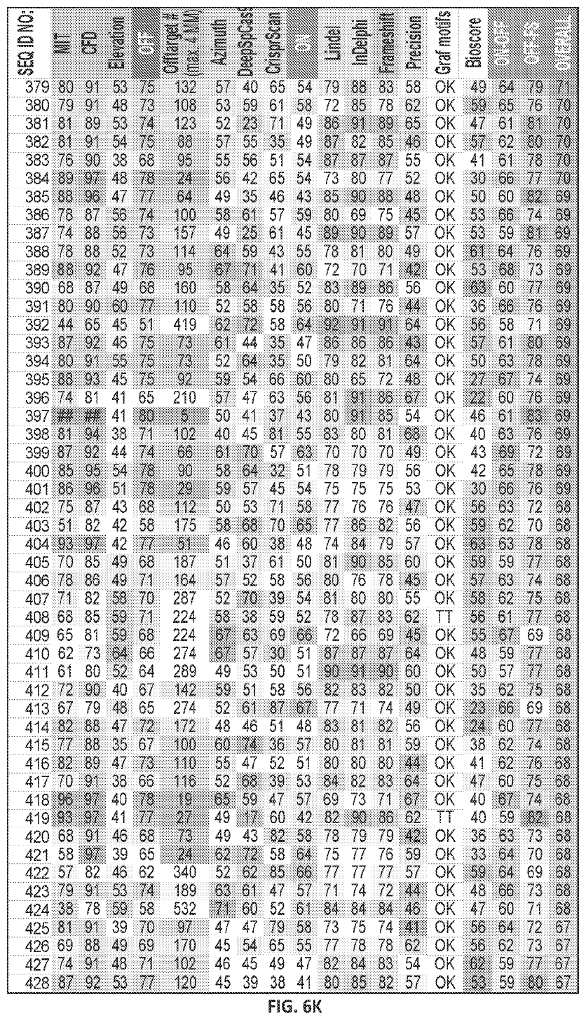

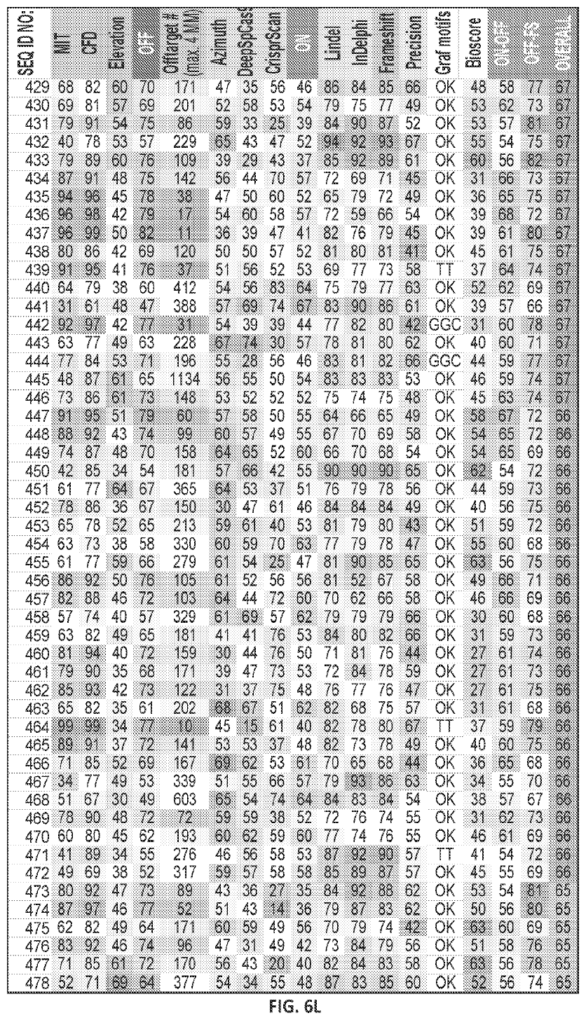

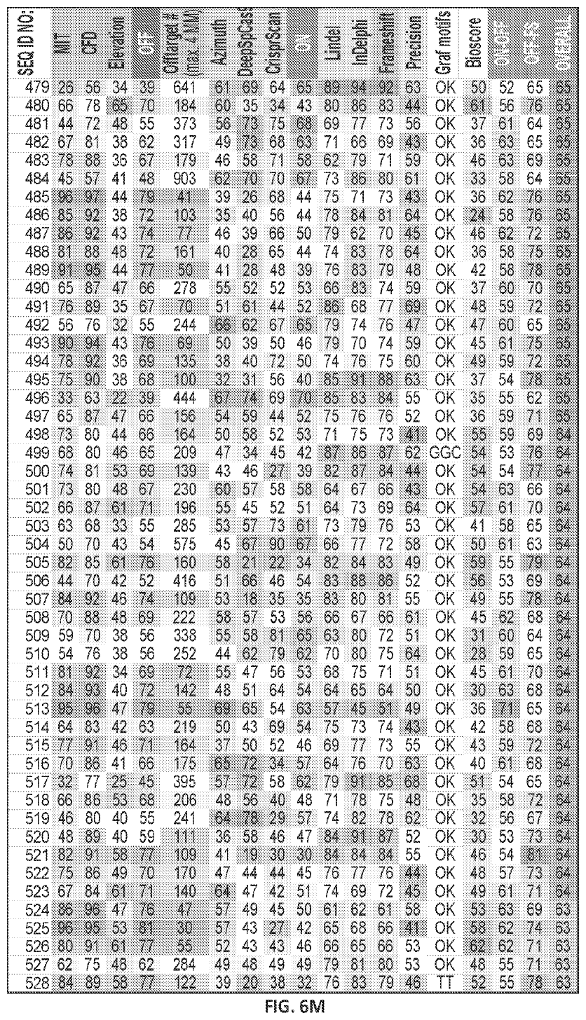

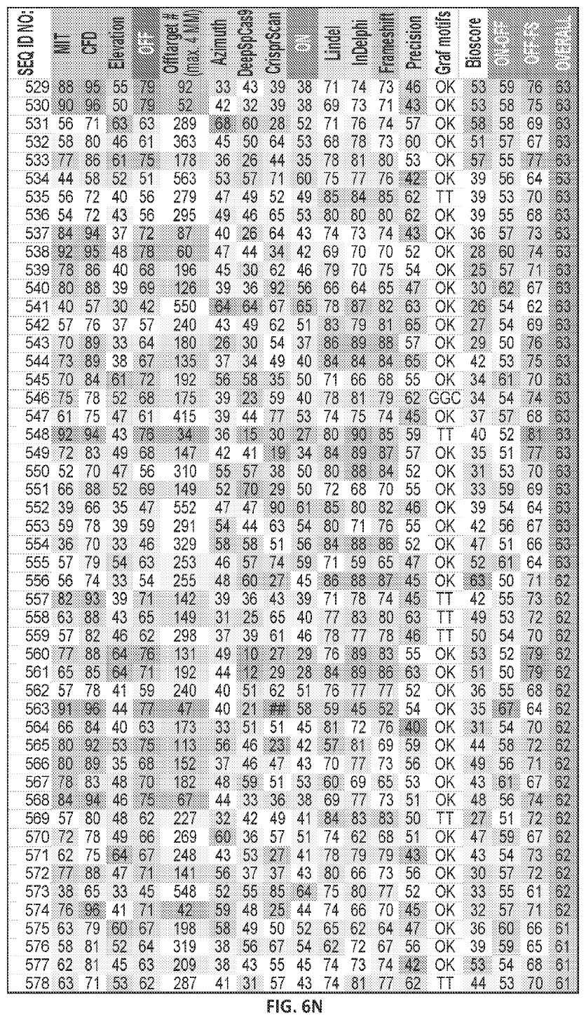

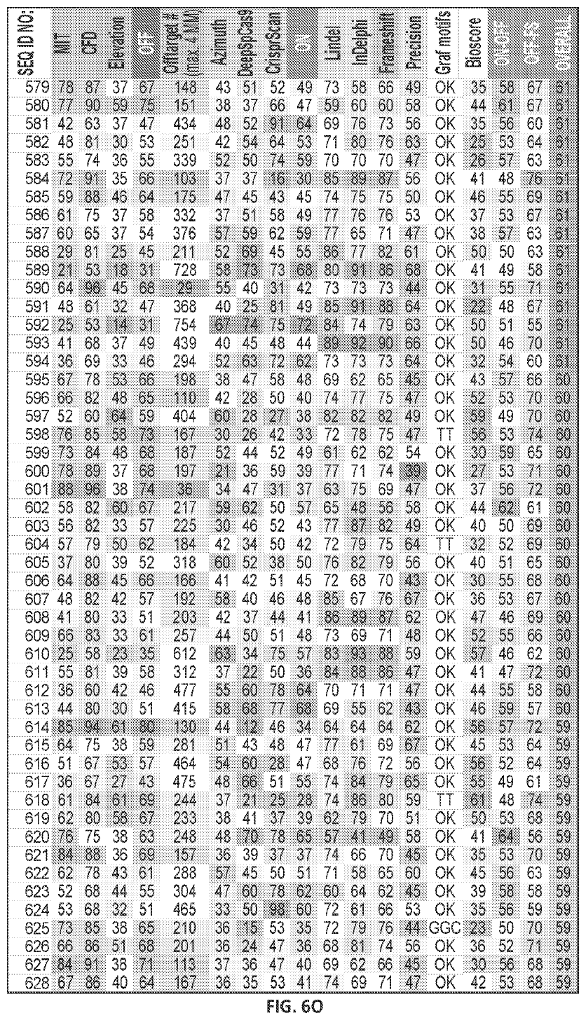

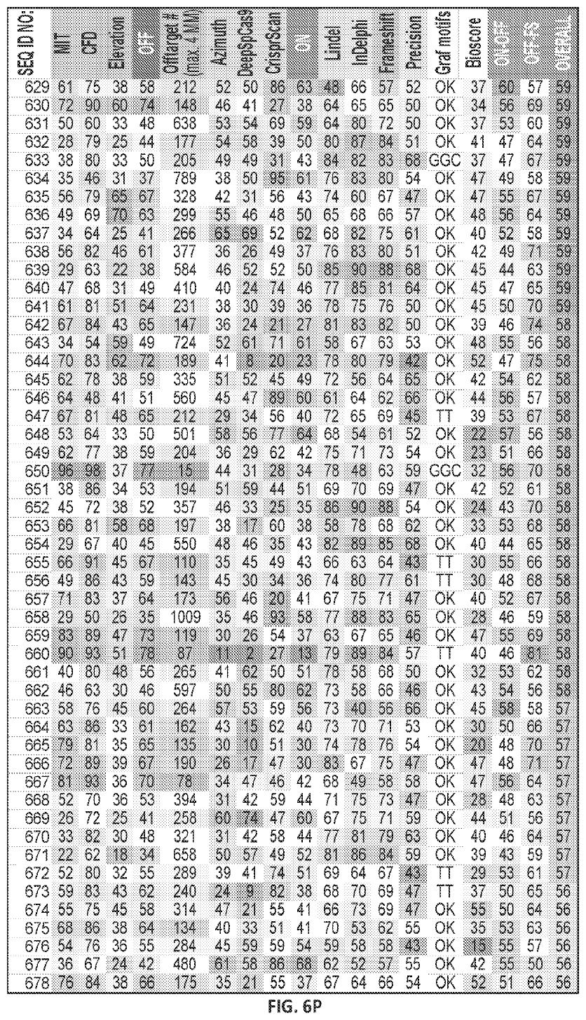

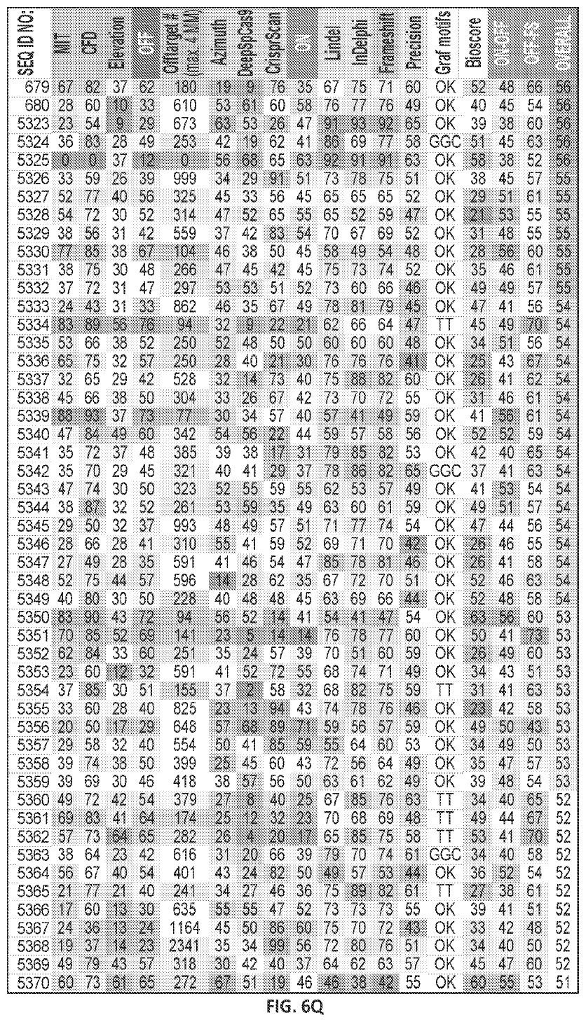

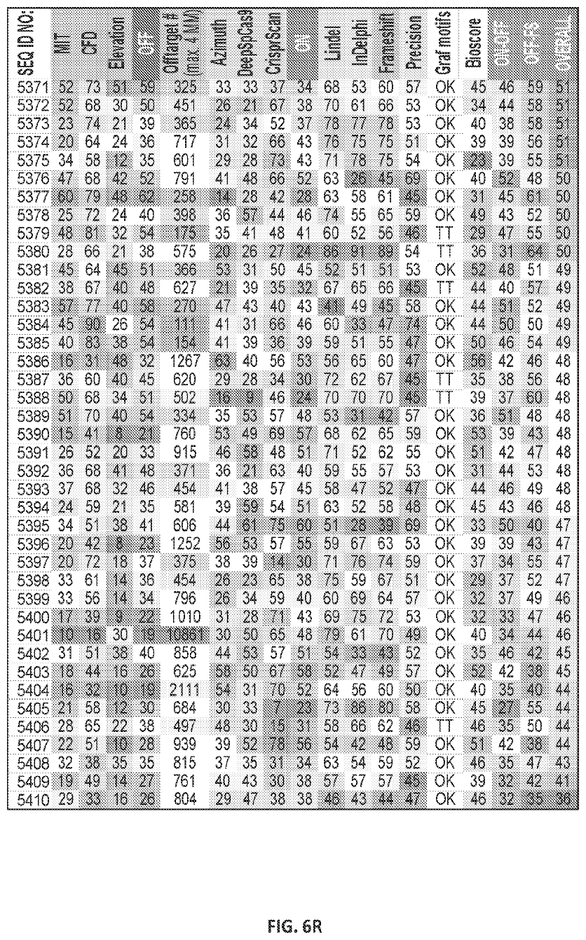

In some embodiments, the at least one guide RNA comprises a crRNA sequence selected from the group consisting of those sequences shown in FIG. 6 (SEQ ID NOS: 329-680 and 5323-5410).

In some embodiments, the at least one guide RNA comprises a crRNA sequence selected from the group consisting of SEQ ID NOS: 329-497.

In some embodiments, the at least one guide RNA comprises a crRNA sequence selected from the group consisting of SEQ ID NOS: 329-384.

In some embodiments, the at least one guide RNA comprises a crRNA sequence selected from the group consisting of SEQ ID NOS: 329-335.

In some embodiments, the at least one guide RNA comprises a crRNA sequence that targets exon 1 of the NF-κB2 gene.

In some embodiments, the at least one guide RNA comprises a crRNA sequence that targets exon 2 of the NF-κB2 gene.

In some embodiments, the at least one guide RNA comprises a crRNA sequence that targets exon 3 of the NF-κB2 gene.

In some embodiments, the at least one guide RNA comprises a crRNA sequence that targets exon 4 of the NF-κB2 gene.

In some embodiments, the at least one guide RNA comprises a crRNA sequence that targets exon 5 of the NF-κB2 gene.

In some embodiments, the at least one guide RNA comprises a crRNA sequence that targets exon 6 of the NF-κB2 gene.

In some embodiments, the at least one guide RNA comprises a crRNA sequence that targets exon 7 of the NF-κB2 gene.

In some embodiments, the at least one guide RNA comprises a crRNA sequence that targets exon 8 of the NF-κB2 gene.

In some embodiments, the at least one guide RNA comprises a crRNA sequence that targets exon 9 of the NF-κB2 gene.

In some embodiments, the at least one guide RNA comprises a crRNA sequence that targets exon 10 of the NF-κB2 gene.

In some embodiments, the at least one guide RNA comprises a crRNA sequence that targets exon 11 of the NF-κB2 gene.

In some embodiments, the at least one guide RNA comprises a crRNA sequence that targets exon 12 of the NF-κB2 gene.

In some embodiments, the at least one guide RNA comprises a crRNA sequence that targets exon 13 of the NF-κB2 gene.

In some embodiments, the at least one guide RNA comprises a crRNA sequence that targets exon 14 of the NF-κB2 gene.

In some embodiments, the at least one guide RNA comprises a crRNA sequence that targets exon 15 of the NF-κB2 gene.

In some embodiments, the at least one guide RNA comprises a crRNA sequence that targets exon 16 of the NF-κB2 gene.

In some embodiments, the at least one guide RNA comprises a crRNA sequence that targets exon 17 of the NF-κB2 gene.

In some embodiments, the at least one guide RNA comprises a crRNA sequence that targets exon 18 of the NF-κB2 gene.

In some embodiments, the at least one guide RNA comprises a crRNA sequence that targets exon 19 of the NF-κB2 gene.

In some embodiments, the at least one guide RNA comprises a crRNA sequence that targets exon 20 of the NF-κB2 gene.

In some embodiments, the at least one guide RNA comprises a crRNA sequence that targets exon 21 of the NF-κB2 gene.

In some embodiments, the at least one guide RNA comprises a crRNA sequence that targets exon 22 of the NF-κB2 gene.

In some embodiments, the at least one guide RNA comprises a crRNA sequence that targets exon 23 of the NF-κB2 gene.

In some embodiments, the gene associated with the production, blocking, or removal of ROS is selected from the group consisting of a 6-phosphogluconate dehydrogenase (6PGD) gene, an alcohol dehydrogenase (ADH) gene, an aldehyde dehydrogenase (ALDH2) gene, an AP-1 gene, a B-cell lymphoma-extra large (Bcl-XL) gene, a BCL2 apoptosis regulator (Bcl-2) gene, a Bcl-2-associated X protein (BAX) gene, a catalase (CAT) gene, a c-Jun N-terminal kinase (JNK) gene, a coenzyme Q10 gene, a CYP2E1 gene, a cytochrome c (Cyt c) gene, a F1Fo-ATP synthase gene, a ferritin heavy chain (FHC) gene, a glucose-6-phosphate dehydrogenase (G6PD) gene, a glutamylcysteine synthetase (GCS) gene, a glutathione (GSH) synthase gene, a glutathione peroxidase 1 (GPX1) gene, a glutathione peroxidase 2 (GPX2) gene, a glutathione peroxidase 3 (GPX3) gene, a glutathione peroxidase 4 (GPX4) gene, a glutathione peroxidase 5 (GPX5) gene, a glutathione peroxidase 6 (GPX6) gene, a glutathione peroxidase 7 (GPX7) gene, a glutathione peroxidase 8 (GPX8) gene, a glutathione reductase (GR) gene, a glycerol 3-phosphate dehydrogenase gene, a growth arrest and DNA damage (GADD 45) gene, a hypoxia-inducible factor 1-alpha (HIF-1α) gene, a mitogen-activated protein kinase (MAPK) gene, an NADH-ubiquinone oxidoreductase gene, an NADPH oxidase 4 (NOX4) gene, an NADPH oxidase 5 (NOX5) gene, a nuclear factor kappa-light-chain-enhancer of activated B cells (NF-κB) gene, a nuclear factor κB (NF-κB) essential modulator (NEMO) gene, a p46Shc (SHC isoform) gene, a p52Shc (SHC isoform) gene, a p53 upregulated modulator of apoptosis (PUMA) gene, a p66Shc (SHC isoform) gene, a phosphoinositide 3-kinase (PI3-K) gene, a proline oxidase (PIG6, POX) gene, a quinone oxidoreductase (PIG3, NQO1) gene, a respiratory complexes I gene, a respiratory complexes II gene, a respiratory complexes III gene, a respiratory complexes IV gene, a sestrin 1 (SESN1) gene, a sestrin 2 (SESN2) gene, an SHC adaptor protein 1 (SHC1) gene, a superoxide dismutase 1 (SOD1) gene, a superoxide dismutase 2 (SOD2) gene, a superoxide dismutase 3 (SOD3) gene, a TNF alpha induced protein 3 (TNFAIP3) gene, a tumor protein 53 (p53) gene, a tumor protein p53 inducible nuclear protein 1 (TP53INP1) gene, a ubiquinol-cytochrome c oxidoreductase gene, a 2B4 gene, an ABCA1 gene, an ACP5 gene, an ADAR-1 gene, an ADSS gene, an AIG1 gene, an AIM2 gene, an APOBEC3 gene, an ARRB2 gene, a B2M gene, a BCAS3 gene, a BMP4 gene, a C10orf32 gene, a C21orf33 gene, a CASP1 gene, a CCL5 gene, a CD160 gene, a Cd53 gene, a CDKN2A gene, a CHEK1 gene, a CNNM2 gene, a CNTNAP2 gene, a CSMD1 gene, a CTLA-4 gene, a CTSB gene, a C-type lectin receptors CLRs gene, a CXCL10 gene, a CYP17A1 gene, a DDX60 gene, a DYNC1I1 gene, a FOXO3a gene, a GPC6 gene, a GRN gene, an HCK gene, an HECW1 gene, an HLA gene, an IFI30 gene, an IFI44L gene, an IFI6 gene, an IFITM gene, an IFITM1/3 gene, an IFITM2 gene, an IFITM3 gene, an IL-18 gene, an IL-1α gene, an IL-1β gene, an interferon-γ gene, an interleukin-12 (IL-12) gene, an IRF gene, an IRF-1 gene, an gene, an IRF3 gene, an IRF7 gene, a LAG-3 gene, a LIPC gene, a MDA5/IFIH1 gene, a MPAK gene, a MYH9 gene, a MYO16 gene, a MYO5A gene, an NAIP gene, an NF-κB gene, an NLRC4 gene, an gene, an NLRP3 gene, a NOD2 gene, an gene, an NPL gene, an NR gene, a nucleotide oligomerization and binding domain NOD-like receptors gene, an OAS1 gene, an OAS2 gene, an OASL gene, a parkin gene (PARK2) gene, a PD-1 gene, a PLEKHG1 gene, av PRKCA gene, a PTBP1 gene, a PYCARD gene, a Pyrin-HIN (PYHIN) domain containing receptor gene (e.g. AIM2), a reactive oxygen species (ROS) gene, a retinoic acid inducible gene-I (RIG-I)-like receptor (RLRs) gene, an RFC3 gene, a RGS1 gene, a RIG-I/DDX58 gene, a SAMHD1 gene, a SF3A1/SF3B1 gene, a SFXN2 gene, a SLAMF7 gene, a SLC41A1 gene, a SLC8A1 gene, a SLCO3A1 gene, a STAT1 gene, a tetherin gene, a TLR5 gene, a TLR7 gene, a TLR9 gene, a Toll-like receptor (TLR) gene, a TREM2 gene, a TREX1 gene, a TRIM5 gene, a TTLL7 gene, and a TYROBP gene.

In some embodiments, the RNA-guided nuclease or a nucleic acid encoding an RNA-guided nuclease is the RNA-guided nuclease.

In some embodiments, the RNA-guided nuclease or a nucleic acid encoding an RNA-guided nuclease is DNA encoding the RNA-guided nuclease.

In some embodiments, the RNA-guided nuclease or a nucleic acid encoding an RNA-guided nuclease is mRNA encoding the RNA-guided nuclease.

In some embodiments, the RNA-guided nuclease is a Cas protein.

In some embodiments, the Cas protein is a Cas9 protein.

In some embodiments, the Cas9 protein is an S. pyogenes Cas9 polypeptide.

In some embodiments, the Cas9 protein is selected from the group consisting of esCas9, hfCas9, peCas9, and ARCas9.

In some embodiments, the at least one guide RNA or a nucleic acid encoding at least one guide RNA is the at least one guide RNA.

In some embodiments, the at least one guide RNA or a nucleic acid encoding at least one guide RNA is DNA encoding the at least one guide RNA.

In some embodiments, comprising a nucleic acid encoding both the RNA-guided nuclease and the at least one guide RNA.

In some embodiments, the at least one guide RNA is a single guide RNA (sgRNA).

In some embodiments, the at least one guide RNA targets a human gene.

In some embodiments, the at least one guide RNA targets a canine gene.

In some embodiments, the at least one guide RNA targets an equine gene.

In some embodiments, the at least one guide RNA targets a feline gene.

In some embodiments, the at least one guide RNA targets a mammalian gene.

In some embodiments, the composition comprises one or more viral vectors collectively comprising the (i) RNA-guided nuclease or a nucleic acid encoding an RNA-guided nuclease, and (ii) at least one guide RNA or a nucleic acid encoding at least one guide RNA targeting a gene encoding the transmembrane receptor.

In some embodiments, the one of more viral vectors comprise a recombinant virus selected from a retrovirus, an adenovirus, an adeno-associated virus, a lentivirus, and a herpes simplex virus-1.

In some embodiments, the one of more viral vectors comprise a recombinant adeno-associated virus (AAV).

In some embodiments, the recombinant AAV is of serotype 5 (AAV5).

In some embodiments, the recombinant AAV is of serotype 6 (AAV6).

In some embodiments, the composition comprises one or more lipid nanoparticles (LNP) collectively comprising the (i) RNA-guided nuclease or a nucleic acid encoding an RNA-guided nuclease, and (ii) at least one guide RNA or a nucleic acid encoding at least one guide RNA targeting a gene encoding the transmembrane receptor.

In some embodiments, the one or more LNP comprises a first plurality of LNP encapsulating the RNA-guided nuclease or a nucleic acid encoding an RNA-guided nuclease; and a second plurality of LNP encapsulating the at least one guide RNA or a nucleic acid encoding at least one guide RNA.

In some embodiments, the one or more LNP comprises a plurality of LNP encapsulating both the (i) RNA-guided nuclease or a nucleic acid encoding an RNA-guided nuclease, and (ii) at least one guide RNA or a nucleic acid encoding at least one guide RNA targeting a gene encoding the transmembrane receptor.

In some embodiments, the one or more LNP comprises a component selected from the group consisting of 3-(didodecylamino)-N1,N1,4-tridodecyl-1-piperazineethanamine (KL10), N1-[2-(didodecylamino)ethyl]-N1,N4,N4-tridodecyl-1,4-piperazinediethanamine (KL22), 14,25-ditridecyl-15,18,21,24-tetraaza-octatriacontane (KL25), 1,2-dilinoleyloxy-N,N-dimethylaminopropane (DLin-DMA), 2,2-dilinoleyl-4-dimethylaminomethyl-[1,3]-dioxolane (DLin-K-DMA), heptatriaconta-6,9,28,31-tetraen-19-yl 4-(dimethylamino)butanoate (DLin-MC3-DMA), 2,2-dilinoleyl-4-(2-dimethylaminoethyl)-[1,3]-dioxolane (DLin-KC2-DMA), 1,2-dioleyloxy-N,N-dimethylaminopropane (DODMA), 2-({8-[(3.beta.)-cholest-5-en-3-yloxy]octyl}oxy)-N,N-dimethyl-3-[(9Z,12Z)—octadeca-9,12-dien-1-yloxy]propan-1-amine (Octyl-CLinDMA), (2R)-2-({8-[(3.beta.)-cholest-5-en-3-yloxy]octyl}oxy)-N,N-dimethyl-3-[(9Z-,12Z)-octadeca-9,12-dien-1-yloxy]propan-1-amine (Octyl-CLinDMA (2R)), (2S)-2-({8-[(3.beta.)-cholest-5-en-3-yloxy]octyl}oxy)-N,N-dimethyl-3-[(9Z-,12Z)-octadeca-9,12-dien-1-yloxy]propan-1-amine (Octyl-CLinDMA (2S)), a lipid including a cyclic amine group, and a mixture thereof.

In some embodiments, the LNP comprises a component selected from the group consisting of 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-dioleoyl-sn-glycero-3-phosphoethanolamine (DOPE), 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), sphingomyelin (SM), and a mixture thereof.

In some embodiments, the LNP comprises a component selected from the group consisting of PEG-modified phosphatidylethanolamines, PEG-modified phosphatidic acids, PEG-modified ceramides, PEG-modified dialkylamines, PEG-modified diacylglycerols, PEG-modified dialkylglycerols, and mixtures thereof. For example, a PEG lipid may be PEG-c-DOMG, PEG-DMG, PEG-DLPE, PEG-DMPE, PEG-DPPC, PEG-DMA, a PEG-DSPE lipid, and a mixture thereof.

In some embodiments, the LNP comprises a component selected from the group consisting of a cholesterol, fecosterol, stigmasterol, stigmastanol, sitosterol, β-sitosterol, lupeol, betulin, ursolic acid, oleanolic acid, campesterol, fucosterol, brassicasterol, ergosterol, 9,11-dehydroergosterol, tomatidine, tomatine, α-tocopherol, and a mixture thereof.

In some embodiments, the composition comprises one or more liposomes collectively comprising the (i) RNA-guided nuclease or a nucleic acid encoding an RNA-guided nuclease, and (ii) at least one guide RNA or a nucleic acid encoding at least one guide RNA targeting a gene encoding the transmembrane receptor.

In some embodiments, the one or more liposomes comprises a first plurality of liposomes encapsulating the RNA-guided nuclease or a nucleic acid encoding an RNA-guided nuclease; and a second plurality of liposomes encapsulating the at least one guide RNA or a nucleic acid encoding at least one guide RNA.

In some embodiments, the one or more liposomes comprises a plurality of liposomes encapsulating both the (i) RNA-guided nuclease or a nucleic acid encoding an RNA-guided nuclease, and (ii) at least one guide RNA or a nucleic acid encoding at least one guide RNA targeting a gene encoding the transmembrane receptor.

In some embodiments, the composition comprises one or more virus-like particles collectively comprising the (i) RNA-guided nuclease or a nucleic acid encoding an RNA-guided nuclease, and (ii) at least one guide RNA or a nucleic acid encoding at least one guide RNA targeting a gene encoding the transmembrane receptor.

In some embodiments, the one or more virus-like particles comprises:

In some embodiments, a first plurality of virus-like particles encapsulating the RNA-guided nuclease or a nucleic acid encoding an RNA-guided nuclease; and a second plurality of virus-like particles encapsulating the at least one guide RNA or a nucleic acid encoding at least one guide RNA.

In some embodiments, the one or more virus-like particles comprises a plurality of virus-like particles encapsulating both the (i) RNA-guided nuclease or a nucleic acid encoding an RNA-guided nuclease, and (ii) at least one guide RNA or a nucleic acid encoding at least one guide RNA targeting a gene encoding the transmembrane receptor.

In some embodiments, the composition is formulated for parenteral administration.

In some embodiments, the composition is formulated for intra-articular injection within a joint of the subject.

In some embodiments, the disclosure provides a method for treating or preventing a joint disorder in a subject in need thereof, comprising administering a therapeutically effective amount of a pharmaceutical composition described above to the subject.

In some embodiments, the disclosure provides a method for treating or preventing a joint disorder in a subject in need thereof, comprising administering a therapeutically effective amount of a pharmaceutical composition described above to the subject.

### VII. Administration Routes

The methods and compositions herein described encompass the use of pharmaceutical compositions comprising a CRISPR gene-editing system as an active ingredient.

Depending on the method/route of administration, pharmaceutical dosage forms come in several types. These include many kinds of liquid, solid, and semisolid dosage forms. Common pharmaceutical dosage forms include pill, tablet, or capsule, drink or syrup, and natural or herbal form such as plant or food of sorts, among many others. Notably, the route of administration (ROA) for drug delivery is dependent on the dosage form of the substance in question. A liquid pharmaceutical dosage form is the liquid form of a dose of a chemical compound used as a drug or medication intended for administration or consumption.

As used herein, “dermal delivery” or “dermal administration” can refer to a route of administration wherein the pharmaceutical dosage form is taken to, or through, the dermis (i.e., layer of skin between the epidermis (with which it makes up the cutis) and subcutaneous tissues). “Subcutaneous delivery” can refer to a route of administration wherein the pharmaceutical dosage form is to or beneath the subcutaneous tissue layer.

Methods of formulating suitable pharmaceutical compositions are known in the art, see, e.g., Remington: The Science and Practice of Pharmacy, 21st ed., 2005; and the books in the series Drugs and the Pharmaceutical Sciences: a Series of Textbooks and Monographs (Dekker, N.Y.). For example, solutions or suspensions used for parenteral, intradermal, or subcutaneous application can include the following components: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfate; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent that delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yield a powder of the active ingredient plus any additional desired ingredient from a previously sterile-filtered solution thereof.

Therapeutic compounds that are or include nucleic acids can be administered by any method suitable for administration of nucleic acid agents, such as a DNA vaccine. These methods include gene guns, bio injectors, and skin patches as well as needle-free methods such as the micro-particle DNA vaccine technology disclosed in U.S. Pat. No. 6,194,389, and the mammalian transdermal needle-free vaccination with powder-form vaccine as disclosed in U.S. Pat. No. 6,168,587. Additionally, intranasal delivery is possible, as described in, inter alia, Hamajima et al., Clin. Immunol. Immunopathol., 88(2), 205-10 (1998). Liposomes (e.g., as described in U.S. Pat. No. 6,472,375) and microencapsulation can also be used. Biodegradable targetable microparticle delivery systems can also be used (e.g., as described in U.S. Pat. No. 6,471,996).

Therapeutic compounds can be prepared with carriers that will protect the therapeutic compounds against rapid elimination from the body, such as a controlled release formulation, including implants and microencapsulated delivery systems. Biodegradable, biocompatible polymers can be used, such as collagen, ethylene vinyl acetate, polyanhydrides (e.g., poly[1,3-bis(carboxyphenoxy)propane-co-sebacic-acid](PCPP-SA) matrix, fatty acid dimer-sebacic acid (FAD-SA) copolymer, poly(lactide-co-glycolide)), polyglycolic acid, collagen, polyorthoesters, polyethylene glycol-coated liposomes, hyaluronic acid and polylactic acid. Such formulations can be prepared using standard techniques, or obtained commercially, e.g., from Alza Corporation and Nova Pharmaceuticals, Inc. Liposomal suspensions (including liposomes targeted to selected cells with monoclonal antibodies to cellular antigens) can also be used as pharmaceutically acceptable carriers. These can be prepared according to methods known to those skilled in the art, for example, as described in U.S. Pat. No. 4,522,811. Semisolid, gelling, soft-gel, or other formulations (including controlled release) can be used, e.g., when administration to a surgical site is desired. Methods of making such formulations are known in the art and can include the use of biodegradable, biocompatible polymers. See, e.g., Sawyer et al., Yale J Biol Med. 2006 December; 79(3-4): 141-152.

The pharmaceutical compositions described herein may be included in a container, kit, pack, or dispenser together with instructions for administration.

### A. Systemic Administration

In some embodiments, a pharmaceutical composition comprising a CRISPR gene-editing system is administered systemically to a mammal in need thereof. In some embodiments, the composition is formulated for intravenous injection. In some embodiments, the composition is formulated for oral administration. In some embodiments, the composition is formulated for parenteral administration.

### B. Local Administration

In some embodiments, a pharmaceutical composition comprising a CRISPR gene-editing system is administered locally to a mammal in need thereof. In some embodiments, the local administration is an intra-articular injection.

In some embodiments, a pharmaceutical composition comprising a CRISPR gene-editing system is administered locally to a mammal in need thereof during a surgical procedure. In some embodiments, a pharmaceutical composition comprising a CRISPR gene-editing system is administered locally to a mammal in need thereof 1, 2, 3, 4, 5, 6, 7, 8, 9, 10, 20, or 30 days after a surgical procedure.

### EXAMPLES

The embodiments encompassed herein are now described with reference to the following examples. These examples are provided for the purpose of illustration only and the disclosure encompassed herein should in no way be construed as being limited to these examples, but rather should be construed to encompass any and all variations which become evident as a result of the teachings provided herein.

### Example 1: MSU Treatment of Canine Monocytes Triggers Upregulation of Multiple Inflammatory Effectors

To understand the potential nexus between inflammatory signaling (and, by extension, induction of ROS) and joint disease, the transcriptional response to monosodium urate (MSU) crystals—a proxy for the build-up of uric acid observed in gout—were investigated in canine DH82 monocytes (biological triplicates, N=3), using bulk RNA-Seq and differential expression analysis DESeq2. Cells were exposed to either saline solution PBS (negative control) or 400 μg/ml MSU crystals, and total RNA was extracted after 24 hours.

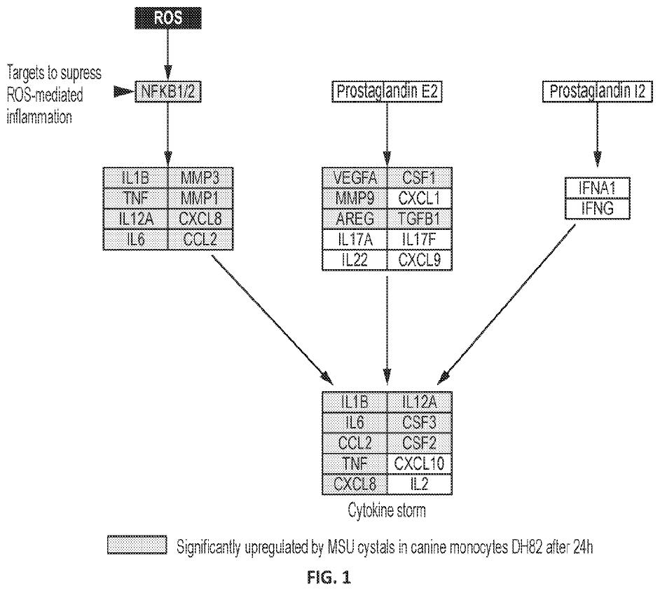

The results found that, compared to PBS controls, MSU-treated cells exhibited strong transcriptional induction of NFKB1 and NFKB2, both of which are downstream effectors of ROS detection and upstream regulators of pro-inflammatory signaling (FIG. 1). These results demonstrated the importance of the expression of NFKB1 and NFKB2 at the transcriptional level in an in vitro disease model and suggested that targeted interventions that could abrogate the inflammatory response to ROS detection, thereby treating any number of conditions that are caused or exacerbated by ROS.

### Example 2: Design and Selection of Guides for Editing of NFKB1

A pipeline of in silico and in vitro screening was developed to identify candidate CRISPR-Cas guides for gene editing. The pipeline was applied to identify guides that effectively edit the human NFKB1 gene (hNFKB1), thereby disrupting the pathway the results in ROS production before it can be activated. The first step in the pipeline was to identify all possible crRNA sequences for a particular CRISPR-Cas protein in the coding portions of the hNFKB1. Many algorithms for identifying such sequences are known in the art. Generally, these algorithms function by identifying a protospacer adjacent motif (PAM) sequence for the particular CRISPR-Cas protein and then locate the sequence spaced according to the requirements of the particular Cas protein, typically directly 5′ of the PAM site. For example, the S. pyogenes Cas9 (SpCas9) protein, which was used in this Example, recognizes a 5′-NGG-3′ PAM sequence. Thus, all sequences directly 5′ of an NGG trinucleotide are possible crRNA sequences. crRNA sequences identified in this fashion as shown in FIG. 5, along with biographical information about the crRNA sequence.

Next, each identified crRNA sequence was evaluated across three different metrics: possible off-target editing at locations in the genome other than the target gene, on-target editing efficiency, and the likelihood of editing causing frameshift mutations, using multiple algorithms for each metric. The basis of the combinatorial approach used lies in the assumption that every model has blind spots that may skew the fitness of a particular guide RNA. Weighting these scores to obtain a consensus score for each of these properties allows for much better prediction of sgRNA fitness.

Off-target editing effects were predicted by averaging scores generated by the MIT, CFD and Elevation (human only) model. The MIT algorithm, also known as Hsu-Zhang score. Hsu, P. D. et al. DNA targeting specificity of RNA-guided Cas9 nucleases. Nature Biotechnology 31, 827-832 (2013). This model is based on a positional penalty matrix (1×20) generated from 15 EMX1 sgRNA libraries with mismatches against target at every position. The CFD algorithm (Cutting Frequency Determination) is based on threat matrix (12×20) considering both position and mismatch type and PAM integrity (27,897 ‘CD33’ sgRNAs+10,618 negative control sgRNAs). Doench, J. G. et al. Optimized sgRNA design to maximize activity and minimize off-target effects of CRISPR-Cas9. Nature Biotechnology 34, 184-191 (2016). The Elevation score using machine learning algorithms trained by genome-wide (GUIDE-Seq) and other aggregated off-target profiling data. Listgarten, J. et al. Prediction of off-target activities for the end-to-end design of CRISPR guide RNAs. Nature Biomedical Engineering 2, 38-47 (2018). The column labelled “OFF” shows the mean of the scores provided by the two or three models, respectively. The column labelled “OffTarget #” shows the number of potential off-targets with up to four mismatches as calculated by CRISPOR. See, Haeussler, M. et al. Evaluation of off-target and on-target scoring algorithms and integration into the guide RNA selection tool CRISPOR. Genome Biology 17, 148 (2016).

On-target editing efficiencies were predicted by averaging scores generated by the Azimuth model, the DeepSpCas9 model, and the CrisprScan model. The Azimuth model is a boosted regression tree model, trained with 881 sgRNAs (MOLM13/NB4/TF1 cells and additional unpublished data) delivered by lentivirus. Doench, J. G. et al. Optimized sgRNA design to maximize activity and minimize off-target effects of CRISPR-Cas9. Nature Biotechnology 34, 184-191 (2016). DeepSpCas9 is a deep learning model trained using editing data from 12,832 sgRNA. Kim, H. K. et al. SpCas9 activity prediction by DeepSpCas9, a deep learning-based model with high generalization performance. Science Advances 5(11)(2019). CrisprScan is a linear regression model, trained using editing data from 1000 sgRNAs injected into zebrafish embryos targeting >100 genes. Moreno-Mateos, M. A. et al. CRISPRscan: designing highly efficient sgRNAs for CRISPR-Cas9 targeting in vivo. Nature Methods 12, 982-988 (2015). The column labelled “ON” shows the mean of the scores provided by the three models.

The putative guides' potential to generate frameshift mutations were predicted by averaging scores generated by the Lindel model and the InDelphi model. Lindel is a machine learning model trained using profile data of 1.16 million independent mutational events triggered by CRISPR/Cas9-mediated cleavage and non-homologous end joining-mediated double strand break repair of 6872 synthetic target sequences, introduced into a human cell line via lentiviral infection. Chen, W. et al. Massively parallel profiling and predictive modeling of the outcomes of CRISPR/Cas9-mediated double-strand break repair. Nucleic Acids Research 47, 7989-8003 (2019). InDelphi is machine learning model trained with indels generated by 1872 sgRNAs. Shen, M. W. et al. Predictable and precise template-free CRISPR editing of pathogenic variants. Nature 563, 646-651 (2018). The column labelled “Frameshift” shows the mean of the scores provided by the two models. The column labelled “Precision” shows the frequency distribution of indels estimated by inDelphi. High precision is closer to 100 and represents sgRNAs that are characterised by one or a very low number of repair outcomes.

The candidate crRNA sequences were then evaluated for the presence of Graf motifs, TT or GCC present in the 4 PAM proximal bases of the crRNA sequence, as indicated in, e.g., FIGS. 26-42, as either TT or GCC. Graf, R. et al. sgRNA Sequence Motifs Blocking Efficient CRISPR/Cas9-Mediated Gene Editing. Cell Reports, 26(5), 1098-103 (2019). Graf et al. reported that TT- and GCC-motifs are a hallmark of inefficient sgRNAs. If possible, crRNA with Graf motifs and in particular the GCC motif were avoided. In contrast to the TT motif, the GCC motif remains critical if sgRNAs are synthesized de novo rather than by transcription.

As shown in, e.g., FIGS. 5-6, three consensus scores were then calculated for each crRNA sequence. The “ON-OFF” score represents the mean of the “ON” and “OFF” score. The “OFF-FS” score represents the mean of the “OFF” and “Frameshift” score. The OVERALL score represents the mean of the “ON”, “OFF” and “Frameshift” score. Additionally, for human only, VBC's Bioscore was used to predict whether a possible in-frame mutation could disturb protein function. This is more likely to occur in conserved genomic DNA sequences coding for critical protein domains. Thus, Bioscore is based on protein domain annotations, phylogenetic conservation, amino acid identities and exon size. Michlits, G. et al. Multilayered VBC score predicts sgRNAs that efficiently generate loss-of-function alleles. Nature Methods 17, 708-716 (2020). The final criteria for selecting candidates is mainly based on the OVERALL score (in most cases >70), the relative low counts of potential off-targets (in most cases <200 off-targets), the absence of Graf motifs (if possible) and the genomic cut position within the coding sequence to produce knockouts or truncated proteins with reduced functionality.

Indeed, this in silico analysis established that several candidate sgRNAs were predicted to yield genomic edits that would create a genetic knockout of the NFKB1, thereby impacting intracellular ROS production and pro-inflammatory signaling. These predictive studies demonstrated the feasibility of inducing an indel within the genome at the NFKB1 locus as a unique approach to tamping down ROS in cells and provided a high-throughput method for validating candidates without the need for time- and resource-intensive brute force methods.

### Example 3: Design and Selection of Guides for Editing of NFKB2

A pipeline of in silico and in vitro screening was developed to identify candidate CRISPR-Cas guides for gene editing. The pipeline was applied to identify guides that effectively edit the human NFKB2 gene (hNFKB2), thereby disrupting the pathway that results in ROS production before it can be activated. The first step in the pipeline was to identify all possible crRNA sequences for a particular CRISPR-Cas protein in the coding portions of the hNFKB2. The approach to design and in silico analysis is described in detail in Example 2.

As shown in FIG. 6, this approach established that several candidate sgRNAs were predicted to yield genomic edits that would create a genetic knockout of the NFKB2, thereby impacting intracellular ROS production and pro-inflammatory signaling. These predictive studies demonstrated the feasibility of inducing an indel within the genome at the NFKB2 locus as a unique approach to tamping down ROS in cells and provided a high-throughput method for validating candidates without the need for time- and resource-intensive brute force methods.

### Example 4: Validation of NFKB1 Editing in Human Cells

Having designed and analyzed numerous sgRNA candidates that were predicted to edit the NFKB1 gene to produce a knockout (see FIG. 5A), the next step is to assess their ability to act in vitro. Of particular interest is whether the preferred (i.e., on-target) edit will be observed in edited cells.

To test this, 80 pmoles of sgRNA candidates (SEQ ID NOs: 1-328) are introduced into human THP-1 monocytes via electroporation as part of a ribonucleoprotein (i.e., pre-assembled with 25 pmoles or either wtCas9 or AR-Cas9 protein at room temperature for at least 5 min). In brief, around 600,000 cells resuspended in 20 μl PKM buffer (2.7 mM KCl, 137 mM NaCl, 10 mM Na2HPO4/1.8 mM NaH2PO4 (pH 7.4) and 50 mM D-Mannitol) are added to the pre-assembled Cas9 ribonucleoprotein complex, transferred into 16-well nucleocuvettes and electroporated with pulse code CM-137 using the 4D nucleofector (Lonza). Electroporated cells are kept in PKM buffer for about 10 min before transferring them into 6-well tissue culture plates with complete culture media. After 2-5 days in culture, cells are dissociated for genomic DNA extraction using the DNeasy Blood & Tissue Kit (Qiagen). sgRNA target regions are amplified by PCR and editing efficacy is then deduced from Sanger sequencing traces using Inference of CRISPR Edits (ICE v1.2). Conant, D. et al. (2022). The CRISPR Journal, 5(1), 123-130 (2022).

It is anticipated that results of these studies will confirm the robustness of the in silico analysis described above and identify multiple candidates that are indeed able to induce mutation in the NFKB1 gene at high efficacy. It is further expected that use of AR-Cas9 will improve editing efficacy (i.e., increase on-target editing) as compared to wtCas9.

### Example 5: In Vitro Delivery of NFKB1-Editing Guide RNA to Human Cells Via Lipid Nanoparticles

Because all validation experiments are to be conducted via electroporation of cell cultures to introduce RNA-dependent nucleases and sgRNAs, additional experiments will assess the suitability of using lipid nanoparticle (LNP) to deliver mRNA to human cells. 0.025 mg/mL of a LNP containing either 80 pmoles of sgRNA (SEQ ID NOs: 1-328) or 80 pmoles of Cas9 mRNA (alternatively, sgRNA may be pre-assembled with 25 pmoles or either wtCas9 or AR-Cas9 protein at room temperature for at least 5 min and encapsulated as an RNP) are exposed to THP-1 monocytes for 8 to 24 hours under typical cell culture conditions. Sanger sequencing as described in Example 4 will be used to validate editing. Upon confirmation of editing, various functional assays, e.g., those described in Example 1, will be performed with the edited cells.

### Example 6: In Vitro Delivery of NFKB1-Editing Guide RNA to Human Cells Via Adeno-Associated Virus

Because all validation experiments are to be conducted via electroporation of cell cultures to introduce RNA-dependent nucleases and sgRNAs, additional experiments will assess the suitability of using adeno-associated virus (AAV) vectors to deliver sgRNA to human cells.

TABLE 3
Exemplary AAV vectors for introduction of sgRNA to human cells.

AAVPrimeTM Adeno-Associated Virus - Serotype6 (AAV-6) Particles for sgRNA
(GeneCopoeia ™, catalog No. AA06-MCP001682-AD01-2-200-a, lot No. GC03182K2001)
AAVPrimeTM Adeno-Associated Virus - Serotype6 (AAV-6) Particles for sgRNA
(GeneCopoeia ™, catalog No. AA06-MCP001682-AD01-2-200-b, lot No. GC03202K2001)
AAVPrimeTM Adeno-Associated Virus - Serotype6 (AAV-6) Particles for sgRNA
(GeneCopoeia ™, catalog No. AA06-CCPCTR01-AD01-200, lot No. GC03112K2002)
AAVPrimeTM Adeno-Associated Virus - Serotype5 (AAV-5) Particles for sgRNA
(GeneCopoeia ™, catalogue No. AA05-MCP001682-AD01-2-200A, lot No. GC-
03182K2002)
AAVPrimeTM Adeno-Associated Virus - Serotype5 (AAV-5) Particles for sgRNA
(GeneCopoeia ™, catalogue No. AA05-MCP001682-AD01-2-200B, lot No.
GC03182K2003)
AAVPrimeTM Adeno-Associated Virus - Serotype5 (AAV-5) Particles for sgRNA
(GeneCopoeiaTM, catalogue No. AA05-CCPCTR01-AD01-200, lot No. GC03032K2003)

Exemplary AAV vectors are shown in Table 3. After being prepared according to manufacturer specifications, such that one or more of the sgRNAs containing SEQ ID NOs: 1-328 are integrated into the viral vector, said vectors are incubated (MOI=0.1 to 10) with approximately 500,000 U2OS cells that have been stably transfected with Cas9 (see, e.g., Rojas-Fernandez, A., et al. (2015). Scientific Reports, 5(1), 1-6). At 2-7 days post-infection, Sanger sequencing as described in Example 4 will be used to validate editing. Upon confirmation of editing, various functional assays, e.g., those described in Example 1, will be performed with the edited cells.

### Example 7: Validation of NFKB2 Editing in Human Cells

Having designed and analyzed numerous sgRNA candidates that were predicted to edit the human NFKB2 gene to produce a genetic knockout (see FIG. 6A), the next step is to assess their ability to act in vitro. Of particular interest is whether the preferred (i.e., on-target) edit will be observed in edited cells.

To test this, 80 pmoles of sgRNA candidates (SEQ ID NOs: 329-680 and 5323-5410) are introduced into human THP-1 monocytes via electroporation as part of a ribonucleoprotein as previously described in Example 4.

It is anticipated that results of these studies will confirm the robustness of the in silico analysis and identify multiple candidates are indeed able to induce mutation in the IL1RAP gene at high efficacy. It is further expected that use of AR-Cas9 will improve editing efficacy (i.e., increase on-target editing) as compared to wtCas9.

### Example 8: In Vitro Delivery of NFKB2-Editing Guide RNA to Human Cells Via Lipid Nanoparticles

Because all validation experiments are to be conducted via electroporation of cell cultures to introduce RNA-dependent nucleases and sgRNAs, additional experiments will assess the suitability of using lipid nanoparticle (LNP) to deliver mRNA to human cells. LNPs containing either sgRNA (SEQ ID NOs: 329-680 and 5323-5410) or Cas9 mRNA are incubated with THP-1 monocytes, followed by validation of editing as described in Example 5. Upon confirmation of editing various functional assays, e.g., those described in Example 1, will be performed with the edited cells.

### Example 9: In Vitro Delivery of NFKB2-Editing Guide RNA to Human Cells Via Adeno-Associated Virus

Because all validation experiments are to be conducted via electroporation of cell cultures to introduce RNA-dependent nucleases and sgRNAs, additional experiments will assess the suitability of using adeno-associated virus (AAV) vectors to deliver sgRNA to human cells. Exemplary AAV vectors are shown in Table 3. After being prepared according to manufacturer specifications, such that one or more of the sgRNAs containing SEQ ID NOs: 329-680 or SEQ ID NOs: 5323-5410 are integrated into the viral vector, said vectors are introduced to human U2OS cells and editing is validated as previously described in Example 6. Upon confirmation of editing, various functional assays, e.g., those described in Example 1, will be performed.

The examples set forth above are provided to give those of ordinary skill in the art a complete disclosure and description of how to make and use the embodiments of the compositions, systems and methods of the disclosure, and are not intended to limit the scope of what the inventors regard as their invention. Modifications of the above-described modes for carrying out the embodiments of the disclosure that are obvious to persons of skill in the art are intended to be within the scope of the following claims. All patents and publications mentioned in the specification are indicative of the levels of skill of those skilled in the art to which the disclosure pertains.

All headings and section designations are used for clarity and reference purposes only and are not to be considered limiting in any way. For example, those of skill in the art will appreciate the usefulness of combining various aspects from different headings and sections as appropriate according to the spirit and scope of the disclosure described herein.

It is to be understood that the methods described herein are not limited to the particular methodology, protocols, subjects, and sequencing techniques described herein and as such can vary. It is also to be understood that the terminology used herein is for the purpose of describing particular embodiments only, and is not intended to limit the scope of the methods and compositions described herein, which will be limited only by the appended claims. While some embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein can be employed in practicing the disclosure. It is intended that the following claims define the scope of the disclosure and that methods and structures within the scope of these claims and their equivalents be covered thereby.

The examples set forth above are provided to give those of ordinary skill in the art a complete disclosure and description of how to make and use the embodiments of the compositions, systems and methods of the disclosure, and are not intended to limit the scope of what the inventors regard as their invention. Modifications of the above-described modes for carrying out the embodiments of the disclosure that are obvious to persons of skill in the art are intended to be within the scope of the following claims. All patents and publications mentioned in the specification are indicative of the levels of skill of those skilled in the art to which the disclosure pertains.

All headings and section designations are used for clarity and reference purposes only and are not to be considered limiting in any way. For example, those of skill in the art will appreciate the usefulness of combining various aspects from different headings and sections as appropriate according to the spirit and scope of the disclosure described herein.

It is to be understood that the methods described herein are not limited to the particular methodology, protocols, subjects, and sequencing techniques described herein and as such can vary. It is also to be understood that the terminology used herein is for the purpose of describing particular embodiments only, and is not intended to limit the scope of the methods and compositions described herein, which will be limited only by the appended claims. While some embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein can be employed in practicing the disclosure. It is intended that the following claims define the scope of the disclosure and that methods and structures within the scope of these claims and their equivalents be covered thereby.

Several aspects are described with reference to example applications for illustration. Unless otherwise indicated, any embodiment can be combined with any other embodiment. It should be understood that numerous specific details, relationships, and methods are set forth to provide a full understanding of the features described herein. A skilled artisan, however, will readily recognize that the features described herein can be practiced without one or more of the specific details or with other methods. The features described herein are not limited by the illustrated ordering of acts or events, as some acts can occur in different orders and/or concurrently with other acts or events. Furthermore, not all illustrated acts or events are required to implement a methodology in accordance with the features described herein.

While some embodiments have been shown and described herein, it will be obvious to those skilled in the art that such embodiments are provided by way of example only. It is not intended that the disclosure be limited by the specific examples provided within the specification. While the disclosure has been described with reference to the aforementioned specification, the descriptions and illustrations of the embodiments herein are not meant to be construed in a limiting sense. Numerous variations, changes, and substitutions will now occur to those skilled in the art without departing from the disclosure.

Furthermore, it shall be understood that all aspects of the disclosure are not limited to the specific depictions, configurations or relative proportions set forth herein which depend upon a variety of conditions and variables. It should be understood that various alternatives to the embodiments of the disclosure described herein can be employed in practicing the disclosure. It is therefore contemplated that the disclosure shall also cover any such alternatives, modifications, variations or equivalents. It is intended that the following claims define the scope of the disclosure and that methods and structures within the scope of these claims and their equivalents be covered thereby.

All publications, patents, and patent applications herein are incorporated by reference to the same extent as if each individual publication, patent, or patent application was specifically and individually indicated to be incorporated by reference. In the event of a conflict between a term herein and a term in an incorporated reference, the term herein controls.

What is claimed is:

1. A pharmaceutical composition for treating or preventing a joint disorder, comprising:
(i) an RNA-guided nuclease or a nucleic acid encoding an RNA-guided nuclease; and
(ii) at least one guide RNA or a nucleic acid encoding at least one guide RNA targeting a gene associated with the production, blocking, or removal of reactive oxygen species (ROS).

2. The pharmaceutical composition of claim 1, wherein the joint disorder is arthritis.

3. The pharmaceutical composition of claim 1, wherein the joint disorder is osteoarthritis.

4. The pharmaceutical composition of claim 1, wherein the joint disorder is rheumatoid arthritis.

5. The pharmaceutical composition of claim 1, wherein the joint disorder is post-traumatic arthritis.

6. The pharmaceutical composition of claim 1, wherein the joint disorder is gout

7. The pharmaceutical composition of claim 1, wherein the joint disorder is pseudogout.

8. The pharmaceutical composition of claim 1, wherein the joint disorder is canine, equine, or feline lameness.

9. The pharmaceutical composition of claim 1, wherein the joint disorder is tendinopathy.

10. The pharmaceutical composition of any one of claims 1-9, wherein the gene associated with the production, blocking, or removal of ROS is a nuclear factor kappa B subunit 1 (NF-κB1) gene.

11. The pharmaceutical composition of claim 10, wherein the at least one guide RNA comprises a crRNA sequence selected from the group consisting of those sequences shown in FIG. 5 (SEQ ID NOS: 1-328).

12. The pharmaceutical composition of claim 10, wherein the at least one guide RNA comprises a crRNA sequence selected from the group consisting of SEQ ID NOS: 1-149.

13. The pharmaceutical composition of claim 10, wherein the at least one guide RNA comprises a crRNA sequence selected from the group consisting of SEQ ID NOS: 1-45.

14. The pharmaceutical composition of claim 10, wherein the at least one guide RNA comprises a crRNA sequence selected from the group consisting of SEQ ID NOS: 1-13.

15. The pharmaceutical composition of any one of claims 10-14, wherein the at least one guide RNA comprises a crRNA sequence that targets exon 1 of the NF-κB1 gene.

16. The pharmaceutical composition of claim 10-14, wherein the at least one guide RNA comprises a crRNA sequence that targets exon 2 of the NF-κB1 gene.

17. The pharmaceutical composition of claim 10-14, wherein the at least one guide RNA comprises a crRNA sequence that targets exon 3 of the NF-κB1 gene.

18. The pharmaceutical composition of claim 10-14, wherein the at least one guide RNA comprises a crRNA sequence that targets exon 4 of the NF-κB1 gene.

19. The pharmaceutical composition of claim 10-14, wherein the at least one guide RNA comprises a crRNA sequence that targets exon 5 of the NF-κB1 gene.

20. The pharmaceutical composition of claim 10-14, wherein the at least one guide RNA comprises a crRNA sequence that targets exon 6 of the NF-κB1 gene.

21. The pharmaceutical composition of claim 10-14, wherein the at least one guide RNA comprises a crRNA sequence that targets exon 7 of the NF-κB1 gene.

22. The pharmaceutical composition of claim 10-14, wherein the at least one guide RNA comprises a crRNA sequence that targets exon 8 of the NF-κBd1 gene.

23. The pharmaceutical composition of claim 10-14, wherein the at least one guide RNA comprises a crRNA sequence that targets exon 9 of the NF-κBd1 gene.

24. The pharmaceutical composition of claim 10-14, wherein the at least one guide RNA comprises a crRNA sequence that targets exon 10 of the NF-κB1 gene.

25. The pharmaceutical composition of claim 10-14, wherein the at least one guide RNA comprises a crRNA sequence that targets exon 11 of the NF-κB1 gene.

26. The pharmaceutical composition of claim 10-14, wherein the at least one guide RNA comprises a crRNA sequence that targets exon 12 of the NF-κB1 gene.

27. The pharmaceutical composition of claim 10-14, wherein the at least one guide RNA comprises a crRNA sequence that targets exon 13 of the NF-κB1 gene.

28. The pharmaceutical composition of claim 10-14, wherein the at least one guide RNA comprises a crRNA sequence that targets exon 14 of the NF-κB1 gene.

29. The pharmaceutical composition of claim 10-14, wherein the at least one guide RNA comprises a crRNA sequence that targets exon 15 of the NF-κB1 gene.

30. The pharmaceutical composition of claim 10-14, wherein the at least one guide RNA comprises a crRNA sequence that targets exon 16 of the NF-κB1 gene.

31. The pharmaceutical composition of claim 10-14, wherein the at least one guide RNA comprises a crRNA sequence that targets exon 17 of the NF-κB1 gene.

32. The pharmaceutical composition of claim 10-14, wherein the at least one guide RNA comprises a crRNA sequence that targets exon 18 of the NF-κB1 gene.

33. The pharmaceutical composition of claim 10-14, wherein the at least one guide RNA comprises a crRNA sequence that targets exon 19 of the NF-κB1 gene.

34. The pharmaceutical composition of claim 10-14, wherein the at least one guide RNA comprises a crRNA sequence that targets exon 20 of the NF-κB1 gene.

35. The pharmaceutical composition of claim 10-14, wherein the at least one guide RNA comprises a crRNA sequence that targets exon 21 of the NF-κB1 gene.

36. The pharmaceutical composition of claim 10-14, wherein the at least one guide RNA comprises a crRNA sequence that targets exon 22 of the NF-κB1 gene.

37. The pharmaceutical composition of claim 10-14, wherein the at least one guide RNA comprises a crRNA sequence that targets exon 23 of the NF-κB1 gene.

38. The pharmaceutical composition of claim 10-14, wherein the at least one guide RNA comprises a crRNA sequence that targets exon 24 of the NF-κB1 gene.

39. The pharmaceutical composition of any one of claims 1-9, wherein the gene associated with the production, blocking, or removal of ROS is a nuclear factor kappa B subunit 2 (NF-κB2) gene.

40. The pharmaceutical composition of claim 39, wherein the at least one guide RNA comprises a crRNA sequence selected from the group consisting of those sequences shown in FIG. 6 (SEQ ID NOS: 329-680 and 5323-5410).

41. The pharmaceutical composition of claim 39, wherein the at least one guide RNA comprises a crRNA sequence selected from the group consisting of SEQ ID NOS: 329-497.

42. The pharmaceutical composition of claim 39, wherein the at least one guide RNA comprises a crRNA sequence selected from the group consisting of SEQ ID NOS: 329-384.

43. The pharmaceutical composition of claim 39, wherein the at least one guide RNA comprises a crRNA sequence selected from the group consisting of SEQ ID NOS: 329-335.

44. The pharmaceutical composition of any one of claims 39-43, wherein the at least one guide RNA comprises a crRNA sequence that targets exon 1 of the NF-κB2 gene.

45. The pharmaceutical composition of claim 39-43, wherein the at least one guide RNA comprises a crRNA sequence that targets exon 2 of the NF-κB2 gene.

46. The pharmaceutical composition of claim 39-43, wherein the at least one guide RNA comprises a crRNA sequence that targets exon 3 of the NF-κB2 gene.

47. The pharmaceutical composition of claim 39-43, wherein the at least one guide RNA comprises a crRNA sequence that targets exon 4 of the NF-κB2 gene.

48. The pharmaceutical composition of claim 39-43, wherein the at least one guide RNA comprises a crRNA sequence that targets exon 5 of the NF-κB2 gene.

49. The pharmaceutical composition of claim 39-43, wherein the at least one guide RNA comprises a crRNA sequence that targets exon 6 of the NF-κB2 gene.

50. The pharmaceutical composition of claim 39-43, wherein the at least one guide RNA comprises a crRNA sequence that targets exon 7 of the NF-κB2 gene.

51. The pharmaceutical composition of claim 39-43, wherein the at least one guide RNA comprises a crRNA sequence that targets exon 8 of the NF-κB2 gene.

52. The pharmaceutical composition of claim 39-43, wherein the at least one guide RNA comprises a crRNA sequence that targets exon 9 of the NF-κB2 gene.

53. The pharmaceutical composition of claim 39-43, wherein the at least one guide RNA comprises a crRNA sequence that targets exon 10 of the NF-κB2 gene.

54. The pharmaceutical composition of claim 39-43, wherein the at least one guide RNA comprises a crRNA sequence that targets exon 11 of the NF-κB2 gene.

55. The pharmaceutical composition of claim 39-43, wherein the at least one guide RNA comprises a crRNA sequence that targets exon 12 of the NF-κB2 gene.

56. The pharmaceutical composition of claim 39-43, wherein the at least one guide RNA comprises a crRNA sequence that targets exon 13 of the NF-κB2 gene.

57. The pharmaceutical composition of claim 39-43, wherein the at least one guide RNA comprises a crRNA sequence that targets exon 14 of the NF-κB2 gene.

58. The pharmaceutical composition of claim 39-43, wherein the at least one guide RNA comprises a crRNA sequence that targets exon 15 of the NF-κB2 gene.

59. The pharmaceutical composition of claim 39-43, wherein the at least one guide RNA comprises a crRNA sequence that targets exon 16 of the NF-κB2 gene.

60. The pharmaceutical composition of claim 39-43, wherein the at least one guide RNA comprises a crRNA sequence that targets exon 17 of the NF-κB2 gene.

61. The pharmaceutical composition of claim 39-43, wherein the at least one guide RNA comprises a crRNA sequence that targets exon 18 of the NF-κB2 gene.

62. The pharmaceutical composition of claim 39-43, wherein the at least one guide RNA comprises a crRNA sequence that targets exon 19 of the NF-κB2 gene.

63. The pharmaceutical composition of claim 39-43, wherein the at least one guide RNA comprises a crRNA sequence that targets exon 20 of the NF-κB2 gene.

64. The pharmaceutical composition of claim 39-43, wherein the at least one guide RNA comprises a crRNA sequence that targets exon 21 of the NF-κB2 gene.

65. The pharmaceutical composition of claim 39-43, wherein the at least one guide RNA comprises a crRNA sequence that targets exon 22 of the NF-κB2 gene.

66. The pharmaceutical composition of claim 39-43, wherein the at least one guide RNA comprises a crRNA sequence that targets exon 23 of the NF-κB2 gene.

67. The pharmaceutical composition of any one of claims 1-9, wherein the gene associated with the production, blocking, or removal of ROS is selected from the group consisting of a 6-phosphogluconate dehydrogenase (6PGD) gene, an alcohol dehydrogenase (ADH) gene, an aldehyde dehydrogenase (ALDH2) gene, an AP-1 gene, a B-cell lymphoma-extra large (Bcl-XL) gene, a BCL2 apoptosis regulator (Bcl-2) gene, a Bcl-2-associated X protein (BAX) gene, a catalase (CAT) gene, a c-Jun N-terminal kinase (JNK) gene, a coenzyme Q10 gene, a CYP2E1 gene, a cytochrome c (Cyt c) gene, a F1Fo-ATP synthase gene, a ferritin heavy chain (FHC) gene, a glucose-6-phosphate dehydrogenase (G6PD) gene, a glutamylcysteine synthetase (GCS) gene, a glutathione (GSH) synthase gene, a glutathione peroxidase 1 (GPX1) gene, a glutathione peroxidase 2 (GPX2) gene, a glutathione peroxidase 3 (GPX3) gene, a glutathione peroxidase 4 (GPX4) gene, a glutathione peroxidase 5 (GPX5) gene, a glutathione peroxidase 6 (GPX6) gene, a glutathione peroxidase 7 (GPX7) gene, a glutathione peroxidase 8 (GPX8) gene, a glutathione reductase (GR) gene, a glycerol 3-phosphate dehydrogenase gene, a growth arrest and DNA damage (GADD 45) gene, a hypoxia-inducible factor 1-alpha (HIF-1α) gene, a mitogen-activated protein kinase (MAPK) gene, an NADH-ubiquinone oxidoreductase gene, an NADPH oxidase 4 (NOX4) gene, an NADPH oxidase 5 (NOX5) gene, a nuclear factor kappa-light-chain-enhancer of activated B cells (NF-κB) gene, a nuclear factor κB (NF-κB) essential modulator (NEMO) gene, a p46Shc (SHC isoform) gene, a p52Shc (SHC isoform) gene, a p53 upregulated modulator of apoptosis (PUMA) gene, a p66Shc (SHC isoform) gene, a phosphoinositide 3-kinase (PI3-K) gene, a proline oxidase (PIG6, POX) gene, a quinone oxidoreductase (PIG3, NQO1) gene, a respiratory complexes I gene, a respiratory complexes II gene, a respiratory complexes III gene, a respiratory complexes IV gene, a sestrin 1 (SESN1) gene, a sestrin 2 (SESN2) gene, an SHC adaptor protein 1 (SHC1) gene, a superoxide dismutase 1 (SOD1) gene, a superoxide dismutase 2 (SOD2) gene, a superoxide dismutase 3 (SOD3) gene, a TNF alpha induced protein 3 (TNFAIP3) gene, a tumor protein 53 (p53) gene, a tumor protein p53 inducible nuclear protein 1 (TP53INP1) gene, a ubiquinol-cytochrome c oxidoreductase gene, a 2B4 gene, an ABCA1 gene, an ACP5 gene, an ADAR-1 gene, an ADSS gene, an AIG1 gene, an AIM2 gene, an APOBEC3 gene, an ARRB2 gene, a B2M gene, a BCAS3 gene, a BMP4 gene, a C10orf32 gene, a C21orf33 gene, a CASP1 gene, a CCL5 gene, a CD160 gene, a Cd53 gene, a CDKN2A gene, a CHEK1 gene, a CNNM2 gene, a CNTNAP2 gene, a CSMD1 gene, a CTLA-4 gene, a CTSB gene, a C-type lectin receptors CLRs gene, a CXCL10 gene, a CYP17A1 gene, a DDX60 gene, a DYNC1I1 gene, a FOXO3a gene, a GPC6 gene, a GRN gene, an HCK gene, an HECW1 gene, an HLA gene, an IFI30 gene, an IFI44L gene, an IFI6 gene, an IFITM gene, an IFITM1/3 gene, an IFITM2 gene, an IFITM3 gene, an IL-18 gene, an IL-1α gene, an IL-1β gene, an interferon-γ gene, an interleukin-12 (IL-12) gene, an IRF gene, an IRF-1 gene, a gene, an IRF3 gene, an IRF7 gene, a LAG-3 gene, a LIPC gene, a MDA5/IFIH1 gene, a MPAK gene, a MYH9 gene, a MYO16 gene, a MYO5A gene, an NAIP gene, an NF-κB gene, an NLRC4 gene, a gene, an NLRP3 gene, a NOD2 gene, a gene, an NPL gene, an NR gene, a nucleotide oligomerization and binding domain NOD-like receptors gene, an OAS1 gene, an OAS2 gene, an OASL gene, a parkin gene (PARK2) gene, a PD-1 gene, a PLEKHG1 gene, av PRKCA gene, a PTBP1 gene, a PYCARD gene, a Pyrin-HIN (PYHIN) domain containing receptor gene (e.g. AIM2), a reactive oxygen species (ROS) gene, a retinoic acid inducible gene-I (RIG-I)-like receptor (RLRs) gene, an RFC3 gene, a RGS1 gene, a RIG-I/DDX58 gene, a SAMHD1 gene, a SF3A1/SF3B1 gene, a SFXN2 gene, a SLAMF7 gene, a SLC41A1 gene, a SLC8A1 gene, a SLCO3A1 gene, a STAT1 gene, a tetherin gene, a TLR5 gene, a TLR7 gene, a TLR9 gene, a Toll-like receptor (TLR) gene, a TREM2 gene, a TREX1 gene, a TRIM5 gene, a TTLL7 gene, and a TYROBP gene.

68. The pharmaceutical composition of any one of claims 1-67, wherein the RNA-guided nuclease or a nucleic acid encoding an RNA-guided nuclease is the RNA-guided nuclease.

69. The pharmaceutical composition of any one of claims 1-67, wherein the RNA-guided nuclease or a nucleic acid encoding an RNA-guided nuclease is DNA encoding the RNA-guided nuclease.

70. The pharmaceutical composition of any one of claims 1-67, wherein the RNA-guided nuclease or a nucleic acid encoding an RNA-guided nuclease is mRNA encoding the RNA-guided nuclease.

71. The pharmaceutical composition of any one of claims 1-70, wherein the RNA-guided nuclease is a Cas protein.

72. The pharmaceutical composition of claim 71, wherein the Cas protein is a Cas9 protein.

73. The pharmaceutical composition of claim 71, wherein the Cas9 protein is an S. pyogenes Cas9 polypeptide.

74. The pharmaceutical composition of claim 73, wherein the Cas9 protein is selected from the group consisting of esCas9, hfCas9, peCas9, and ARCas9.

75. The pharmaceutical composition of any one of claims 1-74, wherein the at least one guide RNA or a nucleic acid encoding at least one guide RNA is the at least one guide RNA.

76. The pharmaceutical composition of any one of claims 1-74, wherein the at least one guide RNA or a nucleic acid encoding at least one guide RNA is DNA encoding the at least one guide RNA.

77. The pharmaceutical composition of any one of claims 1-74, comprising a nucleic acid encoding both the RNA-guided nuclease and the at least one guide RNA.

78. The pharmaceutical composition of any one of claims 1-77, wherein the at least one guide RNA is a single guide RNA (sgRNA).

79. The pharmaceutical composition of any one of claims 1-78, wherein the at least one guide RNA targets a human gene.

80. The pharmaceutical composition of any one of claims 1-78, wherein the at least one guide RNA targets a canine gene.

81. The pharmaceutical composition of any one of claims 1-78, wherein the at least one guide RNA targets an equine gene.

82. The pharmaceutical composition of any one of claims 1-78, wherein the at least one guide RNA targets a feline gene.

83. The pharmaceutical composition of any one of claims 1-78, wherein the at least one guide RNA targets a mammalian gene.

84. The pharmaceutical composition of any one of claims 1-83, wherein the composition comprises one or more viral vectors collectively comprising the (i) RNA-guided nuclease or a nucleic acid encoding an RNA-guided nuclease, and (ii) at least one guide RNA or a nucleic acid encoding at least one guide RNA targeting a gene encoding the transmembrane receptor.

85. The pharmaceutical composition of claim 84, wherein the one of more viral vectors comprise a recombinant virus selected from a retrovirus, an adenovirus, an adeno-associated virus, a lentivirus, and a herpes simplex virus-1.

86. The pharmaceutical composition of claim 84, wherein the one of more viral vectors comprise a recombinant adeno-associated virus (AAV).

87. The pharmaceutical composition of claim 86, wherein the recombinant AAV is of serotype 5 (AAV5).

88. The pharmaceutical composition of claim 86, wherein the recombinant AAV is of serotype 6 (AAV6).

89. The pharmaceutical composition of any one of claims 1-83, wherein the composition comprises one or more lipid nanoparticles (LNP) collectively comprising the (i) RNA-guided nuclease or a nucleic acid encoding an RNA-guided nuclease, and (ii) at least one guide RNA or a nucleic acid encoding at least one guide RNA targeting a gene encoding the transmembrane receptor.

90. The pharmaceutical composition of claim 89, wherein the one or more LNP comprises:
a first plurality of LNP encapsulating the RNA-guided nuclease or a nucleic acid encoding an RNA-guided nuclease; and
a second plurality of LNP encapsulating the at least one guide RNA or a nucleic acid encoding at least one guide RNA.

91. The pharmaceutical composition of claim 89, wherein the one or more LNP comprises a plurality of LNP encapsulating both the (i) RNA-guided nuclease or a nucleic acid encoding an RNA-guided nuclease, and (ii) at least one guide RNA or a nucleic acid encoding at least one guide RNA targeting a gene encoding the transmembrane receptor.

92. The pharmaceutical composition of any one of claims 89-91, wherein the one or more LNP comprises a component selected from the group consisting of 3-(didodecylamino)-N1,N1,4-tridodecyl-1-piperazineethanamine (KL10), N1-[2-(didodecylamino)ethyl]-N1,N4,N4-tridodecyl-1,4-piperazinediethanamine (KL22), 14,25-ditridecyl-15,18,21,24-tetraaza-octatriacontane (KL25), 1,2-dilinoleyloxy-N,N-dimethylaminopropane (DLin-DMA), 2,2-dilinoleyl-4-dimethylaminomethyl-[1,3]-dioxolane (DLin-K-DMA), heptatriaconta-6,9,28,31-tetraen-19-yl 4-(dimethylamino)butanoate (DLin-MC3-DMA), 2,2-dilinoleyl-4-(2-dimethylaminoethyl)-[1,3]-dioxolane (DLin-KC2-DMA), 1,2-dioleyloxy-N,N-dimethylaminopropane (DODMA), 2-({8-[(3.beta.)-cholest-5-en-3-yloxy]octyl}oxy)-N,N-dimethyl-3-[(9Z,12Z)—octadeca-9,12-dien-1-yloxy]propan-1-amine (Octyl-CLinDMA), (2R)-2-({8-[(3.beta.)-cholest-5-en-3-yloxy]octyl}oxy)-N,N-dimethyl-3-[(9Z-,12Z)-octadeca-9,12-dien-1-yloxy]propan-1-amine (Octyl-CLinDMA (2R)), (2S)-2-({8-[(3.beta.)-cholest-5-en-3-yloxy]octyl}oxy)-N,N-dimethyl-3-[(9Z-,12Z)-octadeca-9,12-dien-1-yloxy]propan-1-amine (Octyl-CLinDMA (2S)), a lipid including a cyclic amine group, and a mixture thereof.

93. The pharmaceutical composition of any one of claims 89-92, wherein the LNP comprises a component selected from the group consisting of 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-dioleoyl-sn-glycero-3-phosphoethanolamine (DOPE), 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), sphingomyelin (SM), and a mixture thereof.

94. The pharmaceutical composition of any one of claims 89-93, wherein the LNP comprises a component selected from the group consisting of PEG-modified phosphatidylethanolamines, PEG-modified phosphatidic acids, PEG-modified ceramides, PEG-modified dialkylamines, PEG-modified diacylglycerols, PEG-modified dialkylglycerols, and mixtures thereof. For example, a PEG lipid may be PEG-c-DOMG, PEG-DMG, PEG-DLPE, PEG-DMPE, PEG-DPPC, PEG-DMA, a PEG-DSPE lipid, and a mixture thereof.

95. The pharmaceutical composition of any one of claims 89-94, wherein the LNP comprises a component selected from the group consisting of a cholesterol, fecosterol, stigmasterol, stigmastanol, sitosterol, β-sitosterol, lupeol, betulin, ursolic acid, oleanolic acid, campesterol, fucosterol, brassicasterol, ergosterol, 9,11-dehydroergosterol, tomatidine, tomatine, α-tocopherol, and a mixture thereof.

96. The pharmaceutical composition of any one of claims 1-83, wherein the composition comprises one or more liposomes collectively comprising the (i) RNA-guided nuclease or a nucleic acid encoding an RNA-guided nuclease, and (ii) at least one guide RNA or a nucleic acid encoding at least one guide RNA targeting a gene encoding the transmembrane receptor.

97. The pharmaceutical composition of claim 96, wherein the one or more liposomes comprises:
a first plurality of liposomes encapsulating the RNA-guided nuclease or a nucleic acid encoding an RNA-guided nuclease; and
a second plurality of liposomes encapsulating the at least one guide RNA or a nucleic acid encoding at least one guide RNA.

98. The pharmaceutical composition of claim 96, wherein the one or more liposomes comprises a plurality of liposomes encapsulating both the (i) RNA-guided nuclease or a nucleic acid encoding an RNA-guided nuclease, and (ii) at least one guide RNA or a nucleic acid encoding at least one guide RNA targeting a gene encoding the transmembrane receptor.

99. The pharmaceutical composition of any one of claims 1-83, wherein the composition comprises one or more virus-like particles collectively comprising the (i) RNA-guided nuclease or a nucleic acid encoding an RNA-guided nuclease, and (ii) at least one guide RNA or a nucleic acid encoding at least one guide RNA targeting a gene encoding the transmembrane receptor.

100. The pharmaceutical composition of claim 99, wherein the one or more virus-like particles comprises:
a first plurality of virus-like particles encapsulating the RNA-guided nuclease or a nucleic acid encoding an RNA-guided nuclease; and
a second plurality of virus-like particles encapsulating the at least one guide RNA or a nucleic acid encoding at least one guide RNA.

101. The pharmaceutical composition of claim 99, wherein the one or more virus-like particles comprises a plurality of virus-like particles encapsulating both the (i) RNA-guided nuclease or a nucleic acid encoding an RNA-guided nuclease, and (ii) at least one guide RNA or a nucleic acid encoding at least one guide RNA targeting a gene encoding the transmembrane receptor.

102. The pharmaceutical composition of any one of claims 1-101, wherein the composition is formulated for parenteral administration.

103. The pharmaceutical composition of any one of claims 1-101, wherein the composition is formulated for intra-articular injection within a joint of the subject.

104. A method for treating or preventing a joint disorder in a subject in need thereof, comprising:
administering a therapeutically effective amount of a pharmaceutical composition according to any one of claims 1-103 to the subject.

105. A method for treating or preventing free oxygen radicals in a subject in need thereof, comprising:
administering a therapeutically effective amount of a pharmaceutical composition according to any one of claims 1-103 to the subject.
